(19) United States
(12) Patent Application Publication
(10) Pub. No.: US 2025/0270663 A1
(43) Pub. Date: Aug. 28, 2025

(54) CAS13-BASED COMPOSITIONS AND METHODS OF USE THEREOF

(71) Applicant: King Abdullah University of Science and Technology, Thuwal (SA)

(72) Inventors: Magdy Mahmoud Mahfouz, Thuwal (SA); Ahmed Ibrahim Mahas, Thuwal (SA)

(21) Appl. No.: 18/564,572

(22) PCT Filed: May 27, 2022
(86) PCT No.: PCT/IB2022/055036

§ 371 Date: Nov. 27, 2023

Related U.S. Application Data
(63) Continuation-in-part of application No. 18/000,055, filed on Nov. 28, 2022.
(60) Provisional application No. 63/194,099, filed on May 27, 2021.
(60) Provisional application No. 63/030,796, filed on May 27, 2020.

(30) Foreign Application Priority Data
May 27, 2021 (WO) PCT/IB2021/054664

(51) Int. Cl. C12Q 1/70; C12N 9/22; C12N 15/11; C12Q 1/6844

(52) U.S. Cl. CPC C12Q 1/701; C12N 9/226; C12N 15/11; C12Q 1/6844; C12N 2310/20

(57) ABSTRACT

Compositions of previously uncharacterized thermostable class II, type VI CRISPR/Cas effector proteins are provided. The compositions include polynucleotides and vectors for expressing the effector proteins and one or more associated crRNAs, as well as cells that contain the effector proteins. Methods of preparation and use of the compositions are also disclosed. The compositions and methods are especially applicable to rapid and facile detection of nucleic acids in assays requiring elevated temperatures, RNA knockdown, RNA editing, antiviral activity, and RNA imaging.

### CROSS-REFERENCE TO RELATED APPLICATIONS

This application claims the benefit of and priority to U.S. Ser. No. 63/194,099, filed May 27, 2021, and PCT/IB2021/054664, filed May 27, 2021, which claims the benefit of U.S. Provisional Application No. 63/030,796 filed May 27, 2020, each of which is hereby incorporated by reference herein in its entirety.

### REFERENCE TO SEQUENCE LISTING

The Sequence Listing submitted Jul. 24, 2024, as a text file named “KAUST2021-164-03_371_ST25.txt”, created on Jul. 24, 2024, and having a size of 143,761 bytes is hereby incorporated by reference pursuant to 37 C.F.R. § 1.52 (e) (5).

### FIELD KAUST OF THE INVENTION

The field of the invention is generally related compositions and methods for targeting and editing nucleic acids, and in particular, CRISPR-Cas13 based compositions and methods of use thereof.

### BACKGROUND OF THE INVENTION

The rapid spread of SARS-COV-2 poses significant challenges to the health care systems and represents a great burden on economies around the world. The causal agent of COVID-19, Severe acute respiratory syndrome coronavirus 2019 (SARS-COV-2), belongs to the Coronaviridae family that represents positive-stranded RNA viruses with some of the largest known RNA genomes [1]. Members of this family pose a continuous threat to the global community due to their propensity for causing disease outbreaks as witnessed for the third time in the 21^{st} century. Rapid mutation in their genomic RNA along with frequent recombination events facilitates inter-species transmission that often leads to unexpected and uncontrollable epidemic outbreaks. Therefore, early detection and identification of viral transmission is necessary for implementation of control measures that ensure low transmission rates and prevent the loss of human life during present and future pandemics.

The advancements in synthetic biology in synergy with COVID-19 pandemic spurred a wave of innovations in the field of molecular diagnostics [2]. Point-of-care (POC) or at-home testing kits capable of detecting the presence of a pathogen's nucleic acid or infectious markers at low cost present a particularly desirable end goal. In order to be viable for POC application, any developed technology must meet the ASSURED criteria (Accurate, Specific, Sensitive, User-friendly, Rapid, Equipment-free, and Deliverable to end-users) as defined by the WHO for effective POC test to control and manage infectious diseases [3]. Therefore, significant efforts are directed toward developing an easy to use, point-of-care module that would enable large scale, high turnover SARS-COV-2 screening on a massive scale. Promising approaches towards this goal involve CRISPR/Cas type V and VI systems [4, 5]. These modalities paved the way for developing the next generation of diagnostic platforms that exploit highly specific target recognition and cleavage by Cas enzymes followed by in trans cleavage of reporters. For example, RT-LAMP and RT-RPA were coupled with CRISPR Cas9, Cas12, and Cas13 enzymes for sensitive and specific detection of nucleic acids and viruses, including SARS-COV2 [32-38]. The Cas12 and Cas13 enzymes complex with their corresponding single-guide RNA (sgRNA) and scan a DNA or RNA template for a complementary sequence. Once a complementary sequence is recognized, the enzyme cleaves the nucleic acid in cis and, once activated by the initial recognition, exhibits collateral trans cleavage activities, cleaving single-stranded (ss) DNA or RNA molecules present in the reaction [39, 40]. This collateral cleavage activity of Cas12 and Cas13 has been harnessed for nucleic acid detection. For example, the CRISPR-Cas13 system coupled with RPA was used for virus detection via SHERLOCK (Specific High-sensitivity Enzymatic Reporter unLOCKing) [41-43]. To bypass the need for nucleic acid purification, HUDSON (Heating Unextracted Diagnostic Samples to Obliterate Nucleases) was developed and coupled with SHERLOCK [44]. When coupled to isothermal amplification methods such as recombinase polymerase amplification (RPA) or loop-mediated isothermal amplification (LAMP), CRISPR/Cas detection assays facilitate target nucleic acid detection at attomolar levels [6, 7].

There is a need to identify and characterize new Cas effectors to increase utility and develop new tools for in vivo and in vitro applications. In particular, there is a need to identify and characterize the known class II/type VI Cas proteins that exclusively target ssRNA substrates to expand the Cas13-based toolbox for diagnostic and other applications.

Thus, it is an object of the invention to provide Cas13-based compositions and methods of use thereof.

### SUMMARY OF THE INVENTION

Compositions of class II, type VI CRISPR/Cas effector proteins and methods of preparation and use thereof are disclosed. The compositions and methods are especially applicable to rapid and facile detection of nucleic acids, perturbation of gene expression and RNA modification.

In particular, a polynucleotide containing a nucleotide sequence encoding a class II, type VI CRISPR/Cas effector protein (Cas13) and optionally including a heterologous sequence is disclosed. In some embodiments, the Cas effector protein includes the amino acid sequence encoded by SEQ ID NO:63, SEQ ID NO:65, or SEQ ID NO:67 (i.e., SEQ ID NO: 64, SEQ ID NO:66 or SEQ ID NO:68), or a sequence with at least 70% sequence identity thereto. In some embodiments, the sequence encoding the Cas effector protein is or includes SEQ ID NO:63, SEQ ID NO:65 or SEQ ID NO: 67, or a sequence with at least 70% sequence identity to SEQ ID NO: 63, SEQ ID NO:65 or SEQ ID NO:67. In some embodiments, the nucleotide sequence does not include SEQ ID NO:63, 65 or 67. In some embodiments, the nucleotide sequence does not include SEQ ID NO:63, 65 or 67 even when it encodes the same protein encoded by SEQ ID NO:63, 65 or 67. In some embodiments, the sequence encoding the Cas effector protein is codon optimized for expression in a prokaryotic or eukaryotic cell.

The heterologous sequence can be, for example, an expression control sequence, and can include a promoter, transcription terminator, multiple cloning site, drug resistance marker(s), one or more protease recognition sites, one or more epitope tags, or a combination thereof. Typically, the heterologous sequence is operably linked to the sequence encoding the Cas effector protein.

In some embodiments, the polynucleotide further includes a sequence encoding a crRNA. In preferred embodiments, the crRNA is capable of complexing with the Cas effector protein and hybridizing to a target RNA sequence. In some embodiments, the Cas effector protein can originate or be derived from Thermoclostridium caenicola or a Proteobacteria bacterium.

In some embodiments, the polynucleotide is or is contained in a vector, such as an expression vector. Non-limiting examples of suitable vectors include viral vectors or plasmids.

Cells harboring the polynucleotides and/or vectors are also provided. For example, prokaryotic or eukaryotic cells containing the polynucleotide and/or vector are disclosed.

Also provided are methods of producing a class II, type VI CRISPR/Cas effector protein. In some embodiments, the method involves introducing the disclosed vector into a prokaryotic or eukaryotic cell under conditions suitable for expression of the sequence encoding the Cas effector protein. In some embodiments, the method further involves isolating and/or purifying the Cas effector protein.

Isolated class II, type VI CRISPR/Cas effector proteins produced by the foregoing method are also provided. In some embodiments, disclosed is an isolated class II, type VI CRISPR/Cas effector protein having the amino acid sequence of SEQ ID NO:64, SEQ ID NO:66, or SEQ ID NO:68, or a sequence with at least 70% sequence identity to SEQ ID NO:64, SEQ ID NO: 66, or SEQ ID NO:68.

In some embodiments, the Cas effector protein is associated with a crRNA. Thus, ribonucleoprotein complexes containing the Cas effector protein complexed with a crRNA, optionally wherein the crRNA is capable of hybridizing to a target RNA sequence are disclosed.

Also provided are compositions including a Cas13 protein having or including the amino acid sequence of SEQ ID NO:64, SEQ ID NO:66, or SEQ ID NO:68, or a sequence with at least 70% sequence identity to SEQ ID NO: 64, SEQ ID NO:66 or SEQ ID NO:68. In preferred embodiments, the Cas13 protein contains one or more Higher Eukaryotes and Prokaryotes Nucleotide-binding (HEPN) domains, preferably two HEPN domains. An exemplary HEPN domain is a RxxxxH (SEQ ID NO:192) motif sequence, wherein X represents any amino acid.

In some embodiments, the Cas13 protein is complexed with a crRNA, which optionally, but not necessarily, is capable of hybridizing to a target RNA sequence. The crRNA can include a spacer sequence that is capable of hybridizing to the target RNA sequence and a direct repeat sequence.

In some preferred embodiments, the crRNA comprises a spacer of about 20-30 nucleotides, preferably 24-28 nucleotides.

In some embodiments, the Cas13 protein can cleave the target RNA sequence at a temperature of about 37-70° C., about 50-70° C., about 47-60° C., or about 60° C. In some embodiments, the Cas13 protein can cleave the target RNA sequence at a temperature of about 37° C.-42° C., preferably about 37° C.

The composition may be present in a cell, such as a prokaryotic or eukaryotic cell.

Methods of using the disclosed polynucleotides, vectors, proteins, protein complexes, and compositions are also provided. In particular, disclosed is a method of performing targeted knockdown of an RNA transcript involving introducing the disclosed Cas13 compositions to a cell, wherein the crRNA complexed to the Cas13 hybridizes to the RNA transcript, thereby inducing cleavage of the RNA transcript by the Cas13 protein. The RNA transcript can be any ssRNA sequence including coding (e.g., an mRNA) or non-coding (e.g., lincRNA) RNA. In some embodiments, the RNA transcript is derived from a viral gene, such as a bacteriophage. Thus, the compositions can be used in methods of inhibiting viral gene expression or viral replication.

Methods of using the compositions to detect target nucleic acids are also provided. For example, disclosed is a method of detecting the presence of an RNA transcript in a nucleic acid sample by contacting the sample with the Cas13 composition in the presence of an activatable single stranded RNA (ssRNA) oligonucleotide that includes a reporter moiety. In some embodiments, the reporter moiety includes a fluorophore linked to a quencher via the ssRNA. Fluorescence can be emitted upon cleavage of the ssRNA oligonucleotide since the quencher is no longer in proximity to the fluorophore. Preferably, the crRNA is designed to hybridize to the RNA transcript and the Cas13 cleaves the ssRNA oligonucleotide upon binding of the Cas13 crRNA complex to the RNA transcript. Detecting cleavage of the ssRNA oligonucleotide indicates the presence of the RNA transcript.

The RNA transcript could have been generated by transcription from a dsDNA molecule, and in some embodiments, the dsDNA molecule could have been generated by reverse transcription coupled (isothermal) amplification of a target RNA.

Methods of monitoring RNA trafficking are also provided. For example, described herein is a method of determining the localization of an RNA transcript in a cell by introducing the Cas13 composition to the cell. In such embodiments, the Cas13 is preferably catalytically inactive (e.g., the Cas13 functions as an RNA binding protein without nuclease activity). In such embodiments, the Cas13 preferably further includes a detectable marker. The crRNA is designed to hybridize to the RNA transcript, and the Cas13 crRNA complex binds to the RNA transcript, thereby indicating the location of the RNA transcript. Exemplary detectable markers include GFP, eGFP, RFP, YFP, BFP, CFP, mNeonGreen, mCherry, mOrange, mRaspberry, mPlum, mKO, mRFP, mRFPruby, mRuby, tagRFP, mKate2, and DsRed.

Also disclosed is a method for performing targeted editing of an RNA transcript by introducing a composition of a catalytically inactive Cas13 that further includes a deaminase domain of an RNA-dependent Adenosine Deaminase (ADAR) to a cell. The crRNA complexed to the Cas13 is capable of hybridizing with a region in the RNA transcript that contains an A nucleotide to be edited. The crRNA forms an RNA duplex with the RNA transcript that contains an A-C mismatch at the target A nucleotide resulting in the target A nucleotide being deaminated by the deaminase domain (resulting in A-I).

Suitable deaminase domains include deaminase domains from ADAR1, ADAR2, and ADAR3, such as human ADAR1, ADAR2, and ADAR3.

Additional advantages of the disclosed methods and compositions will be set forth in part in the description which follows, and in part will be understood from the description, or can be learned by practice of the disclosed methods and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed method and compositions.

FIGS. 1A-1B are heatmaps showing the screening and selection of the optimal ssRNA reporter for TccCas13a and HheCas13a in-trans activity. Reactions containing Cas13 and two different crRNAs targeting the SARS-COV-2 N gene or non-specific crRNA (NS) control were performed in the presence of one of three FAM-reporters with different RNA sequences. Data are shown as the mean of three replicates. FIG. 1C is a maximum-likelihood phylogenetic tree of Cas13 proteins from different organisms. The tree was generated using MEGA X software. Most selected proteins were isolated from mesophilic bacteria, although several have been cultivated as thermophiles, and thus offer an interesting collection of high-temperature stable proteins. TccCas13a and HheCas13a were selected as potentially thermophilic Cas13 proteins. FIG. 1D is a series of plots showing differential scanning fluorimetry (DSF) profiles of protein melting point using a conventional real-time PCR instrument. The peak in the left graph indicates protein denaturation. The right-side graph is the derivative of the left-side graph. FIG. 1E is a plot showing denaturation temperature of TccCas13a, HheCas13a, and LwaCas13a proteins, as determined by DSF in FIG. 1D. Data are shown as mean±standard deviation (SD) (n=3). FIG. 1F is a heat map showing TccCas13a collateral cleavage preference for the ssRNA reporter. Reactions consisting of TccCas13a and its respective cognate crRNAs or non-specific crRNA (NS) control were performed in the presence of ssRNA target and one of ten ssRNA reporters. NS: non-specific crRNA. Data are shown as mean (n=3). Reactions were incubated at 56° C. and the endpoint fluorescent signal was measured after 30 min. ssRNA reporter sequences are shown on top of the heatmap. A: Poly A reporter, C: Poly C reporter, G: Poly G reporter, U: Poly U reporter, UA: LwaCas13a reporter, AC: 3 (AC) reporter, AG: 3 (AG) reporter, UG: 3 (UG) reporter, CG: 3 (CG) reporter, Mix: Mix reporter (UGACGU). FIG. 1G is a heat map showing HheCas13a collateral cleavage preference for the ssRNA reporter. Reactions consisting of HheCas13a and its respective cognate crRNAs or non-specific crRNA (NS) control were performed in the presence of ssRNA target and one of six ssRNA reporters. NS: non-specific crRNA. Data are shown as mean (n=3). Reactions were incubated at 56° C. and endpoint fluorescence signal detection was measured after 30 min. ssRNA reporter sequences are shown on top of the panel, A: Poly A reporter, U: Poly U reporter, G: Poly 6G reporter, UG: 3 (UG) reporter, CG: 3 (CG) reporter, Mix: Mix reporter.

FIGS. 2A-2B are graphs showing the in-trans activity of HheCas13a and TccCas13a assessed at varying target concentrations and two different temperatures. FIG. 2C is a heatmap depicting the in-trans activity of HheCas13a and TccCas13a assessed at different temperatures with two crRNAs targeting the N SARS-COV-2 gene and one non-specific (NS) crRNA. Reactions were run for 30 mins. Data are shown as mean with n=3. FIG. 2D is a bar graph showing the in-trans activity of TccCas13a assessed at 60° C. with two crRNAs of different spacer lengths targeting the N SARS-COV-2 gene and one non-specific (NS) crRNA. Reactions were run for 30 mins. Values shown as mean±SD (n=3). FIG. 2E is a plot showing end-point activity of LwaCas13a, HheCas13a and TccCas13a at different temperatures using their preferred reporter. One crRNA and a non-specific crRNA were tested for each. Values are shown as mean±S.D and represent endpoint fluorescence at 30 min. FIG. 2F is predicted secondary RNA structure of the direct repeat sequence of TccCas13a crRNA. RNAfold ran.tbi.univie.ac.at/ was used to predict the crRNA secondary structure.

FIGS. 3A-3D are bar graphs showing the results of the screening of the indicated HheCas13a and TccCas13a crRNAs paired with various primers in the two-pot assay. crRNAs designed to target the RNA substrate produced from T7-mediated in vitro transcription of the RT-LAMP amplification products using STOPCovid (SC) primer sets with T7 promoter appended to the FIP primer (T7-FIP) or to the BIP primer (T7-BIP). RT-LAMP amplification was performed first and the RT-LAMP product was added to the T7-in vitro transcription and Cas13-based detection reaction (two-pots). The in-trans activity of HheCas13a and TccCas13a were assessed at 56° C. NS: non-specific crRNA. Reactions were run for 30-60 mins.

FIGS. 4A-4D are bar graphs showing the results of the screening of the indicated HheCas13a and TccCas13a crRNAs paired with various primers in the one-pot assay. crRNAs designed to target the RNA substrate produced from T7-mediated in vitro transcription of the RT-LAMP amplification using STOPCovid (SC) primer sets with T7 promoter appended to the FIP primer (T7-FIP) or to the BIP primer (T7-BIP). RT-LAMP amplification, T7-in vitro transcription and Cas13-based detection was performed in single step at single temperature, 55° C. (one-pot). NS: non-specific crRNA. Reactions were run for 30-60 mins. Values shown as mean±SD (n=3).

FIGS. 5A-5E are graphs showing the results from a second round of screening of the indicated HheCas13a and TccCas13a crRNAs and different primer sets for the establishment of one-pot assay. RT-LAMP amplification, T7-in vitro transcription and Cas13-based detection was performed in single step at single temperature, 55° C. (one-pot). NS: non-specific crRNA. Reactions were run for 80 mins. Values shown as mean (n=3).

FIG. 6 is a heatmap showing the in-trans activity of the one-pot detection assay with synthetic RNA using different Bst DNA polymerases from different vendors. NTC: non-template control. Reactions were run for 80 mins at 55° C. Values shown as mean (n=3).

FIGS. 7A-7D are bar graphs showing the cleavage activity towards the reporter probe in the one-pot assay with different concentrations of Bst DNA polymerase (FIG. 7A), MgSO4 (FIG. 7B), Hi-T7 RNA polymerase (FIG. 7C), and TccCas13a and crRNA (RNP) concentrations (FIG. 7D). NTC: non-template control. Reactions were run for 80 mins at 55° C. Values shown as mean±SD (n=3).

FIG. 8 is a bar graph quantifying the limit of detection (LoD) of the thermophilic Cas13-based one pot detection assay using synthetic SARS-CoV-2 RNA as an input. Reactions were run for 80 mins at 56° C. Values shown as mean±SD (n=3).

FIG. 9 is a graph quantifying the performance of the thermophilic Cas13-based one-pot detection assay on SARS-COV-2 patient samples. RNA extracted from clinical samples from 8 patients with SARS-COV-2 infection (Table 6) were analyzed with the one-pot detection assay. NTC: no template control. Reactions were run for 80 mins at 56° C.

FIG. 10A is a schematic of specific Cas13-based detection. Specific target recognition by Cas13 RNP triggers non-specific, collateral activity that cleaves the reporters, resulting in a detectable fluorescent signal.

FIGS. 10B-10F are bar graphs showing cleavage activity across various reporter and crRNA sequences. Reactions containing mCas13 and four different crRNAs targeting the SARS-COV-2 N gene or non-specific crRNA (NS) control were performed in the presence of one of five reporters. F-NNNNN-Q represents the RNaseAlert v2 reporter (Thermofisher). NS: non-specific (not targeting SARS-COV-2) crRNA. Values shown as mean±SD (n=3). FIG. 10G is a bar graph quantifying the effect of mismatches between mCas13 crRNA and target RNA on mCas13 activity. Left, crRNA nucleotide sequence with the positions of mismatches (red) on the crRNA. Right, the fluorescence intensity, relative to the no-template control (NTC, gray) or crRNA with no mismatches (purple), resulting from mCas13 collateral cleavage activity on each tested crRNA. FIG. 10H is a schematic illustration of the sequence similarity searching strategies for the identification of miniature Cas13 effectors. BLASTp analysis was performed before January 2021. FIG. 10I is a bar graph showing titration of mCas13 and crRNA concentrations for optimal performance. 100 nM of in vitro transcribed RNA of SARS-COV-2 N gene was used as the target sequence in mCas13 reactions. Values represent endpoint fluorescence at 30 mins. Values shown as mean±SD (n=3). FIG. 10J is a bar graph quantifying mCas13 collateral activity at the indicated temperatures. Collateral activity was measured as end point fluorescence after incubation with 100 nM of in vitro transcribed RNA of SARS-COV-2 N gene for 45 mins. Values shown as mean±SD (n=3). FIG. 10K shows multiple sequence alignment of mCas13 and compact Cas13e and Cas13f orthologues. Protein sequences of mCas13, Cas13e.1, Cas13e.2, Cas13f.1, Cas13f.2, Cas13f.3, Cas13f.4 and Cas13f.5 were aligned using ClustalW in MEGAX and ESPript was used to generate the alignment visualization. Strictly conserved residues (% Strict) are shown in red text within blue rectangles. Conserved RxxxxH (SEQ ID NO: 192) motifs of the HEPN domains are highlighted with yellow background. FIG. 10L a bar graph showing activity assessment of mCas13 with different crRNA sequences. Reactions containing mCas13 and one of the different crRNAs targeting the SARS-COV-2 N gene were preferred in the presence of the F-UUAUU-Q reporters. NS: non-specific (not SARS-COV-2 targeting) crRNA. Values shown as mean+/−SD (n=3). FIG. 10M is an illustration of the sequence of crRNA 4 and the position of synthetically introduced single mismatches. FIG. 10N is a graph showing real-time fluorescence measurements of the collateral cleavage activity of mCas13 with different crRNAs with different mismatches relative to no template control (NTC) and crRNA with no mismatch (No Mis). Values shown as mean+/−SD (n=3).

FIG. 11A is a schematic of assay workflow of mCas13-based detection. Following sample collection and RNA extraction, pathogen RNA is reverse transcribed and amplified via RT-LAMP isothermal reaction. RT-LAMP primer sets (in which the FIP contains a T7 promoter sequence) are used, resulting in amplicons containing the T7-promoter sequence, which serve as templates for in vitro transcription of target RNA. Upon target recognition, mCas13 cleaves specially designed reporters in trans, leading to fluorescent signal output. FIG. 11B is a graph showing measurement of real time fluorescence output of T7-mediated in vitro transcription and mCas13-based detection. Synthetic SARS-COV-2 RNA was reverse transcribed and LAMP amplified with STOPCovid (SC) and DETECTR (DT) primer sets. crRNA 4: targeting crRNA #4 identified in FIG. 10. NS: non-specific crRNA. Values shown as mean (n=3) FIGS. 11C-11D are bar graphs showing the limit of detection (LoD) of mCas13-based detection assay. LoD was determined using synthetic SARS-COV-2 RNA, which was reverse transcribed and LAMP-amplified with STOPCovid and DETECTR primer sets. LAMP product was subsequently used for T7 in vitro transcription and concurrent mCas13 detection. Values shown as mean+SD (n=3). FIG. 11E is a graph showing results of the mCas13-based detection assay across different viruses. Detection of non-specific viral targets including SARS-COV-1, MERS-COV, as well as two plant viruses, tobacco mosaic virus (TMV) and turnip mosaic virus (TuMV) was attempted using the STOPCovid primer set. Values shown as mean±SD (n=3).

FIG. 12A is a graph showing validation of mCas13-based detection assay on RT-qPCR-validated SARS-COV-2 clinical samples with different Ct values. Pink bars represent mCas13-based detection fluorescence output. FAM: RNA reporter labeled with FAM fluorophore used in mCas13 detection assays. Blue dots represent N gene Ct values. FIG. 12B is a schematic representation of mCas13-based visual detection with a handheld fluorescence visualizer. FIGS. 12C-12D are images showing visual readouts of the limit of detection (LoD) of mCas13-based detection assay. LoD was determined using synthetic SARS-COV-2 RNA (FIG. 12C) or RT-qPCR-validated SARS-COV-2 clinical samples with different Ct values (FIG. 12D), which were reverse transcribed and LAMP-amplified with the STOPCovid primer set. LAMP product was used for T7 in vitro transcription and concurrent mCas13 detection. FIG. 12E is a series of images showing RT-LAMP mCas13 visual detection reactions of 24 SARS-COV-2 RT-qPCR positive samples and no template control (NTC) reactions used to generate the heatmap in FIG. 12F. Left, reaction tubes with fluorescence readouts are shown with the clinical sample ID. Right, reaction tubes with fluorescence readouts are shown with RT-qPCR Ct values indicated. FIG. 12F is a heat map displaying the validation of the mCas13-based visual detection assay of 24 SARS-COV-2 RT-qPCR positive samples. RFU: random fluorescence units, showing the signal intensity that was obtained by the TECAN plate reader for the mCas13 reactions. FIG. 12G is an image showing fluorescent reporter (HEX reporter) concentration for clear visual detection of mCas13 collateral activity. mCas13 was incubated with crRNA 4 or non-specific (NS) crRNA and 100 nM of in vitro transcribed RNA of SARS-CoV-2 N gene using different concentrations of RNA reporter molecule with the sequence UUAUU that is conjugated to 5′ HEX for 45 mins. Reactions were visualized with p51 fluorescence visualizer and photo was taken with a smart phone. FIG. 12H is an image showing a different repeat of the experiment done in FIG. 3D. Picture was taken with different smart phone used in the other figures. NTC: non-template control. FIGS. 12I and 12J are images of RT-LAMP mCas13 visual detection reactions of 24 SARS-COV-2 RT-qPCR positive samples and no template control (NTC) reactions used to generate the heatmap in FIG. 12F. FIG. 12I shows reaction tubes with fluorescence readouts are shown with the clinical sample ID. FIG. 12J reaction tubes with fluorescence readouts are shown with RT-qPCR Ct values indicated.

FIG. 13 is schematic illustrating identification of mCas13 and development of its use in a nucleic acid detection assay.

FIGS. 14A-14D show In vitro characterization of thermostable Cas13a crRNAs sequence requirements. Evaluating the effect of single (14A), double (14B), and stretches of 4 mismatches (14C) between crRNA and target RNA on TccCas13a activity. Left: crRNA nucleotide sequence with the positions of mismatches on the crRNA spacer. Right: the fluorescence intensity, relative to the non-specific crRNA control (NS) or crRNA with no mismatches (green), resulting from TccCas13a collateral cleavage activity on each tested crRNA. Reactions were incubated at 56° C. and the endpoint fluorescent signal was measured after 30 min. FIG. 14D shows TccCas13a RNA detection activity with different crRNA spacer lengths. Left: crRNA spacer nucleotide sequences with the length of each spacer shown on the left of the sequence. Right: the fluorescence intensity, relative to the non-specific crRNA control (NS) resulting from TccCas13a collateral cleavage activity on each tested crRNA. Reactions were incubated at 56° C. and the endpoint fluorescent signal was measured after 30 min. FIG. 14E is a plot showing representative Michaelis-Menten plot for TccCas13a-catalyzed ssRNA trans cleavage activity. Enzyme kinetic data and the measured K_{cat}, Kₘ, and K_{cat}/Kₘ are shown on the top of plot. Values are shown as mean±S.D (n=3). FIG. 14F-14I are plots showing: 14F) single mismatches; 14G) double mismatches; 14H) 4 mismatches; 14I) crRNA spacer truncations. Left: crRNA nucleotide sequence with the positions of mismatches on the crRNA spacer. Right: the fluorescence intensity, relative to the non-specific crRNA control (NS) or crRNA with no mismatches, resulting from HheCas13a collateral cleavage activity on each tested crRNA. Reactions were incubated at 56° C. and endpoint fluorescence signal detection was measured after 30 min. Values are shown as mean±S.D.

FIGS. 15A-15D illustrate exemplary one-pot SARS-COV-2 detection using the thermophilic TccCas13a protein. FIG. 15A is a schematic representation of the SARS-COV-2 genome showing the region targeted by RT-LAMP amplification and the crRNA target sequence. The small arrow on the T7-FIP primer indicates the T7 promoter sequence. SC region: genomic region of SARS-COV-2 N gene targeted with STOPCovid primers. FIG. 15B is an overview of an exemplary assay workflow. The detection protocol includes three distinct steps, all carried out in the same tube and at the same temperature (56° C.). Following the extraction of viral RNA, specific target sequences within the viral RNA are reverse-transcribed (RT) into cDNA and amplified with RT-LAMP isothermal amplification using LAMP primers containing the T7 promoter sequence (small red arrow on the T7-FIP primer). The resulting RT-LAMP amplicons are used for in vitro transcription using the thermostable Hi-T7 RNA polymerase, producing RNA transcripts that are recognized and targeted simultaneously by the thermophilic TccCas13a protein. Recognition of the RNA transcripts by TccCas13a triggers Cas13 collateral cleavage activity, resulting in trans-cleavage of the reporter probe conjugated to the HEX or FAM fluorophores. FIGS. 15C and 15D are plots showing performance of one-pot detection assay at different temperatures: as determined by real-time fluorescence at a given target RNA concentration (100 cp/μL), data are shown as mean (n=3) (15C); and endpoint fluorescent signal measured after 30 min, values are shown as mean±S.D. (15D) The best performance was achieved at 56° C.

FIGS. 16A-16D illustrate evaluation of OPTIMA-dx for the detection of SARS-COV-2. FIG. 16A a schematic representation of SARS-COV-2 RNA detection in one-pot assays and visual detection using the P51 Molecular Fluorescence Viewer. As the test is performed in a single pot, there is no need to open the reaction tube so it can be discarded without opening, thus avoiding the possibility of contamination at the point of care site. FIG. 16B is a series of images showing assessment of the sensitivity of OPTIMA-dx and the effect of reaction incubation time on performance using fluorescence-based visual detection. Fluorescence rises above background after 45 min with little improvement as time increases. Three replicates were performed for each treatment. FIG. 16C is a plot showing SARS-COV-2 detection from 100 clinical COVID-19 samples with one-pot RT-LAMP-TccCas13a detection assay. RT-qPCR cycle threshold (CT) plotted against fluorescent readout from the detection of SARS-COV-2 positive samples (n=73) and SARS-COV-2 negative samples n=27). Detection reactions were incubated at 56° C. and the endpoint fluorescent signal was measured after 1 hour. Light blue data points in negative samples represent no template controls (NTC). FIG. 16D is a plot showing detection of RNase P internal control with one-pot RT-LAMP-TccCas13a detection assay. All 100 clinical samples in FIG. 16A were tested for the detection of RNase P gene. Detection reactions were incubated at 56° C. and the endpoint fluorescent signal was measured after 1 hour. NTC: no template control.

FIGS. 17A-17D illustrate multiplexed OPTIMA-dx detection with TccCas13a and AapCas12b thermostable Cas enzymes. FIG. 17A is a schematic representation of one-pot multiplexed OPTIMA-dx detection reaction. The unique collateral activity of Cas12 and Cas13 orthologues enables the use of different reporter molecules with different fluorophore, RNA reporter with FAM fluorophore for Cas13 and DNA reporter with HEX fluorophore for Cas12, and multi-channel detection. FIG. 17B is a plot showing the performance of multiplexed detection of SARS-COV-2 (at 400 cp/μL) and isolated human RNA (for RNase P detection) as measured by real-time fluorescence. Data are shown as means±SD (n=3). FIG. 17C is a bar graph showing multiplexed detection of SARS-COV-2 and the human internal control (RNase P) in RNA extracted from 14 clinical COVID-19 samples. Detection reactions were incubated at 56° C. and the endpoint fluorescent signal was measured with FAM and HEX channels after 1 hour. SARS-COV-2: synthetic SARS-COV-2 RNA used at 400 cp/μL, RNase P: isolated total human RNA. NTC: no template control. FIG. 17D is a heat map showing multiplexed detection of SARS-COV-2 and the human internal control (RNase P) from 16 clinical oropharyngeal swabs processed with the quick extraction method. Detection reactions were incubated at 56° C. and the endpoint fluorescent signal was measured with FAM and HEX channels after 1 hour. −Ve: SARS-COV-2 negative samples as determined with RT-qPCR. NTC no template control.

FIGS. 18A-18D are bar graphs showing HheCas13a and TccCas13a crRNA screening in two-pot detection reaction. Trans cleavage activity of HheCas13a and TccCas13a using different crRNAs when incubated with RT-LAMP product of amplified SARS-COV-2 genomic standards. Assay was performed as described in material and methods section. Endpoint fluorescence signal detection was measured after 1 hour. NS: non-specific crRNA. T7-FIP: RT-LAMP primers with modified FIP primer carrying T7 promoter sequence. T7-BIP: RT-LAMP primers with modified BIP primer carrying T7 promoter sequence. The location of the targeted sequence of each crRNA (orange) relative to the RT-LAMP primers (F3, FIP-T7, BIP-T7, B3) is depicted on top of each graph. FIP/BIP-T7: primers containing T7 promoter sequence.

FIG. 19 is a heat map showing establishment of one-pot SARS-CoV-2 detection using the thermophilic Cas13 proteins. Trans cleavage activity of HheCas13a and TccCas13a using different crRNAs in one-pot reactions using SARS-COV-2 genomic standards as input. The assay was performed as described in material and methods. End-point fluorescence signal detection was carried out after 80 min. NTC: No template control. T7-FIP: modified RT-LAMP FIP primer with T7 promoter sequence. T7-BIP: modified RT-LAMP BIP primer with T7 promoter sequence. Data are shown as mean (n=3).

FIGS. 20A-20D are plots showing establishment of one-pot SARS-CoV-2 detection using the thermophilic Cas13 proteins. Trans cleavage activity of HheCas13a and TccCas13a using different crRNAs in one-pot reactions using SARS-COV-2 genomic standards as input. The assay was performed as described in material and methods. End-point fluorescence signal detection was carried out after 80 min. NTC: No template control. T7-FIP: modified RT-LAMP FIP primer with T7 promoter sequence. T7-BIP: modified RT-LAMP BIP primer with T7 promoter sequence. Data are shown as mean (n=3).

FIG. 21A is a plot showing activity screening of three different AapCas12b sgRNAs in One-pot RT-LAMP Cas12b detection of RNase P template as measured by real-time fluorescence signal produced from HEX reporter cleavage with AapCas12b collateral activity. Data are shown as means±SD (n=3). RNase P: total human RNA. NTC: No template control. FIG. 21B is a heat map showing analysis of the activity of Cas12 and Cas13 with different reporter molecules and different targets for one-pot OPTIMA-dx multiplex detection. Endpoint fluorescent signal measured after 60 min, values are shown as mean (n=3).

FIGS. 22A-22C illustrate the adaptability of OPTIMA-dx for specific detection of different pathogens. FIG. 22A is a bar graph showing detection of major HCV genotypes with OPTIMA-dx. In vitro transcribed RNA was used as RNA template in the OPTIMA-dx detection reactions at concentrations of 500 pM. Values are shown as mean±S.D. and represent endpoint fluorescence at 60 min. FIG. 22B is a bar graph showing detection of TYLCV DNA virus with OPTIMA-dx. DNA isolated from two different TYLCV infected plants and one healthy (not infected) plant was diluted 1:10 or 1:100 in water and used as template in the OPTIMA-dx detection reactions. A plasmid containing TYLCV genome was used as a control at concentrations of 1 ng/reaction. NTC: no template control. Values are shown as mean+S.D and represent endpoint fluorescence at 60 min. FIG. 22C is a plot showing performance of multiplexed detection of HCV at concentrations of 500 pM and isolated human RNA (for RNAseP detection) as measured by real-time fluorescence. Data are shown as means±SD (n=3).

### DETAILED DESCRIPTION OF THE INVENTION

The disclosed methods and compositions can be understood more readily by reference to the following detailed description of particular embodiments and the Examples included therein and to the Figures and their previous and following description.

Robust, sensitive and specific diagnostic platforms for early SARS-CoV-2 detection are important to facilitate early identification of afflicted individuals, thus supporting the efforts to curb the ongoing COVID-19 pandemic. CRISPR systems have been repurposed for biosensing applications including virus detection. Different two-pot modalities have been developed for SARS-COV-2 detection including Cas13. Despite many existing diagnostic strategies involving CRISPR/Cas, robust one-pot detection assays that involves minimal equipment and handling steps are still elusive. The working Examples demonstrate identification and characterization of a thermophilic Cas13a enzyme and show its trans and collateral activities at temperatures between 60-70° C. The thermostability features of this enzyme was harnessed to build an assay for sensitive and specific detection of SARS-COV2. The one-pot, isothermal assay couples RT-LAMP, in vitro transcription by thermostable Hi-T7 RNA polymerase, and specific amplicon recognition by thermostable Cas13a effector. This method facilitates handling by eliminating the need for separating amplification and detection steps utilizing only minimal equipment, thereby allowing practical POC application. The assay can detect down to 20 copies per microliter of synthetic SARS-COV-2 genomic RNA. Upon evaluating 8 clinical samples from COVID-19 patients, the assay shows 100% agreement with RT-qPCR. Hence, RT-LAMP CRISPR/Cas13a assay is a practical, sensitive and robust platform with POC capacity for SARS-COV2 detection, and for pathogen detection in general.

The working Examples also describe identification of a miniature Cas13 (mCas13) variant and characterization of its catalytic activity. The mCas13 was employed to design, build, and test a SARS-COV-2 detection module coupling reverse transcription loop-mediated isothermal amplification (RT-LAMP) with the mCas13 system to detect SARS-COV-2 in synthetic and clinical samples. The mCas13 system exhibited sensitivity and specificity comparable to other CRISPR systems.

Also provided are thermostable orthologues of the Cas13a family from the thermophilic organism Thermoclostridium caenicola (TccCas13a) and Herbinix hemicellulosilytica (HheCas13a). These Cas13 proteins share several properties such as thermostability and inability to process its own pre-crRNA. Use of the proteins in various capacities, including SARS-COV-2 diagnostic assays, are exemplified.

This work expands the repertoire and application of Cas13 enzymes in diagnostics and for potential in vivo applications, including RNA knockdown and editing. The mCas13, TccCas13a, and HheCas13a systems can be adapted and used in large-scale testing for diverse pathogens, including RNA and DNA viruses, and bacteria.

### I. Definitions

“Introduce” in the context of genome modification refers to bringing into contact. For example, to introduce a vector (e.g., encoding an Cas13 effector) to a cell is to provide contact between the cell and the composition. The term encompasses penetration of the contacted composition to the interior of the cell by any suitable means, e.g., via transfection, electroporation, transduction, gene gun, nanoparticle delivery, etc.

The term “operably linked” or “operationally linked” refers to functional linkage between a regulatory sequence and a heterologous nucleic acid sequence permitting them to function in their intended manner (e.g., resulting in expression of the latter). The term encompasses positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter.

The term “expression” encompasses the transcription and/or translation of a particular nucleotide sequence driven by a promoter. “Expression vector” or “expression cassette” refers to a vector containing a recombinant polynucleotide having expression control sequences operatively linked to a nucleotide sequence to be expressed. An expression vector contains sufficient cis-acting elements for expression; other elements for expression can be supplied by the host cell or in an in vitro expression system. Expression vectors include all those known in the art, such as cosmids, plasmids (e.g., naked or contained in liposomes), phagemids, BACs, YACs, and viral vectors (e.g., vectors derived from lentiviruses, retroviruses, adenoviruses, and adeno-associated viruses) that incorporate the recombinant polynucleotide.

As used here, the term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompass “exogenous” and “non-native” elements.

As used herein, the term “polynucleotide” generally refers to any polyribonucleotide or polydeoxribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term “nucleic acid” or “nucleic acid sequence” also encompasses a polynucleotide as defined above.

A “mutation” refers to a change in a nucleotide (e.g., DNA) or amino acid sequence resulting in an alteration from a given reference sequence. The mutation can be a deletion, insertion, duplication, and/or substitution of at least one deoxyribonucleic acid base such as a purine (adenine and/or guanine) and/or a pyrimidine (thymine, uracil and/or cytosine). Mutations may or may not produce discernible changes in the observable characteristics (phenotype) of an subject.

“Encoding” or “encode” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment in a genome in which it naturally occurs. The term also applies to nucleic acids which have been substantially purified from other components which naturally accompany the nucleic acid (e.g., RNA or DNA or proteins, which naturally accompany it in the cell). The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences.

In the context of cells, the term “isolated” refers to a cell altered or removed from its natural state. An isolated cell is thus in an environment different from that in which the cell naturally occurs, e.g., separated from its natural milieu such as by concentrating to a concentration at which it is not found in nature. “Isolated cell” is meant to include cells that are within samples that are substantially enriched for the cell of interest and/or in which the cell of interest is partially or substantially purified.

The terms “transformed,” “transduced,” and “transfected” encompass the introduction of a nucleic acid or other material into a cell by one of a number of techniques known in the art.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “percent (%) sequence identity” describes the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

“Identity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48:1073 (1988). Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48:443-453, 1970) algorithm (e.g., NBLAST, and XBLAST). In some forms, the default parameters can be used to determine the identity for the polynucleotides or polypeptides of the present disclosure.

In some forms, the % sequence identity of a given nucleic acid or amino acid sequence C to, with, or against a given nucleic acid or amino acid sequence D (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D.

It will be appreciated that where the length of sequence C is not equal to the length of sequence D, the % sequence identity of C to D will not equal the % sequence identity of D to C.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +/−10%; in other forms the values may range in value either above or below the stated value in a range of approximately +/−5%; in other forms the values may range in value either above or below the stated value in a range of approximately +/−2%; in other forms the values may range in value either above or below the stated value in a range of approximately +/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, including, but not limited to those provided in the exemplary experiments, and that each such combination is specifically contemplated and should be considered disclosed. All methods described herein can be performed in any suitable order unless otherwise indicated or otherwise clearly contradicted by context. The use of any and all examples and experiments, and exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Compositions

Compositions of class II, type VI CRISPR/Cas effector proteins or nucleic acids encoding thereof are provided. The compositions can include polynucleotides, vectors (e.g., expression vectors), isolated proteins, and/or ribonucleoprotein complexes related to class II, type VI CRISPR/Cas effectors.

### Class II, Type VI CRISPR/Cas System

Clustered Regularly Interspaced Short Palindromic Repeats (CRISPR)-CRISPR-associated (Cas) (CRISPR-Cas) systems originate from Prokaryotes, where they serve primarily as a defensive mechanism against mobile genetic elements like phages and plasmids. These systems contain two components. The first is a genomic locus called CRISPR that contains a series of short sequences of foreign origin called spacers that enable recognition of specific mobile genetic elements that were previously encountered. Spacers are separated by repetitive regulatory sequences named repeats. Together with the leader sequence, these spacers and repeats constitute the CRISRP array. The second component of CRISPR-Cas systems are Cas proteins., which are encoded by cas genes, usually located in the proximity of a CRISPR array. See Burmistrz, M., et al., Int J Mol Sci., 21 (3): 1122 (2020).

The mechanism of CRISPR-Cas systems includes three phases: adaptation, maturation, and interference. During the adaptation phase, new spacers are incorporated into the CRISPR array into its leader end. During the maturation phase the CRISPR array is transcribed. The resulting transcript called pre-CRISPR RNA (pre-crRNA) is further processed into a set of CRISPR RNA (crRNA) molecules, each containing a single spacer flanked by fragments of a repeat sequence. Subsequently, crRNAs are incorporated into ribonucleoprotein (RNP) complexes together with Cas proteins. RNP complexes scan nucleic acids searching for a sequence complementary to that encoded by crRNA.

These CRISPR systems are classified based on the structure of CRISPR-associated (Cas) genes that are typically adjacent to the CRISPR arrays. Generally, there are two classes of CRISPR systems, each containing multiple CRISPR types. Class 1 contains type I and type III CRISPR systems that are commonly found in Archaea. Class 2 contains type II, IV, V, and VI CRISPR systems (Adli, M., Nat Commun., 9:1911 (2018)).

The type VI CRISPR-Cas systems present a relatively simple structure, as they require only one Cas13 protein and crRNA molecule for activity. To date, four subtypes have been distinguished: VI-A (that uses Cas13a variant, alternatively known as C2c2), VI-B (Cas13b/C2c6), VI-C (Cas13c/C2c7), and VI-D (Cas13d). Although the Cas13 of these subtypes differ in size and sequence, they all share a common feature, which is the presence of two HEPN domains. These domains are responsible for RNA-targeted nucleolytic activity. HEPN domains are usually located close to different terminal ends of the Cas13 protein.

In general, processing of pre-crRNA into crRNA is performed by Cas13 itself in a metal-independent manner (with the exception of type VI-D) without the help of other host factors. In subtypes VI-A, VI-C, and VI-D crRNAs contain a repeat-derived handle on their 5′ end. On the contrary, subtype VI-B generates crRNAs with handle on the 3′ end. Secondary processing is presumably performed by other host nucleases. crRNA maturation is not necessary for type VI activity, and even unprocessed pre-crRNA is sufficient for recognition of targeted RNA. The complex of Cas13 and cRNA presents no nucleolytic activity until it binds to targeted ssRNA. Binding between crRNA and targeted RNA triggers the conformation change of the RNP complex. Both HEPN domains are moved closer to each other, creating a single catalytic site. As this site is located at some distance to crRNA-targeted RNA duplex, it is expected to cleave not only the targeted RNA but also any other ssRNA, including the host's own RNA, in close proximity of a RNP complex. This is sometimes referred to as ‘collateral damage’ or ‘collateral cleavage’. Burmistrz, M., et al., 2020.

As used herein, CRISPR/Cas composition refers to the elements of a CRISPR system needed to carry out CRISPR/Cas-mediated activity. CRISPR/Cas-mediated compositions typically include one or more nucleic acids encoding a crRNA, a tracrRNA (or chimeric thereof also referred to a guide RNA or single guide RNA) and a Cas enzyme, preferably Cas13.

The methods of delivery disclosed herein are suitable for use with numerous variations on the CRISPR/Cas system. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence can be any polynucleotide, such as DNA or RNA polynucleotides. In some embodiments, a target sequence is located in the nucleus or cytoplasm of a cell.

All type VI systems require a so called Protospacer Flanking Sequence (PFS) located in the direct vicinity of a protospacer sequence. This sequence varies between different subtypes. For example: LshCas13a uses a non-G PFS located at 3′ end, whereas BzCas13b requires the double-sided PFS of non-C upstream of the target site and NAN or NNA downstream of the target site. The tracrRNA duplex directs Cas to the target consisting of the protospacer and the requisite PSF via heteroduplex formation between the spacer region of the crRNA and the protospacer.

Typically, formation of a CRISPR complex (including a guide sequence hybridized to a target sequence and complexed with one or more Cas proteins) results in cleavage of the RNA strands in or near (e.g., within 1, 2, 3, 4, 5, 6, 7, 8, 9, 10, 20, 50, or more base pairs from) the target sequence.

There are many resources available for helping practitioners determine suitable target sites once a desired target sequence is identified, e.g., SnapGene.

### A. Polynucleotides and Expression Vectors

Isolated nucleic acid sequences encoding the disclosed Cas13 polypeptides, variants thereof and fusion proteins thereof are disclosed. Nucleic acids can be in sense or antisense orientation, or can be complementary to a reference sequence encoding a Cas13 or variant thereof such as those discussed below.

Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained.

Particularly, polynucleotides containing a nucleotide sequence encoding a class II, type VI CRISPR/Cas effector protein (Cas13) and a heterologous sequence are disclosed. In some embodiments, the CRISPR/Cas effector is an RNA editing enzyme (e.g., RNA endonuclease) with RNA (e.g., ssRNA) cleavage activity. In a preferred embodiment, the Cas13 enzyme is a Cas13a protein derived from thermophilic bacteria, preferably from Thermoclostridium caenicola (TccCas13a) or Herbinix hemicellulosilytica (HheCas13a).

DNA sequences for HheCas13a are known in the art. See e.g., Addgene plasmid No.: 91871 and East-Seletsky, et al., Mol Cell, 2017, which are hereby incorporated by reference in their entirety.

An exemplary DNA sequence encoding HheCas13a is:

(SEQ ID NO: 67)
ATGAAGTTGACGCGTCGCCGCATCAGTGGCAATTCAGTCGACCAGAAGATT
ACAGCTGCGTTTTATCGTGATATGTCGCAAGGGTTATTATATTATGATTCG
GAGGACAATGACTGTACGGACAAGGTCATCGAAAGTATGGATTTCGAACGC
TCTTGGCGTGGACGCATTCTGAAGAACGGCGAGGATGATAAGAATCCGTTT
TACATGTTCGTTAAAGGTTTAGTTGGTTCCAACGATAAGATCGTGTGCGAA
CCTATCGACGTCGACTCAGACCCTGATAACTTAGATATCTTGATTAACAAA
AATCTGACGGGGTTCGGACGCAACTTGAAAGCACCTGACAGTAACGATACC
TTAGAAAATCTTATCCGCAAAATCCAGGCCGGCATTCCCGAAGAAGAGGTT
CTTCCGGAGCTGAAGAAAATTAAAGAGATGATCCAGAAAGACATCGTTAAC
CGCAAAGAGCAGTTGCTTAAATCCATCAAAAACAACCGTATTCCTTTTTCA
TTGGAAGGTTCCAAGCTGGTCCCTTCCACCAAAAAGATGAAGTGGCTTTTC
AAGTTGATTGACGTTCCTAATAAAACCTTCAATGAAAAAATGTTGGAGAAG
TACTGGGAGATCTACGACTATGACAAGCTTAAGGCGAATATCACGAACCGC
CTGGACAAAACGGATAAGAAGGCACGTTCAATTTCGCGTGCGGTAAGCGAG
GAGTTACGTGAGTACCATAAAAACTTGCGCACAAACTATAATCGCTTCGTA
TCTGGAGACCGTCCAGCAGCCGGGCTTGACAATGGGGGGAGTGCTAAATAT
AATCCCGATAAGGAGGAGTTCTTATTGTTTCTTAAAGAAGTTGAGCAGTAC
TTCAAGAAATACTTCCCAGTCAAGAGCAAGCATTCGAACAAGTCCAAAGAC
AAATCATTGGTTGATAAGTACAAAAATTATTGCAGTTACAAAGTGGTCAAG
AAAGAGGTCAACCGCAGCATCATCAATCAATTGGTGGCTGGACTTATTCAG
CAGGGTAAGCTTCTGTATTACTTCTACTATAACGATACCTGGCAGGAGGAT
TTCCTGAATTCCTACGGTCTTAGTTATATTCAGGTTGAGGAGGCATTTAAA
AAGAGTGTAATGACCAGTCTTTCGTGGGGAATCAACCGTCTGACCTCGTTC
TTTATCGACGACTCGAACACTGTGAAGTTTGATGATATCACAACGAAAAAG
GCTAAGGAGGCGATTGAGTCTAATTATTTCAACAAATTACGCACATGCTCC
CGTATGCAGGACCACTTCAAAGAGAAATTGGCCTTCTTTTACCCGGTGTAT
GTGAAGGACAAGAAGGATCGCCCGGATGATGATATCGAAAACTTAATTGTA
CTGGTTAAGAACGCCATTGAGAGCGTGAGCTATTTGCGCAATCGCACGTTT
CACTTTAAAGAGAGCTCCTTATTGGAGCTGCTTAAAGAGTTAGACGATAAA
AATTCTGGTCAAAATAAAATCGACTATAGCGTAGCCGCTGAATTTATTAAA
CGCGATATCGAGAACTTGTATGACGTCTTCCGTGAACAGATCCGCTCACTT
GGTATTGCTGAGTACTACAAGGCGGACATGATTTCGGACTGTTTTAAGACA
TGCGGGTTAGAATTCGCGCTTTATTCGCCAAAAAATTCGTTGATGCCCGCT
TTTAAGAACGTTTATAAACGCGGAGCAAATCTTAACAAGGCGTATATTCGC
GATAAAGGGCCAAAGGAAACAGGAGACCAGGGTCAGAACTCGTACAAAGCT
TTGGAGGAATATCGTGAGCTTACATGGTATATTGAGGTGAAGAATAACGAC
CAGTCGTACAACGCCTACAAGAATTTGCTTCAACTGATTTATTATCATGCG
TTTCTTCCGGAGGTTCGTGAAAATGAGGCTCTTATCACTGACTTCATTAAC
CGCACTAAAGAGTGGAATCGTAAGGAAACAGAGGAGCGTCTGAACACTAAA
AACAATAAAAAACACAAGAATTTCGACGAGAATGACGATATTACGGTAAAT
ACGTATCGTTACGAATCTATTCCGGATTACCAGGGGGAATCCCTTGATGAT
TATCTTAAGGTGTTACAGCGCAAGCAAATGGCGCGTGCGAAGGAGGTAAAT
GAGAAGGAGGAGGGCAACAATAACTATATCCAATTCATTCGTGACGTCGTA
GTATGGGCGTTCGGGGCCTACTTGGAAAACAAGCTGAAGAATTATAAGAAC
GAACTGCAACCACCGTTGAGCAAAGAGAACATTGGCTTAAATGATACCTTA
AAGGAATTGTTTCCCGAAGAAAAAGTAAAGTCACCATTTAATATCAAGTGC
CGTTTCTCCATTAGTACATTTATCGACAACAAAGGTAAGTCCACCGATAAT
ACCTCGGCCGAGGCCGTCAAGACTGATGGCAAGGAGGACGAAAAGGACAAA
AAGAATATTAAACGCAAAGACTTGCTTTGCTTTTACCTGTTCTTACGCTTG
CTTGATGAAAACGAAATTTGCAAATTACAACACCAATTTATTAAATACCGT
TGCAGTTTAAAAGAGCGTCGTTTTCCAGGTAACCGCACCAAACTGGAGAAG
GAAACTGAACTTTTGGCCGAGCTGGAAGAACTGATGGAACTGGTACGTTTT
ACGATGCCATCAATTCCTGAAATCTCTGCAAAGGCGGAATCAGGTTATGAC
ACAATGATTAAGAAGTATTTTAAAGACTTCATCGAGAAAAAAGTCTTTAAG
AATCCGAAGACGTCCAACCTGTACTACCACTCCGACTCTAAAACTCCGGTC
ACCCGCAAATATATGGCTCTGCTGATGCGTTCTGCGCCTTTGCATCTCTAC
AAGGATATCTTCAAGGGCTACTATTTGATCACTAAGAAGGAGTGTTTGGAG
TATATTAAATTAAGCAACATCATTAAAGACTATCAAAACTCATTGAATGAA
TTGCACGAGCAGCTGGAGCGCATTAAATTAAAGAGTGAGAAACAAAACGGG
AAAGACTCTCTGTATCTTGACAAAAAGGACTTCTATAAAGTAAAGGAGTAC
GTCGAAAATTTAGAACAAGTGGCGTGTTACAAGCACTTGCAGCACAAAATC
AACTTCGAAAGCTTATACCGTATTTTTCGTATTCACGTGGACATCGCTGCG
CGTATGGTCGGTTATACCCAAGACTGGGAGCGTGATATGCACTTTCTTTTC
AAGGCGTTGGTCTATAATGGCGTACTTGAGGAGCGCCGTTTTGAAGCAATC
TTTAACAATAACGATGACAACAACGACGGGCGTATCGTAAAAAAGATCCAG
AATAACTTAAATAACAAGAACCGCGAATTAGTATCTATGTTATGTTGGAAT
AAAAAGCTGAACAAGAACGAATTTGGTGCTATTATTTGGAAGCGTAACCCT
ATCGCCCATCTGAACCACTTTACACAAACGGAACAAAACTCAAAGTCGAGC
CTTGAATCTCTGATCAACTCGCTGCGCATCCTGTTAGCGTACGACCGTAAA
CGTCAAAACGCTGTGACAAAGACTATCAATGACCTTCTGTTAAACGACTAT
CACATCCGCATTAAGTGGGAAGGGCGCGTGGATGAAGGGCAGATCTACTTT
AATATCAAGGAAAAGGAAGACATCGAGAACGAGCCTATTATCCACTTGAAA
CACCTGCATAAAAAGGACTGCTACATTTACAAGAACAGCTATATGTTCGAC
AAGCAAAAGGAGTGGATTTGTAATGGCATTAAAGAAGAAGTTTATGACAAG
TCCATTCTTAAGTGTATCGGAAACTTGTTCAAGTTTGACTATGAGGACAAA
AACAAATCCTCAGCAAATCCTAAGCACACC.

An exemplary DNA sequence encoding TccCas13a is:

(SEQ ID NO: 63)
ATGAAGATCACGAAAAGGAAATGGGGAGAGCATCATCCGCCGCTTTACTTC
TACCGGGATGAGGACTCCGGCAGGCTCCTGGCACAGAACGACAGAAAGCAG
GATTATACCGATACCCTGTTTAATGATATTGCGCAGGATACATTTGAAAGA
TCGCTGAGAAACCGGCTTTTGAAAACACCCGAAAAGGGAGACAAAAGATTC
TACAGCAACGAGATCGTCAAGCTGGTGGAGAAACTGTGCCAGGGTGCGGAT
GTGGCGGAGATCATGAAAAGCATGGAGAGGAACGAAAAGCTGCGCCCCAAG
AATGAAAAAGAGATTAAAAATCTGAAAAAGCAATTGGACGGTACCCTTTCC
GAATACGGTAAAAGGTATACCGCCCCAGAAGGCGCCATGACCCTCAACGAT
GCCTTGTTTTACCTGGTAGAAGGAAACCCTTTAAAGCAGGCCATGGCCAAA
GCTGAACTGGGCAAAATCCGGGAGGCATTAATAAAGGAAAAGGAAAACCGC
ATCAACCGCGTCCGGTATTCCATCAAGAACAATAAAATACCGCTGAGAATC
CAAGAGGATGGCGGCATTACACCCAATAATGACCGTGCGGCCTGGCTGCTG
GGGCTCATGAAGCCGGCGGACCCGGCAAAAGGAATAACCGACTGCTACCCG
CTCTTAGGCGAGTTGGAAGAAGTATTCGACTTTGACAAGCTGTCCAAAACG
CTGCACGAAAAGATAAGCCGTTGCCAAGGCCGACCCCGTTCCATAGCCATG
GCGGTCGATGAGGCCCTGAAGCAATATCTCCGGGAGCTTTGGGAGAAGTCT
CCCTCGCGACAGCAGGATCTGAAGTATTACTTTCAGGCCGTACAGGAGTAC
TTTAAGGACAATTTTCCCATCCGGACAAAACGGATGGGCGCCCGCCTGCGG
CAGGAATTGCTCAAGGATAAGACGTCCCTTTCCCGTCTGCTGGAACCCAAG
CATATGGCTAATGCCGTTCGCCGCAGGCTGATCAACCAGAGTACCCAGATG
CACATCCTTTATGGAAAGCTTTATGCATATTGCTGCGGGGAAGACGGCAGG
CTTTTGGTAAACAGCGAGACGCTGCAAAGGATACAGGTCCATGAAGCGGTA
AAAAAGCAGGCCATGACGGCCGTGCTGTGGTCCATATCCCGTCTGCGCTAT
TTTTACCAGTTTGAAGACGGCGATATCTTGAGCAATAAAAACCCGATTAAA
GATTTCAGAGATAAATTTCTCAGAGACACGAATAAATATACCCATGAAGAT
GTTGAGGCCTGCAAGGAAAAACTGCAGGACTTCTTCCCGCTGAAAGAATTG
CAGGAAAAGATTAAGGAGGATGCAAAAGGATTACAGGAAACAGACAATAAG
CAGGCTGATACAACGGATTTCAAAGCGATCGGGCACATCGTCAGGGATGAT
CGGAAGCTCTGCAACCAGTTGCTGGCGGAGTGCGTTTCCTGCATCGGGGAG
CTGAGACATCATATCTTTCATTATAAAAATGTGACCTTGATACAGGCGCTC
AAAAGGATCGCCGATAAAGTGAAACCGGAGGATTTGTCTGTGCTCCGGGCC
ATTTACCTGTTGGACAGGAGAAACCTCAAAAAGGCGTTTGCCAAAAGAATC
AGCAGCATGAACCTTCCGCTGTATTACAGGGAGGATCTATTATCCCGCATT
TTCAAAAAAGAAGGGACGGCGTTTTTCCTGTACAGCGCCAAGATCCAGATG
ACACCGTCCTTTCAGAGGGTCTATGAGCGTGGTAAAAATCTGCGCCGGGAG
TTCGAGTGCGAACGCATGAAAGCCGAGGCATCCAACGGACAGAACGGGCAG
GATGGCGACCGGCTGAAATGGTTCCGACAGCTTGCGGCTGGGGATAGCGCC
GATACCCATTTCAACTGGGCTGTGGAGGCCTATGCGGAATCGGCAGCCGAT
GTGGAAAACAACGTTGAATTCGATACCGATGTGGATGCCCAGCGTGCCCTG
CGGAACCTTCTGCTGCTGATATATAGGCACCATTTCCTGCCGGAGGTGCAG
AAGGATGAAACCCTGGTGACCGGCAAGATCCATAAGGTTTTGGAAAGAAAC
AGGCAGCTGTCTGAAGGCCAAGGGCCAAATCAAGGTAAGGCCCATGGATAC
AGCGTTATAGAAGAGCTGTATCATGAAGGCATGCCGCTTTCCGATCTCATG
AAGCAATTGCAGCGGAGGATCTCCGAAACCGAAAGGGAGAGCCGGGAACTG
GCACAGGAAAAAACGGATTATGCCCAGCGCTTTATCCTCGATATCTTTGCC
GAGGCCTTTAACGATTTCCTGGAGGCGCACTATGGCGAAGAATACCTTGAG
ATCATGAGCCCCAGGAAAGATGCCGAAGCGGCGAAGAAATGGGTAAAAGAA
AGCAAAACGGTGGATTTGAAAACATCTATAGACGAAAAAGAACCGGAAGGG
CATCTCCTGGTGCTCTATCCCGTCCTGCGCCTGCTGGACGAAAGGGAACTG
GGAGAGCTTCAGCAGCAGATGATCCGTTATCGGACATCCCTGGCCAGTTGG
CAGGGTGAGAGCAATTTCAGTGAAGAAATAAGGATAGCCGGGCAGATTGAG
GAATTGACCGAACTGGTCAAGCTGACGGAACCGGAGCCCCAGTTTGCGGAG
GAAGTATGGGGGAAACGGGCTAAAGAAGCGTTTGAAGACTTTATTGAAGGA
AACATGAAAAATTATGAGGCTTTTTATCTTCAGAGCGACAACAACACGCCG
GTATATCGGAGGAACATGAGCCGGTTGCTGCGCTCGGGGCTTATGGGAGTG
TACCAAAAGGTGCTTGCCAGCCACAAGCAGGCGCTCAAAAGAGATTACTTG
CTCTGGTCGGAAAAACATTGGAACGTGAAGGATGAGAATGGAGCGGATATC
TCTTCCGCTGAACAGGCCCAATGCCTTTTGCAGCGGCTCCATAGGAAGTAC
GCCGAATCCCCGTCCCGATTTACGGAGGAGGACTGCAAACTGTATGAGAAG
GTTCTCCGGCGGCTTGAGGACTATAACCAGGCCGTGAAAAACCTGTCCTTC
AGTTCATTGTATGAAATATGCGTCCTCAATCTTGAAATCCTCTCCCGATGG
GTGGGGTTTGTCCAGGACTGGGAGCGGGATATGTACTTCCTCCTTTTGGCG
TGGGTCAGACAGGGCAAACTGGACGGGATAAAGGAGGAAGATGTCAGAGAT
ATCTTCTCTGAAGGCAACATTATTCGCAATCTGGTGGATACGCTGAAAGGC
GAAAACATGAATGCCTTTGAAAGCGTTTATTTCCCTGAGAATAAGGGGTCT
AAGTATTTAGGTGTGCGGAATGATGTTGCCCATCTGGATCTGATGCGGAAA
AACGGATGGCGGTTGGAGGCTGGCAAAACCTGCAGCGTGATGGAAGATTAC
ATCAACCGTTTGAGGTTTCTCCTGTCCTATGACCAGAAACGCATGAACGCC
GTCACCAAGACCTTGCAGCAGATATTTGATAGGCATAAAGTCAAAATCCGG
TTCACCGTGGAGAAAGGAGGAATGCTGAAGATAGAGGATGTGACTGCCGAC
AAGATCGTGCATCTGAAAGGTTCCAGGTTGAGCGGCATTGAAATTCCAAGT
CACGGGGAGAGGTTTATTGACACGTTGAAAGCGCTGATGGTATACCCGAGA
GGATGA.

In some preferred embodiments, the Cas13 effector is a Cas13 protein derived from a bacterium in the Proteobacteria phylum. An exemplary Proteobacteria-derived Cas13 is mCas13 encoded by the following DNA sequence:

ATGGGTATTGATTATTCGCTTACAAGTGACTGTTATCGAGGCATCAACAAG
TCTTGTTTTGCAGTTGCTTTGAATATTGCATATGATAACTGTGATCATAAA
GGATGTAGGACTCTTCTGAGTGAGGTGCTGCGCAGCAAGGGAGGGATTTCT
GATGAGCAAATAAAATCACAAGTAGTGGATGGAATTCAGAAGCGACTTAAA
GACATTCGCAATTATTTCTCGCATTATTACCACGCAGAAGACTGTCTGCGG
TTTGGAGACCAAGATGCCGTCAAGGTTTTTCTGGAAGAAATATACAAGAAC
GCGGAATCAAAGACTGTCGGAGCGACAAAAGAAAGCGACTATAAAGGCGIT
GTGCCGCCATTATTTGAATTGCATAACGGTACATATATGATTACGGCGGCG
GGGGTTATTTTTCTGGCATCATTTTTCTGCCATCGGAGTAATGTCTATCGG
ATGCTGGGAGCGGTGAAAGGATTTAAACATACCGGAAAAGAGCAATTGAGC
GATGGGCAGAAACGAGATTATGGTTTCACTCGCCGGCTGCTGGCTTATTAT
GCGCTCCGGGACAGCTATTCTGTGGGGGCGGAAGACAAGACACGATGTTTC
CGCGAGATATTAAGCTATTTGTCGAGAGTACCGCAATTGGCAGTAGATTGG
CTGAATGAACAACAGCTGCTTACACCAGAAGAAAAAGAAGCTTTTTTGAAT
CAACCCGCCGAAGATGAGGGCGGGGATATTTCGGACTCTTCGTCCAGTGAT
AAAAATAAAAAAAGCAAAGAAAAGAGGCGTAGTCTCCGCAGAGATGAAAAA
TTCATTTTATTTGCTATCCAATTTATTGAGGGGTGGGCGGCTGAACAGGGA
TTAGATGTGACATTTGCACGCTACCAAAAAACAGTGGAAAAAGCGGAGAAT
AAGAATCAGGATGGCAAACAGGCCAGAGCGGTGCAATTAAAATACAGAAAC
CAAGGACTCAATCCGGATTTCAATAACGAATGGATGTATTACATTCAGAAT
GAACACGCAATTATCCAGATTAAACTGAATAATAAAAAAGCCGTTGCTGCC
CGTATTTCTGAAAATGAATTAAAATATCTGGTGCTGCTGATTTTTGAGGAG
AAGGGCAATGACGCCGTCCAAAAGCTGAATTGTTATATTTACAGTATGAGC
CAAAAAATCGAGGGCGAATGGAAACACAGGCCGGAGGATGAGCGATGGATG
CCGTCGTTTACCAAGCGTGCCGACAGGACGGTTACGCCGGAGGCAGTGCAG
AGCCGGTTAAGCTATATTCGCAAACAACTTCAGGAGACGATAGAGAAAATC
GGTCAGGAAGAGCCGCGGAATAATAAGTGGCTGATATACAAAGGCAAAAAA
ATATCAATGATACTGAAGTTTATCTCCGACAGCATTCGCGATATTCAAAGG
CGGCCGAATGTGAAACAATATCATATTTTACGCGATGCGCTTCAGAGGCTC
GACTTTGATGGATTTTATAAGGAACTTCAAAATTACGTCAATGACGGCCGG
ATTGCGGTTTCATTATACGATCAGATCAAGGGTGTCAATGACATCAGCGGG
CTTTGTAAAAAAGTCTGCGAACTGACACTTGAAAGACTGGCTGGGCTGGAG
GCAAAGAATGGCTCCGAGCTGAGGCGTTATATTGGGCTTGAAGCGCAGGAA
AAACATCCGAAGTACGGAGAGTGGAACACACTGCAGGAAAAGGCCAAGCGA
TTTCTGGAGTCGCAGTTTTCTATCGGGAAAAACTTTTTGCGGAAAATGTTT
TATGGCGATTGTTGTCAAAAGCGGTGCTTTGACGAAGAAAAAGGTTACAAT
ACACAAGCGAAAGAGCGAAAAAGTCTGTATAGCATTGTGAAAGAAAAACTC
AAGGACATCAAGCCCATACACGATGACCGGTGGTATCTTATCGACAGGAAT
CCGAAGAACTATGACAATAAACACAGCAGGATTATCCGACAGATGTGCAAC
ACCTATATACAAGATGTTCTCTGTATGAAGATGGCGATGTGGCATTATGAG
AAATTAATCTCCGCGACTGAATTCAGGAATAAATTGGAATGGAATTGTATT
GGGCAGGGAAATATGGGTTATGAGCGATATTCGCTGTGGTATAAGACAGGC
TGCGGGGTTGTCATACAATTTACGCCGGCGGATTTTTTGCGACTGGATATC
ATTGAAAAACCTGCGATGATAGAAAATATTTGCCAGTGTTTTGTGCTCGGA
AACAAAAAACTCAATTCGGGTGCCGAAAAGAAGATAACCTGGGATAAATTC
AATAAAGACGGCATTGCAAAATACAGAAAGCGGCAGGCCGAGGCTGTGCGT
GCGATATTTGCGTTTGAGGAAGGTTTAAAAATACAAGAGGATAAATGGAGT
CATGAGAGATACTTTCCATTTTGCAATATTCTTGACGAGGCGGTAAAACAA
GGTAAAATAAAAGATACAGGCAAAGACAAGGAGGCTCTGAATCGCGGCAGA
AATGATTTTTTTCATGAGGAATTTAAGTCAACAGAAGATCAGCAGGCGATT
TTTCAGAAATACTTTCCGATTGTCGAACGAAAAGACGACACAAAAAAACGG
CGAGATAAAAAGCAAAAGTGA
(SEQ ID NO: 65; GenBank accession No.:
DSVK01000191.1).

In some embodiments, the sequence encodes the Cas13 effector protein of SEQ ID NO:64, SEQ ID NO:66 or SEQ ID NO:68. In some embodiments, the sequence encodes the Cas13 effector protein with at least 60%, 65%, 70%, 75%, 80%, 85%, 90%, 95%, 96%, 97%, 98%, or 99% sequence identity to Cas13 encoded by SEQ ID NO:63, SEQ ID NO:65, or SEQ ID NO:67.

In some embodiments, the sequence encoding the Cas13 effector protein is or includes SEQ ID NO:63, SEQ ID NO:65 or SEQ ID NO:67. In some embodiments, the sequence encoding the Cas13 effector protein is or includes a sequence with at least 60%, 65%, 70%, 75%, 80%, 85%, 90%, 95%, 96%, 97%, 98%, or 99% sequence identity to SEQ ID NO:63, SEQ ID NO: 65 or SEQ ID NO:67.

Preferably, the sequence encoding the Cas13 effector protein is codon optimized for expression in a prokaryotic or eukaryotic cell, such as a bacterial, yeast, or mammalian cell. The eukaryotic cells can be those of or derived from a particular organism, such as a mammal, including but not limited to human, mouse, rat, rabbit, cat, dog, horse, pig, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules.

The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al., Nucl. Acids Res., 28:292 (2000). Computer algorithms for codon optimizing a particular sequence for expression in a particular host cell, for example Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a CRISPR enzyme correspond to the most frequently used codon for a particular amino acid.

In some embodiments, the heterologous sequence includes one or more expression control sequences. In some embodiments, the heterologous sequence includes a promoter, transcription terminator, multiple cloning site, drug resistance marker(s), one or more protease recognition sites, one or more epitope tags, or a combination thereof. Typically, the heterologous sequence, such as an expression control sequence, is operably linked to the sequence encoding the Cas13 effector protein.

In some embodiments, the polynucleotide further includes a sequence encoding one or more crRNAs or guide RNA (gRNA) or single guide (sgRNA). In preferred embodiments, the crRNAs are capable of complexing with the Cas13 effector protein and hybridizing to one or more target RNA sequences.

In some embodiments, the polynucleotide is or is contained in a vector, such as an expression vector. Nucleic acids, such as those described above, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA). In some preferred embodiments, suitable vectors include viral vectors (e.g., retroviral, lentiviral, adenoviral, adeno-associated or herpes simplex viral vectors) or plasmids.

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, CT), maltose E binding protein and protein A.

In some embodiments, one or more vectors driving expression of one or more elements of a CRISPR/Cas13 system are introduced into a target cell such that expression of the elements of the CRISPR system direct formation of a CRISPR complex at one or more target RNA sequences. For example, a Cas13 protein and one or more guide sequences could each be operably linked to separate regulatory elements on separate vectors. Alternatively, two or more of the elements expressed from the same or different regulatory elements may be combined in a single vector, with one or more additional vectors providing any components of the CRISPR/Cas13 system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element can be located on the same or opposite strand of the coding sequence of a second element, and oriented in the same or opposite direction.

In some embodiments, a single promoter drives expression of a transcript encoding a Cas13 protein and one or more of the crRNA sequences. In some embodiments, one or more of the elements of CRISPR/Cas13 system are under the control of an inducible promoter, which can include inducible Cas13.

In some embodiments, a vector includes one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector includes an insertion site upstream of a crRNA sequence, and optionally downstream of a regulatory element operably linked to the crRNA sequence, such that following insertion of a Cas13 sequence into the insertion site and upon expression the crRNA sequence directs sequence-specific binding of a Cas13 RNP complex to a target RNA sequence in a cell. In some embodiments, a vector includes two or more insertion sites. In such an arrangement, the two or more crRNA sequences can include two or more copies of a single crRNA sequence, two or more different crRNA sequences, or combinations of these. When multiple different crRNA sequences are used, a single expression construct may be used to target Cas13 activity to multiple different, corresponding target RNA sequences within a cell. For example, a single vector can include about or more than about 1, 2, 3, 4, 5, 6, 7, 8, 9, 10, 15, 20 crRNA sequences. In some embodiments, about or more than about 1, 2, 3, 4, 5, 6, 7, 8, 9, 10, such crRNA-sequence-containing vectors may be provided, and optionally delivered to a cell.

In some embodiments, the relatively small size of mCas13 allows for easier packaging into commonly used delivery vectors such as adeno-associated viruses (AAV). Thus, delivery of mCas13 protein (or TccCas13 or HheCas13a) in vivo for various applications (e.g., RNA targeting and manipulation as well as therapeutic interventions) can be accomplished by use of an AAV vector. The AAV vector can provide one or more elements of the gene editing compositions (e.g., crRNA expression cassette, CAR expression cassette, homology arms).

AAV is a non-pathogenic, single-stranded DNA virus that has been actively employed over the years for delivering therapeutic genes in both in vitro and in vivo systems (Choi, et al., Curr. Gene Ther., 5:299-310, (2005)). AAV belongs to the parvovirus family and is dependent on co-infection with other viruses, mainly adenoviruses, in order to replicate. Initially distinguished serologically, molecular cloning of AAV genes has identified hundreds of unique AAV strains in numerous species. Each end of the single-stranded DNA genome contains an inverted terminal repeat (ITR), which is the only cis-acting element required for genome replication and packaging. The single-stranded AAV genome contains three genes, Rep (Replication), Cap (Capsid), and aap (Assembly). These three genes give rise to at least nine gene products through the use of three promoters, alternative translation start sites, and differential splicing. These coding sequences are flanked by the ITRs. The Rep gene encodes four proteins (Rep78, Rep68, Rep52, and Rep40), while Cap expression gives rise to the viral capsid proteins (VP; VP1/VP2/VP3), which form the outer capsid shell that protects the viral genome, as well as being actively involved in cell binding and internalization. It is estimated that the viral coat is comprised of 60 proteins arranged into an icosahedral structure with the capsid proteins in a molar ratio of 1:1:10 (VP1:VP2:VP3).

Suitable AAV vectors can be a naturally occurring serotype of AAV including, but not limited to, AAV1, AAV2, AAV3, AAV4, AAV5, AAV6, AAV7, AAV8, AAV9, AAV10, AAV11, AAV12, artificial variants such as AAV.rh10, AAV.rh32/33, AAV.rh43, AAV.rh64R1, rAAV2-retro, AAV-DJ, AAV-PHP.B, AAV-PHP.S, AAV-PHP.eB, or other natural or engineered versions of AAV.

Twelve natural serotypes of AAV have thus far been identified, with the best characterized and most commonly used being AAV2. These serotypes differ in their tropism, or the types of cells they infect, making AAV a very useful system for preferentially transducing specific cell types. For example, AAV serotypes 1, 2, 5 or a hybrid capsid AAV1, AAV2, AAV5 or any combination thereof can be used for targeting brain or neuronal cells; AAV4 can be selected for targeting cardiac cells. AAV8 is useful for delivery to the liver cells. Researchers have further refined the tropism of AAV through pseudotyping, or the mixing of a capsid and genome from different viral serotypes. These serotypes are denoted using a slash, so that AAV2/5 indicates a virus containing the genome of serotype 2 packaged in the capsid from serotype 5. Use of these pseudotyped viruses can improve transduction efficiency, as well as alter tropism. For example, AAV2/5 targets neurons that are not efficiently transduced by AAV2/2, and is distributed more widely in the brain, indicating improved transduction efficiency.

An exemplary sequence of an expression vector, a plasmid in particular, containing a nucleic acid sequence encoding TccCas13a is:

(SEQ ID NO: 69)
tcatgaccaaaatcccttaacgtgagttttcgttccactgagcgtcagacc
ccgtagaaaagatcaaaggatcttcttgagatcctttttttctgcgcgtaa
tctgctgcttgcaaacaaaaaaaccaccgctaccagcggtggtttgtttgc
cggatcaagagctaccaactctttttccgaaggtaactggcttcagcagag
cgcagataccaaatactgtccttctagtgtagccgtagttaggccaccact
tcaagaactctgtagcaccgcctacatacctcgctctgctaatcctgttac
cagtggctgctgccagtggcgataagtcgtgtcttaccgggttggactcaa
gacgatagttaccggataaggcgcagcggtcgggctgaacggggggttcgt
gcacacagcccagcttggagcgaacgacctacaccgaactgagatacctac
agcgtgagctatgagaaagcgccacgcttcccgaagggagaaaggcggaca
ggtatccggtaagcggcagggtcggaacaggagagcgcacgagggagcttc
cagggggaaacgcctggtatctttatagtcctgtcgggtttcgccacctct
gacttgagcgtcgatttttgtgatgctcgtcaggggggcggagcctatgga
aaaacgccagcaacgcggcctttttacggttcctggccttttgctggcctt
ttgctcacatgttctttcctgcgttatcccctgattctgtggataaccgta
ttaccgcctttgagtgagctgataccgctcgccgcagccgaacgaccgagc
gcagcgagtcagtgagcgaggaagcggaagagcgcctgatgcggtattttc
tccttacgcatctgtgcggtatttcacaccgcaatggtgcactctcagtac
aatctgctctgatgccgcatagttaagccagtatacactccgctatcgcta
cgtgactgggtcatggctgcgccccgacacccgccaacacccgctgacgcg
ccctgacgggcttgtctgctcccggcatccgcttacagacaagctgtgacc
gtctccgggagctgcatgtgtcagaggttttcaccgtcatcaccgaaacgc
gcgaggcagctgcggtaaagctcatcagcgtggtcgtgaagcgattcacag
atgtctgcctgttcatccgcgtccagctcgttgagtttctccagaagcgtt
aatgtctggcttctgataaagcgggccatgttaagggcggttttttcctgt
ttggtcactgatgcctccgtgtaagggggatttctgttcatgggggtaatg
ataccgatgaaacgagagaggatgctcacgatacgggttactgatgatgaa
catgcccggttactggaacgttgtgagggtaaacaactggcggtatggatg
cggcgggaccagagaaaaatcactcagggtcaatgccagcgcttcgttaat
acagatgtaggtgttccacagggtagccagcagcatcctgcgatgcagatc
cggaacataatggtgcagggcgctgacttccgcgtttccagactttacgaa
acacggaaaccgaagaccattcatgttgttgctcaggtcgcagacgttttg
cagcagcagtcgcttcacgttcgctcgcgtatcggtgattcattctgctaa
ccagtaaggcaaccccgccagcctagccgggtcctcaacgacaggagcacg
atcatgcgcacccgtggccaggacccaacgctgcccgagatgcgccgcgtg
cggctgctggagatggcggacgcgatggatatgttctgccaagggttggtt
tgcgcattcacagttctccgcaagaattgattggctccaattcttggagtg
gtgaatccgttagcgaggtgccgccggcttccattcaggtcgaggtggccc
ggctccatgcaccgcgacgcaacgcggggaggcagacaaggtatagggcgg
cgcctacaatccatgccaacccgttccatgtgctcgccgaggcggcataaa
tcgccgtgacgatcagcggtccaatgatcgaagttaggctggtaagagccg
cgagcgatccttgaagctgtccctgatggtcgtcatctacctgcctggaca
gcatggcctgcaacgcgggcatcccgatgccgccggaagcgagaagaatca
taatggggaaggccatccagcctcgcgtcgcgaacgccagcaagacgtagc
ccagcgcgtcggccgccatgccggcgataatggcctgcttctcgccgaaac
gtttggtggcgggaccagtgacgaaggcttgagcgagggcgtgcaagattc
cgaataccgcaagcgacaggccgatcatcgtcgcgctccagcgaaagcggt
cctcgccgaaaatgacccagagcgctgccggcacctgtcctacgagttgca
tgataaagaagacagtcataagtgcggcgacgatagtcatgccccgcgccc
accggaaggagctgactgggttgaaggctctcaagggcatcggtcgagatc
ccggtgcctaatgagtgagctaacttacattaattgcgttgcgctcactgc
ccgctttccagtcgggaaacctgtcgtgccagctgcattaatgaatcggcc
aacgcgcggggagaggcggtttgcgtattgggcgccagggtggtttttctt
ttcaccagtgagacgggcaacagctgattgcccttcaccgcctggccctga
gagagttgcagcaagcggtccacgctggtttgccccagcaggcgaaaatcc
tgtttgatggtggttaacggcgggatataacatgagctgtcttcggtatcg
tcgtatcccactaccgagatatccgcaccaacgcgcagcccggactcggta
atggcgcgcattgcgcccagcgccatctgatcgttggcaaccagcatcgca
gtgggaacgatgccctcattcagcatttgcatggtttgttgaaaaccggac
atggcactccagtcgccttcccgttccgctatcggctgaatttgattgcga
gtgagatatttatgccagccagccagacgcagacgcgccgagacagaactt
aatgggcccgctaacagcgcgatttgctggtgacccaatgcgaccagatgc
tccacgcccagtcgcgtaccgtcttcatgggagaaaataatactgttgatg
ggtgtctggtcagagacatcaagaaataacgccggaacattagtgcaggca
gcttccacagcaatggcatcctggtcatccagcggatagttaatgatcagc
ccactgacgcgttgcgcgagaagattgtgcaccgccgctttacaggcttcg
acgccgcttcgttctaccatcgacaccaccacgctggcacccagttgatcg
gcgcgagatttaatcgccgcgacaatttgcgacggcgcgtgcagggccaga
ctggaggtggcaacgccaatcagcaacgactgtttgcccgccagttgttgt
gccacgcggttgggaatgtaattcagctccgccatcgccgcttccactttt
tcccgcgttttcgcagaaacgtggctggcctggttcaccacgcgggaaacg
gtctgataagagacaccggcatactctgcgacatcgtataacgttactggt
ttcacattcaccaccctgaattgactctcttccgggcgctatcatgccata
ccgcgaaaggttttgcgccattcgatggtgtccgggatctcgacgctctcc
cttatgcgactcctgcattaggaagcagcccagtagtaggttgaggccgtt
gagcaccgccgccgcaaggaatggtgcatgcaaggagatggcgcccaacag
tcccccggccacggggcctgccaccatacccacgccgaaacaagcgctcat
gagcccgaagtggcgagcccgatcttccccatcggtgatgtcggcgatata
ggcgccagcaaccgcacctgtggcgccggtgatgccggccacgatgcgtcc
ggcgtagaggatcgagatctcgatcccgcgaaattaatacgactcactata
ggggaattgtgagcggataacaattcccctctagaaataattttgtttaac
tttaagaaggagatataccatgggcagcagccatcatcatcatcatcacag
cagcggcctggtgccgcgcggcagccatatggctagctggagccatccgca
gtttgaaaaaggtggtggtagcggtggtggttcaggtggtagtgcatggtc
acaccctcagtttgagaaaatgtcggactcagaagtcaatcaagaagctaa
gccagaggtcaagccagaagtcaagcctgagactcacatcaatttaaaggt
gtccgatggatcttcagagatcttcttcaagatcaaaaagaccactccttt
aagaaggctgatggaagcgttcgctaaaagacagggtaaggaaatggactc
cttaagattcttgtacgacggtattagaatccaagctgatcagacccctga
agatttggacatggaggataacgatattattgaggctcacagagaacagat
tggtggatccATGAAGATTACCAAACGCAAGTGGGGTGAACACCATCCGCC
GCTGTATTTCTACCGTGATGAAGATAGCGGTCGTCTGCTGGCGCAAAACGA
CCGTAAGCAAGATTACACCGACACCCTGTTCAACGACATCGCGCAGGACAC
CTTTGAACGTAGCCTGCGTAACCGTCTGCTGAAAACCCCGGAGAAGGGCGA
CAAACGTTTCTATAGCAACGAAATCGTGAAACTGGTTGAGAAGCTGTGCCA
GGGTGCGGATGTTGCGGAGATTATGAAGAGCATGGAACGTAACGAGAAACT
GCGTCCGAAGAACGAGAAAGAAATTAAGAACCTGAAGAAACAGCTGGACGG
CACCCTGAGCGAGTACGGCAAGCGTTATACCGCGCCGGAAGGTGCGATGAC
CCTGAACGATGCGCTGTTTTACCTGGTTGAGGGCAACCCGCTGAAACAGGC
GATGGCGAAGGCGGAACTGGGTAAAATCCGTGAGGCGCTGATTAAGGAGAA
AGAAAACCGTATCAACCGTGTGCGTTATAGCATTAAAAACAACAAGATCCC
GCTGCGTATTCAGGAAGATGGTGGCATCACCCCGAACAACGACCGTGCGGC
GTGGCTGCTGGGCCTGATGAAGCCGGCGGACCCGGCGAAAGGCATTACCGA
CTGCTACCCGCTGCTGGGTGAACTGGAGGAAGTTTTCGACTTTGATAAACT
GAGCAAGACCCTGCACGAGAAAATCAGCCGTTGCCAAGGTCGTCCGCGTAG
CATTGCGATGGCGGTGGACGAGGCGCTGAAACAGTACCTGCGTGAACTGTG
GGAGAAGAGCCCGAGCCGTCAGCAAGACCTGAAATACTATTTCCAAGCGGT
TCAGGAATATTTCAAGGATAACTTTCCGATCCGTACCAAACGTATGGGCGC
GCGTCTGCGTCAGGAACTGCTGAAGGACAAAACCAGCCTGAGCCGTCTGCT
GGAGCCGAAGCACATGGCGAACGCGGTGCGTCGTCGTCTGATCAACCAAAG
CACCCAGATGCACATTCTGTACGGTAAACTGTACGCGTATTGCTGCGGCGA
GGATGGCCGTCTGCTGGTTAACAGCGAAACCCTGCAACGTATCCAGGTGCA
CGAGGCGGTTAAGAAACAAGCGATGACCGCGGTGCTGTGGAGCATTAGCCG
TCTGCGTTACTTCTATCAGTTTGAGGACGGCGACATCCTGAGCAACAAAAA
CCCGATTAAGGATTTCCGTGACAAGTTTCTGCGTGATACCAACAAATACAC
CCACGAAGACGTTGAGGCGTGCAAGGAAAAACTGCAAGATTTCTTTCCGCT
GAAGGAACTGCAGGAGAAGATCAAAGAGGATGCGAAGGGCCTGCAAGAAAC
CGACAACAAACAGGCGGATACCACCGACTTCAAGGCGATCGGTCACATTGT
TCGTGACGATCGTAAACTGTGCAACCAACTGCTGGCGGAGTGCGTGAGCTG
CATCGGTGAACTGCGTCACCACATTTTTCACTACAAGAACGTGACCCTGAT
CCAGGCGCTGAAACGTATTGCGGATAAGGTGAAACCGGAGGACCTGAGCGT
TCTGCGTGCGATCTATCTGCTGGACCGTCGTAACCTGAAGAAAGCGTTCGC
GAAGCGTATTAGCAGCATGAACCTGCCGCTGTACTATCGTGAGGACCTGCT
GAGCCGTATCTTTAAGAAAGAGGGCACCGCGTTCTTTCTGTACAGCGCGAA
AATTCAAATGACCCCGAGCTTCCAGCGTGTTTATGAACGTGGCAAGAACCT
GCGTCGTGAGTTTGAATGCGAGCGTATGAAAGCGGAGGCGAGCAACGGTCA
AAACGGCCAGGACGGTGATCGTCTGAAGTGGTTTCGTCAGCTGGCGGCGGG
TGATAGCGCGGACACCCACTTTAACTGGGCGGTTGAAGCGTACGCGGAGAG
CGCGGCGGATGTGGAAAACAACGTTGAGTTCGATACCGATGTGGACGCGCA
GCGTGCGCTGCGTAACCTGCTGCTGCTGATCTATCGTCACCACTTTCTGCC
GGAAGTGCAAAAGGATGAAACCCTGGTTACCGGCAAAATTCACAAGGTGCT
GGAGCGTAACCGTCAGCTGAGCGAAGGTCAAGGCCCGAACCAGGGCAAAGC
GCACGGTTACAGCGTGATCGAGGAACTGTATCACGAGGGTATGCCGCTGAG
CGACCTGATGAAGCAACTGCAGCGTCGTATTAGCGAAACCGAGCGTGAAAG
CCGTGAACTGGCGCAAGAGAAAACCGATTACGCGCAGCGTTTCATCCTGGA
CATTTTTGCGGAGGCGTTCAACGATTTTCTGGAAGCGCACTACGGCGAGGA
ATATCTGGAAATCATGAGCCCGCGTAAAGACGCGGAGGCGGCGAAGAAATG
GGTGAAAGAAAGCAAGACCGTTGATCTGAAGACCAGCATTGACGAGAAAGA
ACCGGAGGGTCACCTGCTGGTGCTGTACCCGGTTCTGCGTCTGCTGGATGA
ACGTGAGCTGGGTGAACTGCAGCAACAGATGATCCGTTATCGTACCAGCCT
GGCGAGCTGGCAAGGCGAGAGCAACTTCAGCGAGGAAATCCGTATTGCGGG
TCAGATTGAGGAACTGACCGAACTGGTTAAGCTGACCGAACCGGAGCCGCA
ATTTGCGGAGGAAGTGTGGGGCAAACGTGCGAAGGAAGCGTTCGAGGACTT
TATCGAGGGTAACATGAAAAACTACGAAGCGTTCTATCTGCAGAGCGATAA
CAACACCCCGGTTTACCGTCGTAACATGAGCCGTCTGCTGCGTAGCGGCCT
GATGGGTGTGTACCAAAAGGTTCTGGCGAGCCACAAACAGGCGCTGAAGCG
TGACTATCTGCTGTGGAGCGAGAAGCACTGGAACGTGAAAGATGAAAACGG
CGCGGACATCAGCAGCGCGGAACAAGCGCAGTGCCTGCTGCAACGTCTGCA
CCGTAAGTACGCGGAGAGCCCGAGCCGTTTCACCGAGGAAGACTGCAAACT
GTACGAGAAGGTTCTGCGTCGTCTGGAAGATTATAACCAGGCGGTGAAAAA
CCTGAGCTTTAGCAGCCTGTACGAAATCTGCGTGCTGAACCTGGAAATTCT
GAGCCGTTGGGTGGGTTTCGTTCAAGATTGGGAGCGTGACATGTATTTTCT
GCTGCTGGCGTGGGTTCGTCAGGGCAAGCTGGACGGTATCAAAGAGGAAGA
TGTGCGTGACATTTTCAGCGAAGGCAACATCATTCGTAACCTGGTTGATAC
CCTGAAAGGTGAAAACATGAACGCGTTCGAGAGCGTGTACTTTCCGGAAAA
CAAAGGCAGCAAGTATCTGGGTGTGCGTAACGATGTTGCGCACCTGGACCT
GATGCGTAAGAACGGCTGGCGTCTGGAAGCGGGTAAAACCTGCAGCGTTAT
GGAGGACTACATCAACCGTCTGCGTTTCCTGCTGAGCTATGATCAGAAGCG
TATGAACGCGGTGACCAAAACCCTGCAACAGATTTTCGACCGTCACAAGGT
GAAAATTCGTTTTACCGTTGAGAAGGGTGGCATGCTGAAAATCGAAGATGT
GACCGCGGACAAGATTGTTCACCTGAAAGGCAGCCGTCTGAGCGGTATCGA
GATTCCGAGCCACGGCGAGCGTTTTATTGACACCCTGAAGGCGCTGATGGT
TTACCCGCGTGGTTAAgcggccgcactcgaggcccgaaaggaagctgagtt
ggctgctgccaccgctgagcaataactagcataaccccttggggcctctaa
acgggtcttgaggggttttttgctgaaaggaggaactatatccggatatcc
cgcaagaggcccggcagtaccggcataaccaagcctatgcctacagcatcc
agggtgacggtgccgaggatgacgatgagcgcattgttagatttcatacac
ggtgcctgactgcgttagcaatttaactgtgataaactaccgcattaaagc
ttatcgatgataagctgtcaaacatgagaattcttgaagacgaaagggcct
cgtgatacgcctatttttataggttaatgtcatgataataatggtttctta
gacgtcaggtggcacttttcggggaaatgtgcgcggaacccctatttgttt
atttttctaaatacattcaaatatgtatccgctcatgagacaataaccctg
ataaatgcttcaataatattgaaaaaggaagagtatgagtattcaacattt
ccgtgtcgcccttattcccttttttgcggcattttgccttcctgtttttgc
tcacccagaaacgctggtgaaagtaaaagatgctgaagatcagttgggtgc
acgagtgggttacatcgaactggatctcaacagcggtaagatccttgagag
ttttcgccccgaagaacgttttccaatgatgagcacttttaaagttctgct
atgtggcgcggtattatcccgtgttgacgccgggcaagagcaactcggtcg
ccgcatacactattctcagaatgacttggttgagtactcaccagtcacaga
aaagcatcttacggatggcatgacagtaagagaattatgcagtgctgccat
aaccatgagtgataacactgcggccaacttacttctgacaacgatcggagg
accgaaggagctaaccgcttttttgcacaacatgggggatcatgtaactcg
ccttgatcgttgggaaccggagctgaatgaagccataccaaacgacgagcg
tgacaccacgatgcctgcagcaatggcaacaacgttgcgcaaactattaac
tggcgaactacttactctagcttcccggcaacaattaatagactggatgga
ggcggataaagttgcaggaccacttctgcgctcggcccttccggctggctg
gtttattgctgataaatctggagccggtgagcgtgggtctcgcggtatcat
tgcagcactggggccagatggtaagccctcccgtatcgtagttatctacac
gacggggagtcaggcaactatggatgaacgaaatagacagatcgctgagat
aggtgcctcactgattaagcattggtaactgtcagaccaagtttactcata
tatactttagattgatttaaaacttcatttttaatttaaaaggatctaggt
gaagatcctttttgataatc.

An exemplary sequence of an expression vector, a plasmid in particular, containing a nucleic acid sequence encoding mCas13a is:

(SEQ ID NO: 70)
tttcctgtttggtcactgatgcctccgtgtaagggggatttctgttcatgg
gggtaatgataccgatgaaacgagagaggatgctcacgatacgggttactg
atgatgaacatgcccggttactggaacgttgtgagggtaaacaactggcgg
tatggatgcggcgggaccagagaaaaatcactcagggtcaatgccagcgct
tcgttaatacagatgtaggtgttccacagggtagccagcagcatcctgcga
tgcagatccggaacataatggtgcagggcgctgacttccgcgtttccagac
tttacgaaacacggaaaccgaagaccattcatgttgttgctcaggtcgcag
acgttttgcagcagcagtcgcttcacgttcgctcgcgtatcggtgattcat
tctgctaaccagtaaggcaaccccgccagcctagccgggtcctcaacgaca
ggagcacgatcatgcgcacccgtggccaggacccaacgctgcccgagatgc
gccgcgtgcggctgctggagatggcggacgcgatggatatgttctgccaag
ggttggtttgcgcattcacagttctccgcaagaattgattggctccaattc
ttggagtggtgaatccgttagcgaggtgccgccggcttccattcaggtcga
ggtggcccggctccatgcaccgcgacgcaacgcggggaggcagacaaggta
tagggcggcgcctacaatccatgccaacccgttccatgtgctcgccgaggc
ggcataaatcgccgtgacgatcagcggtccaatgatcgaagttaggctggt
aagagccgcgagcgatccttgaagctgtccctgatggtcgtcatctacctg
cctggacagcatggcctgcaacgcgggcatcccgatgccgccggaagcgag
aagaatcataatggggaaggccatccagcctcgcgtcgcgaacgccagcaa
gacgtagcccagcgcgtcggccgccatgccggcgataatggcctgcttctc
gccgaaacgtttggtggcgggaccagtgacgaaggcttgagcgagggcgtg
caagattccgaataccgcaagcgacaggccgatcatcgtcgcgctccagcg
aaagcggtcctcgccgaaaatgacccagagcgctgccggcacctgtcctac
gagttgcatgataaagaagacagtcataagtgcggcgacgatagtcatgcc
ccgcgcccaccggaaggagctgactgggttgaaggctctcaagggcatcgg
tcgagatcccggtgcctaatgagtgagctaacttacattaattgcgttgcg
ctcactgcccgctttccagtcgggaaacctgtcgtgccagctgcattaatg
aatcggccaacgcgcggggagaggcggtttgcgtattgggcgccagggtgg
tttttcttttcaccagtgagacgggcaacagctgattgcccttcaccgcct
ggccctgagagagttgcagcaagcggtccacgctggtttgccccagcaggc
gaaaatcctgtttgatggtggttaacggcgggatataacatgagctgtctt
cggtatcgtcgtatcccactaccgagatatccgcaccaacgcgcagcccgg
actcggtaatggcgcgcattgcgcccagcgccatctgatcgttggcaacca
gcatcgcagtgggaacgatgccctcattcagcatttgcatggtttgttgaa
aaccggacatggcactccagtcgccttcccgttccgctatcggctgaattt
gattgcgagtgagatatttatgccagccagccagacgcagacgcgccgaga
cagaacttaatgggcccgctaacagcgcgatttgctggtgacccaatgcga
ccagatgctccacgcccagtcgcgtaccgtcttcatgggagaaaataatac
tgttgatgggtgtctggtcagagacatcaagaaataacgccggaacattag
tgcaggcagcttccacagcaatggcatcctggtcatccagcggatagttaa
tgatcagcccactgacgcgttgcgcgagaagattgtgcaccgccgctttac
aggcttcgacgccgcttcgttctaccatcgacaccaccacgctggcaccca
gttgatcggcgcgagatttaatcgccgcgacaatttgcgacggcgcgtgca
gggccagactggaggtggcaacgccaatcagcaacgactgtttgcccgcca
gttgttgtgccacgcggttgggaatgtaattcagctccgccatcgccgctt
ccactttttcccgcgttttcgcagaaacgtggctggcctggttcaccacgc
gggaaacggtctgataagagacaccggcatactctgcgacatcgtataacg
ttactggtttcacattcaccaccctgaattgactctcttccgggcgctatc
atgccataccgcgaaaggttttgcgccattcgatggtgtccgggatctcga
cgctctcccttatgcgactcctgcattaggaagcagcccagtagtaggttg
aggccgttgagcaccgccgccgcaaggaatggtgcatgcaaggagatggcg
cccaacagtcccccggccacggggcctgccaccatacccacgccgaaacaa
gcgctcatgagcccgaagtggcgagcccgatcttccccatcggtgatgtcg
gcgatataggcgccagcaaccgcacctgtggcgccggtgatgccggccacg
atgcgtccggcgtagaggatcgagatctcgatcccgcgaaattaatacgac
tcactataggggaattgtgagcggataacaattcccctctagaaataattt
tgtttaactttaagaaggagatataccatgggcagcagccatcatcatcat
catcacagcagcggcctggtgccgcgcggcagccatatggctagctggagc
catccgcagtttgaaaaaggtggtggtagcggtggtggttcaggtggtagt
gcatggtcacaccctcagtttgagaaaatgtcggactcagaagtcaatcaa
gaagctaagccagaggtcaagccagaagtcaagcctgagactcacatcaat
ttaaaggtgtccgatggatcttcagagatcttcttcaagatcaaaaagacc
actcctttaagaaggctgatggaagcgttcgctaaaagacagggtaaggaa
atggactccttaagattcttgtacgacggtattagaatccaagctgatcag
acccctgaagatttggacatggaggataacgatattattgaggctcacaga
gaacagattggtggatccATGGGAATAGACTATAGTCTAACATCAGATTGC
TACCGTGGTATCAATAAATCTTGTTTCGCTGTGGCGCTGAACATTGCTTAC
GACAACTGCGATCATAAAGGCTGCCGTACCCTGCTGTCGGAGGTTTTGCGC
TCTAAAGGCGGTATTTCGGACGAACAGATTAAGTCCCAAGTTGTCGATGGT
ATCCAGAAGCGCCTGAAGGATATCCGTAACTATTTCAGCCATTATTATCAT
GCAGAGGATTGCCTGCGCTTTGGTGACCAGGATGCGGTGAAAGTGTTTCTG
GAGGAGATTTATAAGAATGCAGAGAGCAAAACGGTCGGTGCGACCAAAGAG
TCTGACTACAAGGGCGTTGTACCTCCGCTCTTTGAATTACATAATGGTACA
TACATGATTACCGCAGCGGGTGTCATCTTTCTCGCCAGCTTCTTCTGCCAT
CGTAGCAACGTTTATCGCATGCTGGGTGCCGTCAAAGGCTTCAAACACACC
GGCAAAGAGCAGCTGTCCGATGGCCAGAAACGTGACTATGGCTTTACGCGT
CGCCTGCTCGCTTACTATGCTCTGCGTGACTCCTACAGCGTTGGCGCAGAA
GATAAAACGCGTTGCTTTCGTGAAATTCTGTCGTATCTGTCACGTGTTCCA
CAGTTGGCGGTGGACTGGCTGAACGAACAACAATTGTTGACGCCGGAAGAA
AAGGAGGCGTTTCTGAACCAGCCGGCAGAGGACGAGGGCGGTGACATCTCT
GACAGCAGTAGCTCCGACAAAAACAAAAAGTCCAAAGAGAAAAGACGCAGC
CTGCGCCGTGATGAGAAGTTCATTTTGTTTGCCATCCAATTTATCGAGGGC
TGGGCAGCAGAGCAGGGTCTGGATGTTACCTTCGCTCGTTACCAGAAGACC
GTTGAAAAGGCTGAGAACAAAAACCAGGACGGTAAACAAGCGCGTGCAGTT
CAGCTCAAGTACCGCAACCAAGGCCTGAACCCGGATTTCAACAACGAGTGG
ATGTATTACATCCAGAACGAGCACGCGATTATTCAGATTAAACTGAATAAT
AAGAAAGCGGTTGCAGCGCGTATTAGCGAAAATGAACTGAAGTATTTGGTC
CTGTTGATCTTCGAGGAGAAGGGTAATGACGCGGTGCAGAAGTTGAATTGC
TACATCTACAGCATGAGCCAGAAGATTGAGGGTGAATGGAAACACCGTCCG
GAAGATGAGCGTTGGATGCCGAGCTTCACCAAACGAGCGGACCGCACCGTG
ACCCCGGAAGCCGTCCAAAGCCGTCTGAGCTATATCCGCAAACAGCTGCAA
GAAACCATTGAGAAGATTGGTCAAGAAGAACCGCGTAACAACAAGTGGCTG
ATCTACAAGGGCAAAAAGATCTCTATGATTTTGAAGTTCATCAGTGATTCG
ATCCGTGATATTCAACGCCGTCCGAATGTTAAACAATATCACATTTTGCGT
GACGCACTGCAACGTCTCGACTTCGATGGTTTTTATAAGGAACTTCAGAAT
TATGTTAATGACGGCCGCATTGCCGTAAGCCTGTATGACCAAATCAAGGGC
GTGAATGACATCAGCGGTCTGTGTAAAAAAGTTTGTGAACTGACCTTGGAA
AGGCTAGCGGGTCTGGAGGCTAAAAACGGCAGCGAACTGAGACGTTACATC
GGCCTGGAAGCGCAGGAGAAGCACCCGAAGTACGGTGAATGGAATACCCTG
CAAGAAAAAGCGAAACGCTTTCTGGAGTCTCAGTTCTCCATTGGTAAGAAT
TTTTTGCGTAAAATGTTTTACGGCGATTGTTGTCAAAAGCGCTGCTTCGAT
GAGGAGAAAGGCTACAACACCCAGGCGAAGGAGCGCAAAAGCCTGTATTCC
ATCGTTAAGGAGAAGCTGAAGGATATCAAGCCGATCCATGATGACCGTTGG
TATCTGATTGATCGTAACCCGAAAAACTACGATAATAAGCACAGCCGCATC
ATCCGTCAGATGTGCAACACGTACATCCAAGATGTGTTATGCATGAAGATG
GCGATGTGGCACTACGAGAAATTAATCTCCGCGACTGAGTTCCGTAACAAG
TTGGAGTGGAACTGCATTGGTCAGGGTAACATGGGTTATGAACGTTACTCA
CTGTGGTACAAGACCGGTTGCGGTGTGGTGATCCAATTCACGCCAGCTGAT
TTCCTGCGGCTGGACATTATTGAAAAACCGGCAATGATCGAGAACATCTGC
CAGTGTTTCGTGCTGGGCAACAAAAAGCTAAACAGCGGCGCGGAAAAGAAA
ATCACTTGGGATAAATTCAACAAGGACGGCATAGCGAAATACCGTAAGCGC
CAGGCCGAGGCGGTGCGTGCGATCTTCGCGTTTGAAGAGGGTCTCAAGATC
CAAGAAGACAAATGGTCACACGAACGTTACTTCCCGTTTTGTAATATCTTG
GATGAAGCCGTGAAGCAGGGGAAAATCAAAGATACCGGTAAGGACAAAGAA
GCCTTAAATCGTGGTCGTAACGACTTTTTTCATGAAGAATTTAAAAGCACC
GAAGACCAGCAAGCGATTTTTCAAAAATACTTCCCGATTGTTGAACGGAAG
GACGACACCAAAAAACGTCGCGATAAGAAACAGAAGTAAgcggccgcactc
gaggcccgaaaggaagctgagttggctgctgccaccgctgagcaataacta
gcataaccccttggggcctctaaacgggtcttgaggggttttttgctgaaa
ggaggaactatatccggatatcccgcaagaggcccggcagtaccggcataa
ccaagcctatgcctacagcatccagggtgacggtgccgaggatgacgatga
gcgcattgttagatttcatacacggtgcctgactgcgttagcaatttaact
gtgataaactaccgcattaaagcttatcgatgataagctgtcaaacatgag
aattcttgaagacgaaagggcctcgtgatacgcctatttttataggttaat
gtcatgataataatggtttcttagacgtcaggtggcacttttcggggaaat
gtgcgcggaacccctatttgtttatttttctaaatacattcaaatatgtat
ccgctcatgagacaataaccctgataaatgcttcaataatattgaaaaagg
aagagtatgagtattcaacatttccgtgtcgcccttattcccttttttgcg
gcattttgccttcctgtttttgctcacccagaaacgctggtgaaagtaaaa
gatgctgaagatcagttgggtgcacgagtgggttacatcgaactggatctc
aacagcggtaagatccttgagagttttcgccccgaagaacgttttccaatg
atgagcacttttaaagttctgctatgtggcgcggtattatcccgtgttgac
gccgggcaagagcaactcggtcgccgcatacactattctcagaatgacttg
gttgagtactcaccagtcacagaaaagcatcttacggatggcatgacagta
agagaattatgcagtgctgccataaccatgagtgataacactgcggccaac
ttacttctgacaacgatcggaggaccgaaggagctaaccgcttttttgcac
aacatgggggatcatgtaactcgccttgatcgttgggaaccggagctgaat
gaagccataccaaacgacgagcgtgacaccacgatgcctgcagcaatggca
acaacgttgcgcaaactattaactggcgaactacttactctagcttcccgg
caacaattaatagactggatggaggcggataaagttgcaggaccacttctg
cgctcggcccttccggctggctggtttattgctgataaatctggagccggt
gagcgtgggtctcgcggtatcattgcagcactggggccagatggtaagccc
tcccgtatcgtagttatctacacgacggggagtcaggcaactatggatgaa
cgaaatagacagatcgctgagataggtgcctcactgattaagcattggtaa
ctgtcagaccaagtttactcatatatactttagattgatttaaaacttcat
ttttaatttaaaaggatctaggtgaagatcctttttgataatctcatgacc
aaaatcccttaacgtgagttttcgttccactgagcgtcagaccccgtagaa
aagatcaaaggatcttcttgagatcctttttttctgcgcgtaatctgctgc
ttgcaaacaaaaaaaccaccgctaccagcggtggtttgtttgccggatcaa
gagctaccaactctttttccgaaggtaactggcttcagcagagcgcagata
ccaaatactgtccttctagtgtagccgtagttaggccaccacttcaagaac
tctgtagcaccgcctacatacctcgctctgctaatcctgttaccagtggct
gctgccagtggcgataagtcgtgtcttaccgggttggactcaagacgatag
ttaccggataaggcgcagcggtcgggctgaacggggggttcgtgcacacag
cccagcttggagcgaacgacctacaccgaactgagatacctacagcgtgag
ctatgagaaagcgccacgcttcccgaagggagaaaggcggacaggtatccg
gtaagcggcagggtcggaacaggagagcgcacgagggagcttccaggggga
aacgcctggtatctttatagtcctgtcgggtttcgccacctctgacttgag
cgtcgatttttgtgatgctcgtcaggggggcggagcctatggaaaaacgcc
agcaacgcggcctttttacggttcctggccttttgctggccttttgctcac
atgttctttcctgcgttatcccctgattctgtggataaccgtattaccgcc
tttgagtgagctgataccgctcgccgcagccgaacgaccgagcgcagcgag
tcagtgagcgaggaagcggaagagcgcctgatgcggtattttctccttacg
catctgtgcggtatttcacaccgcaatggtgcactctcagtacaatctgct
ctgatgccgcatagttaagccagtatacactccgctatcgctacgtgactg
ggtcatggctgcgccccgacacccgccaacacccgctgacgcgccctgacg
ggcttgtctgctcccggcatccgcttacagacaagctgtgaccgtctccgg
gagctgcatgtgtcagaggttttcaccgtcatcaccgaaacgcgcgaggca
gctgcggtaaagctcatcagcgtggtcgtgaagcgattcacagatgtctgc
ctgttcatccgcgtccagctcgttgagtttctccagaagcgttaatgtctg
gcttctgataaagcgggccatgttaagggcggttt.

mCas13 coding sequence is in capital letters, preceded by vector sequence and sequence encoding 6× His affinity tag, Thrombin site, Strep-tag II, SUMO, and followed by additional vector sequence in lowercase letters.

Suitable expression vectors for expressing the disclosed Cas13 effectors include vectors having a nucleotide sequence with at least 60% sequence identity to SEQ ID NO:69 or 70.

While the specifics can be varied in different engineered CRISPR systems, the overall methodology is similar. A practitioner interested in using the CRISPR/Cas13 technology to target an RNA sequence (identified using one of the many available online tools) can insert a short DNA fragment containing the target sequence into a guide RNA expression plasmid. The expression plasmid contains the target sequence (or spacer), one or more direct repeat sequences, as well as a suitable promoter and necessary elements for proper processing in a cell of interest. Such vectors are commercially available (see, for example, Addgene). Many of the systems rely on custom, complementary oligos that are annealed to form a double stranded DNA and then cloned into the crRNA expression plasmid. Co-expression of the crRNA(s) and the appropriate Cas13 enzyme from the same or separate plasmids in transfected cells can result in a target RNA cleavage (depending of the activity of the Cas enzyme) at the desired target site.

### B. Cells

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the antigenic polypeptides described herein.

Cells harboring the polynucleotides, vectors, proteins, RNPs, and/or other compositions disclosed herein are also provided. For example, prokaryotic or eukaryotic cells containing the polynucleotide and/or vector are disclosed. The cell can be a mammalian cell. The mammalian cell can be human or non-human mammal, e.g., primate, bovine, ovine, porcine, canine, rodent, monkey, rat, or mouse cell. The cell can be a non-mammalian eukaryotic cell such as poultry bird (e.g., chicken), vertebrate fish (e.g., salmon) or shellfish (e.g., oyster, claim, lobster, shrimp) cell. The cell can also be a plant cell.

In preferred embodiments, the cell is a human cell including, but not limited to, skin cells, lung cells, heart cells, kidney cells, pancreatic cells, muscle cells, neuronal cells, human embryonic stem cells, blood cells (e.g., white blood cells), and pluripotent stem cells. The cell can be T cells (e.g., CD8+ T cells such as effector T cells, memory T cells, central memory T cells, and effector memory T cells; or CD4+ T cells such as Th1 cells, Th2 cells, Th3 cells, Th9 cells, Th17 cells, Tfh cells, and Treg cells; or gamma-delta T cells/gdT cells), hematopoietic stem cells (HSC), macrophages, natural killer cells (NK), B cells, dendritic cells (DC), or other immune cells.

In some embodiments, the cell can be from established cell lines or they can be primary cells, where “primary cells,” refers to cells and cells cultures that have been derived from a subject and allowed to grow in vitro for a limited number of passages or splittings of the culture.

The Cas13 compositions can be introduced to the cell through a variety of techniques, including viral or non-viral approaches. For example, the Cas13 and/or crRNA can be introduced via a viral vector (e.g., a retrovirus such as a lentivirus, adenovirus, poxvirus, Epstein-Barr virus, or adeno-associated virus (AAV)). Non-viral approaches such as physical and/or chemical methods can also be used, including, but not limited to cationic liposomes and polymers, DNA nanoclew, gene gun, microinjection, transfection, electroporation, nucleofection, particle bombardment, ultrasound utilization, magnetofection, conjugation to cell penetrating peptides, and/or nanoparticle mediated delivery. Such methods are described for example, in Nayerossadat N., et al., Adv. Biomed. Res., 1:27 (2012) and Lino C A, et al., Drug Deliv., 25 (1): 1234-1257 (2018). In view of the respective advantages and disadvantages of each method, a skilled artisan would be able to determine an optimal method for introduction of the elements of the Crispr/Cas13 system.

### C. Exemplary Cas13 Proteins, Modifications and RNP Complexes

Isolated Cas13 effector proteins are also provided. In some embodiments, the cas 13 protein is produced by expressing a disclosed polynucleotide or expression vector in a desired cell, and optionally purifying the protein from the cell.

Preferably, the Cas13 proteins are thermostable enzymes. The working examples demonstrate that HheCas13a exhibits robust activity at temperatures up to 60° C. while TccCas13a maintained a robust activity at temperatures as high as ˜ 70° C. Thus, in some embodiments, the Cas13 enzyme exhibits ssRNA cleavage activity at a temperature of about 37-70° C. In some embodiments, the Cas13 enzyme exhibits ssRNA cleavage activity at a temperature of about 47-60° C. In some embodiments, the Cas13 enzyme exhibits ssRNA cleavage activity at a temperature of about 37-42° C. In some embodiments, the Cas13 enzyme exhibits ssRNA cleavage activity at a temperature of about 60° C.

In preferred embodiments, the Cas13 protein contains one or more Higher Eukaryotes and Prokaryotes Nucleotide-binding (HEPN) domains. In a preferred embodiment, the Cas13 protein includes two HEPN domains. An exemplary HEPN domain is a RxxxxH (SEQ ID NO:192) motif sequence, wherein X represents any amino acid. A HEPN domain can include at least one RxxxxH (SEQ ID NO:192) motif having the sequence of R[N/H/K]X₁X₂X₃H. In some embodiments, an HEPN domain includes a RxxxxH (SEQ ID NO:192) motif having the sequence of R [N/H]X₁X₂X₃H (SEQ ID NO:193). In an embodiment, a HEPN domain has the sequence of R [N/K]X₁X₂X₃H (SEQ ID NO:194). In certain embodiments, X₁ is R, S, D, E, Q, N, G, Y, or H. In certain embodiments, X₂ is I, S, T, V, or L. In certain embodiments, X₃ is L, F, N, Y, V, I, S, D, E, or A.

i. Exemplary Sequences

Preferably, the Cas13 protein is a Cas13a protein derived from thermophilic bacteria, preferably from Thermoclostridium caenicola (TccCas13a) or Herbinix hemicellulosilytica (HheCas13a). An exemplary amino acid sequence for HheCas13a is:

(SEQ ID NO: 68)
MKLTRRRISGNSVDQKITAAFYRDMSQGLLYYDSEDNDCTDKVIESMDFER
SWRGRILKNGEDDKNPFYMFVKGLVGSNDKIVCEPIDVDSDPDNLDILINK
NLTGFGRNLKAPDSNDTLENLIRKIQAGIPEEEVLPELKKIKEMIQKDIVN
RKEQLLKSIKNNRIPFSLEGSKLVPSTKKMKWLFKLIDVPNKTFNEKMLEK
YWEIYDYDKLKANITNRLDKTDKKARSISRAVSEELREYHKNLRTNYNRFV
SGDRPAAGLDNGGSAKYNPDKEEFLLFLKEVEQYFKKYFPVKSKHSNKSKD
KSLVDKYKNYCSYKVVKKEVNRSIINQLVAGLIQQGKLLYYFYYNDTWQED
FLNSYGLSYIQVEEAFKKSVMTSLSWGINRLTSFFIDDSNTVKFDDITTKK
AKEAIESNYFNKLRTCSRMQDHFKEKLAFFYPVYVKDKKDRPDDDIENLIV
LVKNAIESVSYLRNRTFHFKESSLLELLKELDDKNSGONKIDYSVAAEFIK
RDIENLYDVFREQIRSLGIAEYYKADMISDCFKTCGLEFALYSPKNSLMPA
FKNVYKRGANLNKAYIRDKGPKETGDQGQNSYKALEEYRELTWYIEVKNND
QSYNAYKNLLQLIYYHAFLPEVRENEALITDFINRTKEWNRKETEERLNTK
NNKKHKNFDENDDITVNTYRYESIPDYQGESLDDYLKVLQRKQMARAKEVN
EKEEGNNNYIQFIRDVVVWAFGAYLENKLKNYKNELQPPLSKENIGLNDTL
KELFPEEKVKSPFNIKCRFSISTFIDNKGKSTDNTSAEAVKTDGKEDEKDK
KNIKRKDLLCFYLFLRLLDENEICKLQHQFIKYRCSLKERRFPGNRTKLEK
ETELLAELEELMELVRFTMPSIPEISAKAESGYDTMIKKYFKDFIEKKVFK
NPKTSNLYYHSDSKTPVTRKYMALLMRSAPLHLYKDIFKGYYLITKKECLE
YIKLSNIIKDYQNSLNELHEQLERIKLKSEKONGKDSLYLDKKDFYKVKEY
VENLEQVACYKHLQHKINFESLYRIFRIHVDIAARMVGYTQDWERDMHFLF
KALVYNGVLEERRFEAIFNNNDDNNDGRIVKKIQNNLNNKNRELVSMLCWN
KKLNKNEFGAIIWKRNPIAHLNHFTQTEQNSKSSLESLINSLRILLAYDRK
RQNAVTKTINDLLLNDYHIRIKWEGRVDEGQIYFNIKEKEDIENEPIIHLK
HLHKKDCYIYKNSYMFDKQKEWICNGIKEEVYDKSILKCIGNLFKFDYEDK
NKSSANPKHT.

An exemplary amino acid sequence for TecCas13a is:

MKITKRKWGEHHPPLYFYRDEDSGRLLAQNDRKQDYTDTLFNDIAQDTFER
SLRNRLLKTPEKGDKRFYSNEIVKLVEKLCQGADVAEIMKSMERNEKLRPK
NEKEIKNLKKOLDGTLSEYGKRYTAPEGAMTLNDALFYLVEGNPLKQAMAK
AELGKIREALIKEKENRINRVRYSIKNNKIPLRIQEDGGITPNNDRAAWLL
GLMKPADPAKGITDCYPLLGELEEVFDFDKLSKTLHEKISRCQGRPRSIAM
AVDEALKQYLRELWEKSPSRQQDLKYYFQAVQEYFKDNFPIRTKRMGARLR
QELLKDKTSLSRLLEPKHMANAVRRRLINQSTQMHILYGKLYAYCCGEDGR
LLVNSETLORIQVHEAVKKQAMTAVLWSISRLRYFYQFEDGDILSNKNPIK
DFRDKFLRDTNKYTHEDVEACKEKLQDFFPLKELQEKIKEDAKGLQETDNK
QADTTDFKAIGHIVRDDRKLCNOLLAECVSCIGELRHHIFHYKNVTLIQAL
KRIADKVKPEDLSVLRAIYLLDRRNLKKAFAKRISSMNLPLYYREDLLSRI
FKKEGTAFFLYSAKIQMTPSFQRVYERGKNLRREFECERMKAEASNGONGQ
DGDRLKWFRQLAAGDSADTHENWAVEAYAESAADVENNVEFDTDVDAQRAL
RNLLLLIYRHHFLPEVQKDETLVTGKIHKVLERNRQLSEGQGPNQGKAHGY
SVIEELYHEGMPLSDLMKQLQRRISETERESRELAQEKTDYAQRFILDIFA
EAFNDFLEAHYGEEYLEIMSPRKDAEAAKKWVKESKTVDLKTSIDEKEPEG
HLLVLYPVLRLLDERELGELQQQMIRYRTSLASWQGESNFSEEIRIAGQIE
ELTELVKLTEPEPQFAEEVWGKRAKEAFEDFIEGNMKNYEAFYLQSDNNTP
VYRRNMSRLLRSGLMGVYQKVLASHKQALKRDYLLWSEKHWNVKDENGADI
SSAEQAQCLLORLHRKYAESPSRFTEEDCKLYEKVLRRLEDYNQAVKNLSF
SSLYEICVLNLEILSRWVGFVQDWERDMYFLLLAWVRQGKLDGIKEEDVRD
IFSEGNIIRNLVDTLKGENMNAFESVYFPENKGSKYLGVRNDVAHLDLMRK
NGWRLEAGKTCSVMEDYINRLRFLLSYDQKRMNAVTKILQQIFDRHKVKIR
FTVEKGGMLKIEDVTADKIVHLKGSRLSGIEIPSHGERFIDTLKALMVYPR
G
(SEQ ID NO: 64; Genbank Accession
No.: WP_149678719.1).

In some preferred embodiments, the Cas13 protein is a Cas13 protein originated, isolated or derived from a bacterium in the Proteobacteria phylum. An exemplary Proteobacteria-derived Cas13 is mCas13 having the following amino acid sequence:

MGIDYSLTSDCYRGINKSCFAVALNIAYDNCDHKGCRTLLSEVLRSKGGIS
DEQIKSQVVDGIQKRLKDIRNYFSHYYHAEDCLRFGDQDAVKVFLEEIYKN
AESKTVGATKESDYKGVVPPLFELHNGTYMITAAGVIFLASFFCHRSNVYR
MLGAVKGFKHTGKEQLSDGQKRDYGFTRRLLAYYALRDSYSVGAEDKTRCF
REILSYLSRVPQLAVDWLNEQQLLTPEEKEAFLNQPAEDEGGDISDSSSSD
KNKKSKEKRRSLRRDEKFILFAIQFIEGWAAEQGLDVTFARYQKTVEKAEN
KNQDGKQARAVQLKYRNQGLNPDFNNEWMYYIQNEHAIIQIKLNNKKAVAA
RISENELKYLVLLIFEEKGNDAVOKLNCYIYSMSQKIEGEWKHRPEDERWM
PSFTKRADRTVTPEAVQSRLSYIRKQLQETIEKIGQEEPRNNKWLIYKGKK
ISMILKFISDSIRDIQRRPNVKQYHILRDALQRLDFDGFYKELQNYVNDGR
IAVSLYDQIKGVNDISGLCKKVCELTLERLAGLEAKNGSELRRYIGLEAQE
KHPKYGEWNTLQEKAKRFLESQFSIGKNFLRKMFYGDCCQKRCFDEEKGYN
TQAKERKSLYSIVKEKLKDIKPIHDDRWYLIDRNPKNYDNKHSRIIRQMCN
TYIQDVLCMKMAMWHYEKLISATEFRNKLEWNCIGQGNMGYERYSLWYKTG
CGVVIQFTPADFLRLDIIEKPAMIENICQCFVLGNKKLNSGAEKKITWDKF
NKDGIAKYRKRQAEAVRAIFAFEEGLKIQEDKWSHERYFPFCNILDEAVKQ
GKIKDTGKDKEALNRGRNDFFHEEFKSTEDQQAIFQKYFPIVERKDDTKKR
RDKKQK
(SEQ ID NO: 66; GenBank accession No.: HFH51004.1).

Thus, in some embodiments, the Cas13 protein includes the amino acid sequence of SEQ ID NO:64, SEQ ID NO:66, or SEQ ID NO:68. In some embodiments, the Cas13 protein includes an amino acid sequence with at least 60%, 65%, 70%, 75%, 80%, 85%, 90%, 95%, 96%, 97%, 98%, or 99% sequence identity to SEQ ID NO:64, SEQ ID NO:66, or SEQ ID NO:68 or Uniprot accession numbers disclosed herein, and including nucleic acid sequences encoding amino acid sequences thereof.

ii. Cas13 Variants and Modifications

It should be appreciated that also disclosed are Cas13 effector protein variants including one or more mutations (e.g., conservative or non-conservative mutations) relative to any of the Cas effector proteins disclosed herein. For example, it is also contemplated that other Cas13 variants can be evolved from those disclosed herein, for example, by targeted mutation of one or more amino acid residues in specific regions of the enzyme. Such mutation(s) may alter substrate binding, alter conformation of bound substrate, alter substrate accessibility to the active site, alter tolerance to non-optimal presentation of a target sequence to the active site, and/or alter target sequence specificity (recognition). In some embodiments, a suitable Cas13 effector protein has an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NO:64, 66 or 68.

In some embodiments, a disclosed Cas13 protein (e.g., encoded by a polynucleotide or vector, contained in an RNP, or expressed in a cell) is mutated with respect to a corresponding wild-type protein. In certain embodiments, the Cas13 is engineered and can include one or more mutations that reduce or eliminate a nuclease activity. Mutations can also be made at neighboring residues, e.g., at amino acids near those that participate in the nuclease activity. In some embodiments, one HEPN domain is inactivated. In other embodiments, a second HEPN domain is inactivated.

The mutated Cas13 enzyme can have abolished or reduced ability to cleave a target polynucleotide containing a target sequence. In some embodiments, a Cas13 enzyme is considered to substantially lack all cleavage activity when the RNA cleavage activity of the mutated enzyme is less than about 25%, 10%, 5%>, 1%>, 0.1%>, 0.01%, or lower with respect to its non-mutated form.

RNA cleavage is mediated by catalytic residues in the two conserved HEPN domains of Cas13. Therefore, catalytically dead or inactive Cas13 mutants can be generated by mutating one or more catalytic residues within the HEPN domains (e.g., via Alanine substitution). Such mutations can render the Cas13 as a catalytically inactive RNA-binding protein. In particular embodiments, the one or more mutated residues are one or more residues in the disclosed Cas13 proteins that correspond to R597, H602, R1278 and H1283 of Leptotrichia shahii Cas13 (LshCas13), such as mutations R597A, H602A, R1278A and H1283A.

The Cas13 proteins may be modified in various ways. In some embodiments, the modification(s) may render the protein more stable (e.g., resistant to degradation in vivo) or more capable of penetrating into cells or subcellular compartments, or other desirable characteristic as will be appreciated by one skilled in the art. Such modifications include, without limitation, chemical modification, N terminus modification, C terminus modification, peptide bond modification, backbone modifications, residue modification, D-amino acids, or non-natural amino acids or others. In some embodiments, one or more modifications may be used simultaneously.

The modifications may or may not cause an altered functionality. By means of example, modifications which do not result in an altered functionality include for instance codon optimization for expression into a particular host, or providing the Cas13 with a particular marker or epitope tag (e.g. for visualization and/or isolation or purification).

In some embodiments, a disclosed Cas13 protein can be fused to or operably linked to domains which include but are not limited to a transcriptional activator, a transcriptional repressor, a recombinase, a transposase, a histone remodeler, a DNA methyltransferase, a cryptochrome, a light inducible/controllable domain, a chemically inducible/controllable domain, a detectable marker, or a deaminase domain. Exemplary detectable markers include fluorescent proteins, such as, GFP, eGFP, RFP, YFP, BFP, CFP, mNeonGreen, mCherry, mOrange, mRaspberry, mPlum, mKO, mRFP, mRFPruby, mRuby, tagRFP, mKate2, and DsRed.

In some embodiments, the Cas13 is covalently linked to the one or more domains. In some embodiments, the Cas13 is covalently linked to the one or more domains via one or more linkers (e.g., peptide linkers). In some embodiments, the Cas13 protein is a fusion protein including the Cas13 effector and one or more domains, such as those described herein.

Exemplary deaminase domains include deaminase domains of the RNA-dependent Adenosine Deaminases (ADAR) such as ADAR1, ADAR2, and ADAR3, e.g., human ADAR1, ADAR2, and ADAR3. The ADARs catalyze the hydrolytic deamination of adenosine to inosine in double-stranded RNA (dsRNA) in a process generally referred to as A-to-I RNA editing. Modified ADAR deaminase domains in which the adenosine deaminase activity is converted to cytidine deaminase activity are known and are contemplated herein. See for example Published PCT Application No.: WO 2019/071048, which is hereby incorporated by reference in its entirety.

In some embodiments, the Cas13 protein includes one or more nuclear localization sequences (NLSs). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In general, the one or more NLSs are of sufficient strength to drive accumulation of the Cas13 enzyme in a detectable amount in the nucleus of a eukaryotic cell. In general, strength of nuclear localization activity may derive from the number of NLSs in the CRISPR enzyme, the particular NLS(s) used, or a combination of these factors.

In some embodiments, the Cas13 protein can be modified with one or more (e.g., two or more, three or more, or four or more) mitochondrial localization sequences (MLSs). Any convenient mitochondrial localization sequence can be used. Examples of mitochondrial localization sequences include a mitochondrial localization sequence of SDHB, mono/di/triphenylphosphonium or other phosphoniums, VAMP 1A, VAMP 1B, the 67 N-terminal amino acids of DGAT2, and the 20 N-terminal amino acids of Bax. The MLS(s) can be placed at the N- or C-termini of the Cas13 protein.

iii. Cas13 RNPs

In some embodiments, the Cas13 protein is complexed with an RNA including or consisting of a crRNA sequence (e.g., a gRNA, sgRNA, etc.), thereby forming a ribonucleoprotein (RNP) complex. The crRNA may be capable of hybridizing to a target RNA sequence, such as an mRNA, lincRNA, or viral RNA (e.g., SARS-Cov-2 N or E gene transcript). The crRNA can include a spacer sequence that is capable of hybridizing to the target RNA sequence and a direct repeat sequence.

In certain embodiments, the guide RNA or crRNA is or includes a direct repeat sequence linked to a guide sequence or spacer sequence. In certain embodiments the guide RNA or mature crRNA includes about 19 nucleotides of partial direct repeat followed by 18, 19, 20, 21, 22, 23, 24, 25, 26, 27, 28, 29, 30 or more nucleotides of guide or spacer sequence, such as 18-30, 20-25, 25-30, 21-25, 22-25, 23-25, or 25-28 nucleotides of guide or spacer sequence. In some preferred embodiments, the crRNA includes a spacer of about 20-30 nucleotides. In some preferred embodiments, the crRNA includes a spacer of about 24-28 nucleotides. In some embodiments, the crRNA contains a stem loop or other secondary structure. In preferred embodiments the crRNA contains a stem loop or other secondary structure in the direct repeat sequence, wherein the stem loop or secondary structure is important for cleavage activity.

Exemplary crRNAs that can be included in the Cas13 RNP complex are crRNAs encoded by the nucleic acid sequence of SEQ ID NOs: 20-37 and 41-61, sequences having at least 70%, 75%, 80%, 85%, 90%, 95%, 96%, 97%, 98%, or 99% sequence identity thereto, nucleic acid sequences having 1, 2, 3, 4, 5, 6, 7, 8, 9, or 10 nucleic acid substitution(s), addition(s), deletion(s), or a combination thereof relative thereto, or the reverse complement of any of the foregoing.

### III. Methods of Manufacture

### A. Methods for Producing Cas13 Proteins

Isolated Cas13 polypeptides and fusions proteins thereof can be obtained by, for example, chemical synthesis or by recombinant production in a host cell. To recombinantly produce the protein, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding a Cas13 polypeptide. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well known in the art and include, for example, Escherichia coli strains such as BL-21, and cultured mammalian cells such as CHO cells.

In eukaryotic host cells, a number of viral-based expression systems can be utilized to express antigenic polypeptides or fusions proteins. Viral based expression systems are well known in the art and include, but are not limited to, baculoviral, SV40, retroviral, or vaccinia based viral vectors.

Mammalian cell lines that stably express Cas13 polypeptides or fusions proteins can be produced using expression vectors with appropriate control elements and a selectable marker. For example, the eukaryotic expression vectors pCR3.1 (Invitrogen Life Technologies) and p91023 (B) (see Wong et al. (1985) Science 228:810-815) are suitable for expression of variant costimulatory polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, a antigenic polypeptides and fusion proteins can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Cas13 polypeptides and fusion proteins can be isolated using, for example, chromatographic methods such as DEAE ion exchange, gel filtration, and hydroxylapatite chromatography. For example, Cas13 proteins in a cell culture supernatant or a cytoplasmic extract can be isolated using a protein G column. In some embodiments, Cas13 proteins are “engineered” to contain an amino acid sequence that allows the polypeptides to be captured onto an affinity matrix. For example, a tag such as c-myc, hemagglutinin, polyhistidine, or Flag™ (Kodak) can be used to aid polypeptide purification. Such tags are typically inserted at either the carboxyl or amino terminus. Other fusions that can be useful include enzymes that aid in the detection of the polypeptide, such as alkaline phosphatase. Immunoaffinity chromatography also can be used to purify antigenic polypeptides and fusions proteins.

### B. Methods for Producing Isolated Nucleic Acid Molecules

Isolated nucleic acid molecules encoding Cas13 polypeptides and fusion proteins can be produced by standard techniques, including, without limitation, common molecular cloning and chemical nucleic acid synthesis techniques. For example, polymerase chain reaction (PCR) techniques can be used to obtain an isolated nucleic acid encoding an Cas13 polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector.

### IV. Methods of Use

Disclosed herein are various methods related to the disclosed compositions and reagents (including Cas13 proteins, Cas13 fusion proteins and other engineered variants, crRNAs, etc.) and their use. For example, disclosed are methods of perturbing (e.g., reducing or increasing) gene expression, deaminating a target nucleic acid (RNA editing), monitoring trafficking of RNA molecules in cells, etc.

Disclosed is a method of modifying a target locus of interest, in particular in eukaryotic cells, tissues, organs, or organisms, more in particular in mammalian cells, tissues, organs, or organisms. The method involves delivering to said locus a disclosed Cas13 effector protein and one or more crRNA components (i.e., an RNA containing a crRNA), wherein the Cas13 protein forms an RNP complex with the one or more crRNAs components. Upon binding of the complex to the locus of interest, the Cas13 effector protein induces the modification of the target locus of interest depending on the Cas13's activity. In a preferred embodiment, the modification is the introduction of a strand break (e.g., cleavage). In some embodiments, the modification is a deamination of a target nucleotide. The complex can be formed in vitro or ex vivo and introduced into a cell or contacted with RNA; or can be formed in vivo.

Cas13 is capable of robust RNA detection. In certain embodiments, Cas13 is converted to an RNA binding protein (“dead Cas13”) by inactivation of its nuclease activity. Converted to an RNA binding protein, Cas13 is useful for localizing other functional components to RNA in a sequence dependent manner. The components can be natural or synthetic. Dead Cas13 can be used to (i) bring effector modules to specific transcripts to modulate the function or translation, which could be used for large-scale screening, construction of synthetic regulatory circuits and other purposes; (ii) fluorescently tag specific RNAs to visualize their trafficking and/or localization; (iii) alter RNA localization through domains with affinity for specific subcellular compartments; and (iv) capture specific transcripts (through direct pull down of dead Cas13 or use of dead Cas13 to localize biotin ligase activity to specific transcripts) to enrich for proximal molecular partners, including RNAs and proteins.

It will be appreciated that the small size of the mCas13 protein can allow easier packaging into commonly used delivery vectors, such as adeno-associated viruses (AAV) and thus easier delivery of this protein in vivo for various RNA targeting and manipulation as well as therapeutic applications. Such applications include, without limitation, RNA knockdown, RNA editing, antiviral activity, and RNA tagging and imaging.

Furthermore, the discovery of the thermostable enzymes has allowed the development of various biotechnological applications at elevated temperature, such as, the development of nucleic acid detection methods. Besides the one-pot assay described herein, the thermophilic TccCas13a is useful in developing different detection modules for nucleic acids, and possibly other molecules. In particular, the disclosed TccCas13a protein allows for various RNA targeting and manipulation applications in various prokaryotic and eukaryotic organisms, such as RNA knockdown, RNA editing, antiviral activity, and RNA tagging and imaging. The robust thermostability of TccCas13a can also allow all of the aforementioned applications in thermophilic organisms. This is an advantage of TccCas13a over all other known Cas13 enzymes.

Besides SARS-COV-2 detection, both mCas13 and TccCas13 nucleic acid detection modules can be developed for the detection of other pathogens, including any DNA and RNA viruses, bacteria and others.

### A. Targeted RNA Knockdown

The disclosed Cas13 based compositions can be used to reduce or inhibit expression of an RNA transcript. RNA knockdown with Cas13 can be applied to perturbing RNAs in multiple biological contexts, including genome-wide pooled knockdown screening, interrogation of LincRNA and nascent transcript function, allele-specific knockdown, and RNA viral therapeutics.

Such inhibitory effects (i.e., targeted knockdown of an RNA transcript) can be achieved by introducing a Cas13 to a cell, wherein the Cas13 is complexed with a crRNA that targets (e.g., is complementary to or hybridizes to) the RNA transcript, thereby inducing cleavage of the RNA transcript by the Cas13 protein. Any RNA can be targeted for cleavage. For example, in some embodiments, the RNA transcript can be endogenous or exogenous to a cell. In some embodiments, the RNA transcript can be coding (e.g., an mRNA) or non-coding (e.g., lincRNA). In some embodiments, the RNA transcript is derived from a viral gene, such as a bacteriophage. Thus, the Cas13 compositions can be used to inhibit or reduce viral gene expression or viral replication.

Current RNA knockdown strategies such as siRNA have the disadvantage that they are mostly limited to targeting cytosolic transcripts since the protein machinery is cytosolic. An advantage of a Cas13 mediated knockdown is that it is an exogenous system that is not essential to cell function, and it can be used in any compartment in the cell. By fusing a NLS signal to the Cas13, it can be guided to the nucleus, allowing nuclear RNAs to be targeted, e.g., to probe the function of lincRNAs. Long intergenic non-coding RNAs (lincRNAs) are a vastly underexplored area of research. Most lincRNAs have as of yet unknown functions which could be studies using the RNA targeting effector protein of the invention. Knockdown of RNA transcript relies on cleavage of the targeted transcripts by the endogenous RNase activity of the dual HEPN domains of the Cas13 protein, the efficiency of which varies between different orthologs and subtypes of Cas13.

In certain embodiments, modulation of cleavage efficiency can be exploited by introduction of mismatches, e.g., 1 or more mismatches, such as 1 or 2 mismatches between the spacer sequence and target sequence, including the position of the mismatch along the spacer/target. In some embodiments, the more central (i.e., not 3′ or 5′) for instance a mismatch is, the more cleavage efficiency is affected. In some embodiments, the more terminal (i.e., 3′ or 5′) a mismatch is, the more cleavage efficiency is affected. Accordingly, by choosing a mismatch position along the spacer, cleavage efficiency can be modulated. By means of example, if less than 100% cleavage of targets is desired (e.g. in a cell population), 1 or more, such as 2 mismatches between spacer and target sequence may be introduced in the spacer sequences.

B. RNA Imaging and/or Localization

Methods to monitor RNA trafficking are also provided. For example, the Cas13 proteins can be used to effectively fluorescently tag specific RNAs to visualize their trafficking and/or localization. Typically, described herein is a method of determining the localization of an RNA transcript in a cell by introducing the Cas13 composition to the cell. In such embodiments, the Cas13 is preferably catalytically inactive (e.g., the Cas13 functions as an RNA binding protein without nuclease activity). The Cas13 can be linked (e.g., as a fusion protein with or without a linker) to a detectable marker. The Cas13-crRNA RNP targets and hybridizes to the targeted RNA transcript, thereby indicating the location and/or movement of the RNA transcript within the cell. Exemplary detectable markers that can be used to visualize the targeted RNA transcript include GFP, eGFP, RFP, YFP, BFP, CFP, mNeonGreen, mCherry, mOrange, mRaspberry, mPlum, mKO, mRFP, mRFPruby, mRuby, tagRFP, mKate2, and DsRed.

C. RNA editing

Also disclosed is a method for performing targeted editing of an RNA transcript by introducing a catalytically inactive Cas13 that further includes a deaminase domain of an ADAR to a cell. The Cas13-crRNA RNP is capable of hybridizing with a region in an RNA transcript that contains an A nucleotide to be edited. The crRNA forms an RNA duplex with the RNA transcript wherein the duplex contains an A-C mismatch at the target A nucleotide resulting in the target A nucleotide being deaminated by the deaminase domain (resulting in A-I). Suitable deaminase domains include deaminase domains from ADAR1, ADAR2, and ADAR3, such as human ADAR1, ADAR2, and ADAR3. Modified ADAR deaminase domains in which the adenosine deaminase activity is converted to cytidine deaminase activity are known and are contemplated herein. Transitions in the RNA bases of a given transcript can be directly induced by using adenine (ADAR1/2), APOBEC) or cytosine deaminases (AID) which convert A to I or C to U, respectively.

### D. RNA Isolation or Purification, Enrichment or Depletion

The Cas13 can be used as an RNA targeting effector protein when complexed to a target RNA to isolate and/or purify the target RNA. The Cas13 protein can for instance be fused to an affinity tag that can be used to isolate and/or purify the RNA-Cas13 protein complex. Such applications are for instance useful in the analysis of gene expression profiles in cells. In particular embodiments, the Cas13 proteins can be used to target a specific noncoding RNA (ncRNA) thereby blocking its activity, providing a useful functional probe. In certain embodiments, the Cas13 protein may be used to specifically enrich for a particular RNA (including but not limited to increasing stability, etc.), or alternatively to specifically deplete a particular RNA (such as without limitation for instance particular splice variants, isoforms, etc.).

### E. Identification of RNA Binding Proteins

Identifying proteins bound to specific RNAs can be useful for understanding the roles of many RNAs. For instance, many lincRNAs associate with transcriptional and epigenetic regulators to control transcription. Understanding what proteins bind to a given lincRNA can help elucidate the components in a given regulatory pathway. A Cas13 effector protein can be designed to recruit a biotin ligase to a specific transcript in order to label locally bound proteins with biotin. The proteins can then be pulled down and analyzed by mass spectrometry to identify them.

### F. Detection of Nucleic Acids

The Cas13 proteins can be used for detection of nucleic acids or proteins in a biological sample. The samples can be cellular or cell-free. For example, northern blotting involves the use of electrophoresis to separate RNA samples by size. The Cas13 protein can be used to specifically bind and detect the target RNA sequence.

Cas13 protein complexes with the guide RNA via recognition of a short hairpin in the crRNA, and target specificity is encoded by the spacer that is complementary to the target region. In addition to programmable RNase activity, all Cas13s exhibit “collateral activity” after recognition and cleavage of a target transcript, leading to non-specific degradation of any nearby transcripts regardless of complementarity to the spacer. This collateral activity can be referred to as trans activity because the Cas13 is activated to cleave other RNA species in trans without sequence specificity. Conversely, the activity toward the targeted RNA transcript (e.g., having some complementarity to the spacer sequence of the crRNA) can be referred to as cis activity. The disclosed Cas13 based detection assay take advantage of this cis and trans Cas13 activity.

Cas13 proteins such as mCas13, TccCas13a, and HheCas13a can cleave non-targeted single stranded RNA (ssRNA) once activated by detection of a target RNA. Once a type VI CRISPR/Cas effector protein (e.g., mCas13, TccCas13a, or HheCas13a) is activated by a crRNA, which occurs when the crRNA hybridizes to a target sequence of a target RNA (i.e., the sample includes the targeted RNA), the protein becomes a nuclease that cleaves non-target ssRNAs (i.e., ssRNAs to which the spacer sequence of the crRNA does not hybridize). Thus, when the target DNA or RNA transcribed therefrom is present in the sample (e.g., in some cases above a threshold amount), the result is cleavage of ssRNAs in the sample, which can be detected using any convenient detection method (e.g., using a labeled ssRNA oligonucleotide).

Disclosed is a method of detecting the presence of an RNA transcript in a nucleic acid sample by contacting the sample with the Cas13 composition in the presence of an activatable single stranded RNA (ssRNA) oligonucleotide that includes a reporter moiety. In some embodiments, the reporter moiety includes a fluorophore linked to a quencher via the ssRNA. Fluorescence can be emitted upon cleavage of the ssRNA oligonucleotide since the quencher is no longer in proximity to the fluorophore. Preferably, the crRNA is designed to hybridize to the RNA transcript and the Cas13 cleaves the ssRNA oligonucleotide upon binding of the Cas13 crRNA complex to the RNA transcript. Detecting cleavage of the ssRNA oligonucleotide indicates the presence of the RNA transcript. The RNA transcript could have been generated by transcription from a dsDNA molecule, and in some embodiments, the dsDNA molecule could have been generated by reverse transcription coupled amplification of a target RNA.

In a specific embodiment, a Cas13-based method of detecting the presence of a nucleic acid in a sample involves (a) performing an amplification reaction (e.g., LAMP, RPA) on nucleic acids derived from the sample to generate a specific amplification product; (b) transcribing the amplification product to generate an RNA transcript; (c) contacting the RNA transcript with a Cas13-crRNA RNP complex having a crRNA complementary to the RNA transcript; in the presence of an activatable ssRNA oligonucleotide; and (d) detecting cleavage of the ssRNA oligonucleotide by the Cas13 enzyme. In some embodiments, transcription of the amplification product in step (b) is mediated by a T7 RNA polymerase in vitro. Preferably, cleavage of the ssRNA oligonucleotide is dependent on or subsequent to binding of the RNP to the RNA transcript. In some embodiments, cleavage of the ssRNA oligonucleotide results in release of a previously quenched fluorescent signal, thereby indicating the presence of the nucleic acid.

The Cas13-based detection assay can be performed as a one-pot or two-pot assay depending on the Cas13 enzyme that is used. In some embodiments when TccCas13a or HheCas13a is used, the assay can be performed as a one-pot assay in which isothermal amplification (e.g., LAMP, optionally coupled with reverse transcription (i.e., RT-LAMP)) of the target nucleic acids, coupled with T7-mediated in vitro transcription and Cas13-based detection of the amplified and in vitro-transcribed target RNA is carried out in the same tube.

In some embodiments, because of its incompatibility with the elevated temperatures required for the isothermal amplification (e.g., RT-LAMP) reaction, mCas13 can be preferably used in a two-pot assay. In the two-pot assay, the RT-LAMP reaction is performed in a first tube while T7-mediated in vitro transcription and mCas13-based detection of the amplified and in vitro-transcribed target RNA is performed in a second tube.

In preferred embodiments, the sample is or the nucleic acids are derived from mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, sputa/deep throat saliva, feces, mucosal excretions, plasma, serum, or whole blood. In some embodiments, the sample is a nucleic acid isolated and/or derived from any of the foregoing biological samples.

The methods may be used for any purpose for which detection of viral, bacterial or other nucleic acids is desirable, including diagnostic and prognostic applications, such as in laboratory and clinical settings. In some embodiments, the methods may be used for detection of a nucleic acid for genotyping. In some embodiments, the nucleic acid to be detected is diagnostic for a disease state. The disease state can be an infection, an organ disease, a blood disease, an immune system disease, a cancer, a brain and nervous system disease, an endocrine disease, a pregnancy or childbirth-related disease, an inherited disease, or an environmentally-acquired disease, cancer, or a fungal infection, a bacterial infection, a parasite infection, or a viral infection. Thus, in some embodiments, the method is useful for detecting a nucleic acid (e.g., DNA or RNA) from a bacterium, fungus, virus, or parasite.

Exemplary viruses that can be detected include, without limitation, Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In some embodiments, the virus is coronavirus (e.g., SARS-Cov-2), SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In some embodiments, the nucleic acid to be detected can be associated with a pathogen, including pathogenic bacteria such as, E. faecalis, E. faecium Listeria monocytogenes, Campylobacter jejuni, Staphylococcus aureus (e.g., MRSA), E. coli O157:H7, Borrelia burgdorferi, Helicobacter pylori, Ehrlichia chaffeensis, Clostridium difficil, Vibrio cholerae O139, Salmonella enterica, Bartonella henselae, Streptococcus pyogenes, Chlamydia pneumoniae, Clostridium botulinum, Corynebacterium amycolatum, Klebsiella pneumoniae Vibrio vulnificus, and Parachlamydia.

The discloses compositions and methods can also be understood through the following numbered paragraphs:

1. A polynucleotide comprising a nucleotide sequence
encoding a class II, type VI CRISPR/Cas effector protein (Cas13) and optionally a heterologous sequence, wherein the Cas effector protein comprises an amino acid sequence encoded by SEQ ID NO:63, SEQ ID NO: 65 or SEQ ID NO:67, or a sequence with at least 70% sequence identity thereto;
optionally wherein the sequence encoding the Cas effector protein comprises SEQ ID NO:63, SEQ ID NO:65 or SEQ ID NO:67, or a sequence with at least 70% sequence identity to SEQ ID NO:63, SEQ ID NO:65 or SEQ ID NO:67.

2. The polynucleotide of paragraph 1, wherein the sequence encoding the Cas effector protein is codon optimized for expression in a prokaryotic or eukaryotic cell.

3. The polynucleotide of paragraph 1 or 2, comprising a heterologous sequence, wherein the heterologous sequence comprises a promoter, transcription terminator, multiple cloning site, drug resistance marker, one or more protease recognition sites, one or more epitope tags, or a combination thereof.

4. The polynucleotide of paragraph 3, wherein the heterologous sequence is operably linked to the sequence encoding the Cas effector protein.

5. The polynucleotide of any one of paragraphs 1˜4 further comprising a sequence encoding an RNA comprising a crRNA sequence, wherein the RNA is capable of complexing with the Cas effector protein and hybridizing to a target RNA sequence.

6. The polynucleotide of any one of paragraphs 1-5, wherein the Cas effector protein is derived from Thermoclostridium caenicola or a Proteobacteria bacterium.

7. A vector comprising the polynucleotide of any one of paragraphs 1-6, optionally wherein the vector comprises the nucleotide sequence of SEQ ID NO:69 or SEQ ID NO:70, or a sequence having at least 60% sequence identity to SEQ ID NO:69 or SEQ ID NO:70.

8. The vector of paragraph 7, wherein the vector is a viral vector or plasmid.

9. A prokaryotic or eukaryotic cell comprising the vector of paragraph 7 or 8.

10. A method of producing a class II, type VI CRISPR/Cas effector protein comprising contacting the vector of paragraph 7 or 8 with a prokaryotic or eukaryotic cell under conditions suitable for expression of the sequence encoding the Cas effector protein.

11. The method of paragraph 10 further comprising isolating and/or purifying the Cas effector protein.

12. An isolated class II, type VI CRISPR/Cas effector protein, wherein the Cas effector protein is produced by the method of paragraph 10 or 11.

13. An isolated class II, type VI CRISPR/Cas effector protein comprising the amino acid sequence of SEQ ID NO:64 or SEQ ID NO:66, or a sequence with at least 70% sequence identity to SEQ ID NO:64, SEQ ID NO: 66 or SEQ ID NO:68.

14. A ribonucleoprotein complex comprising the Cas effector protein of paragraph 12 or 13 complexed with an RNA comprising crRNA sequence, optionally wherein the RNA is capable of hybridizing to a target RNA sequence.

15. A composition comprising a Cas13 protein, wherein the Cas13 protein comprises the amino acid sequence of SEQ ID NO:64, SEQ ID NO: 66 or SEQ ID NO:68, or a sequence with at least 70% sequence identity to SEQ ID NO:64, SEQ ID NO:66, or SEQ ID NO:68.

16. The composition of paragraph 15, wherein the Cas13 protein comprises one or more HEPN (Higher Eukaryotes and Prokaryotes Nucleotide-binding) domains, preferably two HEPN domains.

17. The composition of paragraph 16, wherein the HEPN domain comprises a RxxxxH (SEQ ID NO:192) motif sequence, wherein X represents any amino acid.

18. The composition of any one of paragraphs 15-17, wherein the Cas13 protein is complexed with and RNA comprising a crRNA sequence, optionally wherein the RNA is capable of hybridizing to a target RNA sequence.

19. The composition of paragraph 18, wherein the crRNA sequence comprises a spacer sequence that is capable of hybridizing to the target RNA sequence, and a direct repeat sequence.

20. The composition of paragraph 18 or 19, wherein the crRNA sequence comprises a spacer of about 20-30 nucleotides, preferably 24-28 nucleotides.

21. The composition of any one of paragraphs 18-20, wherein the Cas13 protein can cleave the target RNA sequence at a temperature of about 37-70° C., about 50-70° C., about 47-60° C., or about 60° C.

22. The composition of any one of paragraphs 18-20, wherein the Cas13 protein can cleave the target RNA sequence at a temperature of about 37° C.-42° C., preferably about 37° C.

23. The composition of any one of paragraphs 18-22 comprised in a prokaryotic or eukaryotic cell.

24. A method of performing targeted knockdown of an RNA transcript comprising introducing the composition of any one of paragraphs 18-22 to a cell, wherein the crRNA sequence hybridizes to the RNA transcript, thereby inducing cleavage of the RNA transcript by the Cas13 protein.

25. The method of paragraph 24, wherein RNA transcript is a mRNA or lincRNA.

26. The method of paragraph 24, wherein RNA transcript is derived from a viral gene, preferably a bacteriophage.

27. A method of detecting the presence of an RNA transcript in a nucleic acid sample comprising contacting the sample with the composition of any one of paragraphs 18-22 in the presence of an activatable single stranded RNA (ssRNA) oligonucleotide comprising a reporter moiety,
wherein the crRNA is designed to hybridize to the RNA transcript, wherein the Cas13 cleaves the ssRNA oligonucleotide upon binding of the Cas13 crRNA complex to the RNA transcript, wherein detection of the cleavage of the ssRNA oligonucleotide indicates the presence of the RNA transcript.

28. The method of paragraph 27, wherein the RNA transcript is generated by transcription from a dsDNA molecule, optionally wherein the dsDNA molecule is generated by reverse transcription coupled isothermal amplification of a target RNA.

29. The method of paragraph 27 or 28, wherein the reporter moiety comprises a fluorophore linked to a quencher via the ssRNA, wherein fluorescence is emitted upon cleavage of the ssRNA oligonucleotide.

30. A method of determining the localization of an RNA transcript in a cell comprising introducing the composition of any one of paragraphs 18-22 to the cell, wherein the Cas13 is catalytically inactive and further comprises a detectable marker,
wherein the crRNA is designed to hybridize to the RNA transcript, and wherein the Cas13 crRNA complex binds to the RNA transcript, thereby indicating the location of the RNA transcript.

31. The method of paragraph 30, wherein the detectable marker is selected from GFP, eGFP, RFP, YFP, BFP, CFP, mNeonGreen, mCherry, mOrange, mRaspberry, mPlum, mKO, mRFP, mRFPruby, mRuby, tagRFP, mKate2, and DsRed.

32. A method for performing targeted editing of an RNA transcript comprising introducing the composition of any one of paragraphs 18-22 to a cell,
wherein the Cas13 is catalytically inactive and further comprises a deaminase domain of an RNA-dependent Adenosine Deaminase (ADAR),
wherein the crRNA is capable of hybridizing with a region in the RNA transcript comprising a target A nucleotide to form an RNA duplex, wherein the duplex comprises an A-C mismatch at the target A nucleotide,
wherein the target A nucleotide is deaminated by the deaminase domain.

33. A composition or methods as described herein including but not limited to the text and figures, and including combinations thereof include that associated with the working examples.

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure.

### EXAMPLES

### Example 1: Development of a Cas13-Based, One-Pot Assay for SARS-CoV-2 Detection

This Example reports the identification and characterization of a thermostable Cas13a ortholog from Thermoclostridium caenicola (TccCas13a) that is highly active at high temperatures required for some amplification methods, such as RT-LAMP. Besides TccCas13a, other thermophilic Cas13a orthologues, including Cas13a from Herbinix hemicellulosilytica (HheCas13a) also exhibited strong activity at elevated temperatures. These Cas13s were characterized with regards to temperature, reporter cleavage preference and optimal reaction conditions. RT-LAMP utilizing a SARS-COV-2 N-gene specific primer set with FIP primer carrying a T7 promoter was coupled to in vitro transcription mediated by the thermostable Hi-T7 RNA polymerase and the reaction conditions were optimized for one-pot detection assay. The data described below demonstrates that this protocol can robustly detect SARS-COV-2 in clinical samples derived from COVID-19 patients. The data indicate that the thermophilic Cas13-based one-pot detection assay holds great promise for use as a prospective POC modality to help address increased demand for SARS-COV-2 testing.

### Materials and Methods

### Computational Identification of a Thermophilic CRISPR/Cas13a

Various existing Cas13 enzymes and their bacterial hosts were manually interrogated to identify potential thermophilic Cas13s originating from thermophilic organisms. After the identification of HheCas13a as a potential thermophilic Cas13 protein, the protein sequence of HheCas13a was used as a query in the Basic Local Alignment Search Tool (BLAST) against the NCBI non-redundant (nr) protein database using default settings. Only subject sequences with query coverage (Query cover) above 80% were considered for second round of host interrogation (analyzing growth conditions using BacDive data base and other resources). TccCas13a protein (accession #WP_149678719.1) from Thermoclostridium caenicola was identified as a potential thermophilic Cas13 protein.

Phylogenetic tree was reconstructed using protein sequences of different Cas13s belonging to different families/subtypes of Class II/type VI CRISPR-Cas systems. All protein sequences were organized in a single.txt file and aligned using MUSCLE in MEGAX software with default settings. The phylogenetic reconstruction was based on the Maximum-Likelihood method using MEGAX with WAG+G+F model and 1000 bootstrap samplings. The generated output file (.nwk) was visualized using TreeGraph_2.

CRISPRCasFinder [1] was performed on the genomic DNA sequence (GenBank #NZ_FQZP01000023.1) to identify the associated CRISPR array. CRISPRDetect [2] was then used to predict the orientation of the direct repeat in the TccCas13a CRISPR array.

### Cas13 Protein Expression and Purification

The expression vector for HheCas13a “p2CT-His-MBP-Hhe_Cas13a_WT” was obtained from Addgene (plasmid #91871), and the purification of HheCas13a was performed following a previously published protocol [10]. To produce the expression plasmid for TccCas13a expression and purification, the E. coli codon-optimized TccCas13 coding sequence was synthesized (GenScript) de novo and subcloned in frame with His and SUMO tags on the N-terminus into the His6-TwinStrep-SUMO bacterial expression vector (Addgene #115267) using BamHI and NotI (Table 4).

Purification of TccCas13a protein was performed following the protocol of Kellner et al. (2019) with a few modifications. Briefly, the TccCas13a expression vector was transformed into BL21 E. coli cells. Starter cultures were prepared by growing single colonies in LB broth supplemented with 100 g/mL ampicillin for around 12 h at 37° C. Next, 25 mL of starter culture was used to inoculate 1 L of Terrific Broth medium (TB) (IBI scientific) supplemented with 100 μg/mL ampicillin, and the 1 L cultures (4 L total) were incubated at 37° C. until an OD₆₀₀ of 0.5. Cells were incubated at 4° C. 30 mins, and the expression was induced with 0.5 mM IPTG. Cultures were then incubated at 16° C. at 180 rpm for overnight expression. Next, cells were harvested by centrifugation for 20 min at 4° C. at 4000 rpm. Cell pellets were resuspended in lysis buffer (50 mM Tris-Cl pH 7.5, 300 mM NaCl, 5% glycerol, 1 mM TCEP, 4.5 mM MgCl2, 1 mM PMSF, EDTA-free protease inhibitor (Roche)) and supplemented with 1 mg/mL lysozyme (L6876, Sigma). Cells were then lysed by sonication and clarified by centrifugation at 12,000 rpm for 60 min. The soluble 6×His-SUMO-TccCas13a in cleared lysate was then purified with an affinity chromatography column (HiTrap Q HP, 5 mL GE Healthcare) (AKTA PURE, GE Healthcare) followed by concurrent removal of the 6×His-SUMO tag by SUMO protease and overnight dialysis in dialysis buffer (50 mM Tris-HCL pH 7.5, 200 mM KCL, 5% glycerol, 1 mM TCEP). Cleaved protein was concentrated to 1.5 mL by Amicon Ultra-15 Centrifugal Filter Units (100 kDa NMWL, UFC905024, Millipore) and further purified via size-exclusion chromatography on a S200 column (GE Healthcare) in gel filtration buffer (50 mM Tris-HCl, 200 mM KCL, 10% glycerol, 1 mM TCEP, pH 7.5). The protein-containing fractions resulting from the gel filtration were pooled, snap frozen, and stored at −80° C.

### Nucleic Acid Preparation

A short region of the SARS-COV-2 N gene sequence was used as the target sequence in all preliminary thermophilic Cas13 characterization and optimization experiments to screen collateral reporters and assess thermostability of Cas13 proteins. The N gene target RNA sequence was prepared by in vitro transcription of PCR amplicons containing the T7 promoter sequence using the 2019-nCOV_N_Positive Control plasmid as a PCR template (10006625, IDT) (primers used are listed in table 2). Purified PCR amplicons (QIAquick PCR Purification Kit, QIAGEN) were transcribed in vitro using HiScribe T7 Quick High Yield RNA Synthesis Kit (E2050, NEB). The transcripts were then purified by using Direct-zol RNA Miniprep Kits (R2050, Zymo Research) following the manufacturer's instructions, and the purified RNA was stored at −80° C.

HheCas13a and TccCas13a crRNAs were designed to target the N gene sequence of the SARS-COV-2 genome. For crRNA production, templates for in vitro transcription were generated using single-stranded DNA oligos containing a T7 promoter, scaffold, and spacer in reverse complement orientation (IDT), and were then annealed to T7 forward primer in Taq DNA polymerase buffer (Invitrogen) (Table 5). The annealed oligos were then used as templates for the subsequent in vitro transcription as described above.

To establish the thermophilic Cas13-based one-pot assay, control synthetic SARS-COV-2 viral genomic sequences were ordered as synthetic RNA from Twist Bioscience, and were diluted to 10,000 RNA copies/uL and used at indicated concentrations. For RT-LAMP amplification (described below), previously published LAMP primers designed to amplify the SARS-CoV-2 N gene (Joung et al., 2020 [12], Broughton et al., 2020 [4]) were used, with the following modifications. The FIP or BIP primers were designed with the T7 promoter sequence appended at the 5′ end of the first half of the primers (Table 1).

### Differential Scanning Fluorimetry

DSF was performed using 5 to 15 uM of the purified Cas13 proteins in gel filtration buffer (with 5% glycerol) containing 10% SYPRO Orange fluorescent dye (ThermoFisher, S6650) in final reaction volume of 35 uL. Proteins were tested in triplicates and the fluorescence was monitored using a 96-well Real-Time PCR detection system (CFX96 qPCR machine, Bio-Rad), from 25 to 95° C., with a gradual temperature increase of 1° C. every 10 S.

### Protein Thermostability Assay

LwaCas13a, HheCas13a and TccCas13a proteins were diluted to approximately 0.2 mg/mL in protein storage buffer (50 mM Tris-HCL pH 7.5, 600 mM NaCl, 5% glycerol, 2 mM DTT) and incubated at a range of temperatures (37, 60, 70 and 90° C.) for 30 minutes. Samples were spun down in a microcentrifuge at 14200 rpm for 25 minutes. A total of 5 μL of the supernatant was mixed with the same volume of protein sample loading buffer and heated at 95° C. for 10 minutes. The samples were cooled on ice for 3 minutes and run on NuPAGE (10%) Bis-Tris polyacrylamide gel (ThermoFisher, NP0301BOX).

Protein thermostability assay for HheCas13a and TccCas13a RNPs was performed in the same way with the exception of incubating the aforementioned proteins with 1 μM of their cognate crRNAs for 5 minutes at 37° C. in order to assemble the RNP before subjecting them to a range of different temperatures.

### In Vitro Cis Cleavage Assays

HheCas13a and TccCas13a cleavage reactions were performed at 37° C. and 60° C. with synthetic, in vitro transcribed RNA target. Briefly, for both HheCas13a or TccCas13a cleavage assays, cleavage reactions were carried out in 20 μL reaction volume with 50 nM of Cas13a protein, 50 nM of their cognate crRNAs, and 100 nM of target RNA in 1× isothermal buffer (20 mM Tris-HCl, 50 mM KCl, 10 mM (NH₄)₂SO₄, 2 mM MgSO₄, 0.1% Tween 20, pH 8.8) supplemented with additional 6 mM MgSo4 (final of 8 mM MgSo2), and the reactions were then incubated at the indicated temperatures for 1 hr (no pre-assembly of Cas13a protein and crRNA to form RNP was performed). The samples were then boiled at 70° C. for 3 minutes in 2×RNA Loading Dye (NEB, B0363S) and cooled on ice for 3 minutes before loading into 6% polyacrylamide-urea denaturing gel. The electrophoresis was run for 45 minutes at 25W. The gel was stained with SYBR™ Gold Nucleic Acid Gel Stain (ThermoFisher, S11494) for 10 minutes, briefly washed with 1×TBE buffer and visualized using Bio-Rad Molecular Imager® Gel Doc™ system.

Fluorescent ssRNA Cleavage Assays

For all reporter screening and thermostability assays, 50 nM of HheCas13a or TccCas13a proteins were incubated with 50 nM of the respective crRNAs, 250 nM of ssRNA reporter (either poly A, poly U, or mixed sequence reporter) in 1× isothermal buffer (20 mM Tris-HCl, 50 mM KCl, 10 mM (NH₄)₂SO₄, 2 mM MgSO₄, 0.1% Tween 20, pH 8.8) supplemented with additional 6 mM MgSo4 (final of 8 mM MgSo2), 0.8 U/uL RNaseOUT (10777019, Invitrogen) and 2 μL of (1-100 nM) of target RNA in 20 uL reaction volume. These reactions were incubated in a 96 well-plate (BioRad) at different temperatures for 1 hour in a PCR (C1000 touch thermal cycler, BioRad) machine using the FAM channel, with fluorescence measurements taken every 2 min.

### One-Pot Detection Reactions

For Bst DNA polymerase screening and other optimization reactions, reverse transcription and LAMP isothermal amplification of the target nucleic acids, coupled with T7-mediated in vitro transcription and Cas13-based detection of the amplified and in vitro-transcribed target RNA was carried out in the same tube. The reaction was performed using RT-LAMP primers with final concentrations of 1.6 μM FIP/BIP primers (with the T7 promoter sequence fused to either the FIP or BIP primer), 0.2 μM F3/B3 primers, and 0.4 μM LF/LB primers, 1× Isothermal Amplification Buffer (from different vendors in Bst DNA polymerase screening reactions) or from Lucigen (30027, Lucigen) in other optimization experiments), 1.4 mM dNTPs, 0.32 U/uL Bst DNA Polymerase (from different vendors in Bst DNA polymerase screening reactions) or from Lucigen (30027, Lucigen)), 0.45 U/uL of WarmStart RTx Reverse Transcriptase (M0380, NEB) or 2 U/uL SuperScript IV reverse transcriptase (18090010, Invitrogen), 6 mM MgSO₄, 0.1 U/uL thermostable RNAseH (M0523S, NEB), 0.8 U/uL RNaseOUT or RNasin plus (N2611, Promega), 0.5 mM NTPS, 4 U/μL Hi-T7 RNA polymerase (M0658S, NEB), 0.4 U/μL thermostable inorganic pyrophosphatase (M0296, NEB), 250 nM RNA reporter, 50 nM Cas13, 50 nM crRNA, and 2 μL of template RNA in 25-μL reactions. These reactions were incubated in a 96 well-plate (BioRad) at 56° C. for 1-2 hours in a PCR (C1000 touch thermal cycler, BioRad) machine using the FAM channel, with fluorescence measurements taken every 2 min.

### Nucleotide and Amino Acid Sequences Used

TABLE 1
RT-LAMP primers used in Examples 1 and 2.

| Name | Sequence | Note |
|---|---|---|
| DE-F3 | GAAATTTGGATCTTTGTCATCC (SEQ ID NO: 1) | Original and |
| DE-B3 | GAAATTTGGATCTTTGTCATCC (SEQ ID NO: 2) | modified DETECTR |
| DE-FIP | TGCGGCCAATGTTTGTAATCAGCCAAGGAAATTT | primers. |
| | TGGGGAC (SEQ ID NO: 3) | Modified part |
| DE-BIP | CGCATTGGCATGGAAGTCACTTTGATGGCACCTG | (T7 promoter |
| | TGTAG (SEQ ID NO: 4) | sequence) shown |
| DE-LF | TTCCTTGTCTGATTAGTTC (SEQ ID NO: 5) | in bold italic. |
| DE-LB | ACCTTCGGGAACGTGGTT (SEQ ID NO: 6) | |
| DE-FIP-T7 | TGCGGCCAATGTTTGTAATCAGTAATACGACTCA | |
| | CTATAGGCCAAGGAAATTTTGGGGAC (SEQ ID | |
| | NO: 7) | |
| DE-BIP-T7 | CGCATTGGCATGGAAGTTAATACGACTCACTATA | |
| | GGCACTTTGATGGCACCTGTGTAG (SEQ ID NO: 8) | |
| SC-F3 | GCTGCTGAGGCTTCTAAG (SEQ ID NO: 9) | Original and |
| SC-B3 | GCGTCAATATGCTTATTCAGC (SEQ ID NO: 10) | modified |
| SC-FIP | GCGGCCAATGTTTGTAATCAGTAGACGTGGTCC | STOPCovid |
| | AGAACAA (SEQ ID NO: 11) | primers. |
| SC-BIP | TCAGCGTTCTTCGGAATGTCGCTGTGTAGGTCAA | Modified part |
| | CCACG (SEQ ID NO: 12) | (T7 promoter |
| SC-LF | CCTTGTCTGATTAGTTCCTGGT (SEQ ID NO: 13) | sequence) shown |
| SC-LB | TGGCATGGAAGTCACACC (SEQ ID NO: 14) | in bold italic. |
| SC-T7-FIP | GCGGCCAATGTTTGTAATCAGTTAATACGACTCA | |
| | CTATAGGGAGACGTGGTCCAGAACAA (SEQ ID | |
| | NO: 15) | |
| SC-T7-BIP | TCAGCGTTCTTCGGAATGTCGCTAATACGACTCA | |
| | CTATAGGGTGTGTAGGTCAACCACG (SEQ ID | |
| | NO: 16) | |

SEQ ID NOs: 9-16 were used in Example 1. SEQ ID NOs: 1-16 were used in Example 2.

| | | |
|---|---|---|
| RNase P-F3 | TTGATGAGCTGGAGCCA (SEQ ID NO: 71) | Original and |
| RNase P- B3 | CACCCTCAATGCAGAGTC (SEQ ID NO: 72) | modified |
| RNase P- | GTGTGACCCTGAAGACTCGGTTTTAGCCACTG | RNase P |
| FIP | ACTCGGATC (SEQ ID NO: 73) | POP primers. |
| RNase P- | CCTCCGTGATATGGCTCTTCGTTTTTTTCTTAC | Modified |
| BIP | ATGGCTCTGGTC (SEQ ID NO: 74) | part (T7 |
| RNase P-LF | ATGTGGATGGCTGAGTTGTT (SEQ ID NO: 75) | promoter |
| RNase P-LB | CATGCTGAGTACTGGACCTC (SEQ ID NO: 76) | sequence) is |
| RNase P- | GTGTGACCCTGAAGACTCGGTTTTTAATACGA | bold italic. |
| T7-FIP | CTCACTATAGGGAGCCACTGACTCGGATC | |
| | (SEQ ID NO: 77) | |
| HCV-F3 | TGTCTTCACGCAGAAAGCG (SEQ ID NO: 78) | HCV RT- |
| HCV-B3 | TACCACAAGGCCTTTCGC (SEQ ID NO: 79) | LAMP |
| HCV- | TCCGCAGACCACTATGGCTCTCTAATACGACT | primers. |
| T7-FIP | CACTATAGGGCCATGGCGTTAGTATGAGT | Modified |
| | (SEQ ID NO: 80) | part (T7 |
| HCV- | AGGACGACCGGGTCCTTTCTACTACTCGGCT | promoter |
| BIP | A GCAGTCTT (SEQ ID NO: 81) | sequence) |
| HCV-LF | GGTCCTGGAGGCTGCACGAC (SEQ ID NO: 82) | |
| HCV-LB | GAGATTTGGGCGTGCCCCCGC (SEQ ID NO: 83) | |
| TYLCV- | GGTAAAGTCTGGATGGATGA (SEQ ID NO: 84) | TYLCV |
| F3 | | LAMP |
| TYLCV- | TGTTCCTTCATTCCAGAGG (SEQ ID NO: 85) | primers. |
| B3 | | Modified |
| TYLCV- | ACCTGTCCAAAATCCATTGTAATACGACTCAC | part (T7 |
| T7-FIP | TATAGGGCAGAATCACACTAATCAGGTC (SEQ | promoter |
| | ID NO: 86) | sequence) is |
| TYLCV- | CAGTACCGCAACCGTGAAGACAATAACTGTA | underlined. |
| BIP | GCATGAAATTTCCT (SEQ ID NO: 87) | -Original |
| TYLCV- | CTATCACGGACCAAGAAGAAC (SEQ ID | primers |
| LF | NO: 88) | [Mahas, et al. |
| TYLCV- | GATTTGCGGGATAGGTTTCAAG (SEQ ID | Viruses, |
| LB | NO: 89) | 2021. 13(3)]. |

TABLE 2
Primers to PCR amplify N gene regions for IVT in Examples 1 and 2.

| | |
|---|---|
| N gene-1-T7-F | GAAATTAATACGACTCACTATAGGGAGGCTTCTAAGAAG |
| | CCTCGGC (SEQ ID NO: 17) |
| N gene-1-R_2 | CTGTCTCTGCGGTAAGGCTTGAG (SEQ ID NO: 18) |
| N gene-target | GAAATTAATACGACTCACTATAGGGTTGCAACTGAG |
| 2-T7-F | GGAGCCTTG (SEQ ID NO: 39) |
| N gene-target- | GCTTCTTAGAAGCCTCAGCAGCAG (SEQ ID NO: 40) |
| 2-R | |
| ORF1-R | GACATACTTATCGGCAATTTTGTTACC (SEQ ID |
| | NO: 195) |
| M13-R | CAGGAAACAGCTATGAC (SEQ ID NO: 196) |

TABLE 3
RNA reporter designs and sequences used in in Examples 1 and 2.

| | |
|---|---|
| UA reporter-FAM | 5′-/56-FAM/UUAUU/3IABKFQ/-3′ |
| UA reporter-HEX | 5′-/HEX/UUAUU/3IABKFQ/-3′ |
| Poly U reporter-FAM | 5′-/56-FAM/UUUUU/3IABKFQ/-3′ |
| Poly A reporter-FAM | 5′-/56-FAM/AAAAA/3IABKFQ/-3′ |
| Mix reporter-FAM | 5′-/56-FAM/UGACGU/3IABKFQ/-3′ |
| Mix reporter-HEX | 5′-/56-HEX/UGACGU/3IABKFQ/-3′ |
| Poly G reporter-FAM | 5′-/56-FAM/rGrGrGrGrGrG/3IABKFQ/-3′ |
| Poly C reporter-FAM | 5′-/56-FAM/rCrCrCrCrCrC/3IABKFQ/-3′ |
| 3(AG) reporter-FAM | 5′-/56-FAM/rArGrArGrArG/3IABKFQ/-3′ |
| 3(AC) reporter-FAM | 5′-/56-FAM/rArCrArCrArC/3IABKFQ/-3′ |
| 3(CG) reporter-FAM | 5′-/56-FAM/rCrGrCrGrCrG/3IABKFQ/-3′ |
| 3(UG) reporter-FAM | 5′-/56-FAM/rUrGrUrGrUrG/3IABKFQ/-3′ |
| LwaCas13a reporter | 5′-/56-FAM/rUrUrArUrU/3IABKFQ/-3′ |
| AapCas12b reporter- | 5′-/HEX/TTTTTTT/3IABKFQ/-3′ |
| HEX | |

TABLE 4
Cas13 protein sequence used in this study.

| Name | Protein sequence |
|---|---|
| TccCas13a | MGSSHHHHHHSSGLVPRGSHMASWSHPQFEKGGGSGGGSGGS |
| | AWSHPQFEKMSDSEVNQEAKPEVKPEVKPETHINLKVSDGSSEI |
| | FFKIKKTTPLRRLMEAFAKRQGKEMDSLRFLYDGIRIQADQTPE |
| | DLDMEDNDIIEAHREQIGGSMKITKRKWGEHHPPLYFYRDEDSG |
| | RLLAQNDRKQDYTDTLFNDIAQDTFERSLRNRLLKTPEKGDKRF |
| | YSNEIVKLVEKLCQGADVAEIMKSMERNEKLRPKNEKEIKNLK |
| | KOLDGTLSEYGKRYTAPEGAMTLNDALFYLVEGNPLKQAMAK |
| | AELGKIREALIKEKENRINRVRYSIKNNKIPLRIQEDGGITPNNDR |
| | AAWLLGLMKPADPAKGITDCYPLLGELEEVFDFDKLSKTLHEKI |
| | SRCQGRPRSIAMAVDEALKQYLRELWEKSPSRQQDLKYYFQAV |
| | QEYFKDNFPIRTKRMGARLRQELLKDKTSLSRLLEPKHMANAV |
| | RRRLINQSTQMHILYGKLYAYCCGEDGRLLVNSETLQRIQVHEA |
| | VKKQAMTAVLWSISRLRYFYQFEDGDILSNKNPIKDFRDKFLRD |
| | TNKYTHEDVEACKEKLQDFFPLKELQEKIKEDAKGLQETDNKQ |
| | ADTTDFKAIGHIVRDDRKLCNQLLAECVSCIGELRHHIFHYKNV |
| | TLIQALKRIADKVKPEDLSVLRAIYLLDRRNLKKAFAKRISSMNL |
| | PLYYREDLLSRIFKKEGTAFFLYSAKIQMTPSFQRVYERGKNLRR |
| | EFECERMKAEASNGQNGQDGDRLKWFRQLAAGDSADTHENW |
| | AVEAYAESAADVENNVEFDTDVDAQRALRNLLLLIYRHHFLPE |
| | VQKDETLVTGKIHKVLERNRQLSEGQGPNQGKAHGYSVIEELY |
| | HEGMPLSDLMKQLQRRISETERESRELAQEKTDYAQRFILDIFAE |
| | AFNDFLEAHYGEEYLEIMSPRKDAEAAKKWVKESKTVDLKTSI |
| | DEKEPEGHLLVLYPVLRLLDERELGELQQQMIRYRTSLASWQG |
| | ESNFSEEIRIAGQIEELTELVKLTEPEPQFAEEVWGKRAKEAFEDF |
| | IEGNMKNYEAFYLQSDNNTPVYRRNMSRLLRSGLMGVYQKVL |
| | ASHKQALKRDYLLWSEKHWNVKDENGADISSAEQAQCLLQRL |
| | HRKYAESPSRFTEEDCKLYEKVLRRLEDYNQAVKNLSFSSLYEI |
| | CVLNLEILSRWVGFVQDWERDMYFLLLAWVRQGKLDGIKEED |
| | VRDIFSEGNIIRNLVDTLKGENMNAFESVYFPENKGSKYLGVRN |
| | DVAHLDLMRKNGWRLEAGKTCSVMEDYINRLRFLLSYDQKRM |
| | NAVTKTLQQIFDRHKVKIRFTVEKGGMLKIEDVTADKIVHLKGS |
| | RLSGIEIPSHGERFIDTLKALMVYPRG* (SEQ ID NO: 19) |

6x His affinity tag: residues 5-10; Thrombin site: residues 14-19; Strep-tag II: residues 24-31 and 44-51; SUMO: residues 52-148; TccCas13a protein: residues 151-1375. For HheCas13 sequence (Ref [10]).

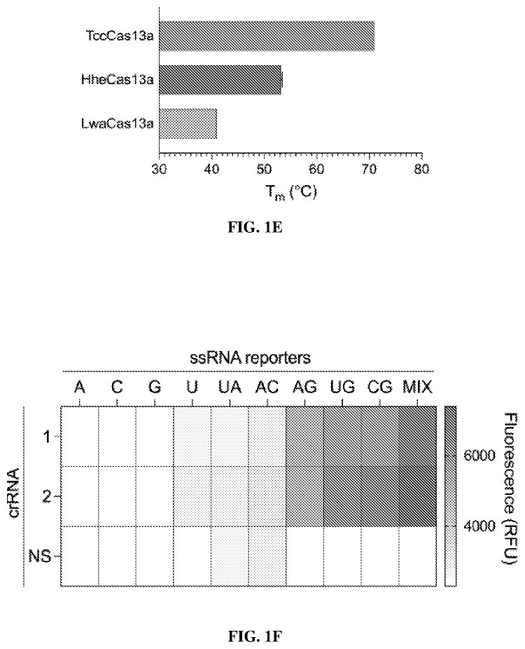

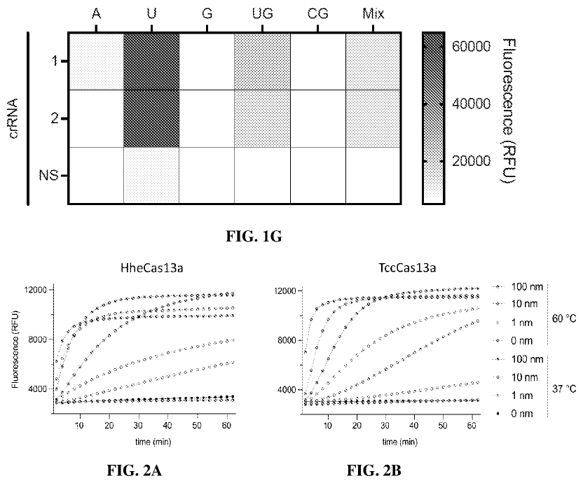

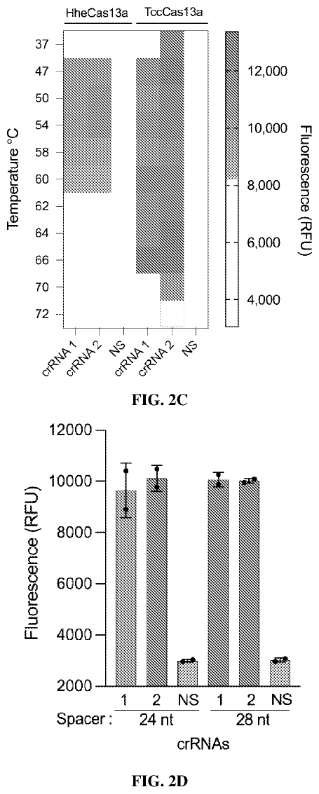

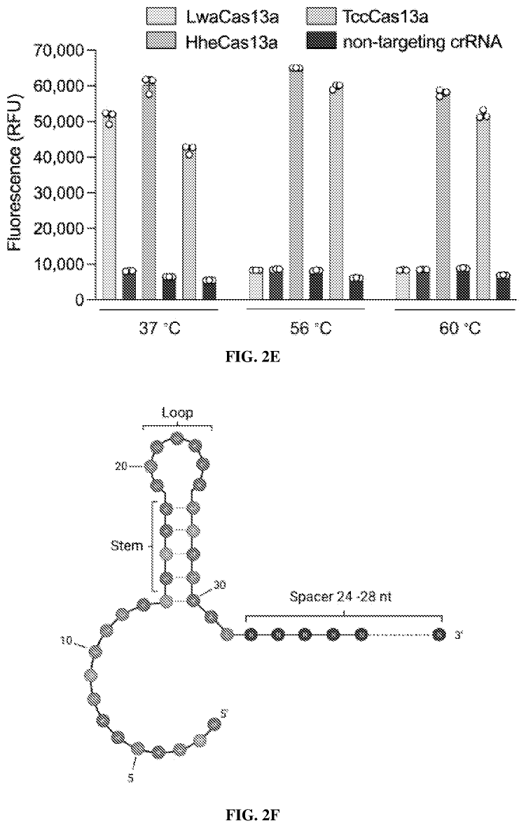

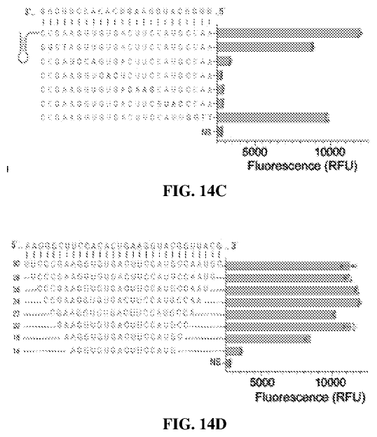

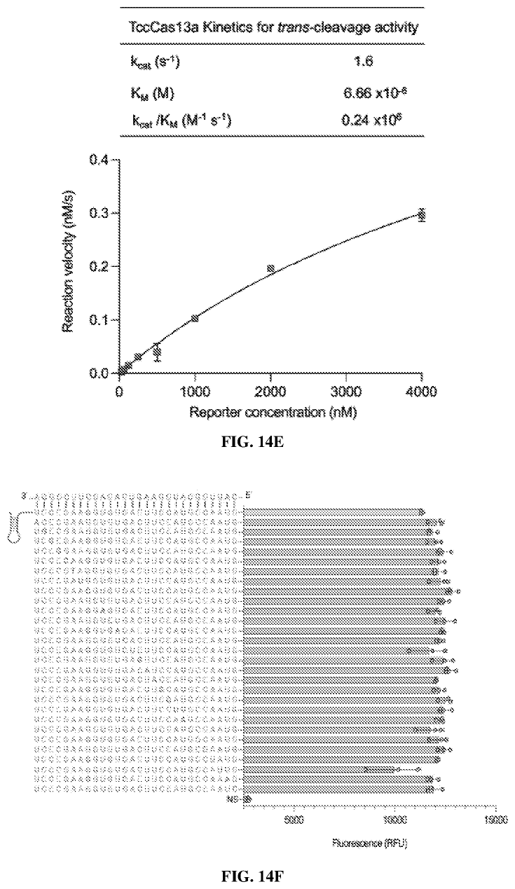

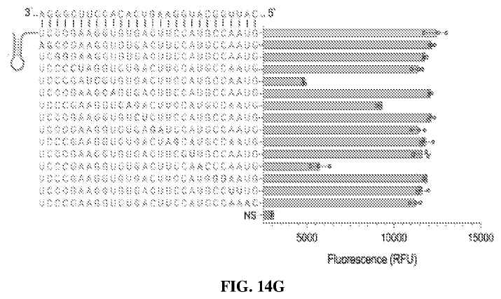

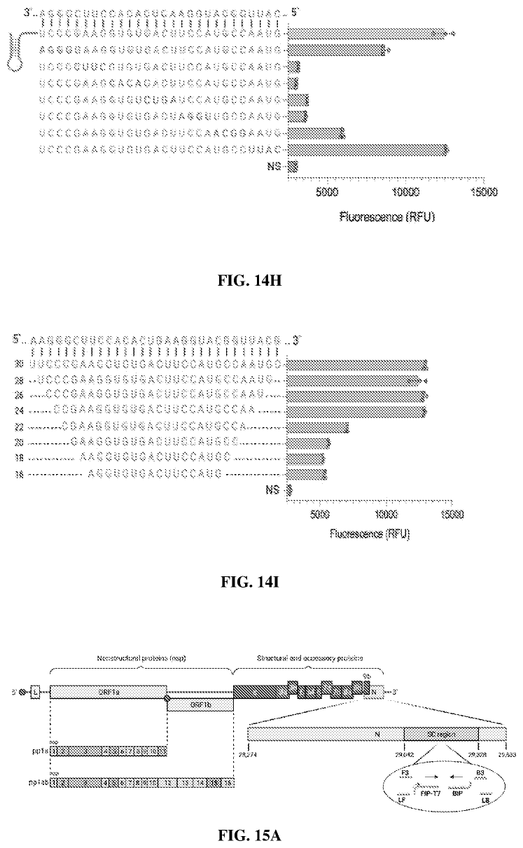

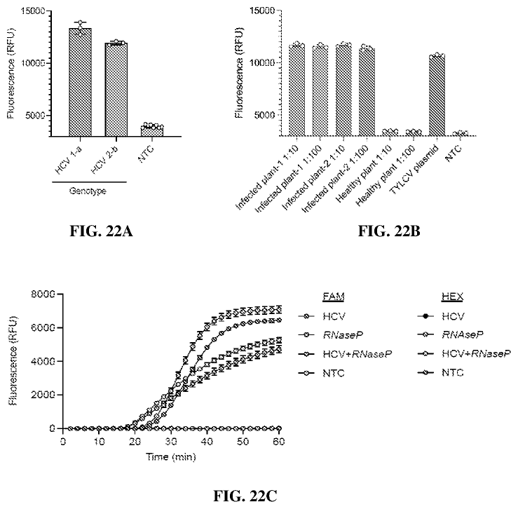

TABLE 5
crRNA sequences used in this study.

| crRNA | | | |
|---|---|---|---|
| # | crRNA Name | crRNA sequence | |
| 1101 | Hhe-Sense-28-1 | ATCAGACAAGGAACTGATTACAAACATTGTT | |
| | | GCAGTTCCCCTGTCTACGGGGATTGTTACCCT | |
| | | ATAGTGAGTCGTATTAATTTC (SEQ ID NO: 20) | |
| 1102 | Hhe-Sense-28-2 | CCGCAAATTGCACAATTTGCCCCCAGCGGTTG | |
| | | CAGTTCCCCTGTCTACGGGGATTGTTACCCTA | |
| | | TAGTGAGTCGTATTAATTTC (SEQ ID NO: 21) | |
| 1103 | Hhe-Sense-28-3 | CGCTTCAGCGTTCTTCGGAATGTCGCGCGTTG | |
| | | CAGTTCCCCTGTCTACGGGGATTGTTACCCTA | |
| | | TAGTGAGTCGTATTAATTTC (SEQ ID NO: 22) | |
| 1104 | Hhe-Sense-28-4 | CATTGGCATGGAAGTCACACCTTCGGGAGTTG | |
| | | CAGTTCCCCTGTCTACGGGGATTGTTACCCTA | |
| | | TAGTGAGTCGTATTAATTTC (SEQ ID NO: 23) | |
| 1105 | Hhe-Sense-28-5 | GAACGTGGTTGACCTACACAGGTGCCATGTTG | |
| | | CAGTTCCCCTGTCTACGGGGATTGTTACCCTA | |
| | | TAGTGAGTCGTATTAATTTC (SEQ ID NO: 24) | |
| 1106 | Hhe-Sense-28-6 | ATCAAATTGGATGACAAAGATCCAAATTGTT | |
| | | GCAGTTCCCCTGTCTACGGGGATTGTTACCCT | |
| | | ATAGTGAGTCGTATTAATTTC (SEQ ID NO: 25) | |
| NS | Hhe-NS | ctccgtgatatggctcttcgcatgctgaGTTGCAGTTCCCCTG | |
| | | TCTACGGGGATTGTTACCCTATAGTGAGTCGT | |
| | | ATTAATTTC (SEQ ID NO: 26) | |
| 1087 | Hhe-AntiSense- | TCCCGAAGGTGTGACTTCCATGCCAATGGTTG | |
| | 28-4 | CAGTTCCCCTGTCTACGGGGATTGTTACCCTA | |
| | | TAGTGAGTCGTATTAATTTC (SEQ ID NO:2 7) | |
| 1088 | Hhe-AntiSense- | AATTTGGATCTTTGTCATCCAATTTGATGTTG | |
| | 28-6 | CAGTTCCCCTGTCTACGGGGATTGTTACCCTA | |
| | | TAGTGAGTCGTATTAATTTC (SEQ ID NO: 28) | |
| 1125 | Tcc-Sense-24-1 | CAGACAAGGAACTGATTACAAACAGTTGCAG | |
| | | TCTCCGCCTACATGGGAGTTGTGACCCTATAG | |
| | | TGAGTCGTATTAATTTC (SEQ ID NO: 29) | |
| 1126 | Tcc-Sense-24-3 | CTTCAGCGTTCTTCGGAATGTCGCGTTGCAGT | |
| | | CTCCGCCTACATGGGAGTIGTGACCCTATAGT | |
| | | GAGTCGTATTAATTTC (SEQ ID NO: 30) | |
| 1127 | Tcc-Sense-24-6 | CAAATTGGATGACAAAGATCCAAAGTTGCAG | |
| | | TCTCCGCCTACATGGGAGTTGTGACCCTATAG | |
| | | TGAGTCGTATTAATTTC (SEQ ID NO: 31) | |
| 1171 | Tcc-Sense-24-2 | GCAAATTGCACAATTTGCCCCCAGGTTGCAGT | |
| | | CTCCGCCTACATGGGAGTTGTGACCCTATAGT | |
| | | GAGTCGTATTAATTTC (SEQ ID NO: 32) | |
| 1172 | Tcc-Sense-24-4 | TTGGCATGGAAGTCACACCTTCGGGTTGCAGT | |
| | | CTCCGCCTACATGGGAGTIGTGACCCTATAGT | |
| | | GAGTCGTATTAATTTC (SEQ ID NO: 33) | |
| 1173 | Tcc-Sense-24-5 | ACGTGGTTGACCTACACAGGTGCCGTTGCAGT | |
| | | CTCCGCCTACATGGGAGTIGTGACCCTATAGT | |
| | | GAGTCGTATTAATTTC (SEQ ID NO: 34) | |
| NS | Tcc-NS | actcagccatccacatccgagtcttcagGTTGCAGTCTCCGCC | |
| | | TACATGGGAGTTGTGACCCTATAGTGAGTCGT | |
| | | ATTAATTTC (SEQ ID NO: 35) | |
| 1174 | Tcc-AntiSense- | TGTTTGTAATCAGTTCCTTGTCTGGTTGCAGTC | |
| | 24-1 | TCCGCCTACATGGGAGTIGTGACCCTATAGTG | |
| | | AGTCGTATTAATTTC (SEQ ID NO: 36) | |
| 1175 | Tcc-AntiSense- | GCGACATTCCGAAGAACGCTGAAGGTTGCAG | |
| | 24-3 | TCTCCGCCTACATGGGAGTTGTGACCCTATAG | |
| | | TGAGTCGTATTAATTTC (SEQ ID NO: 37) | |
| T7 | T7 oligo for IVT | GAAATTAATACGACTCACTATAGGG (SEQ ID | |
| oligo | | NO: 38) | |
| 1243 | Tcc-RNase P-24- | TCAGCCATCCACATCCGAGTCTTCG | (crRNA1) |
| | 1 | TTGCAGTCTCCGCCTACATGGGAG | Selected |
| | | TTGTGACCCTATAGTGAGTCGTATT | crRNA for |
| | | AATTTC (SEQ ID NO: 197) | detection |
| 1244 | Tcc-RNase P-24- | CCGTGATATGGCTCTTCGCATGCTG | (crRNA2) |
| | 2 | TTGCAGTCTCCGCCTACATGGGAG | |
| | | TTGTGACCCTATAGTGAGTCGTATT | |
| | | AATTTC (SEQ ID NO: 198) | |
| 1246 | 1104-Mis 1-1 | GATTGGCATGGAAGTCACACCTTC | FIGS. 14F-14I |
| | | GGGAGTTGCAGTTCCCCTGTCTAC | |
| | | GGGGATTGTTACCCTATAGTGAGT | |
| | | CGTATTAATTTC (SEQ ID NO: 199) | |
| 1247 | 1104-Mis 1-2 | CTTTGGCATGGAAGTCACACCTTC | FIGS. 14F-14I |
| | | GGGAGTTGCAGTTCCCCTGTCTAC | |
| | | GGGGATTGTTACCCTATAGTGAGT | |
| | | CGTATTAATTTC (SEQ ID NO: 200) | |
| 1248 | 1104-Mis 1-3 | CAATGGCATGGAAGTCACACCTTC | FIGS. 14F-14I |
| | | GGGAGTTGCAGTTCCCCTGTCTAC | |
| | | GGGGATTGTTACCCTATAGTGAGT | |
| | | CGTATTAATTTC (SEQ ID NO: 201) | |
| 1249 | 1104-Mis 1-4 | CATAGGCATGGAAGTCACACCTTC | FIGS. 14F-14I |
| | | GGGAGTTGCAGTTCCCCTGTCTAC | |
| | | GGGGATTGTTACCCTATAGTGAGT | |
| | | CGTATTAATTTC (SEQ ID NO: 202) | |
| 1250 | 1104-Mis 1-5 | CATTCGCATGGAAGTCACACCTTC | FIGS. 14F-14I |
| | | GGGAGTTGCAGTTCCCCTGTCTAC | |
| | | GGGGATTGTTACCCTATAGTGAGT | |
| | | CGTATTAATTTC (SEQ ID NO: 203) | |
| 1251 | 1104-Mis 1- | CATTGCCATGGAAGTCACACCTTC | FIGS. 14F-14I |
| | 6 | GGGAGTTGCAGTTCCCCTGTCTAC | |
| | | GGGGATTGTTACCCTATAGTGAGT | |
| | | CGTATTAATTTC (SEQ ID NO: 204) | |
| 1252 | 1104-Mis 1- | CATTGGGATGGAAGTCACACCTTCG | |
| | 7 | GGAGTTGCAGTTCCCCTGTCTACGG | FIGS. 14F- |
| | | GGATTGTTACCCTATAGTGAGTCGT | 14I |
| | | ATTAATTTC (SEQ ID NO: 205) | |
| 1253 | 1104-Mis 1- | CATTGGCTTGGAAGTCACACCTTCGG | FIGS. 14F- |
| | 8 | GAGTTGCAGTTCCCCTGTCTACGGGG | 14I |
| | | ATTGTTACCCTATAGTGAGTCGTATT | |
| | | AATTTC (SEQ ID NO: 206) | |
| 1254 | 1104-Mis 1- | CATTGGCAAGGAAGTCACACCTTCG | FIGS. 14F- |
| | 9 | GGAGTTGCAGTTCCCCTGTCTACGG | 14I |
| | | GGATTGTTACCCTATAGTGAGTCGT | |
| | | ATTAATTTC (SEQ ID NO: 207) | |
| 1255 | 1104-Mis 1- | CATTGGCATCGAAGTCACACCTTCG | FIGS. 14F- |
| | 10 | GGAGTTGCAGTTCCCCTGTCTACGG | 14I |
| | | GGATTGTTACCCTATAGTGAGTCGT | |
| | | ATTAATTTC (SEQ ID NO: 208) | |
| 1256 | 1104-Mis 1- | CATTGGCATGCAAGTCACACCTTCG | FIGS. 14F- |
| | 11 | GGAGTTGCAGTTCCCCTGTCTACGG | 14I |
| | | GGATTGTTACCCTATAGTGAGTCGT | |
| | | ATTAATTTC (SEQ ID NO: 209) | |
| 1257 | 1104-Mis 1- | CATTGGCATGGTAGTCACACCTTCG | FIGS. 14F- |
| | 12 | GGAGTTGCAGTTCCCCTGTCTACGG | 14I |
| | | GGATTGTTACCCTATAGTGAGTCGT | |
| | | ATTAATTTC (SEQ ID NO: 210) | |
| 1258 | 1104-Mis 1- | CATTGGCATGGATGTCACACCTTCG | FIGS. 14F- |
| | 13 | GGAGTTGCAGTTCCCCTGTCTACGG | 14I |
| | | GGATTGTTACCCTATAGTGAGTCGT | |
| | | ATTAATTTC (SEQ ID NO: 211) | |
| 1259 | 1104-Mis 1- | CATTGGCATGGAACTCACACCTTCG | FIGS. 14F- |
| | 14 | GGAGTTGCAGTTCCCCTGTCTACGG | 14I |
| | | GGATTGTTACCCTATAGTGAGTCGT | |
| | | ATTAATTTC (SEQ ID NO: 212) | |
| 1260 | 1104-Mis 1- | CATTGGCATGGAAGACACACCTTC | FIGS. 14F- |
| | 15 | GGGAGTTGCAGTTCCCCTGTCTACG | 14I |
| | | GGGATTGTTACCCTATAGTGAGTCG | |
| | | TATTAATTTC (SEQ ID NO: 213) | |
| 1261 | 1104-Mis 1- | CATTGGCATGGAAGTGACACCTTC | FIGS. 14F- |
| | 16 | GGGAGTTGCAGTTCCCCTGTCTACG | 14I |
| | | GGGATTGTTACCCTATAGTGAGTCG | |
| | | TATTAATTTC (SEQ ID NO: 214) | |
| 1262 | 1104-Mis 1- | CATTGGCATGGAAGTCTCACCTTCG | FIGS. 14F- |
| | 17 | GGAGTTGCAGTTCCCCTGTCTACGG | 14I |
| | | GGATTGTTACCCTATAGTGAGTCGT | |
| | | ATTAATTTC (SEQ ID NO: 215) | |
| 1263 | 1104-Mis 1- | CATTGGCATGGAAGTCAGACCTTC | FIGS. 14F- |
| | 18 | GGGAGTTGCAGTTCCCCTGTCTACG | 14I |
| | | GGGATTGTTACCCTATAGTGAGTCG | |
| | | TATTAATTTC (SEQ ID NO: 90) | |
| 1264 | 1104-Mis 1- | CATTGGCATGGAAGTCACTCCTTCG | FIGS. 14F- |
| | 19 | GGAGTTGCAGTTCCCCTGTCTACGG | 14I |
| | | GGATTGTTACCCTATAGTGAGTCGT | |
| | | ATTAATTTC (SEQ ID NO: 91) | |
| 1265 | 1104-Mis 1- | CATTGGCATGGAAGTCACAGCTTC | FIGS. 14F- |
| | 20 | GGGAGTTGCAGTTCCCCTGTCTACG | 14I |
| | | GGGATTGTTACCCTATAGTGAGTCG | |
| | | TATTAATTTC (SEQ ID NO: 92) | |
| 1266 | 1104-Mis 1- | CATTGGCATGGAAGTCACACGTTC | FIGS. 14F- |
| | 21 | GGGAGTTGCAGTTCCCCTGTCTACG | 14I |
| | | GGGATTGTTACCCTATAGTGAGTCG | |
| | | TATTAATTTC (SEQ ID NO: 93) | |
| 1267 | 1104-Mis 1- | CATTGGCATGGAAGTCACACCATC | FIGS. 14F- |
| | 22 | GGGAGTTGCAGTTCCCCTGTCTACG | 14I |
| | | GGGATTGTTACCCTATAGTGAGTCG | |
| | | TATTAATTTC (SEQ ID NO: 94) | |
| 1268 | 1104-Mis 1- | CATTGGCATGGAAGTCACACCTACG | FIGS. 14F- |
| | 23 | GGAGTTGCAGTTCCCCTGTCTACGG | 14I |
| | | GGATTGTTACCCTATAGTGAGTCGT | |
| | | ATTAATTTC (SEQ ID NO: 95) | |
| 1269 | 1104-Mis 1- | CATTGGCATGGAAGTCACACCTTGG | FIGS. 14F- |
| | 24 | GGAGTTGCAGTTCCCCTGTCTACGG | 14I |
| | | GGATTGTTACCCTATAGTGAGTCGT | |
| | | ATTAATTTC (SEQ ID NO: 96) | |
| 1270 | 1104-Mis 1- | CATTGGCATGGAAGTCACACCTTCC | FIGS. 14F- |
| | 25 | GGAGTTGCAGTTCCCCTGTCTACGG | 14I |
| | | GGATTGTTACCCTATAGTGAGTCGT | |
| | | ATTAATTTC (SEQ ID NO: 97) | |
| 1271 | 1104-Mis 1- | CATTGGCATGGAAGTCACACCTTCG | FIGS. 14F- |
| | 26 | CGAGTTGCAGTTCCCCTGTCTACGG | 14I |
| | | GGATTGTTACCCTATAGTGAGTCGT | |
| | | ATTAATTTC (SEQ ID NO: 98) | |
| 1272 | 1104-Mis 1- | CATTGGCATGGAAGTCACACCTTCG | FIGS. 14F- |
| | 27 | GCAGTTGCAGTTCCCCTGTCTACGG | 14I |
| | | GGATTGTTACCCTATAGTGAGTCGT | |
| | | ATTAATTTC (SEQ ID NO: 99) | |
| 1273 | 1104-Mis 1- | CATTGGCATGGAAGTCACACCTTCG | FIGS. 14F- |
| | 28 | GGTGTTGCAGTTCCCCTGTCTACGG | 14I |
| | | GGATTGTTACCCTATAGTGAGTCGT | |
| | | ATTAATTTC (SEQ ID NO: 100) | |
| 1274 | 1104-Mis- | GTTTGGCATGGAAGTCACACCTTCG | FIGS. 14F- |
| | 2-1 | GGAGTTGCAGTTCCCCTGTCTACGG | 14I |
| | | GGATTGTTACCCTATAGTGAGTCGT | |
| | | ATTAATTTC (SEQ ID NO: 101) | |
| 1275 | 1104-Mis- | CAAAGGCATGGAAGTCACACCTTCG | FIGS. 14F- |
| | 2-2 | GGAGTTGCAGTTCCCCTGTCTACGG | 14I |
| | | GGATTGTTACCCTATAGTGAGTCGT | |
| | | ATTAATTTC (SEQ ID NO: 102) | |
| 1276 | 1104-Mis- | CATTCCCATGGAAGTCACACCTTCG | FIGS. 14F- |
| | 2-3 | GGAGTTGCAGTTCCCCTGTCTACGG | 14I |
| | | GGATTGTTACCCTATAGTGAGTCGT | |
| | | ATTAATTTC (SEQ ID NO: 103) | |
| 1277 | 1104-Mis- | CATTGGGTTGGAAGTCACACCTTCG | FIGS. 14F- |
| | 2-4 | GGAGTTGCAGTTCCCCTGTCTACGG | 14I |
| | | GGATTGTTACCCTATAGTGAGTCGT | |
| | | ATTAATTTC (SEQ ID NO: 104) | |
| 1278 | 1104-Mis- | CATTGGCAACGAAGTCACACCTTCG | FIGS. 14F- |
| | 2-5 | GGAGTTGCAGTTCCCCTGTCTACGG | 14I |
| | | GGATTGTTACCCTATAGTGAGTCGT | |
| | | ATTAATTTC (SEQ ID NO: 105) | |
| 1279 | 1104-Mis- | CATTGGCATGCTAGTCACACCTTCG | FIGS. 14F- |
| | 2-6 | GGAGTTGCAGTTCCCCTGTCTACGG | 14I |
| | | GGATTGTTACCCTATAGTGAGTCGT | |
| | | ATTAATTTC (SEQ ID NO: 106) | |
| 1280 | 1104-Mis- | CATTGGCATGGATCTCACACCTTCG | FIGS. 14F- |
| | 2-7 | GGAGTTGCAGTTCCCCTGTCTACGG | 14I |
| | | GGATTGTTACCCTATAGTGAGTCGT | |
| | | ATTAATTTC (SEQ ID NO: 107) | |
| 1281 | 1104-Mis- | CATTGGCATGGAAGAGACACCTTCG | FIGS. 14F- |
| | 2-8 | GGAGTTGCAGTTCCCCTGTCTACGG | 14I |
| | | GGATTGTTACCCTATAGTGAGTCGT | |
| | | ATTAATTTC (SEQ ID NO: 108) | |
| 1282 | 1104-Mis- | CATTGGCATGGAAGTCTGACCTTCG | FIGS. 14F- |
| | 2-9 | GGAGTTGCAGTTCCCCTGTCTACGG | 14I |
| | | GGATTGTTACCCTATAGTGAGTCGT | |
| | | ATTAATTTC (SEQ ID NO: 109) | |
| 1283 | 1104-Mis- | CATTGGCATGGAAGTCACTGCTTCG | FIGS. 14F- |
| | 2-10 | GGAGTTGCAGTTCCCCTGTCTACGG | 14I |
| | | GGATTGTTACCCTATAGTGAGTCGT | |
| | | ATTAATTTC (SEQ ID NO: 110) | |
| 1284 | 1104-Mis- | CATTGGCATGGAAGTCACACGATCG | FIGS. 14F- |
| | 2-11 | GGAGTTGCAGTTCCCCTGTCTACGG | 14I |
| | | GGATTGTTACCCTATAGTGAGTCGT | |
| | | ATTAATTTC (SEQ ID NO: 111) | |
| 1285 | 1104-Mis- | CATTGGCATGGAAGTCACACCTAGG | FIGS. 14F- |
| | 2-12 | GGAGTTGCAGTTCCCCTGTCTACGG | 14] |
| | | GGATTGTTACCCTATAGTGAGTCGT | |
| | | ATTAATTTC (SEQ ID NO: 112) | |
| 1286 | 1104-Mis- | CATTGGCATGGAAGTCACACCTTCC | FIGS. 14F- |
| | 2-13 | CGAGTTGCAGTTCCCCTGTCTACGG | 14I |
| | | GGATTGTTACCCTATAGTGAGTCGT | |
| | | ATTAATTTC (SEQ ID NO: 113) | |
| 1287 | 1104-Mis- | CATTGGCATGGAAGTCACACCTTCG | FIGS. 14F- |
| | 2-14 | GCTGTTGCAGTTCCCCTGTCTACGGG | 14I |
| | | GATTGTTACCCTATAGTGAGTCGTAT | |
| | | TAATTTC (SEQ ID NO: 114) | |
| 1288 | 1104-Seed- | GTAAGGCATGGAAGTCACACCTTCG | FIGS. 14F- |
| | 1 | GGAGTTGCAGTTCCCCTGTCTACGG | 14I |
| | | GGATTGTTACCCTATAGTGAGTCGT | |
| | | ATTAATTTC (SEQ ID NO: 115) | |
| 1289 | 1104-Seed- | CATTCCGTTGGAAGTCACACCTTCG | FIGS. 14F- |
| | 2 | GGAGTTGCAGTTCCCCTGTCTACGG | 14I |
| | | GGATTGTTACCCTATAGTGAGTCGT | |
| | | ATTAATTTC (SEQ ID NO: 116) | |
| 1290 | 1104-Seed- | CATTGGCAACCTAGTCACACCTTCG | FIGS. 14F- |
| | 3 | GGAGTTGCAGTTCCCCTGTCTACGG | 14I |
| | | GGATTGTTACCCTATAGTGAGTCGT | |
| | | ATTAATTTC (SEQ ID NO: 117) | |
| 1291 | 1104-Seed- | CATTGGCATGGATCAGACACCTTCG | FIGS. 14F- |
| | 4 | GGAGTTGCAGTTCCCCTGTCTACGG | 14I |
| | | GGATTGTTACCCTATAGTGAGTCGT | |
| | | ATTAATTTC (SEQ ID NO:118) | |
| 1292 | 1104-Seed- | CATTGGCATGGAAGTCTGTGCTTCG | FIGS. 14F- |
| | 5 | GGAGTTGCAGTTCCCCTGTCTACGG | 14I |
| | | GGATTGTTACCCTATAGTGAGTCGT | |
| | | ATTAATTTC (SEQ ID NO: 119) | |
| 1293 | 1104-Seed- | CATTGGCATGGAAGTCACACGAAGG | FIGS. 14F- |
| | 6 | GGAGTTGCAGTTCCCCTGTCTACGG | 14I |
| | | GGATTGTTACCCTATAGTGAGTCGT | |
| | | ATTAATTTC (SEQ ID NO: 120) | |
| 1294 | 1104-Seed- | CATTGGCATGGAAGTCACACCTTCC | FIGS. 14F- |
| | 7 | CCTGTTGCAGTTCCCCTGTCTACGG | 14I |
| | | GGATTGTTACCCTATAGTGAGTCGT | |
| | | ATTAATTTC (SEQ ID NO: 121) | |
| 1295 | 1104- | CATGGAAGTCACACCTGTTGCAGTT | FIGS. 14F- |
| | Spacer-16 | CCCCTGTCTACGGGGATTGTTACCCT | 14I |
| | | ATAGTGAGTCGTATTAATTTC (SEQ | |
| | | ID NO: 122) | |
| 1296 | 1104- | GCATGGAAGTCACACCTTGTTGCAG | FIGS. 14F- |
| | Spacer-18 | TTCCCCTGTCTACGGGGATTGTTACC | 14I |
| | | CTATAGTGAGTCGTATTAATTTC | |
| | | (SEQ ID NO: 123) | |
| 1297 | 1104- | GGCATGGAAGTCACACCTTCGTTGC | FIGS. 14F- |
| | Spacer-20 | AGTTCCCCTGTCTACGGGGATTGTTA | 14I |
| | | CCCTATAGTGAGTCGTATTAATTTC | |
| | | (SEQ ID NO: 124) | |
| 1298 | 1104- | TGGCATGGAAGTCACACCTTCGGTT | FIGS. 14F- |
| | Spacer-22 | GCAGTTCCCCTGTCTACGGGGATTG | 14I |
| | | TTACCCTATAGTGAGTCGTATTAAT | |
| | | TTC (SEQ ID NO: 125) | |
| 1299 | 1104- | TTGGCATGGAAGTCACACCTTCGGGT | FIGS. 14F- |
| | Spacer-24 | TGCAGTTCCCCTGTCTACGGGGATTG | 14I |
| | | TTACCCTATAGTGAGTCGTATTAATT | |
| | | TC (SEQ ID NO: 126) | |
| 1300 | 1104- | ATTGGCATGGAAGTCACACCTTCGGG | FIGS. 14F- |
| | Spacer-26 | GTTGCAGTTCCCCTGTCTACGGGGAT | 14I |
| | | TGTTACCCTATAGTGAGTCGTATTAA | |
| | | TTTC (SEQ ID NO: 127) | |
| 1301 | 1104- | gcattggcatggaagtcacaccttcgggaaGTTGCA | FIGS. 14F- |
| | Spacer-30 | GTTCCCCTGTCTACGGGGATTGTTA | 14I |
| | | CCCTATAGTGAGTCGTATTAATTTC | |
| | | (SEQ ID NO: 128) | |
| 1303 | 1172-Mis 1- | ATGGCATGGAAGTCACACCTTCGGGT | FIG. 14A |
| | 1 | TGCAGTCTCCGCCTACATGGGAGTTG | |
| | | TGACCCTATAGTGAGTCGTATTAATT | |
| | | TC (SEQ ID NO: 129) | |
| 1304 | 1172-Mis 1- | TAGGCATGGAAGTCACACCTTCGGGT | FIG. 14A |
| | 2 | TGCAGTCTCCGCCTACATGGGAGTTG | |
| | | TGACCCTATAGTGAGTCGTATTAATT | |
| | | TC (SEQ ID NO: 130) | |
| 1305 | 1172-Mis 1- | TTCGCATGGAAGTCACACCTTCGGG | FIG. 14A |
| | 3 | TTGCAGTCTCCGCCTACATGGGAGT | |
| | | TGTGACCCTATAGTGAGTCGTATTA | |
| | | ATTTC (SEQ ID NO: 131) | |
| 1306 | 1172-Mis 1- | TTGCCATGGAAGTCACACCTTCGGG | FIG. 14A |
| | 4 | TTGCAGTCTCCGCCTACATGGGAGT | |
| | | TGTGACCCTATAGTGAGTCGTATTA | |
| | | ATTTC (SEQ ID NO: 132) | |
| 1307 | 1172-Mis 1- | TTGGGATGGAAGTCACACCTTCGGGT | FIG. 14A |
| | 5 | TGCAGTCTCCGCCTACATGGGAGTTG | |
| | | TGACCCTATAGTGAGTCGTATTAATT | |
| | | TC (SEQ ID NO: 134) | |
| 1308 | 1172-Mis 1- | TTGGCTTGGAAGTCACACCTTCGGG | FIG. 14A |
| | 6 | TTGCAGTCTCCGCCTACATGGGAGT | |
| | | TGTGACCCTATAGTGAGTCGTATTA | |
| | | ATTTC (SEQ ID NO: 135) | |
| 1309 | 1172-Mis 1- | TTGGCAAGGAAGTCACACCTTCGGGT | FIG. 14A |
| | 7 | TGCAGTCTCCGCCTACATGGGAGTTG | |
| | | TGACCCT | |
| | | ATAGTGAGTCGTATTAATTTC (SEQ ID | |
| | | NO: 136) | |
| 1310 | 1172-Mis 1- | TTGGCATCGAAGTCACACCTTCGGG | FIG. 14A |
| | 8 | TTGCAGTCTCCGCCTACATGGGAGT | |
| | | TGTGACCCTATAGTGAGTCGTATTA | |
| | | ATTTC (SEQ ID NO: 137) | |
| 1311 | 1172-Mis 1- | TTGGCATGCAAGTCACACCTTCGGG | FIG. 14A |
| | 9 | TTGCAGTCTCCGCCTACATGGGAGT | |
| | | TGTGACCCTATAGTGAGTCGTATTA | |
| | | ATTTC (SEQ ID NO: 138) | |
| 1312 | 1172-Mis 1- | TTGGCATGGTAGTCACACCTTCGGG | FIG. 14A |
| | 10 | TTGCAGTCTCCGCCTACATGGGAGT | |
| | | TGTGACCCTATAGTGAGTCGTATTA | |
| | | ATTTC (SEQ ID NO: 139) | |
| 1313 | 1172-Mis 1- | TTGGCATGGATGTCACACCTTCGGG | FIG. 14A |
| | 11 | TTGCAGTCTCCGCCTACATGGGAGT | |
| | | TGTGACCCTATAGTGAGTCGTATTA | |
| | | ATTTC (SEQ ID NO: 140) | |
| 1314 | 1172-Mis 1- | TTGGCATGGAACTCACACCTTCGGG | FIG. 14A |
| | 12 | TTGCAGTCTCCGCCTACATGGGAGT | |
| | | TGTGACCCTATAGTGAGTCGTATTA | |
| | | ATTTC (SEQ ID NO: 141) | |
| 1315 | 1172-Mis 1- | TTGGCATGGAAGACACACCTTCGGGT | FIG. 14A |
| | 13 | TGCAGTCTCCGCCTACATGGGAGTTG | |
| | | TGACCCTATAGTGAGTCGTATTAATT | |
| | | TC (SEQ ID NO: 142) | |
| 1316 | 1172-Mis 1- | TTGGCATGGAAGTGACACCTTCGGGT | FIG. 14A |
| | 14 | TGCAGTCTCCGCCTACATGGGAGTTG | |
| | | TGACCCTATAGTGAGTCGTATTAATT | |
| | | TC (SEQ ID NO: 143) | |
| 1317 | 1172-Mis 1- | TTGGCATGGAAGTCTCACCTTCGGG | FIG. 14A |
| | 15 | TTGCAGTCTCCGCCTACATGGGAGT | |
| | | TGTGACCCTATAGTGAGTCGTATTA | |
| | | ATTTC (SEQ ID NO: 144) | |
| 1318 | 1172-Mis 1- | TTGGCATGGAAGTCAGACCTTCGGGT | FIG. 14A |
| | 16 | TGCAGTCTCCGCCTACATGGGAGTTG | |
| | | TGACCCTATAGTGAGTCGTATTAATT | |
| | | TC (SEQ ID NO: 145) | |
| 1319 | 1172-Mis 1- | TTGGCATGGAAGTCACTCCTTCGGG | FIG. 14A |
| | 17 | TTGCAGTCTCCGCCTACATGGGAGT | |
| | | TGTGACCCTATAGTGAGTCGTATTA | |
| | | ATTTC (SEQ ID NO: 146) | |
| 1320 | 1172-Mis 1- | TTGGCATGGAAGTCACAGCTTCGGGT | FIG. 14A |
| | 18 | TGCAGTCTCCGCCTACATGGGAGTTG | |
| | | TGACCCTATAGTGAGTCGTATTAATT | |
| | | TC (SEQ ID NO: 147) | |
| 1321 | 1172-Mis 1- | TTGGCATGGAAGTCACACGTTCGGGT | FIG. 14A |
| | 19 | TGCAGTCTCCGCCTACATGGGAGTTG | |
| | | TGACCCTATAGTGAGTCGTATTAATT | |
| | | TC (SEQ ID NO: 148) | |
| 1322 | 1172-Mis 1- | TTGGCATGGAAGTCACACCATCGGGT | FIG. 14A |
| | 20 | TGCAGTCTCCGCCTACATGGGAGTTG | |
| | | TGACCCTATAGTGAGTCGTATTAATT | |
| | | TC (SEQ ID NO: 149) | |
| 1323 | 1172-Mis 1- | TTGGCATGGAAGTCACACCTACGGGT | FIG. 14A |
| | 21 | TGCAGTCTCCGCCTACATGGGAGTTG | |
| | | TGACCCTATAGTGAGTCGTATTAATT | |
| | | TC (SEQ ID NO: 150) | |
| 1324 | 1172-Mis 1- | TTGGCATGGAAGTCACACCTTGGGGT | FIG. 14A |
| | 22 | TGCAGTCTCCGCCTACATGGGAGTTG | |
| | | TGACCCTATAGTGAGTCGTATTAATT | |
| | | TC (SEQ ID NO: 151) | |
| 1325 | 1172-Mis 1- | TTGGCATGGAAGTCACACCTTCCGG | FIG. 14A |
| | 23 | TTGCAGTCTCCGCCTACATGGGAGT | |
| | | TGTGACCCTATAGTGAGTCGTATTA | |
| | | ATTTC (SEQ ID NO: 152) | |
| 1326 | 1172-Mis 1- | TTGGCATGGAAGTCACACCTTCGCG | FIG. 14A |
| | 24 | TTGCAGTCTCCGCCTACATGGGAGT | |
| | | TGTGACCCTATAGTGAGTCGTATTA | |
| | | ATTTC (SEQ ID NO: 153) | |
| 1327 | 1172-Mis- | AAGGCATGGAAGTCACACCTTCGGGT | FIG. 14B |
| | 2-1 | TGCAGTCTCCGCCTACATGGGAGTTG | |
| | | TGACCCTATAGTGAGTCGTATTAATT | |
| | | TC (SEQ ID NO: 154) | |
| 1328 | 1172-Mis- | TTCCCATGGAAGTCACACCTTCGGG | FIG. 14B |
| | 2-2 | TTGCAGTCTCCGCCTACATGGGAGT | |
| | | TGTGACCCTATAGTGAGTCGTATTA | |
| | | ATTTC (SEQ ID NO: 155) | |
| 1329 | 1172-Mis- | TTGGGTTGGAAGTCACACCTTCGGG | FIG. 14B |
| | 2-3 | TTGCAGTCTCCGCCTACATGGGAGT | |
| | | TGTGACCCTATAGTGAGTCGTATTA | |
| | | ATTTC (SEQ ID NO: 156) | |
| 1330 | 1172-Mis- | TTGGCAACGAAGTCACACCTTCGGGT | FIG. 14B |
| | 2-4 | TGCAGTCTCCGCCTACATGGGAGTTGT | |
| | | GACCCTATAGTGAGTCGTATTAATTTC | |
| | | (SEQ ID NO: 157) | |
| 1331 | 1172-Mis- | TTGGCATGCTAGTCACACCTTCGGGT | FIG. 14B |
| | 2-5 | TGCAGTCTCCGCCTACATGGGAGTTG | |
| | | TGACCCTATAGTGAGTCGTATTAATT | |
| | | TC (SEQ ID NO: 158) | |
| 1332 | 1172-Mis- | TTGGCATGGATCTCACACCTTCGGGT | FIG. 14B |
| | 2-6 | TGCAGTCTCCGCCTACATGGGAGTTG | |
| | | TGACCCTATAGTGAGTCGTATTAATT | |
| | | TC (SEQ ID NO: 159) | |
| 1333 | 1172-Mis- | TTGGCATGGAAGAGACACCTTCGGGT | FIG. 14B |
| | 2-7 | TGCAGTCTCCGCCTACATGGGAGTIGT | |
| | | GACCCTATAGTGAGTCGTATTAATTTC | |
| | | (SEQ ID NO: 160) | |
| 1334 | 1172-Mis- | TTGGCATGGAAGTCTGACCTTCGGGT | FIG. 14B |
| | 2-8 | TGCAGTCTCCGCCTACATGGGAGTTG | |
| | | TGACCCTATAGTGAGTCGTATTAATT | |
| | | TC (SEQ ID NO: 161) | |
| 1335 | 1172-Mis- | TTGGCATGGAAGTCACTGCTTCGGGT | FIG. 14B |
| | 2-9 | TGCAGTCTCCGCCTACATGGGAGTTG | |
| | | TGACCCTATAGTGAGTCGTATTAATT | |
| | | TC (SEQ ID NO: 162) | |
| 1336 | 1172-Mis- | TTGGCATGGAAGTCACACGATCGGGT | FIG. 14B |
| | 2-10 | TGCAGTCTCCGCCTACATGGGAGTTGT | |
| | | GACCCTATAGTGAGTCGTATTAATTTC | |
| | | (SEQ ID NO: 163) | |
| 1337 | 1172-Mis- | TTGGCATGGAAGTCACACCTAGGGGT | FIG. 14B |
| | 2-11 | TGCAGTCTCCGCCTACATGGGAGTTGT | |
| | | GACCCTATAGTGAGTCGTATTAATTTC | |
| | | (SEQ ID NO: 164) | |
| 1338 | 1172-Mis- | TTGGCATGGAAGTCACACCTTCCCGTT | FIG. 14B |
| | 2-12 | GCAGTCTCCGCCTACATGGGAGTTGT | |
| | | GACCCTATAGTGAGTCGTATTAATTTC | |
| | | (SEQ ID NO: 165) | |
| 1339 | 1172-Seed- 1 | AACCCATGGAAGTCACACCTTCGGGT | FIG. 14C |
| | | TGCAGTCTCCGCCTACATGGGAGTTGT | |
| | | GACCCTATAGTGAGTCGTATTAATTTC | |
| | | (SEQ ID NO: 166) | |
| 1340 | 1172-Seed- | TTGGGTACGAAGTCACACCTTCGGGTT | FIG. 14C |
| | 2 | GCAGTCTCCGCCTACATGGGAGTTGT | |
| | | GACCCTATAGTGAGTCGTATTAATTTC | |
| | | (SEQ ID NO: 167) | |
| 1341 | 1172-Seed- | TTGGCATGCTTCTCACACCTTCGGGTT | FIG. 14C |
| | 3 | GCAGTCTCCGCCTACATGGGAGTTGT | |
| | | GACCCTATAGTGAGTCGTATTAATTTC | |
| | | (SEQ ID NO: 168) | |
| 1342 | 1172-Seed- | TTGGCATGGAAGAGTGACCTTCGGGT | FIG. 14C |
| | | TGCAGTCTCCGCCTACATGGGAGTTGT | |
| | 4 | GACCCTATAGTGAGTCGTATTAATTTC | |
| | | (SEQ ID NO: 169) | |
| 1343 | 1172-Seed- | TTGGCATGGAAGTCACTGGATCGGGT | FIG. 14C |
| | 5 | TGCAGTCTCCGCCTACATGGGAGTTGT | |
| | | GACCCTATAGTGAGTCGTATTAATTTC | |
| | | (SEQ ID NO: 170) | |
| 1344 | 1172-Seed- | TTGGCATGGAAGTCACACCTAGCCGT | FIG. 14C |
| | 6 | TGCAGTCTCCGCCTACATGGGAGTIGT | |
| | | GACCCTATAGTGAGTCGTATTAATTTC | |
| | | (SEQ ID NO: 171) | |
| 1345 | 1172- | CATGGAAGTCACACCTGTTGCAGTCTC | FIG. 14D |
| | Spacer-16 | CGCCTACATGGGAGTTGTGACCCTAT | |
| | | AGTGAGTCGTATTAATTTC (SEQ ID | |
| | | NO: 172) | |
| 1346 | 1172- | GCATGGAAGTCACACCTTGTTGCAGT | FIG. 14D |
| | | CTCCGCCTACATGGGAGTTGTGACCCT | |
| | Spacer-18 | ATAGTGAGTCGTATTAATTTC (SEQ ID | |
| | | NO: 173) | |
| 1347 | 1172- | GGCATGGAAGTCACACCTTCGTTGCA | FIG. 14D |
| | Spacer-20 | GTCTCCGCCTACATGGGAGTTGTGAC | |
| | | CCTATAGTGAGTCGTATTAATTTC | |
| | | (SEQ ID NO: 174) | |
| 1348 | 1172- | TGGCATGGAAGTCACACCTTCGGTTG | FIG. 14D |
| | Spacer-22 | CAGTCTCCGCCTACATGGGAGTTGTG | |
| | | ACCCTATAGTGAGTCGTATTAATTTC | |
| | | (SEQ ID NO: 175) | |
| 1349 | 1172- | ATTGGCATGGAAGTCACACCTTCGG | FIG. 14D |
| | Spacer-26 | GGTTGCAGTCTCCGCCTACATGGGA | |
| | | GTTGTGACCCTATAGTGAGTCGTAT | |
| | | TAATTTC (SEQ ID NO: 176) | |
| 1350 | 1172- | CATTGGCATGGAAGTCACACCTTCGG | FIG. 14D |
| | Spacer-28 | GAGTTGCAGTCTCCGCCTACATGGGA | |
| | | GTTGTGACCCTATAGTGAGTCGTATTA | |
| | | ATTTC (SEQ ID NO: 177) | |
| 1351 | 1172- | GCATTGGCATGGAAGTCACACCTTCG | FIG. 14D |
| | Spacer-30 | GGAAGTTGCAGTCTCCGCCTACATGG | |
| | | GAGTTGTGACCCTATAGTGAGTCGTAT | |
| | | TAATTTC (SEQ ID NO: 178) | |
| 1215 | ORF1 | GTTAAACCAGGTGGAACCTCATCAGG | FIG. 2E |
| | LwaCas13a | AGATGTTTTAGTCCCCTTCGTTTTTGG | |
| | crRNA | GGTAGTCTAAATCCCCTATAGTGAGTC | |
| | | GTATTAATTTC (SEQ ID NO: 179) | |
| 1363 | Tcc-crRNA- | TAGTGGTCTGCGGAACCGGTGAGTGT | FIGS. 22A- |
| | HCV-1 | TGCAGTCTCCGCCTACATGGGAGTTGT | 22C |
| | | GACCCTATAGTGAGTCGTATTAATTTC | |
| | | (SEQ ID NO: 180) | |
| 1370 | Tcc-crRNA- | AAGGCCTTATGGAAGCAGCCCAATGT | FIGS. 22A- |
| | TYLCV-2 | TGCAGTCTCCGCCTACATGGGAGTTGT | 22C |
| | | GACCCTATAGTGAGTCGTATTAATTTC | |
| | | (SEQ ID NO: 181) | |
| CV627 | AapCas12b- | AGGGTCACACCCAAGTAATTGTGCCA | FIGS. 21A- |
| | RnaseP- | CTTCTCAGATTTGAG (SEQ ID NO: 182) | 21B |
| | sgRNA-1 | | |
| CV628 | AapCas12b- | CTCGGACCAGAGCCATGTAAGTGCCA | FIGS. 21A- |
| | RnaseP- | CTTCTCAGATTTGAG (SEQ ID NO: 183) | 21B |
| | sgRNA-2 | | |
| CV629 | AapCas12b- | AATTACTTGGGTGTGACCCTGTGCCAC | FIGS. 21A- |
| | RnaseP- | TTCTCAGATTTGAG (SEQ ID NO: 184) | 21B |
| | sgRNA-3 | | |
| CV241 | AapCas12b | GAAATTAATACGACTCACTATAGGG | |
| | sgRNA | TCTAGAGGACAGAATTTTTCAACGG | |
| | scaffold- | GTGTGCCAATGGCCACTTTCCAGGT | |
| | Top | GGCAAAGCCCGTTGAGCTTCTCAAA | |
| | | TCTGAGAAGTGGCAC (SEQ ID | |
| | | NO: 185) | |

Pre-crRNAs of LwaCas13a, HheCas13a, and TccCas13a

| crRNA # | Name | Sequence (5′→3′) |
|---|---|---|
| 1377 | LwaCas13a- | FAM- |
| | pre- | AUCAUGCGAGGAUUUAGACUACCCCAAAA |
| | crRNA-5′ | ACGAAGGGGACUAAAACUUCAGAUAUAGC |
| | FAM | CUGGUGGUUCAGGC |
| | labeled | (SEQ ID NO: 186) |
| 1378 | HheCas13a- | FAM- |
| | pre-crRNA- | ACUGAGGUAUGUAACAAUCCCCGUAGAC |
| | 5′ FAM | AGGGGAACUGCAACCAGAUAUAGCCUGG |
| | labeled | UGGUUCAGGC (SEQ ID NO: 187) |
| 1379 | TccCas13a- | GUCGACACGGGUCACAACUCCCAUGUAG |
| | pre-crRNA- | GCGGAGACUGCAACUAUUACCACUCAGG |
| | 5′ FAM | GUAAUGUCCG (SEQ ID NO: 188) |
| | labeled | |

gBlocks used in this study.

| Name | Sequence (5′→3′) |
|---|---|
| SARS-CoV-2 | GAAATTAATACGACTCACTATAGGGCCTCACTTGTTC |
| ORF1 gBlock | TTGCTCGCAAACATACAACGTGTTGTAGCTTGTCACA |
| | CCGTTTCTATAGATTAGCTAATGAGTGTGCTCAAGTA |
| | TTGAGTGAAATGGTCATGTGTGGCGGTTCACTATATG |
| | TTAAACCAGGTGGAACCTCATCAGGAGATGCCACAA |
| | CTGCTTATGCTAATAGTGTTTTTAACATTTGTCAAGCT |
| | GTCACGGCCAATGTTAATGCACTTTTATCTACTGATG |
| | GTAACAAAATTGCCGATAAGTATGTC (SEQ ID NO: 189) |
| HCV genotype 1a | GAAATTAATACGACTCACTATAGGGTTGGGGGCGAC |
| | ACTCCACCATGAATCACTCCCCTGTGAGGAACTACTG |
| | TCTTCACGCAGAAAGCGTCTAGCCATGGCGTTAGTAT |
| | GAGTGTCGTGCAGCCTCCAGGACCCCCCCTCCCGGGA |
| | GAGCCATAGTGGTCTGCGGAACCGGTGAGTACACCG |
| | GAATTGCCAGGACGACCGGGTCCTTTCTTGGATAAAC |
| | CCGCTCAATGCCTGGAGATTTGGGCGTGCCCCCGCAA |
| | GACTGCTAGCCGAGTAGTGTTGGGTCGCGAAAGGCCT |
| | TGTGGTACTGCCTGATAGGGTGCTTGCGAGTGCCCCG |
| | GGAGGTCTCGTAGACCGTGCACCATGAGCACGAATC |
| | CTAAACCCCAAAGAAAAACCAAACGTAACACCAACC |
| | GTCGCCCACAGGACGTCAAGTTCCCGGGTGGCGGTCA |
| | GATCGTTGGTGGAGTTTACTTGTTGCCGCGCAGGGGC |
| | CGTCATAGCTGTTTC CTG (SEQ ID NO: 190) |
| HCV genotype | GAAATTAATACGACTCACTATAGGGTTGGGGGCGACACTC |
| 2b | CGCCATGAATCACTCCCCTGTGAGGAACTACTGTCTTCACG |
| | CAGAAAGCGTCTAGCCATGGCGTTAGTATGAGTGTCGTAC |
| | AGCCTCCAGGCCCCCCCCTCCCGGGAGAGCCATAGTGGTCT |
| | GCGGAACCGGTGAGTACACCGGAATTACCGGAAAGACTGG |
| | GTCCTTTCTTGGATAAACCCACTCTATGTCCGGTCATTTGG |
| | GCACGCCCCCGCAAGACTGCTAGCCGAGTAGCGTTGGGTT |
| | GCGAAAGGCCTTGTGGTACTGCCTGATAGGGTGCTTGCGA |
| | GTGCCCCGGGAGGTCTCGTAGACCGTGCATCATGAGCACA |
| | AATCCTAAACCTCAAAGAAAAACCAAAAGAAACACAAACC |
| | GCCGCCCACAGGACGTTAAGTTCCCGGGTGGCGGTCAGAT |
| | CGTTGGCGGAGTTTACTTGCTGCCGCGCAGGGGCCGTCATA |
| | GCTGTTTCCTG (SEQ ID NO: 191) |

crRNA sequences shown as (5′→3′) reverse complement to be annealed with T7 oligo for IVT.

TABLE 6
Clinical samples used in this study.

| Sample number | Sample ID | Ct value |
|---|---|---|
| 391 | 160935 | 16 |
| 392 | 160938 | 14 |
| 393 | 160939 | 16 |
| 394 | 160941 | 16 |
| 395 | 160942 | 27 |
| 396 | 160943 | 14 |
| 397 | 160944 | 20 |
| 398 | 160947 | 14 |

| | | | | |
|---|---|---|---|---|
| | Sample | | Ct value | |
| | ID in | Sample | (SARS-CoV-2 | SARS-CoV-2 |
| # | figures | ID | N gene) | strain |

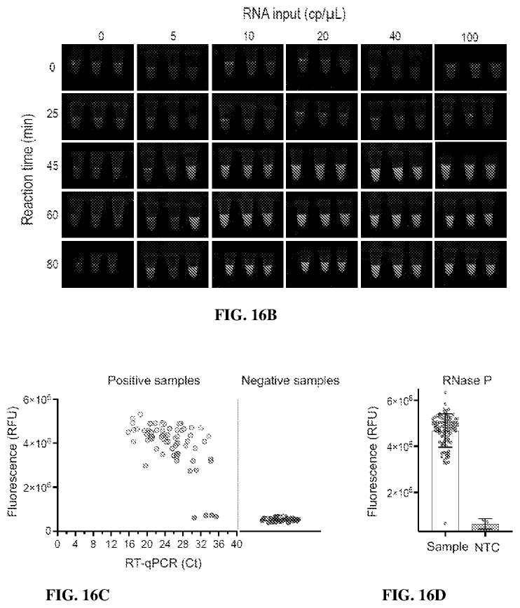

Used in FIG. 16C&16D

| | | | |
|---|---|---|---|
| 95 | A1 | 20.20318222 | Delta |
| 96 | A2 | 23.8019371 | Delta |
| 97 | A3 | 20.38153458 | Delta |
| 98 | A4 | 23.91605568 | Beta |
| 99 | A5 | 21.15932465 | Delta |
| 100 | A6 | 20.17287445 | Beta |
| 101 | A7 | 20.63743782 | Delta |
| 102 | A8 | 26.51057625 | Delta |
| 103 | A9 | 24.15272903 | Delta |
| 104 | A10 | 26.29830933 | Delta |
| 105 | A11 | 22.10408401 | Delta |
| 106 | A12 | 26.71198273 | Delta |
| 107 | B1 | 32.79143906 | Delta |
| 108 | B2 | 30.94537354 | Delta |
| 109 | B3 | 19.86674881 | Delta |
| 110 | B4 | 26.80452538 | Delta |
| 111 | B5 | 23.44918823 | Delta |
| 112 | B6 | 23.47177696 | Delta |
| 113 | B7 | 25.94287682 | Delta |
| 114 | B8 | 26.83697891 | Beta |
| 115 | B9 | 26.61860275 | Delta |
| 116 | B10 | 29.60500526 | Beta |
| 117 | B11 | 28.60080528 | Delta |
| 118 | B12 | 21.05815697 | Delta |
| 119 | C1 | 23.73565865 | Delta |
| 120 | C2 | 20.14953613 | Delta |
| 121 | C3 | 24.65905762 | Delta |
| 122 | C4 | 20.69680595 | Delta |
| 123 | C5 | 24.33411217 | Alpha |
| 124 | C6 | 30.21217537 | Delta |
| 125 | C7 | 30.52635384 | Beta |
| 126 | C8 | 25.62018967 | Delta |
| 127 | C9 | 26.6070385 | Delta |
| 128 | C10 | 16.92820358 | Alpha |
| 129 | C11 | 26.35331726 | Alpha |
| 130 | C12 | 16.99542999 | Beta |
| 131 | D1 | 15.77931976 | Other |
| 132 | D2 | 26.65350533 | Delta |
| 133 | D3 | 18.5001297 | Alpha |
| 134 | D4 | 31.98776627 | Delta |
| 135 | D5 | 16.7693367 | Delta |
| 136 | D6 | 34.24099731 | Delta |
| 137 | D7 | 21.01351547 | Other |
| 138 | D8 | 16.75134087 | Delta |
| 139 | D9 | 29.01878738 | Delta |
| 140 | D10 | 27.75723839 | Delta |
| 141 | D11 | 29.67990685 | Other |
| 142 | D12 | 20.9514904 | Other |
| 143 | E1 | 24.57509232 | Delta |
| 144 | E2 | 20.96550179 | Delta |
| 145 | E3 | 26.76332283 | Beta |
| 146 | E4 | 25.0199337 | Other |
| 147 | E5 | 29.9657135 | Beta |
| 149 | E7 | 27.49075508 | Alpha |
| 150 | E8 | 22.13245583 | Delta |
| 151 | E9 | 23.42307281 | Delta |
| 152 | E10 | 24.99167442 | Delta |
| 153 | E11 | 23.46128845 | Delta |
| 154 | E12 | 19.64938736 | Delta |
| 155 | F1 | 28.25497818 | Delta |
| 156 | F2 | 20.91403008 | Beta |
| 157 | F3 | 23.19413948 | Delta |
| 158 | F4 | 33.09214783 | Alpha |
| 159 | F5 | 34.40586853 | Delta |
| 160 | F6 | 30.73725128 | Other |
| 161 | F7 | 23.17684937 | Other |
| 162 | F8 | 21.75904465 | Alpha |
| 163 | F9 | 34.17617035 | Beta |
| 164 | F10 | 27.16244316 | Alpha |
| 165 | F11 | 19.42002296 | Beta |
| 166 | F12 | 33.7602272 | Alpha |
| 167 | G1 | 35.41335297 | Alpha |
| 168 | G2 | 17.82286072 | Other |
| 169 | G3 | Undetermined | |
| 170 | G4 | Undetermined | |
| 171 | G5 | Undetermined | |
| 172 | G6 | Undetermined | |
| 173 | G7 | Undetermined | |
| 174 | G8 | Undetermined | |
| 175 | G9 | Undetermined | |
| 176 | G10 | Undetermined | |
| 177 | G11 | Undetermined | |
| 178 | G12 | Undetermined | |
| 179 | H1 | Undetermined | |
| 180 | H2 | Undetermined | |
| 181 | H3 | Undetermined | |
| 182 | H4 | Undetermined | |
| 183 | H5 | Undetermined | |
| 184 | H6 | Undetermined | |
| 185 | H7 | Undetermined | |
| 186 | H8 | Undetermined | |
| 187 | H9 | Undetermined | |
| 188 | H10 | Undetermined | |
| 189 | H1 | Undetermined | |
| 190 | H2 | Undetermined | |
| 192 | H4 | Undetermined | |
| 193 | H5 | Undetermined | |
| 194 | H6 | Undetermined | |
| 195 | H7 | Undetermined | |
| 197 | H9 | Undetermined | |

Results described in text

| | | | |
|---|---|---|---|
| 396 | A12 | 160943 | 14 |
| 397 | B1 | 160944 | 20 |
| 398 | B2 | 160947 | 14 |
| 410 | C2 | 160992 | 23 |
| 421 | D1 | 164191 | 17 |
| 444 | E12 | 164515 | 14 |
| 445 | F1 | 164520 | 27 |
| 446 | F2 | 164525 | 27 |
| 447 | F3 | 164527 | 19 |
| 448 | F4 | 164530 | 19 |
| 449 | F5 | 164532 | 27 |
| 450 | F6 | 164537 | 27 |
| 451 | F7 | 164539 | 18 |
| 452 | F8 | 164553 | 20 |
| 453 | F9 | 164580 | 34 |
| 454 | F10 | 164581 | 20 |
| 455 | F11 | 164583 | 18 |
| 456 | F12 | 164589 | 22 |
| 457 | G1 | 164596 | 31 |
| 458 | G2 | 164597 | 15 |
| 459 | G3 | 164598 | 24 |
| 460 | G4 | 164603 | 18 |
| 461 | G5 | 164604 | 18 |
| 462 | G6 | 164605 | 19 |
| 463 | G7 | 164624 | 25 |
| 465 | G9 | 164649 | 21 |
| 466 | G10 | 164650 | 22 |
| 467 | G11 | 164651 | 17 |
| 468 | G12 | 164654 | 26 |
| 469 | H1 | 164661 | 22 |
| 470 | H2 | 164662 | 18 |
| 472 | H4 | 164665 | 25 |
| 473 | H5 | 164667 | 20 |
| 474 | H6 | 166758 | 24 |
| 475 | H7 | 166759 | 19 |
| 476 | H8 | 166760 | 21 |
| 477 | H9 | 166761 | 14 |
| 478 | H10 | 166762 | 23 |
| 479 | H11 | 166763 | 16 |
| 480 | H12 | 166764 | 24 |
| K0002 | −ve 1 | K0002 | Undetermined |
| K0005 | −ve 2 | K0005 | Undetermined |
| K0006 | −ve 3 | K0006 | Undetermined |
| K0007 | −ve 4 | K0007 | Undetermined |
| K0008 | −ve 5 | K0008 | Undetermined |

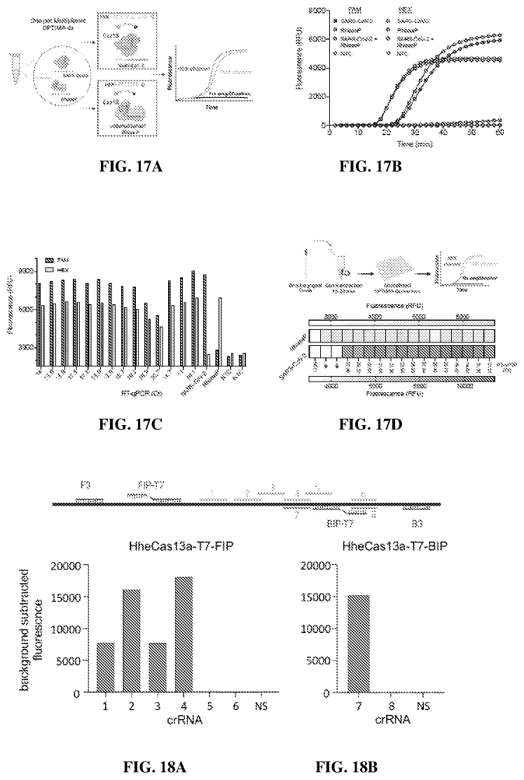

Used in FIG. 17C

| | | |
|---|---|---|
| K0244 | 14 | 14 |
| K0246 | 15.6 | 15.6 |
| K0250 | 16.8 | 16.8 |
| K0247 | 17.5 | 17.5 |
| K0249 | 17.8 | 17.8 |
| K0254 | 15.5 | 15.5 |
| K0252 | 17.5 | 17.5 |
| K0203 | 16.7 | 16.7 |
| K0209 | 19.2 | 19.2 |
| K0200 | 16.9 | 16.9 |
| K0253 | 30.7 | 30.7 |
| K0213 | 14.7 | 14.7 |
| K0068 | 17 | 17 |
| K0265 | 16.1 | 16.1 |

Used in FIG. 17D

| | | |
|---|---|---|
| 1 | 22.23623848 | 22.23623848 |
| 2 | 20.20703506 | 20.20703506 |
| 4 | 14.83642673 | 14.83642673 |
| 6 | 16.92188454 | 16.92188454 |
| 7 | 22.07608795 | 22.07608795 |
| 8 | 20.47973633 | 20.47973633 |
| 9 | 21.8478508 | 21.8478508 |
| 10 | 26.23923874 | 26.23923874 |
| 11 | 22.94090843 | 22.94090843 |
| 12 | 22.38891029 | 22.38891029 |
| 13 | 22.45388031 | 22.45388031 |
| 14 | 22.93203545 | 22.93203545 |
| 15 | 28.23180008 | 28.23180008 |
| 16 | 27.80712509 | 27.80712509 |
| 5 | −Ve | Undetermined |
| 18 | −Ve | Undetermined |

Described in text

| | | |
|---|---|---|
| 1 | 1 | 18.57 |
| 2 | 2 | 24.62 |
| 3 | 3 | 19.65 |
| 4 | 4 | 30.72 |
| 5 | 5 | 32.33 |
| 6 | 6 | 21.14 |
| 7 | 7 | 31.9 |
| 8 | 8 | 34.9 |
| 9 | 9 | 19.93 |
| 10 | 10 | 16.59 |
| 11 | 11 | 18.46 |
| 12 | 12 | 17.81 |
| 13 | 13 | 19.82 |
| 14 | 14 | 18.2 |
| 15 | 15 | 23.64 |
| 16 | 16 | 35.57 |
| 17 | 17 | 20.72 |
| 18 | 18 | 16.57 |
| 19 | 19 | 30 |
| 20 | 20 | 18.98 |
| 21 | 21 | 31.13 |
| 22 | 22 | 27.63 |
| −Ve | −Ve | Undetermined |
| −Ve | −Ve | Undetermined |

### OPTIMA-Dx Reaction

The reaction was performed using RT-LAMP primers at a final concentration of 1.6 μM FIP/BIP primers (with the T7 promoter sequence added to the FIP primer), 0.2 μM F3/B3 primers, and 642 0.4 μM LF/LB primers, in 1× Isothermal Amplification Buffer from Lucigen (30027, Lucigen), 1.4 mM dNTPs, 2.4 U/μL Bst DNA Polymerase (30027, Lucigen), 0.3 U/μL of WarmStart RTx Reverse Transcriptase (M0380, NEB), 6 mM MgSO₄, 0.8 U/μL RNasin plus (N2611, Promega), 0.5 mM NTPs, 4 U/μL Hi-T7 RNA polymerase (M0658S, NEB), 0.4 U/μL thermostable inorganic pyrophosphatase (M0296, NEB), 1 μM ssRNA FAM reporter or 750 nM ssRNA HEX reporter, 50 nM Cas13, 50 nM crRNA, and 4.5 μL of template RNA in 25-μL reactions. A detailed protocol for the OPTIMA-dx reaction setup is provided in supplementary note 1.

### One-Pot Multiplexed OPTIMA-Dx Reaction

The multiplexed reaction was performed as described above (OPTIMA-dx reaction) with the following modification. 50 nM of AapCas12b protein, 50 nM of AapCas12b sgRNAs-1 for RNase P detection, 250 nM HEX ssDNA reporter (the FAM reporter used at 250 nM instead of 1 μM in multiplexed detection), and RT-LAMP primers for RNase P detection (LF and LB were 655 used at 0.2 □M final concentration) were added to OPTIMA-dx SARS-COV-2 or HCV detection 656 components. The final reaction volume is 50 μL. These reactions were incubated in a 96-well 657 plate (BioRad) at 56° C. for 1-2 h in a 96-well Real-Time PCR detection system (CFX96 qPCR 658 machine, Bio-Rad), with fluorescence measurements taken every 2 min using both FAM and 659 HEX channels.

### Pre-crRNA Processing Assays

RNA oligos of 5′ FAM-labelled pre-crRNAs were custom-synthesized (IDT). Pre-crRNA processing assays were performed in 1× isothermal buffer (B0537, NEB) supplemented with an additional 6 mM MgSO4 (final of 8 mM MgSO4) in a 20-μL reaction volume. In all assays, 100 nM of each Cas13a orthologue was incubated with 200 nM of their cognate 5′-FAM labeled pre-crRNAs for 1 hour at different temperatures. The reactions were then heated at 70° C. for 3 min in 1×RNA Loading Dye (B0363S, NEB) and cooled down on ice for 3 min before loading onto a 15% polyacrylamide-urea denaturing gel. Electrophoresis was conducted for 80 min at 25 W. The gels were visualized using fluorescein channel in Bio-Rad Molecular Imager Gel Doc system.

### Two-Pot Detection Reactions

Reverse transcription and LAMP isothermal amplification of target nucleic acids were conducted using the previously reported RT-LAMP primers [Joung N Engl J Med, 2020. 383 (15): p. 1492-1494]. Reactions were performed using 1.6 μM FIP/BIP primers (with the T7 promoter sequence added to either the FIP or BIP primer), 0.2 μM F3/B3 primers, and 0.4 μM LF/LB primers in 1× Isothermal Amplification Buffer (20 mM Tris-HCl pH 8.8, 50 mM KCl, 10 mM (NH4)2SO4, 2 mM MgSO4, 0.1% Tween 20) (B0537, NEB), 1.4 mM dNTPs, 8 U of Bst 2.0 WarmStart DNA Polymerase (M0538, NEB), 7.5 U of WarmStart RTx Reverse Transcriptase (M0380, NEB) and 6 mM MgSO4 (B1003, NEB) in 25-μL reactions containing 100 cp/μL of SARS-COV-2 control standards. The reactions were incubated at 62° C. for 40 min in a PCR machine (C1000 touch thermal cycler, BioRad). For subsequent Cas13a-based detection, 50 nM of recombinant HheCas13a or TccCas13a protein was incubated with 50 nM of the respective crRNA, 250 nM of ssRNA reporter (Poly(U) ssRNA reporter for HheCas13a or mix ssRNA reporter for TccCas13a), 0.8 U/μL RNaseOUT, 2 U/μL Hi-T7 RNA polymerase (M0658S, NEB), 1 mM NTPs, and 2 IL of the RT-LAMP reaction product. Reactions were run in a 96-well plate (BioRad) at 55° C. for 1 h in a 96-well Real-Time PCR detection system (CFX96 qPCR machine, Bio-Rad), with fluorescence measurements taken every 2 min using the FAM channel.

### Michaelis-Menten Enzyme Kinetic Parameters Calculation

The trans cleavage activity of TccCas13a was investigated by measuring Michaelis-Menten enzyme kinetic parameters following the protocol introduced by Ramachandran et al. [4]. The Michaelis-Menten equation represents the relationship between reaction velocity and substrate concentration, which can be obtained from experimental data:

$$v = \frac{d [ P ]}{dt} = {k}_{cat} {E}_{0} \frac{[ S ]}{{K}_{M} + [ S ]}$$

Where v is reaction velocity, [P] is the concentration of reaction product, E0 is the initial enzyme concentration, and [S] is the substrate concentration. The reporter cleaved by TccCas13a is the reaction product in this assay. To estimate the kinetic parameters of TccCas13a, 0.5 nM of activated RNP was treated with different concentrations of FAM Mix reporters. In detail, 100 nM RNP was first prepared by incubating 100 nM TccCas13a protein, 125 nM crRNA (#1172), and 1 U of RNase inhibitor (NEB, M0314L) in 1× isothermal amplification buffer (B0537, NEB) supplemented with 6 mM MgSO4 at 56° C. for 10 minutes. Next, the trans cleavage activity of RNP was activated by mixing 20 nM of N gene target with 2 nM RNP in 1× isothermal amplification buffer supplemented with 6 mM MgSO4 and 1 U of RNase inhibitor and incubated at 56° C. for 15 min. For the trans cleavage assay, FAM Mix reporter at concentrations of 31.25 nM, 62.5 nM, 125 nM, 250 nM, 500 nM, 1 μM, 2 μM and 4 μM was added into 0.5 nM of target-activated RNP together with 6 mM MgSO4 and 1 U of RNase inhibitor in 1× isothermal amplification buffer in 20 μL of the final volume. The fluorescence readout was measured every 30 s at 56° C. (CFX96 qPCR machine, Bio-Rad). The same reactions described above were also carried out in parallel without the addition of crRNA, which were used as controls to subtract the fluorescence background signal. The data were analyzed by GraphPad Prism software (GraphPad, CA, USA) to calculate KM and kcat. First, the data obtained from reactions without crRNA were subtracted from those reactions with crRNA to obtain the true fluorescence generated by enzyme-cleaved reporters. The real-time data from the first 600 s were fitted using linear regression to obtain the initial reaction velocity for different reporter concentrations represented by the increase of fluorescence over time, which can be represented as dF/dt. To convert the fluorescence readout into the concentration of the cleaved product, FAM Mix at 31.25 nM, 62.5 nM, 125 nM, 250 nM, 500 nM, 1 μM, and 2 μM was incubated with 40 μg of RNase A (Invitrogen, cat: 12091-039) in 20 μL reaction at 37° C. for more than 3 hours to ensure complete cleavage. By plotting the end-point data and subtracting it from the background using water-only samples over reporter concentration, the relationship between fluorescence readout and reporter concentration can be obtained: FP=a[P]. Where FP is the fluorescence produced by the cleaved reporter, and a is a constant.

The reaction can then be calculated as follows:

$$v = \frac{dP}{dt} = \frac{1}{a} \times \frac{dF}{dt}$$

The curve for reaction velocity dP/dt over reporter concentration was fitted to the Michaelis-Menten equation to calculate the value of KM and Vmax. The kcat can be calculated as Vmax equals the value of kcatE0. To test the validation of calculated kinetic parameters, the back-of-the-envelope test introduced in Ramachandran et al. [Ramachandran, et al., Anal Chem, 2021. 93 (20): p. 7456-7464] was conducted. For all the tests, an initial linear time portion tlin of 600 s was used to calculate the α, β, and γ values:

| E₀ (nM) | tₗᵢₙ (s) | S₀ (nM) | v (nM/s) | α | β | γ |
|---|---|---|---|---|---|---|
| 0.5 | 600 | 4000 | 0.296 | 0.044 | 0.371 | |
| | | 2000 | 0.195 | 0.059 | 0.245 | |
| | | 1000 | 0.103 | 0.062 | 0.129 | |
| | | 500 | 0.039 | 0.047 | 0.049 | |
| | | 250 | 0.031 | 0.073 | 0.038 | |
| | | 125 | 0.015 | 0.074 | 0.019 | |
| | | 62.5 | 0.008 | 0.073 | 0.010 | |
| | | 31.25 | 0.003 | 0.060 | 0.004 | |

### Optimization of One-Pot Detection Reactions

For Bst DNA polymerase screening and other optimization reactions, reverse transcription and LAMP isothermal amplification of the target nucleic acids, coupled with T7-mediated in vitro transcription and Cas13-based detection of the amplified and in vitro-transcribed target RNA, were carried out in the same tube. Reactions were performed using RT-LAMP primers at a final concentration of 1.6 μM for FIP/BIP primers (with the T7 promoter sequence added to either the FIP or BIP primer), 0.2 μM F3/B3 primers, and 0.4 μM LF/LB primers, in 1× Isothermal Amplification Buffer (from a different vendor from the Bst DNA polymerase screening reactions) or from Lucigen (30027, Lucigen) in other optimization experiments, 1.4 mM dNTPs, 0.32 U/μL Bst DNA Polymerase (from a different vendor from the Bst DNA polymerase screening reactions) or 2.4 U/μL from Lucigen (30027, Lucigen), 0.3 U/μL of WarmStart RTx Reverse Transcriptase (M0380, NEB), 6 mM MgSO4, 0.8 U/μL RNasin plus (N2611, Promega), 0.5 mM NTPS, 2 U/μL Hi-T7 RNA polymerase (M0658S, NEB), 0.4 U/ML thermostable inorganic pyrophosphatase (M0296, NEB), 250 nM RNA reporter, 50 nM Cas13, 50 nM crRNA, and 2 μL of template RNA in 25-μL reactions. These reactions were incubated in a 96-well plate (BioRad) at 56° C. (or as otherwise indicated) for 1-2 h in a 96-well Real-Time PCR detection system (CFX96 qPCR machine, Bio-Rad), with fluorescence measurements taken every 2 min using the FAM channel.

For the detection of HCV, the one-pot detection reactions were performed as described above with the use of 500 pM in vitro transcribed RNA template. For the detection of TYLCV, the reactions were performed as described above, but without the addition of RTx reverse transcriptase and with use of 2 μL of 1:10 or 1:100 diluted extracted DNA as template. 1 ng of TYLCV plasmid was used as positive control. All RT-LAMP primers are listed above.

Screening of AapCas12b sgRNAs in One-Pot Reaction

Reactions were performed using RT-LAMP primers at a final concentration of 1.6 μM for FIP/BIP primers, 0.2 μM F3/B3 primers, and 0.2 μM LF/LB primers, in 1× Isothermal Amplification Buffer (30027, Lucigen), 1.4 mM dNTPs, 2.4 U/μL Bst DNA Polymerase (30027, Lucigen), 0.3 U/μL of WarmStart RTx Reverse Transcriptase (M0380, NEB), 6 mM MgSO4, 0.8 U/μL RNasin plus (N2611, Promega), 0.5 mM NTPS, 4 U/μL Hi-T7 RNA polymerase (M0658S, NEB), 0.4 U/μL thermostable inorganic pyrophosphatase (M0296, NEB), 250 nM ssDNA HEX reporter, 50 nM AapCas12b, 50 nM sgRNAs, and 1 μL of total human RNA template in 25-μL reactions. These reactions were incubated in a 96-well plate (BioRad) at 56° C. for 1 h in a 96-well Real-Time PCR detection system (CFX96 qPCR machine, Bio-Rad), with fluorescence measurements taken every 2 min using the HEX channel. T7 RNA polymerase and NTPs were included here to test the compatibility of Cas12b activity with these reagents for the subsequent multiplex detection reactions.

Agroinfiltration Inoculation of Plants with TYLCV and DNA Extraction

Plant infection with TYLCV infectious clones and subsequent DNA extraction was done following the previous protocol (Mahas et al. Detection of Plant DNA Viruses. Viruses, 2021. 13 (3)).

### Clinical Sample Collection and RNA Extraction.

Oropharyngeal and nasopharyngeal swabs were collected from suspected COVID-19 patients by physicians in Ministry of Health hospitals in Saudi Arabia and placed in 2-mL screw-capped cryotubes containing 1 mL of TRIZOL for inactivation and transport. Each sample tube was sprayed with 70% ethanol, enveloped with absorbent tissues, and then placed and sealed in individually labeled biohazard bags. The bags were then placed in leak-proof boxes and sprayed with 70% ethanol before placement in a dry ice container for transfer to the lab. Total RNA was extracted from the samples following instructions as described in the CDC EUA-approved protocol and using the Direct-zol kit (Direct-zol RNA Miniprep, Zymo Research; catalog #R2070) following the manufacturer's instructions.

### Extraction-Free Sample Processing and Concentration

A beads-based extraction mixture was prepared as follows. First, beads were prepared by washing 1 mL of Sera-Mag SpeedBeads Carboxyl Magnetic Beads Hydrophobic (GE Healthcare 65152105050250) with 1 mL of UltraPure DNase/RNase-free distilled water (1097705, Invitrogen) twice and then resuspended in 50 mL of beads binding buffer (10 mM Tris-HCl pH 8.0, 1M KCl, 18% PEG-8000, and 1 mM EDTA). Next, 50 mL of extraction mixture was prepared by mixing 32.5 mL of beads (resuspended in binding buffer), 12.5 ml of 4× Viral RNA Extraction Buffer (VRE100, Sigma-Aldrich), and 5 mL of UltraPure DNase/RNase-free distilled water. The extraction mixture was aliquoted in 1.5 mL tubes, 400 μL each.

To process clinical samples, 200 μL of VTM of oropharyngeal swabs were transferred into 400 μL of extraction mixture, vortexed vigorously and incubated at room temperature for 5 min. The mixture was then placed on a magnetic rack (Invitrogen DYNAL bead Separator) for 2-3 mins until the solution gets clear. Next, the supernatant is removed, and beads is resuspended and washed in 750 μL of 70% ethanol (v/v). Samples are again placed on the magnetic rack for ˜ 2 mins until solution gets clear. The supernatant is removed completely and the tubes are left open for 5-10 min to dry. Beads are then resuspended in 30 μL of H2O and vortexed vigorously for 5 seconds and incubated at room temperate for 3 mins. The samples are then placed on the magnetic rack to collect beads, and 15 μL of H2O is transferred into 35 μL of OPTIMA-dx master mix.

For experiment with VTM spiked with non-infectious virus particles, 200 μL of VTM of oropharyngeal swabs collected from healthy donors were spiked with the indicated concentration of non-infectious virus particles (NATSARS(CoV2)-ERC, ZeptoMetrix) or with 200 μL of (NATSARS(CoV2)-NEG, ZeptoMetrix) for negative controls, and the spiked VTM were processed as described above.

### Real-Time Reverse Transcription PCR (RT-PCR) for Detecting Positive SARS-COV-2 RNA Samples.

RT-PCR was conducted on extracted RNA samples using the oligonucleotide primer/probe (Integrated DNA Technologies, 641 catalog #10006606) and Superscript III one-step RT-PCR system with Platinum Taq Polymerase (catalog #12574-026) following the manufacturer's protocol.

### Freeze-Drying of Detection Reactions

Multiplexed OPTIMA-dx detection reactions were assembled as described above in a final volume of 50 μL in 1.5 mL tubes. Reactions were snap-frozen in liquid nitrogen and transferred to a LABCONCO Acid-Resistant CentriVap Concentrator (supplemented with LABCONCO CentriVap-105° C. Cold Trap and Vacuubrand CVC 3000 Vacuum pump) Freeze Dry System for 2-3 hours of freeze-drying at a minimal temperature under the pressure of 1 to 10 millibar until the water was completely removed. Rehydration of freeze-dried reactions was accomplished with the RNA isolated from clinical samples (20 μL), 25 H2O, and 5 μL of 10× Isothermal Amplification Buffer from Lucigen (30027, Lucigen).

### Assembly of OPTIMA-Dx Reaction.

1—Dilute TccCas13a protein into 1 μM in 1× isothermal buffer (Lucigen, 30027). 2—Assemble TccCas13a RNP as follows:

| | Concentra- | | Final |
|---|---|---|---|
| Reagents | tion | Amount (μL) | concentration |
| H₂0 | | 12 | |
| Isothermal buffer | 10X | 3 | 1X |
| TccCas13a | 1 μM | 7.5 | 250 nM |
| TccCas13a crRNA#1172 | 1 μM | 7.5 | 250 nM |
| Total | | 30 (enough for | |
| | | 6 reactions) | |

3—Keep the assembly at room temperature while assembling the OPTIMA-dx reaction.

4—Assemble OPTIMA-dx master mix as follows:

| | | | Amount | Final | |
|---|---|---|---|---|---|
| Reagents | Concentration | | (μL) | concentration | |
| H₂O | | | 0.65 | | |
| Isothermal buffer | 10X | | 2.5 | 1X | |
| MgSO₄ | 100 | mM | 1.5 | 6 | mM |
| dNTPs | 25 | mM | 1.4 | 1.4 | mM |
| SC LAMP primers | 10X | | 2.5 | 1X | |
| NTPs | 10 | mM | 1.25 | 0.5 | mM |
| Bst DNA polymerase | 50 | U/μL | 1.2 | 2.4 | U/μL |
| RTx reverse transcriptase | 15 | U/μL | 0.5 | 0.3 | U/μL |
| RNasin Plus RNase inhibitor | 40 | U/μL | 0.5 | 0.8 | U/μL |
| Hi-T7 RNA polymerase | 50 | U/μL | 2 | 4 | U/μL |
| Inorganic pyrophosphatase | 20 | U/μL | 0.5 | 0.4 | U/μL |
| HEX mix reporter | 18.75 | μM | 1 | 750 | nM |
| RNP | 250 | nM | 5 | 50 | nM |
| template | | | 4.5 | | |
| Total | | | 25 | | |

5—Incubate the reaction at 56° C. for 60 mins.

6—Place the reaction tubes in p51 molecular fluorescence viewer and visualize the results.

### Assembly of Multiplexed OPTIMA-Dx Reaction.

1—Dilute TccCas13a protein into 2 μM in 1× isothermal buffer (Lucigen, 30027). 2—Dilute AapCas12b protein into 2 μM in 1× isothermal buffer (Lucigen, 30027). 3—Assemble of TccCas13a and AapCas12b RNPs as follows in 2 different tubes:

### —TccCas13a RNP Assembly:

| | Concentra- | | Final |
|---|---|---|---|
| Reagents | tion | Amount (μL) | concentration |
| H₂0 | | 4.5 | |
| Isothermal buffer | 10X | 3 | 1X |
| TccCas13a | 2 μM | 7.5 | 500 nM |
| TccCas13a crRNA | 1 μM | 15 | 500 nM |
| (#1172 for SARS-CoV2 | | | |
| or #1363 for HCV) | | | |
| Total | | 30 (enough for | |
| | | 6 reactions) | |

—AapCas12b RNP assembly:

| | Concentra- | Amount | Final |
|---|---|---|---|
| Reagents | tion | (μL) | concentration |
| H₂0 | | 4.5 | |
| Isothermal buffer | 10X | 3 | 1X |
| AapCas12b | 2 μM | 7.5 | 500 nM |
| AapCas12b sgRNA-1 for | 1 μM | 15 | 500 nM |
| RNase P detection. | | | |
| Total | | 30 (enough for | |
| | | 6 reactions) | |

4—Keep the RNPs at room temperature while assembling the multiplexed OPTIMA-dx reaction.

5—Assemble multiplexed OPTIMA-dx master mix as follows:

| | Concentra- | | Amount | Final | |
|---|---|---|---|---|---|
| Reagents | tion | | (μL) | concentration | |
| H₂O | | | 0 | | |
| Isothermal buffer | 10X | | 5 | 1X | |
| MgSO₄ | 100 | mM | 3 | 6 | mM |
| dNTPs | 25 | mM | 2.8 | 1.4 | mM |
| SC LAMP primers (or HCV | 10X | | 5 | 1X | |
| primers) | | | | | |
| RNase P primers | 10X | | 5 | 1X | |
| NTPs | 10 | mM | 2.5 | 0.5 | mM |
| Bst DNA polymerase | 50 | U/μL | 2.4 | 2.4 | U/μL |
| RTx reverse transcriptase | 15 | U/μL | 1 | 0.3 | U/μL |
| RNasin Plus RNase inhibitor | 40 | U/μL | 1 | 0.8 | U/μL |
| Hi-T7 RNA polymerase (High | 1000 | U/μL | 0.2 | 4 | U/μL |
| concentration (M0470T, NEB) | | | | | |
| Inorganic pyrophosphatase | 20 | U/μL | 1 | 0.4 | U/μL |
| FAM RNA mix reporter | 25 | μM | 0.5 | 250 | nM |
| HEX ssDNA reporter | 10 | μM | 1.25 | 250 | nM |
| TccCas13a RNP | 500 | nM | 5 | 50 | nM |
| AapCas12b RNP | 500 | nM | 5 | 50 | nM |
| template | | | 9.35 | | |
| Total | | | 50.65 | | |

6. Incubate the reaction at 56° C. for 60 mins in a 96-well Real-Time PCR detection system (CFX96 qPCR machine, Bio-Rad), with fluorescence measurements taken every 2 min using both FAM and HEX channels.

### OPTIMA-Dx Software Implementation

The dataset of fluorescent images used for training the software consisted of many random images annotated manually as positive or negative to set the proper fluorescence intensity threshold. The software was then trained and tested multiple times to reach the best mean average precision (mAP) value with this dataset. The application allows the user to easily take a picture of PCR strips or upload an already captured image of a PCR strip illuminated by a transilluminator. The software then determines the location of each tube, calculates a probability score for each target category and classifies each tube as positive (green bounding box) or negative (red bounding box) samples based on the intensity of the fluorescent signal (FIG. 6d). The entire image processing, from capturing the reaction tubes to the final app output results, takes less than 1 min. Once the image is captured, it can be uploaded and processed by the software.

### Deep Learning Framework

As of today, there are various deep learning frameworks for engineers and researchers to choose to train machine learning models [Abadi, M., et al., arXiv preprint arXiv: 1603.04467, 2016, Chen, T., et al., Mxnet: arXiv preprint arXiv: 1512.01274, 2015, Jia, Y., et al. Caffe: Convolutional architecture for fast feature embedding. in Proceedings of the 22nd ACM international conference on Multimedia. 2014, Paszke, A., et al., Pytorch: An imperative style, high-performance deep learning library. arXiv preprint arXiv: 1912.01703, 2019]. These frameworks abstract the underlying hardware and software stack to expose a simple API in language such as Python. Among these frameworks, TensorFlow is one of the most popular frameworks in deep learning community [Abadi, M., et al., arXiv preprint arXiv: 1603.04467, 2016]. Compared with TensorFlow, TensorFlow-Lite (TensorFlow Lite. tensorflow.org/lite.) is the lightweight version of TensorFlow, which is specifically designed for the mobile platform and embedded devices. In this project, TensorFlow framework was used to train the model on Linux workstation and use TensorFlow-Lite to deploy the trained model on mobile device with Android operating system.

### Object Detection Model

Object detection has been witnessing a rapid revolutionary change in the field of computer vision. Basically, it involves two tasks:

Object localization: determine where objects are located in a given image. Specifically, object detection model will use rectangular bounding boxes to locate all the detected objects in the image.

Object classification: determine which category each detected object belongs to. Specifically, for each detected object, object detection model will calculate a probability for each target category, indicating how likelihood this detected object belongs to this specific category.

Currently, there are many popular object detection models (e.g., SSD [10], RetinaNet [Lin, T.-Y., et al. Focal loss for dense object detection. in Proceedings of the IEEE international conference on computer vision. 2017], Faster R-CNN [Ren, et al., arXiv preprint arXiv: 1506.01497, 2015], Mask R-CNN [He, K., et al. Mask r-cnn. in Proceedings of the IEEE international conference on computer vision. 2017]) available in different deep learning frameworks. All these models have one common part called feature extractor, which focuses on calculating high quality features for object detection tasks. In order to deal with the problem of limited resources on the mobile device (e.g., small storage space and limited battery power), researchers have proposed some efficient feature extraction network architecture specifically tailored for mobile and resource constrained environments, such as MobileNets [Howard, et al., arXiv preprint arXiv: 1704.04861, 2017], MobileNetV2 [Sandler, M., et al. Mobilenetv2: Inverted residuals and linear bottlenecks. in Proceedings of the IEEE conference on computer vision and pattern recognition. 2018]. In this work, the SSD with MobileNetV2 (referred to as SSD-Mobilenet-V2) was the main focus. SSD (Single Shot MultiBox Detector) is a popular algorithm in object detection. Mobilenet-V2 is a convolution neural network used to produce high-level features which can be used as a backbone feature extractor for SSD. It is small, low-latency, low-power and parameterized to meet the resource constraints of a variety of use cases. Specifically, it can be run efficiently on mobile devices with TensorFlow-Lite. SSD-Mobilenet-V2 combines the advantages of the two models, enabling it to efficiently perform target detection tasks on mobile devices.

Transferring well-trained object detection models on one dataset to another new dataset is a common approach called transfer learning. It has several benefits, but the main advantages are saving training time, getting better performance, and not needing a lot of data. Google has trained one SSD-MobileNet-V2 object detection model using COCO dataset (Microsoft. https://cocodataset.org.), which has 90 different categories. This pre-trained model can be used as a good starting point for OPTIMA-dx detection model to help us save time and get better performance. Specifically, since this project only has 2 categories (positive and negative), final layers were modified to make it produce only 2 outputs corresponding to the 2 categories.

### Dataset

To train the model, one OPTIMA-dx image dataset was created using P51™ Molecular Fluorescence Viewer (minipcr. https://www.minipcr.com/product/p51-molecular-glow-lab). Specifically, cameras were used on different mobile devices to take pictures of the tubes shown on this device. At least 391 pictures have been directly taken from mobile device cameras. When taking these pictures, due to some random lighting, angles, jitter and other issues, some pictures are distorted and blurred, which are not suitable for training and testing the model, and need to be deleted. These images were further augmented by randomly modifying the brightness, contrast, color and sharpness, which generated a new set of images. Each image was made square by cropping to make sure each tube in each image can maintain a normal aspect ratio during model training and testing. Next, the images that meet the requirement were randomly divided into a training set (540 pictures) and a test set (66 pictures). Finally, the ground-truth bounding boxes for each picture in training and test set were created by LabelImg (tzutalin. github.com/tzutalin/labelimg).

### Model Training

In order to transfer visual knowledge learned from the large-scale generic dataset COCO to the model, it was initialized using Google's pre-trained SSD-Mobilenet-V2. The model was then trained using the training set. Specifically, it was trained on one GeForce GTX 1080 Ti GPU with batch size 10. RMSprop optimizer was used with initial learning rate as 0.004. A total of 35000 steps/batches were trained and one checkpoint was saved every 10 minutes. Then all the testing can be done using these saved checkpoints.

### Model Testing

The most common metric used to evaluate the performance of object detection model is the mAP (mean average precision) [Howard, A. G., et al., arXiv preprint arXiv: 1704.04861, 2017; Sandler, M., et al. Mobilenetv2: Inverted residuals and linear bottlenecks. in Proceedings of the IEEE conference on computer vision and pattern recognition. 2018.]. In the testing process, mAP was calculated on the test set for each saved check point, and finally take the checkpoint with the largest mAP. Currently, the best mAP is 97.6%.

### Android App Development

The selected checkpoint was converted into a TensorFlow Lite model using the model converter tool (tensorflow.org/lite/convert) provided by TensorFlow. Then it can be deployed and run this TensorFlow Lite model on mobile device. For each input image, the TensorFlow Lite model can output 3 different kind of information: 1) the location of the bounding box for each detected tube, containing four coordinate values on the image plane: left, top, right and bottom. 2) Category for each detected tube and its value is either positive or negative. 3) One confidence score indicating the probability that the model thinks one tube belongs to the category. Finally, the bounding boxes, categories and confidence scores are drawn over the input image and display the final image on screen.

The code for the smart phone app and to download the app is available at: hi-zhengcheng.github.io/optima-dx

### Results

Reverse transcription loop-mediated isothermal amplification (RT-LAMP) is a highly sensitive, robust and practical method that holds great promise in developing a viable POC detection platform. However, when used independently as a method to detect nucleic acids, it suffers from a high rate of false positives due to primer-dimer formation and cross-contamination. In order to address these drawbacks, introducing additional level of specificity is highly desirable. Coupling RT-LAMP to specific CRISPR/Cas target recognition and in trans reporter cleavage ensures signal appearance only in reactions where a correct amplicon has been generated. Nevertheless, most methods reported to date rely on transferring RT-LAMP product to the second tube, which complicates handling and, more importantly, leads to aerosol formation that is detrimental to accuracy of subsequent reactions. In order to address these shortcomings, an assay where all components are added in a single step and the reaction is incubated at a single temperature is needed. To achieve this goal, thermostable reagents are needed that can tolerate the relatively high temperatures needed for the RT-LAMP reactions.

### Identification, Screening and Characterization of Thermophilic Cas13 Enzymes

Recently, Cas13 variants from mesophilic bacteria have been employed for biosensing, including pathogen detection, genotyping, and diagnostics of viruses and disease markers. The SARS-COV-2 pandemic highlighted the need to develop POC diagnostics. RT-LAMP is a practical approach for POC diagnostics. Still, it needs to be developed in a one-pot closed system to avoid cross-contamination, facilitate user-friendliness and enhance sensitivity and specificity. The current Cas13 homologs can only be used in a two-pot assay which is problematic and poses a risk of cross-contamination. Therefore, it was aimed to identify Cas13 proteins from thermophilic bacteria and test their thermostability in a wider temperature range especially at higher temperatures suitable for developing one-pot RT-LAMP assay involving virus genome amplification and CRISPR-mediated detection in a single tube.

Cas13 variants were interrogated to determine whether some of these originate from thermophilic hosts. HheCas13a originating from Herbinix hemicellulosilytica thermophilic bacterium was identified as a potential thermophilic protein. Subsequently, the HheCas13a (1285 amino acids) was used as a query in BLAST-P NCBI searches of non-redundant protein sequences datasets to interrogate databases for potential thermophilic Cas13 homologs. A TccCas13 homolog originating from Thermoclostridium caenicola, sharing 87% identity at the amino acid level, was identified a as another likely thermophilic protein. The gene sequence of TccCas13a was synthesized and the available clone of HheCas13a was used for heterologous expression in E. Coli followed by purifying the proteins to homogeneity. Subsequently, differential scanning Fluorimetry (DSF) was conducted to test the thermostability of the HheCas13 and TccCas13a proteins.

The data showed that both proteins possess a denaturation temperature higher than those of mesophilic bacteria, e.g., LwaCas13a. Next, it was hypothesized that the complexing of sgRNA and the HheCas13 and TccCas13 proteins would further stabilize the proteins at higher temperatures. In silico prediction of the TccCas13a crRNA was performed and the recently reported crRNA of the HheCas13a was used. TccCas13a and HheCas13a were incubated with and without their respective in vitro transcribed sgRNAs at 37, 60, 70 and 90° C. for 30 minutes. The SDS-PAGE analysis demonstrated that the TccCas13a and HheCas13a loaded with sgRNA exhibit higher thermostability. However, TccCas13a exhibited a better thermostability compared to HheCas13a.

The data showed that both proteins exhibit thermostability and thus are promising candidates for downstream applications requiring cis and trans catalytic activities at higher temperatures, e.g., one-pot RT-LAMP for diagnostic applications as well as targeted gene knockdown, virus interference, and RNA editing and imaging.

### Characterization of the Cis and Trans Catalytic Activities of the TccCas13a and HheCas13a Thermophilic Proteins

The data showed that both proteins remain folded at higher temperatures and that loading of sgRNA enhances the thermostability of the proteins. Next, it was tested whether the RNP complex of these proteins is active and mediates cis and trans activities required for the downstream applications of these proteins. sgRNAs were designed to cleave a synthetic target sequence to determine the ability of both proteins to exhibit catalytic cis activities at higher temperatures.

The data showed that both proteins exhibit robust cis catalytic activities at higher temperatures.

In order to couple RT-LAMP with specific CRISPR/Cas based detection, two thermophilic Cas13 enzymes, namely HheCas13a and TccCas13a, were identified and their activity tested at relevant temperatures. When Cas13/crRNA ribonucleoproteins (RNP) recognize and cleave their target sequence, they exhibit collateral cleavage activity that degrades surrounding ssRNAs [8, 9]. Such collateral activity has been harnessed in nucleic acid detection applications, where a ssRNA probe (reporter) molecule present in the Cas13 reaction is degraded by target-dependent Cas13 collateral activity [7]. The ssRNA reporter contains a fluorophore linked by a short ssRNA sequence to a quencher, which emits fluorescence after the ssRNA sequence is cleaved, indicating the presence, and therefore the detection, of the target of interest. Because different Cas13 proteins exhibit different cleavage preferences depending on ssRNA sequences [6], the ssRNA reporter that can be cleaved by TccCas13a and HheCas13a effectors was investigated.

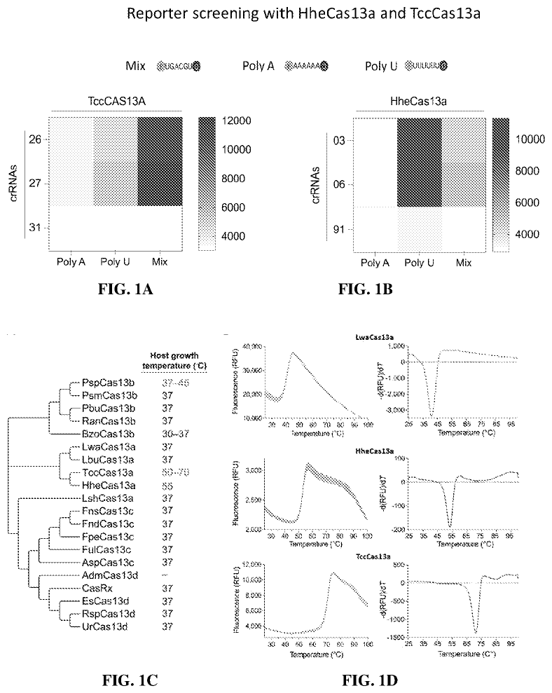

It was determined whether these proteins retain the non-specific trans degradation activities of ssRNA reporter molecules in the presence of ssRNA target at higher temperatures. Different Cas13a variants trigger collateral RNase activity at exposed uridine residues, so the trans-cleavage nucleotide preference of TccCas13a and HheCas13 at higher temperatures was tested. Therefore, 3 different ssRNA probes were screened. Each probe was conjugated to a 5′ fluorescent molecule (FAM) and a 3′ fluorescence quencher (FQ). 6-mers of poly (A) and poly (U) homopolymers and an additional 6-mer probe of mixed U, G, A, C nucleotides were used. Using two targeting crRNAs and one non-specific (NS) crRNA, it was observed that TccCas13a exhibited a trans-ssRNA cleavage preference against a mixed RNA sequence (not homopolymer ssRNA sequence) containing different nucleotides: UGACGU (FIG. 1A). HheCas13 exhibited a cleavage preference for a homo-uridine ssRNA substrate, consistent with previous observations (FIG. 1B) [10]. Moreover, the rate at which the trans-cleavage activity of ssRNA reporters reached saturation was determined using different target, ssRNA activator, concentrations. The data show that TccCas13 exhibited a higher sensitivity with only fM of the ssRNA activator resulting in detectable cleavage of the reporter.

Based on the observation that both HheCas13a and TccCas13a are derived from thermophilic host bacteria, it was determined whether both effectors exhibit cleavage activity at high temperatures. It was observed that both proteins were highly active at high temperature (60° C.), with TccCas13a showing stronger and faster activity at high temperature compared with its activity at 37° C. (FIGS. 2A-2B).

These results prompted the investigation of the activity of both Cas13 effectors at a broad range of elevated temperatures. Although LwaCas13a exhibited very robust and fast signal when incubated at 37° C., no activity was observed at high temperatures. In contrast, both HheCas13a and TccCas13a maintained robust trans cleavage activity at 56° C. and 60° C. HheCas13a maintained strong activity at temperatures up to 60° C. On the other hand, TccCas13a maintained a robust activity at temperatures as high as ˜ 70° C. (FIG. 2C). In addition, crRNAs with increased spacer lengths was tested using 24 and 28 nt long spacers. Subsequently, trans cleavage activity assays using a broad temperature range (37-72 C) was conducted. The data showed that TccCas13a loaded with crRNA2 is active over a wide range of temperature (37-70 C). HheCas13, however, showed, robust activity between 37-60 C. Upon exploring the crRNA spacer length requirements of the previously uncharacterized TccCas13a effector, it was observed that TccCas13a showed comparable activity using 24 or 28 nt long spacer sequences (FIG. 2D). These data provide compelling evidence on the thermostability and catalytic activities of TccCas13a and HheCas13a proteins and their usefulness in applications requiring cis and trans catalytic activities. See also FIGS. 1C-1E, 1F, and 2E.

### Biochemical Characterization of Thermostable TccCas13a and HheCas13a

To further characterize the activity of the identified thermostable Cas13 proteins, the crRNA requirements were tested by introducing various modifications to the crRNA spacer sequences. The effect of single mismatches between crRNA and target RNA on HheCas13a and TccCas13a RNA detection activities was tested. Single bases across the crRNA spacer sequence were mutated to the respective complementary bases. Results show that both HheCas13a and TccCas13a were tolerant to single mismatches across the spacer, as such mismatched spacers enabled RNA detection with similar efficiency as fully matched spacers (FIG. 14A and FIG. 14F).

However, double mismatches resulted in different tolerances for different regions of the spacer sequence with both HheCas13a and TccCas13a proteins (FIG. 14 and FIG. 14G). In addition, when stretches of 4 mismatches were introduced in the spacer, a significant reduction was found in the activity of both Cas13 enzymes for mismatch stretches in the center of the spacer, while 4 consecutive mismatches at the extreme 5′ or 3′ ends of the crRNA spacer had less of an effect, indicating the presence of a seed region in the center of the spacer sequence, similar to previous observations with other Cas13 effectors (FIG. 14C and FIG. 14H) [8].

The minimal spacer length required for efficient Cas13-mediated RNA detection was investigated. A series of spacer truncations ranging from 30 nt to 16 nt long spacers. While HheCas13a required 24 nt or longer spacers to maintain efficient RNA detection activity (FIG. 14I), TccCas13a exhibited robust activity with spacer sequences as short as 20 nt (FIG. 14D).

Notably, previous studies have shown the ability of CRISPR/Cas13 systems to process pre-crRNAs and generate mature crRNAs capable of guiding Cas13 enzymes to the target RNA [9]. Interestingly, HheCas13a is the only known Cas13 orthologue that is incapable of processing pre-crRNAs, at least in vitro [10]. Considering that both HheCas13a and TccCas13a are thermostable proteins and evolutionarily closely related (FIG. 1C), the ability of TccCas13a to process pre-crRNAs in vitro was investigated. Using 5′-FAM labeled pre-crRNA sequences, the pre-crRNA processing activity of TccCas13a was tested in comparison to HheCas13a and LwaCas13a proteins. LwaCas13a exhibited robust pre-crRNA processing activity, but HheCas13a did not process the cognate pre-crRNA when it was incubated at 37 or 60° C., [10]. pre-crRNA processing activity with TccCas13a at the tested temperatures was also not detected. These results, together with previous findings [10], indicate that the thermostable HheCas13a and TccCas13a enzymes are the only pre-crRNA processing defective Cas13 homologs known to date, pointing to a possible relationship between the thermostability of these Cas13 variants and the lack of pre-crRNA processing activities.

The robust activity of TccCas13a observed in the previous experiments led to further study the enzyme kinetics. Michaelis-Menten kinetic measurements of TccCas13a ssRNA trans cleavage activity were performed at elevated temperature and it was found that when TccCas13a is activated with ssRNA targets, it catalyzes trans ssRNA cleavage with catalytic efficiency (kcat/Km) of 0.24×106 M-1 s-1 (FIG. 14E). The observed high value of the Km indicates the low affinity of Cas13 enzyme with the non-specific, in trans, RNA target. Similar observations of high Km values were recently reported with other Cas13 variants [77].

### Use of Thermophilic Cas13s for SARS-COV-2 Detection in One-Pot and Two-Pot Assays

To ensure sensitive detection, pre-amplifying the RNA target of interest is a necessary step [7]. RT-LAMP isothermal amplification was chosen because it possesses several advantages over other amplification methods, including high sensitivity, rapid turnaround time, simple operation, and low cost [11]. Primer sets well-established in previous reports were used to target and amplify conserved regions in the SARS-COV-2 N gene, named here as STOPCovid (SC) primer sets [4, 12]. However, because Cas13s target RNA, these primers were modified by appending a T7 promoter sequence to the 5′ end of the first half of either the forward inner primer (FIP) or the backward inner primer (BIP). Therefore, during LAMP amplification, the T7 promoter sequence should get integrated into the amplified DNA products, providing a suitable template for the T7 RNA polymerase to transcribe the amplified LAMP product in vitro and generate RNA targets for Cas13 detection (Table 1).

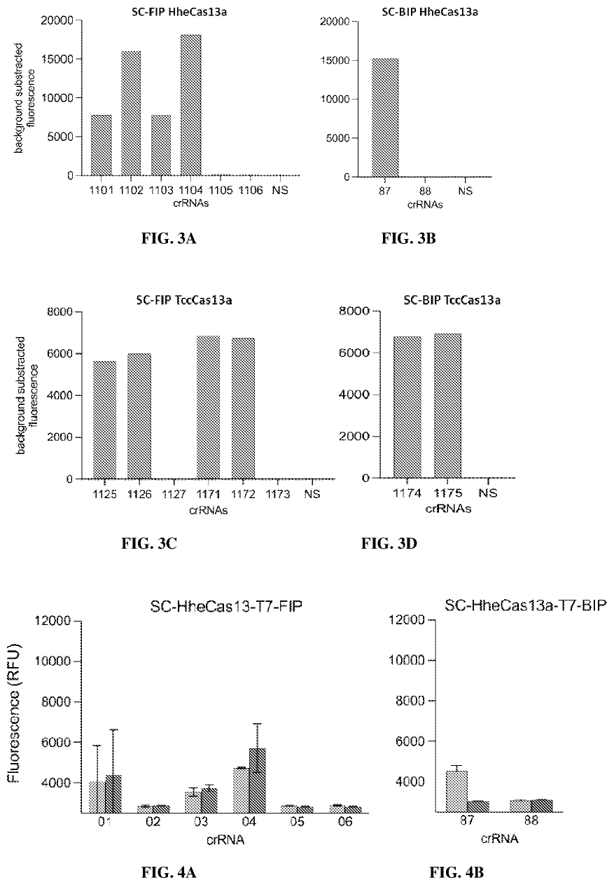

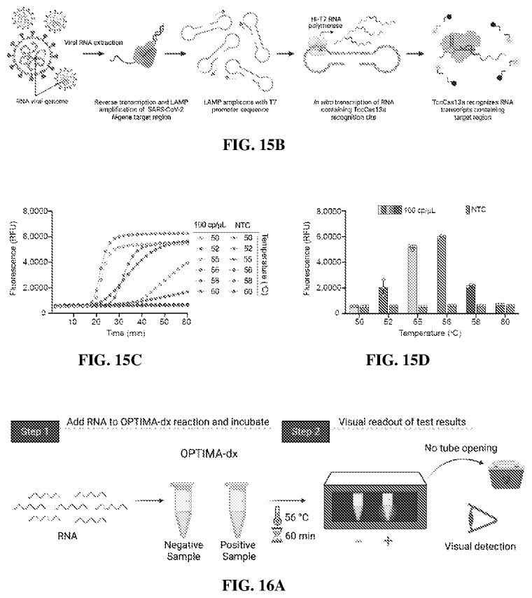

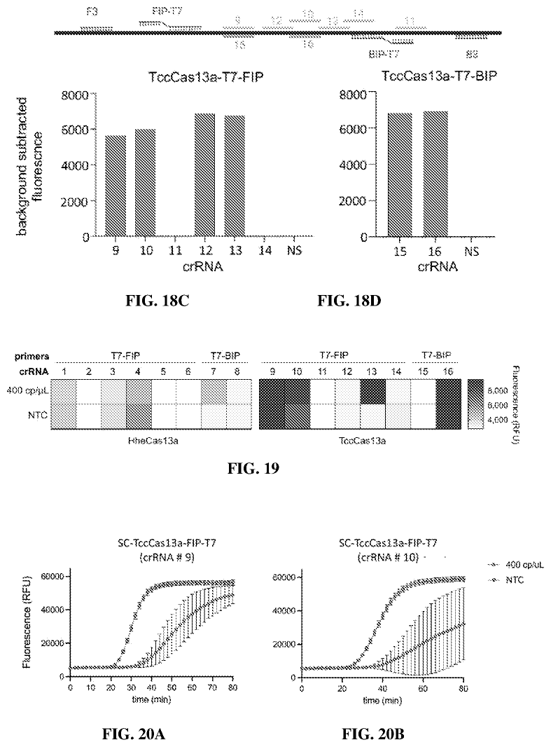

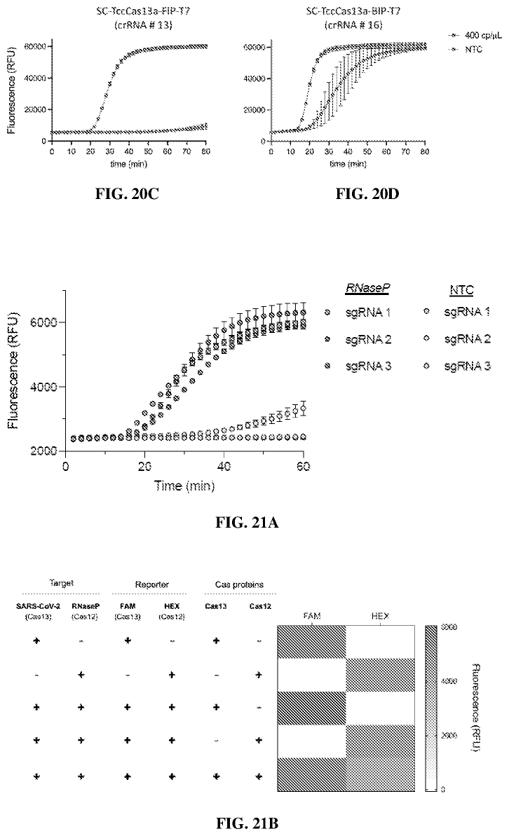

Various reports have shown the utilization of T7 RNA polymerase for subsequent in vitro transcription of the amplified product and Cas13 based detection. However, coupling of the RT-LAMP amplification, T7-mediated transcription, and Cas13 detection in one pot at relatively high temperature suitable for RT-LAMP amplification is an unmet need. Therefore, the thermostable Hi-T7 RNA polymerase, which exhibits optimal performance at temperatures close to where both RT-LAMP and thermophilic Cas13 variants are active, was utilized to accomplish this goal. crRNAs targeting the highly conserved region in the SARS-COV-2 N-gene were designed and tested with primer sets previously reported to be highly efficient and specific. Initial screening of these crRNAs and primer sets in two-pots settings, where RT-LAMP was performed first, and the amplified product was used in the second step for T7 RNA polymerase-mediated in-vitro transcription and Cas13-based detection, identified few crRNAs that were highly active (FIGS. 3A-3D). Most of the tested crRNAs targeting the RNA transcript produced from LAMP products harboring T7 promoter sequence from FIP-T7 or BIP-T7 primers showed robust performance and high detection signal. However, crRNAs targeting regions of LAMP amplicons that are not transcribed showed no activity. These results supported the strong detection of RT-LAMP products when using crRNAs, confirming that 1) the amplification of the synthetic SARS-CoV-2 genome was successful with the modified primers; 2) the T7 promoter was successfully integrated into the amplified products; 3) T7 RNA polymerase could use the amplified amplicons to generate Cas13a substrates that activate Cas13a enzymes to degrade ssRNA reporters and generate signal output (FIG. 15B, FIGS. 18A-18D).

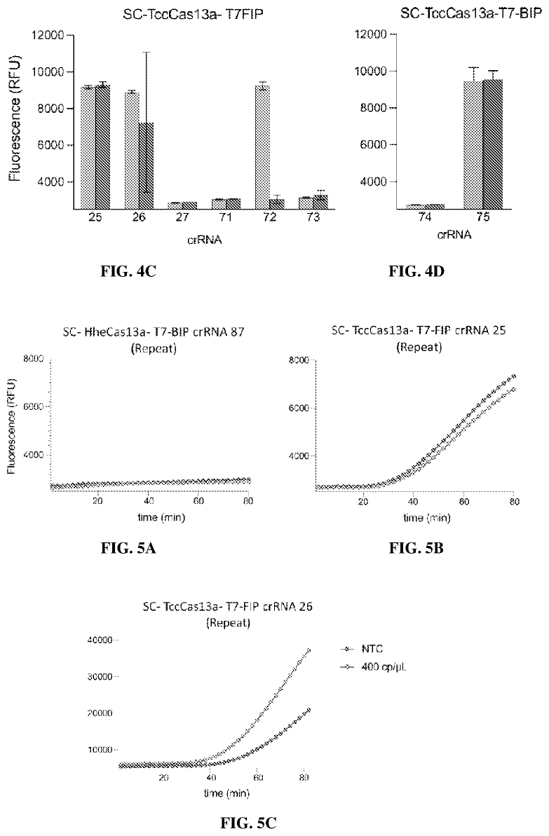

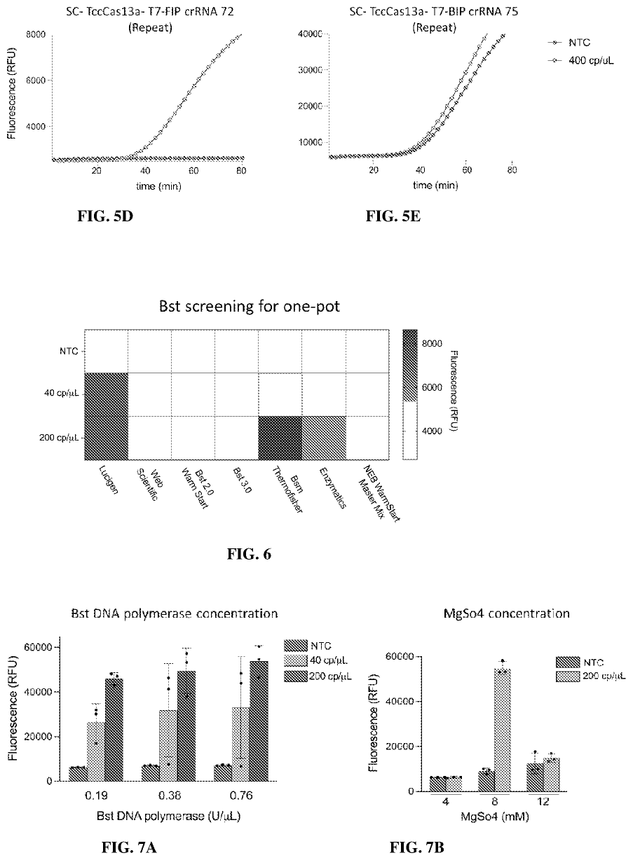

It was then investigated whether target detection in one-pot assay is feasible at a single temperature using all three enzymes. Therefore, all crRNAs and primer sets were rescreened in one pot settings. The screening assays identified a combination of crRNA and primer set that showed the most specific and efficient detection of the SARS-COV-2 RNA in comparison to the other tested crRNAs and primers, namely crRNA #1172 when used with SC T7-FIP modified primer sets (FIGS. 4A-4D). Consistently, this combination of primers and crRNA specifically and efficiently detected SARS-COV-2 target in one-pot (FIGS. 5A-5E). Interestingly, although HheCas13a exhibited strong detection signal in two-pot settings, no significant detection signal was identified in one-pot settings with any crRNA. By contrast, TccCas13a was consistent in specifically and efficiently detecting SARS-COV-2 target in one-pot with the optimized combination of primers and crRNA.

### Optimization of the One-Pot Assay

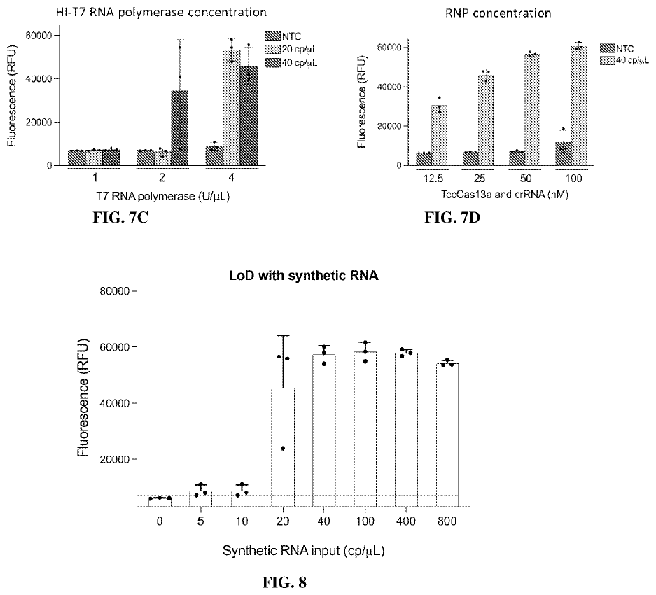

In order to maximize the efficiency of this system, the reaction chemistry was optimized in terms of the type of Bst DNA polymerase used, and the concentrations of Bst DNA polymerase, Hi-T7 RNA polymerase, Mg²⁺ and Cas13 RNP in the reaction (FIG. 6 and FIGS. 7A-7D). It was observed that the optimal sensitivity and efficiency of the one-pot detection assay can be achieved if the system is highly tuned with regards to the type and concentration of enzymes used at every step (FIG. 6 and FIGS. 7A-7D).

With the optimized reaction, the analytical limit of detection (LoD) of the Cas13-based one pot assay was evaluated using synthetic SARS-COV-2 RNA as an input. The LoD of the one pot assay was estimated to be 20 cp/μL, an improved sensitivity relative to reported Cas13-based one pot assays for SARS-COV-2 detection (FIG. 8) [13].

Because different biochemical reactions perform optimally at different temperatures in the one-pot assay, the performance of the one-pot assay was tested at different temperatures. The optimal temperature as determined by this experiment for the one-pot detection assay was 56° C., with diminished performance at higher or lower temperatures, probably due to the reduced performance of LAMP at lower temperatures, and of the Hi-T7 RNA polymerase at higher temperatures (FIGS. 15C, 15D).

### Validation of the Thermophilic Cas13-Based One-Pot Assay on Clinical Samples

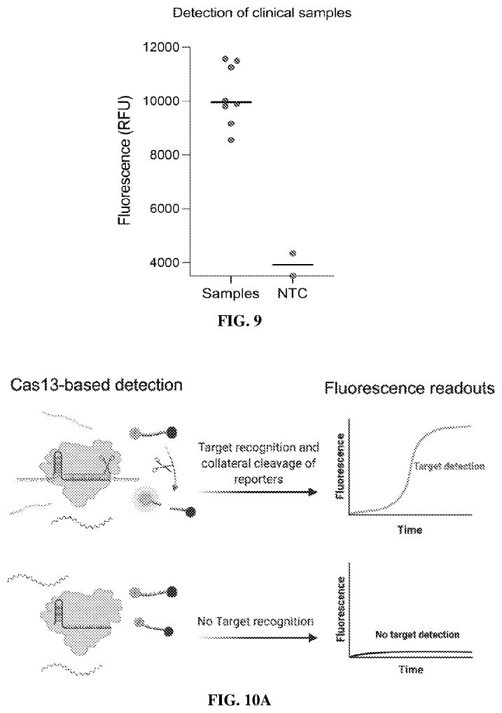

Next, the assay was validated with total RNA extracted from SARS-CoV-2 patient swab samples. Oropharyngeal or nasopharyngeal swab samples were collected from suspected COVID-19 patients. After RNA extraction following the CDC EUA-approved protocol, the samples were confirmed positive for SARS-COV-2 using RT-qPCR. The Cas13-based one-pot assay was first tested on 8 samples (provided in the chart above) with Ct values of 14-27. Using the one-pot Cas13 detection assay, all samples were correctly identified (FIG. 9). These results indicate that this newly developed detection system can reliably detect SARS-COV-2 in clinical samples.

### Evaluation and Clinical Validation of OPTIMA-Dx Assay for SARS-CoV-2 Visual Detection

To provide for large-scale screening during a pandemic, performing diagnostic assays at POC or outside of laboratory settings is important. Since the use of sophisticated fluorescence detection instruments such as qPCR machines or plate readers complicates the achievement of such goal, CRISPR diagnostic approaches have adapted lateral flow detection in an effort to develop a simple visual readout that can expedite accurate diagnostics in POC settings [43]. However, despite the efficiency and simplicity of this approach, the reaction tubes need to be opened for lateral flow detection readouts, thus increasing the chance of aerosols and cross-contamination.

As an alternative, a strategy was sought to couple the assay with a portable device. Using RNA reporter molecules conjugated to a 5′ HEX or FAM fluorophores at the appropriate concentration, TccCas13a collateral cleavage produced a bright signal visible with a hand-held, inexpensive fluorescence visualizer (P51 Molecular Fluorescence Viewer), allowing simple visualization and interpretation of the results (FIG. 16A). This one-pot assay with visual detection was termed OPTIMA-dx (One-pot thermophilic Cas13 and isothermal amplification module for nucleic acid detection).

With these optimized reaction conditions, the analytical limit of detection (LoD) of OPTIMA-dx was examined using synthetic SARS-COV-2 RNA as input. The LoD of OPTIMA-dx assay was estimated to be 10 copies (cp)/uL, which can be achieved within 45-60 min of reaction time (FIG. 16B). To test specificity and absence of cross-reactivity, OPTIMA-dx was challenged with other common human viruses, including SARS-COV-1, MERS-COV, H1N1, HCoV-OC43, HCOV-229E, and HCoV-NL63. OPTIMA-dx showed high specificity to SARS-COV-2, with no cross-reactivity against any of the other tested viruses.

Next, how storage at common storage temperatures influenced the performance of a pre-assembled OPTIMA-dx master mix was assessed. Although the OPTIMA-dx reaction did lose activity after storage for 48 h at 4° C., the detection reaction remained active when stored at −20° C. for at least 10 days and after multiple freeze-thaw cycles.

To ensure reliability of SARS-COV-2 detection kits, the Federal Drug Administration (FDA) guidelines (Catalog #2019-nCoVEUA-01, CDC, 2019) emphasize the importance of including a positive sample, or internal control, as an indicator of proper sample handling, RNA extraction, template quality and integrity, and validity of reagents. In particular, a negative SARS-COV-2 readout should be considered invalid if the internal control is negative as well. Thus human ribonuclease P (RNase P) transcripts were tested as an internal control for OPTIMA-dx SARS-COV-2 detection assay, whereby each sample can be evaluated by two OPTIMA-dx reactions for the detection of SARS-CoV-2 and the RNase P internal control. Accordingly, two crRNAs targeting a region of RNase P were designed amplified with RT-LAMP primers developed in previous reports that were modified with the T7 promoter sequence appended to the FIP primer [78]. Both crRNAs showed efficient and specific detection, with crRNA 1 showing a faster detection signal compared to crRNA 2, prompting selection of crRNA 1 for the OPTIMA-dx RNase P assay.

The one-pot detection assay was also conducted using RNA isolated from patient samples. SARS-COV-2 detection was performed at King Faisal Specialist Hospital & Research Centre using 73 RT-qPCR-positive samples with a broad range of Ct values and different strains of SARS-COV-2 and 27 RT-qPCR-negative samples extracted following the protocol approved by the Center for Disease Control Emergency Use Authorization (CDC EUA). The one-pot SARS-COV-2 detection assay demonstrated 94.5% sensitivity and 100% specificity, showing high concordance with the RT-qPCR data (FIG. 16C). Interestingly, although all 4 samples showing false negative results had Ct values above 30, the detection assay was able to detect other samples with Ct values as high as 34, indicating the high sensitivity of the assay (FIG. 16C). Also tested were all clinical samples for the RNase P gene. OPTIMA-dx detected the RNase P internal control in all tested samples, except one of the negative samples (FIG. 16D).

In addition, OPTIMA-dx was utilized for visual detection of SARS-CoV-2 with total RNA extracted from another set of swab samples collected from suspected SARS-COV-2 patients. The validation assays of OPTIMA-dx were conducted using RNA extracted from 45 randomized samples (different from samples used in FIG. 16C), 40 positive samples with Ct values ranging between 14-34, and 5 negative samples. A positive OPTIMA-dx signal was detected with all tested samples, with the exception of the negative samples and no template control (NTC) within 1 h. However, samples with Ct values above 30 show a weaker signal compared to samples with Ct values below 30. These results indicated that OPTIMA-dx can reliably detect SARS-COV-2 in patient samples within 1 h, with a simple visual readout, for Ct values up to 34.

### Development and Assessment of Simple Extraction Method for POC Sample Processing

An important consideration for rapid and simple POC diagnostics is to avoid complicated nucleic acid extraction procedures and kits that are labor-intensive, costly, and require specialized laboratory equipment and personnel training. Therefore, the development of an extraction-free sample processing protocol is critical to simplify CRISPR-dx and increase their user-friendliness for POC applications. Several extraction-free sample processing protocols have been previously developed and applied with CRISPR-dx, including HUDSON and proteinase K-based treatments [13, 44, 79]. These sample processing methods usually require heating steps for efficient lysis of viral particles and inactivation of nucleases. In addition, unlike conventional extraction methods that usually concentrate the extracted RNA, direct application of the treated sample to detection reactions introduces an upper bound and limited amount of inactivated sample input, resulting in decreased detection sensitivity [12].

Therefore, a simple extraction protocol was developed that can be performed at ambient temperature, thus avoiding the need for heating steps, and allowing the processing of large sample volumes, and increasing input via sample concentration. To this end, a viral RNA extraction buffer (Sigma Aldrich) was employed that provides rapid sample lysis and coupled it with sample concentration using magnetic beads. This RNA extraction buffer has several advantages, including short time of processing (5 minutes), room temperature incubation, stabilizing the released RNA, and compatibility with different sample types such as viral transport medium (VTM) and saliva. To streamline the protocol and allow the processing and RNA concentration from a large sample volume, the sample lysis step was combined with binding and concentration of released RNA using magnetic beads in a single step. This rapid protocol allows both steps (sample lysis and RNA binding to beads) to occur at room temperature in a short period of time (5 minutes).

First investigated was the ability of this method to lyse cells and capture the released RNA by detecting the RNase P template using oropharyngeal swabs collected from healthy donors and stored in VTM. RNA was released and concentrated from 200 μL of VTM into 30 μL final volume. To maximize the amount of sample input, the OPTIMA-dx reaction volume (50 μL instead of 25 μL) was doubled, which allowed use of up to 15 μL of the concentrated RNA sample. It was found that the extraction protocol could efficiently lyse cells and capture target templates, which enabled efficient detection of RNase P internal control with OPTIMA-dx. The performance of the extraction protocol and OPTIMA-dx was tested with oropharyngeal swabs collected from healthy donors and stored in VTM that was spiked with different concentrations of non-infectious SARS-COV-2 virus particles. OPTIMA-dx was able to detect viral load as low as 5000 cp/sample (˜50 cp/μL of reaction), and RNase P in all tested reactions.

Given the encouraging performance of the developed extraction protocol with OPTIMA-dx detection, this assay was conducted on clinical samples. 22 individual oropharyngeal swabs collected from COVID-19 patients and 2 oropharyngeal swabs collected from healthy donors stored in VTM were obtained. Part of these samples had previously undergone RNA extraction and tested with RT-qPCR in a diagnostic laboratory, allowing comparison of test results with the obtained RT-qPCR Ct values. These samples were processed with the developed extraction procedure and OPTIMA-dx was performed. The assay correctly detected all positive clinical samples with Ct values <34, indicating that the performance of OPTIMA-dx with the developed extraction protocol is equivalent to the performance observed with extracted RNA.

One-Pot Multiplexed Nucleic Acid Detection with Thermostable AapCas12b and TccCas13a Enzymes

An ideal diagnostic platform should allow multiplexed detection of more than one target in a single reaction [80]. Most of the developed CRISPR-based detection platforms detect only one pathogen or target in a given reaction. This can be attributed mainly to the non-specific collateral cleavage activity of CRISPR Cas systems that complicates the integration of more than one reporter in a single reaction. Therefore, in many cases, several separate and independent reactions are developed to detect different targets or internal controls [37, 12, 81]. The development of a one-pot multiplexed detection reaction is of great importance to improve the detection efficiency, accuracy, and clinical applicability of CRISPR-based diagnostics. Recently, a Cas12b enzyme from Alicyclobacillus acidiphilus (AapCas12b) was shown to be thermostable and can be coupled with RT-LAMP for the detection of SARS-COV-2 [12]. Different from Cas13, Cas12 collateral activity cleaves ssDNA molecules [82]. Therefore, it was reasoned that since both TccCas13a and AapCas12b are thermostable enzymes and have distinct cis and trans cleavage activities, the specific cleavage preference of these two enzymes could be used to develop a one-pot, RT-LAMP coupled multiplexed detection assay. Therefore, a one-pot multiplexed reaction was developed to detect SARS-COV-2 and the internal control RNase P in the same reaction.

The already established and optimized TccCas13a reaction was used for the detection of SARS-COV-2 using FAM-labelled RNA reporters, and develop AapCas12b-based detection of RNase P with the use of HEX-labelled ssDNA reporters, which would allow the differentiation between the two fluorescent signals using different detection channels (FIG. 17A). To this end, 3 different AapCas12b single guide RNAs (sgRNAs) were designed targeting the LAMP amplicon amplified by the primers used for RNase P detection, but with FIP primer lacking the T7 promoter. The 3 different sgRNAs were used in one-pot, RT-LAMP-coupled AapCas12b RNase P detection reaction using the same OPTIMA-dx reaction components and conditions, and found that all tested sgRNAs showed comparable performance and mediated robust detection as measured from the HEX fluorescence signal (FIG. 21A).

However, sgRNAs 1 and 3 showed faster and more specific signals compared to sgRNA 2. Therefore, sgRNA 1 were selected to develop the one-pot multiplexed OPTIMA-dx reaction. Next, it was tested if multiplexed RT-LAMP reaction is possible and if the Cas12 and Cas13-mediated fluorescent signals can be produced and distinguished from each other. The reaction mix contained both RT-LAMP primer sets for the detection of SARS-COV-2 and RNase P targets and both FAM and HEX reporters. SARS-COV-2 and RNase P were detected in the same reaction using HEX and FAM channels without any fluorescence signal interference from each Cas enzyme's collateral activity (FIG. 17B and FIG. 21B).

Next, the performance of the multiplexed OPTIMA-dx reaction was evaluated for the detection of SARS-COV-2 and the internal control RNase P on RNA extracted from 14 clinical COVID-19 samples. The OPTIMA-dx multiplexed reaction showed an unambiguous positive result for both SARS-CoV-2 and RNase P in all tested clinical samples (FIG. 17C). The multiplexed detection assay was evaluated on clinical swabs using the simple and quick crude sample extraction method developed above. To this end, 14 oropharyngeal swabs from patients with COVID-19 infection and 2 COVID-19 negative swabs were obtained. These samples were processed with the extraction procedure and OPTIMA-dx multiplexed detection was performed of both SARS-COV-2 and RNase P. The multiplexed OPTIMA-dx reaction reliably detected both SARS-COV-2 and RNase P in all SARS-COV-2 positive samples in 60 min (FIG. 17D).

Although the OPTIMA-dx master mix showed strong stability when stored at −20° C., it was sought to test whether the OPTIMA-dx reaction components can be freeze-dried, which would further simplify storage and distribution for POC applications. Therefore, OPTIMA-dx reaction was freeze-dried for multiplexed detection and the reaction was tested with the same samples processed with the quick extraction protocol in FIG. 17D. The OPTIMA-dx reaction remained functional and was able to detect SARS-CoV-2 and RNase P in all tested samples. However, a reduction in the reaction speed and performance was observed.

### Development of a Mobile Phone Application to Collect and Share SARS-COV-2 Test Results

Fluorescence detection devices like any plate reader or real-time PCR machines are choice devices to measure any end-point or real-time signal in a sample. However, at a POC settings with fewer resources and no specialized training, smartphone-based imaging is becoming popular in biomedical applications for easy data accessibility and sharing. To facilitate data collection and sharing of SARS-COV-2 test results as well as interpretation of the OPTIMA-dx readout, a deep learning-based approach was developed to design and develop a mobile phone application capable of collecting and reading OPTIMA-dx results from the low-cost P51 Molecular Fluorescence Viewer at POC settings.

The ability of the OPTIMA-dx smartphone application to identify and call positive and negative readouts from OPTIMA-dx results was validated: the app correctly determined the fluorescence status of each sample with good accuracy. The app was next evaluated on the 45 clinical samples tested with OPTIMA-dx in (discussed above): the app identified 38 out of the 45 samples

as positive. Notably, the two SARS-COV-2 positive samples deemed negative by the app had the highest Ct values (31 and 34) and thus lowest intense fluorescent signal of all samples. OPTIMA-dx was also run for RNase P in the same samples, resulting in 43 samples testing positive for RNase P out of the 45 samples tested, using the OPTIMA-dx one-pot assay and app. It was concluded that OPTIMA-dx can reach a performance of 95% sensitivity and 100% specificity in patient samples when combined with the mobile app, exhibiting high concordance with RT-qPCR data.

### Versatility of OPTIMA-Dx for Pathogen Diagnostics

The one-pot detection assay of OPTIMA-dx can be adapted for the detection of other pathogens. To demonstrate the versatility of OPTIMA-dx, the system was also employed for the detection of the human RNA virus hepatitis C virus (HCV) and the plant ssDNA virus Tomato yellow leaf curl virus (TYLCV). Using in vitro transcribed RNAs of two common HCV genotypes, it was shown that OPTIMA-dx can efficiently detect the two viruses within 1 hour (FIG. 22A). In addition, OPTIMA-dx was used to detect TYLCV ssDNA virus isolated from plants infected with the virus. After DNA isolation from infected, as well as non-infected (healthy), plants, the extracted DNA was diluted 1:10 and 1:100, and the diluted DNA was used as template for OPTIMA-dx reactions. OPTIMA-dx detected the virus only in the DNA extracted from infected plants in both DNA dilutions within 1 hour, indicating the high sensitivity and specificity of the OPTIMA-dx platform for efficient detection of plant DNA viruses (FIG. 22B). Having established multiplexed detection of SARS-COV-2 and RNase P with OPTIMA-dx, also shown was the versatility of OPTIMA-dx for multiplexed detection of HCV and RNase P internal control, further demonstrating the capability of OPTIMA-dx for multiplexed detection (FIG. 22C).

### DISCUSSION

Initially, CRISPR/Cas diagnostic systems were designed as two-pot assays [4, 5, 14], where reverse transcription and isothermal reactions were performed separately. Following the amplification step, a fraction of the reaction mixture was transferred to another tube containing the Cas enzyme and reporter. This approach, however, dramatically increases the chances of carry-over contamination and complicates handling, rendering such modules unfeasible as real-life diagnostic kits. Numerous attempts were reported to address these shortcomings including one-pot reaction systems using Cas12b and amplification-free detection protocols [17, 18]. Amplification-free strategies based on LbuCas13a have the advantage of facile protocol and absence of cross-contamination. However, sensitivity of such approaches remains low, with samples having Ct values above 29 evading detection. Furthermore, all present amplification-free techniques rely on a sophisticated device that must distinguish between genuine signal and the background. An advantage of the thermophilic Cas13-based one-pot detection assay described herein is the straightforward assay workflow that can be performed without sophisticated equipment or trained personnel. The whole assay can be performed in less than 80 minutes with only minimal equipment or risk of cross-contamination.

Frequent, rapid and cost-effective testing without relying on centralized facilities is a key advancement in early screening during pandemic situations [19].

This study shows 1) the first identification and characterization of thermophilic Cas13a enzymes, thereby expanding the molecular engineering toolbox of CRISPR systems for RNA substrates; 2) the first report of a one-pot assay using RT-LAMP coupled to Cas13 for specific and sensitive detection of SARS-COV-2 to facilitate POC applications and limit cross-contamination; 3) the utilization of the identified thermostable Cas13 with a thermostable Cas12b enzyme to develop a one-pot multiplexed detection reaction; 4) the development and use of mobile phone application coupled with the portable, affordable P51 detection to collect and share testing data with central facilities.

Since their discovery, Type VI CRISPR Cas13 systems have provided efficient and versatile tools for RNA manipulation [100, 101]. However, all Cas13 work to date has been restricted to temperatures around or below 42° C. Mining datasets from diverse natural contexts, including thermophiles, helped uncover novel thermostable variants that evolved naturally to provide immunity to their hosts. These thermophilic Cas13a enzymes will open diverse biotechnological applications for RNA-guided Cas13a ribonucleases at a broad temperature range and under harsh experimental or environmental conditions, especially if complexed with their corresponding crRNAs. For example, Cas13 has recently been gaining interest for therapeutics and disease research, including cancer gene therapy and antiviral therapeutics [83-86]. However, in vivo protein stability is important for successful applications and some proteins, such as LwaCas13a, mediate efficient knockdown only when fused to a stabilizing domain [88].

Notably, thermostabilization of proteins results in better stability in vivo [89, 90]. Therefore, the robust activity and thermostability of TccCas13a make this protein a promising candidate for further structural studies and potential in vivo RNA targeting applications. Moreover, thermostable enzymes (including thermostable Cas9) provide important genome editing applications in thermophiles [91]; specific RNA targeting at elevated temperatures beyond the range of previously reported Cas13 proteins is key to creating new tools for use in industrially important thermophiles, for which no CRISPR-Cas13 system has been reported. Phylogenetic analysis showed that HheCas13a and TccCas13a are evolutionarily closely related (FIG. 1C). Despite the strong conservation of pre-crRNA processing activity among Cas13 orthologues, HheCas13a has been shown to be the only known pre-crRNA processing defective Cas13 [10]. Interestingly, in addition to both proteins being thermostable Cas13s, the data show that both HheCas13a and TccCas13a are incapable of processing pre-crRNA. These observations could indicate a possible relationship between thermostability and the lack of pre-crRNA processing activity in these Cas13 variants. Besides the wide use of CRISPR-Cas13 enzymes for in vivo applications [92], Cas13 proteins have been increasingly used to develop various diagnostic platforms [80], and their usefulness for the development of diagnostics has become apparent during the COVID-19 pandemic [102-104, 93-95]. LAMP isothermal amplification was adapted to develop sensitive CRISPR diagnostics for SARS-COV-2 detection, including DETECTR [37], iSCAN [96], DISCOVER (Cas13-based module) and the first and only FDA-authorized CRISPR-Cas13 based diagnostics test (SHERLOCK CRISPR SARS-COV-2 Kit, IDT). However, all the above diagnostic methods are performed in two-pot settings, as their Cas enzymes function at around 37° C. and cannot tolerate the high temperatures needed for LAMP (˜55-65° C.). Such two-pot settings are not ideal for POC settings due to the increased chance of cross-contamination, which can be overcome with the implementation of thermostable DNA-targeting Cas12 proteins [12, 97]. Therefore, the thermophilic Cas13a protein offers a great advance in diagnostics and other applications. Coupling the activity of both thermostable Cas12 and Cas13 enzymes and the specific recognition of correct amplicons by each CRISPR/Cas system, OPTIMA-dx can be used for multiplexed detection of more than one target in a single reaction. The multiplexed detection capability of OPTIMA-dx could enable additional nucleic acid detection applications, including detecting different virus variants or different pathogens, such as other common respiratory viruses or bacteria in the same reaction. Future developments will include a visual readout of multiplex detection reactions for simple applications at POC.

During this work, two different thermostable Cas13 enzymes were characterized and a thermostable Cas13 was identified that could be adapted for a one-pot RT-LAMP coupled Cas13 detection reaction. Although HheCas13a showed good thermostability and activity in two pot system, good performance was not observed in a one-pot reaction. However, this does not exclude the compatibility of HheCas13a for one-pot detection reactions.

OPTIMA-dx detection module has several other advantages that make it suited for POC applications. OPTIMA-dx demonstrated excellent sensitivity with an LoD of 10 cp/μL of synthetic SARS-CoV-2 RNA and can detect samples with Ct values up to 34. Therefore, OPTIMA-dx exhibited robust sensitivity that provides its use for reliable SARS-COV-2 detection in clinical samples. In addition, besides the strong stability of OPTIMA-dx reaction master mix at −20° C. and tolerance to multiple freeze-thaw cycles, it was also demonstrated that OPTIMA-dx reagents can be lyophilized, which would facilitate pre-assembly of OPTIMA-dx reactions for transportation or long-term storage for large-scale screening in POC settings or remote areas. Moreover, OPTIMA-dx detection module does not require RNA extraction and is compatible with simple lysis and extraction methods. The ambient-temperature sample lysis and concentration method further increases the simplicity of the assay for POC applications.

The detection module was also integrated with the portable P51 Molecular Fluorescence Viewer to facilitate sample readout and developed a machine learning module for efficient data collection and sharing of the test results. Overall, the software provides an additional diagnosis validation and enables fast data sharing, making the entire diagnostic process affordable and accessible to a larger section of society. In conclusion, this work provides a thermophilic Cas13a variant and development of a one-pot RT-LAMP-coupled CRISPR-Cas13a assay for sensitive and specific SARS-COV-2 detection. The work provides a viable platform for COVID-19 detection in limited-resource settings. Moreover, the thermophilic Cas13a variants reported in this work have other applications beyond diagnostics, including in RNA knockdown, editing, imaging, and virus interference. This work thus expands the applications of CRISPR-Cas13 systems and offers new possibilities for transcriptome engineering and diagnostics at higher temperatures.

### Example 2: Development of a Miniature CRISPR-Cas13 System that Facilitates SARS-COV-2 Detection

CRISPR/Cas systems possess great potential for various applications, so there are ongoing efforts to search for, identify, and characterize new Cas effectors to increase utility and develop new tools for in vivo and in vitro applications [48, 49]. Recently, computational approaches for metagenomic mining resulted in the discovery of previously unknown CRISPR/Cas systems, including class II/type VI Cas proteins that exclusively target ssRNA substrates [50-53]. Besides repurposing these RNA targeting CRISPR/Cas13s for in vivo applications [54-58], Cas13s can be used in diagnostics that exhibit unprecedented sensitivity, specificity, and speed [40, 42-44, 47]. Different Cas13 variants have been used for nucleic acid detection. For example, Cas13a [41, 59], Cas13b [60], and Cas13d exhibit collateral cleavage activities and work for nucleic acid detection. This Example expands the existing Cas13-based toolbox for diagnostic applications by identifying and characterizing CRISPR/Cas13 effectors. In this Example, an mCas13 variant was identified, characterized, and its utility for SARS-COV-2 detection demonstrated. This work illustrates the untapped potential of mCas13 enzymes in diagnostics and other in vivo RNA applications.

### Materials and Methods

Computational identification of CRISPR/mCas13

Protein sequences of miniature Cas13s in a recent report were kindly provided by Dr. Hui Yang (Chinese Academy of Sciences, Beijing, China). These protein sequences were used as queries in the Basic Local Alignment Search Tool (BLAST) against the NCBI non-redundant (nr) protein database (before January 2021) using default settings. Only subject sequences with query coverage (Query cover) above 90% were considered. Protein sequences of the miniature Cas13f variants did not identify any subject sequences with query coverage above 90%. However, when using protein sequences of the miniature Cas13e variants, especially Cas13e.1 (accession #RKY08123), mCas13 (accession #HFH51004) showed up as the only subject sequence with query coverage above 90% (96%). Protein sequence alignment of mCas13 and the miniature Cas13e and Cas13f variants was performed using ClustalW in MEGAX with default settings, and the alignment was visualized using ESPript [74]. The RxxxxH (SEQ ID NO:192) HEPN motif was subsequently identified in the mCas13 protein sequence on the basis of this alignment and was further confirmed by manually searching for this motif using SnapGene. CRISPRCasFinder was performed on the genomic DNA sequence (GenBank #DSVK01000191.1) to identify the associated CRISPR array. CRISPRDetect was then used to predict the orientation of the direct repeat in the mCas13 CRISPR array.

### Cas13 Protein Expression and Purification

To produce the expression plasmid for Cas13 expression and purification, the E. coli codon-optimized Cas13 coding sequence was synthesized (GenScript) de novo and subcloned in frame with His and SUMO tags on the N-terminus into the His6-TwinStrep-SUMO bacterial expression vector (Addgene #115267) using BamHI and NotI (Table 8). Purification of mCas13 protein was performed following the protocol of Kellner et al. (2019) with a few modifications. Briefly, the mCas13 expression vector was transformed into BL21 E. coli cells. Starter cultures were prepared by growing single colonies in LB broth supplemented with 100 μg/mL ampicillin for 12 h at 37° C. Next, 20 mL of starter culture was used to inoculate 2 L of Terrific Broth medium (TB) (IBI scientific) supplemented with 100 μg/mL ampicillin for growth at 37° C. until an OD₆₀₀ of 0.5. Cells were incubated on ice for 30 mins, expression was induced with 0.5 mM IPTG, and cultures were then transferred to 16° C. for overnight expression. Cells were harvested by centrifugation for 20 min at 4° C. at 4000 rpm. Cell pellets were resuspended in lysis buffer (50 mM Tris-Cl pH 7.5, 500 mM NaCl, 5% glycerol, 1 mM DTT, EDTA-free protease inhibitor (Roche)) and supplemented with 1 mg/mL lysozyme (L6876, Sigma). Cells were lysed by sonication and clarified by centrifugation at 11,000 rpm for 50 min. The soluble 6×His-SUMO-mCas13 in cleared lysate was then purified with an affinity chromatography column (HiTrap Q HP, 5 mL GE Healthcare) (AKTA PURE, GE Healthcare) followed by concurrent removal of the 6×His-SUMO tag by SUMO protease and overnight dialysis in dialysis buffer. Cleaved protein was concentrated to 1.5 mL by Amicon Ultra-15 Centrifugal Filter Units (50 kDa NMWL, UFC905024, Millipore) and further purified via size-exclusion chromatography on a S200 column (GE Healthcare) in gel filtration buffer (50 mM Tris-HCl, 600 mM NaCl, 10% glycerol, 1 mM DTT, pH 7.5). The protein-containing fractions resulting from the gel filtration were pooled, snap frozen, and stored at −80° C.

### Nucleic Acid Preparation

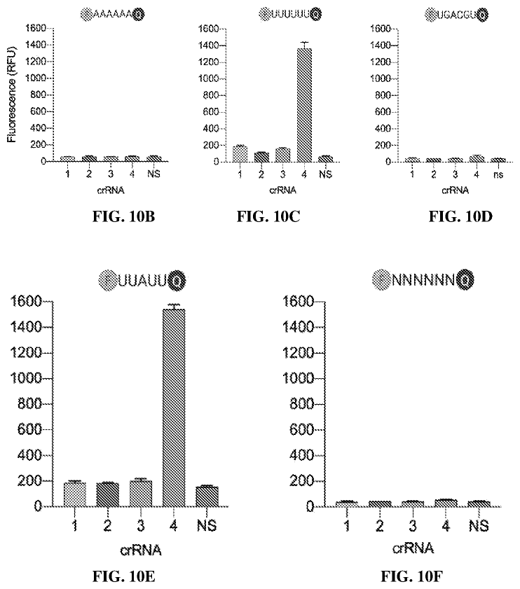

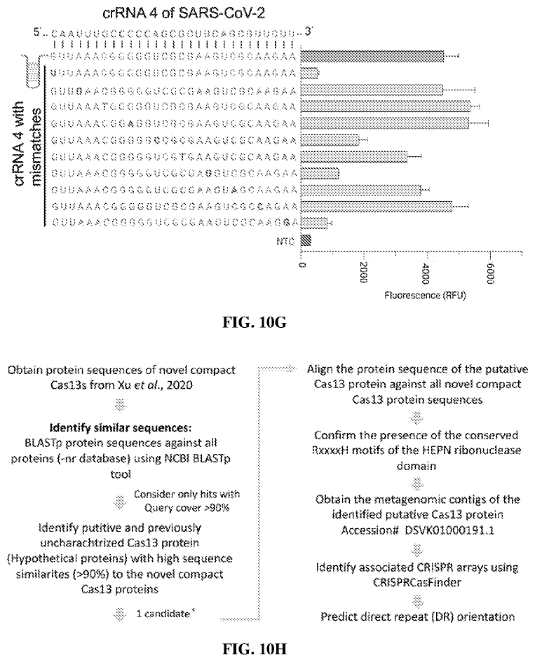

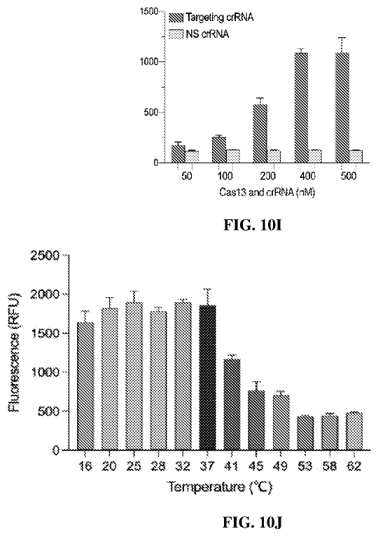

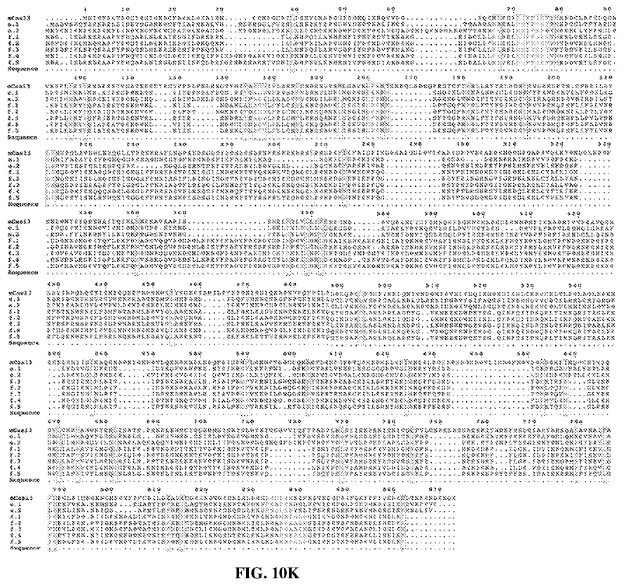

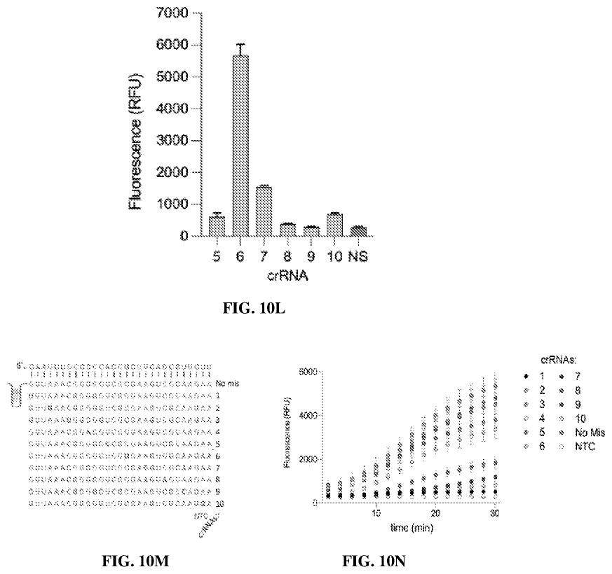

A short region of the SARS-COV-2 N gene sequence was used as a synthetic target in the preliminary mCas13 characterization and optimization experiments to screen crRNAs and collateral reporters and establish mCas13-based detection (FIG. 10). The N gene target RNA sequences were prepared by in vitro transcription of PCR amplicons containing the T7 promoter sequence using the 2019-nCOV_N_Positive Control plasmid as a PCR template (10006625, IDT). Purified PCR amplicons (QIAquick PCR Purification Kit, QIAGEN) were transcribed in vitro using HiScribe T7 Quick High Yield RNA Synthesis Kit (E2050, NEB). The transcripts were purified using Direct-zol RNA Miniprep Kits (R2050, Zymo Research) following the manufacturer's instructions, and the purified RNA was stored at −80° C.

mCas13 crRNAs were designed to target the N gene sequence of the SARS-COV-2 genome. For crRNA preparation, templates for in vitro transcription were generated using single-stranded DNA oligos containing a T7 promoter, scaffold, and spacer in reverse complement orientation (IDT), and were then annealed to T7 forward primer in Taq DNA polymerase buffer (Invitrogen). The annealed oligos were then used as templates for in vitro transcription as described above.

To establish RT-LAMP coupled with T7-mCas13-based detection and LoD range, control synthetic SARS-COV-2 viral genomic sequences used in FIG. 11 were ordered as synthetic RNA from Twist Bioscience, and were diluted to 10,000 RNA copies/uL and used at indicated concentrations to create simulated clinical samples.

For RT-LAMP amplification (described below), previously published LAMP primers designed to amplify the SARS-COV-2 N gene (Joung et al., 2020 [36], Broughton et al., 2020 [37]) were used, with modifications. The FIP or BIP primers were each designed with the appended T7 promoter sequence at the 5′ end of the first half of the primer. Such modification allows the modified primer to integrate the T7 promoter sequence in the LAMP-amplified product for subsequent T7-mediated in vitro transcription. All oligo sequences and substrates are listed in Tables 1, 2 and 7.

Screening of crRNAs and Reporters and Establishing mCas13 Collateral Detection

Activity and collateral assays of Cas13 were performed in 1× cleavage buffer (20 mM HEPES-Na pH 6.8, 50 mM NaCl, 5 mM MgCl₂, 1 mM DTT) in a 20-μL final reaction volume. Cas13 and crRNAs RNPs were first assembled by mixing 500 nM purified Cas13 with 500 nM crRNA (unless otherwise indicated) in 1× cleavage buffer and 20 units RNaseOUT (Invitrogen), followed by incubation at 37° C. for 15 minutes. Next, the assembled RNP was combined on ice with 2 μL of 500 ng/uL in vitro-transcribed target RNA and 250 nM RNA reporter, and reactions incubated for 1 hr at 37° C. (unless otherwise indicated). Real time or end-point fluorescence measurements were collected on a microplate reader M1000 PRO (TECAN) at 2-min intervals (for real time measurements) using 384-well, black/optically clear flat-bottomed plate (Thermofisher).

### RT-LAMP Reactions

Reverse transcription and isothermal amplification of target nucleic acids were performed using final concentrations of 1.6 μM FIP/BIP primers (with the T7 promoter sequence fused to either the FIP or BIP primer), 0.2 μM F3/B3 primers, and 0.4 μM LF/LB primers, 1× Isothermal Amplification Buffer (20 mM Tris-HCl, 50 mM KCl, 10 mM (NH₄)₂SO₄, 2 mM MgSO₄, 0.1% Tween 20, pH 8.8) (B0537, NEB), 1.4 mM dNTPs, 8 units of Bst2.0 WarmStart DNA Polymerase (M0538, NEB), 7.5 units of WarmStart RTx Reverse Transcriptase (M0380, NEB) and 6 mM MgSO₄ (B1003, NEB) in 25-μL reactions containing variable concentrations of SARS-COV-2 control standards, or 4 μL of isolated RNA from clinical samples. LAMP reactions were performed at 62° C. for 35 minutes in a PCR (C1000 touch thermal cycler, BioRad) machine.

One-Step T7 Transcription and mCas13 Detection

The two reactions, T7-mediated in vitro transcription and mCas13-based detection of the amplified and in vitro-transcribed target RNA, were carried out in the same tube. Briefly, 2 μL of the RT-LAMP reaction product was combined with 1× cleavage buffer (described above), 500 nM mCas13/crRNA assembled RNPs, 25 units T7 RNA polymerase (M0251, NEB), 1 mM NTPs, and 250 nM RNA reporter in 20-μL reactions. The reactions were incubated at 37° C. for 20-30 minutes.

### Visual Cas13-Based Detection

For simple visualization of mCas13-based detection, RNA reporters labeled with the HEX fluorophore were used instead of the FAM fluorophore (Table 3). Collateral cleavage of HEX reporters results in a bright signal that can be easily visualized upon excitation with LED light (Ali et al., 2020). Cas13-based reactions were carried out as described above, with modifications. For each reaction, 1 μM of HEX reporter (unless otherwise indicated) was used in 20-μL T7-mCas13 detection reactions. Reactions were incubated at 37° C. for 30 minutes. Reaction tubes were then transferred into the P51 Molecular Fluorescence Viewer (miniPCR) and photos were taken using a smartphone with default settings.

### Clinical Sample Collection and RNA Extraction

Oropharyngeal and nasopharyngeal swabs were collected from suspected COVID-19 patients by physicians in Ministry of Health hospitals in Saudi Arabia and placed in 2-mL screw-capped cryotubes containing 1 mL of TRIZOL for inactivation and transport. Each sample tube was sprayed with 70% ethanol, enveloped with absorbent tissues, then placed and sealed in an individual labeled biohazard bag. The bags were then placed in leak-proof boxes and sprayed with 70% ethanol before placement in a dry ice container for transfer to King Abdullah University of Science and Technology (KAUST). Total RNA was extracted from the samples following instructions as described in the CDC EUA-approved protocol and using the Direct-zol kit (Direct-zol RNA Miniprep, Zymo Research; catalog #R2070) following the manufacturer's instructions.

### Real-Time Reverse Transcription PCR (RT-PCR) for Detecting Positive 5 SARS-COV2 RNA Samples.

RT-PCR was conducted on extracted RNA samples using the oligonucleotide primer/probe (Integrated DNA Technologies, catalog #10,006,606) and Superscript III one-step RT-PCR system with Platinum Taq Polymerase (catalog #12574-026) following the manufacturer's protocol.

### Nucleotide and Amino Acid Sequences Used

TABLE 7
crRNA sequences used in this study.

| Name | Sequence | Note |
|---|---|---|
| Sense-mCas13-T1 | GCTGGAGCAGCCCTCGATTTGCAGGGTAATCACAGCAC | crRNAs for |
| (crRNA 1) | AAGGAACTGATTACAAACATTGGCCGCACCCTATAGTG | SARS-CoV-2 |
| | AGTCGTATTAATTTC (SEQ ID NO: 41) | N gene |
| Sense-mCas13-T2 | GCTGGAGCAGCCCTCGATTTGCAGGGTAATCACAGCAC | Target 1 |
| (crRNA 2) | TGATTACAAACATTGGCCGCAAATTGCACCCTATAGTG | |
| | AGTCGTATTAATTTC (SEQ ID NO: 42) | |
| Sense-mCas13-T3 | GCTGGAGCAGCCCTCGATTTGCAGGGTAATCACAGCCG | |
| (crRNA 3) | TTCTTCGGAATGTCGCGCATTGGCATGGCCCTATAGTGA | |
| | GTCGTATTAATTTC (SEQ ID NO: 43) | |
| Sense-mCas13-T4 | GCTGGAGCAGCCCTCGATTTGCAGGGTAATCACAGCCA | |
| (crRNA 4) was | ATTTGCCCCCAGCGCTTCAGCGTTCTTCCCTATAGTGAG | |
| used for all SARS- | TCGTATTAATTTC (SEQ ID NO: 44) | |
| CoV-2 detection | | |
| experiments | | |
| Sense-mCas13-N | GCTGGAGCAGCCCTCGATTTGCAGGGTAATCACAGCAA | crRNAs for |
| target 2-1 | GATCACATTGGCACCCGCAATCCTGCTACCCTATAGTG | SARS-CoV-2 |
| (crRNA 5) | AGTCGTATTAATTTC (SEQ ID NO: 45) | N gene |
| Sense-mCas13-N | GCTGGAGCAGCCCTCGATTTGCAGGGTAATCACAGCAC | Target 2 |
| target 2-2 | AATGCTGCAATCGTGCTACAACTTCCTCCCTATAGTGAG | |
| (crRNA 6) | TCGTATTAATTTC (SEQ ID NO: 46) | |
| Sense-mCas13-N | GCTGGAGCAGCCCTCGATTTGCAGGGTAATCACAGCCG | |
| target 2-3 | TTCCTCATCACGTAGTCGCAACAGTTCACCCTATAGTGA | |
| (crRNA 7) | GTCGTATTAATTTC (SEQ ID NO: 47) | |
| Sense-mCas13-N | GCTGGAGCAGCCCTCGATTTGCAGGGTAATCACAGCAC | |
| target 2-4 | AGTTCAAGAAATTCAACTCCAGGCAGCACCCTATAGTG | |
| (crRNA 8) | AGTCGTATTAATTTC (SEQ ID NO: 48) | |
| Sense-mCas13-N | GCTGGAGCAGCCCTCGATTTGCAGGGTAATCACAGCGA | |
| target 2-5 | TGCTGCTCTTGCTTTGCTGCTGCTTGACCCTATAGTGAG | |
| (crRNA 9) | TCGTATTAATTTC (SEQ ID NO: 49) | |
| Sense-mCas13-N | GCTGGAGCAGCCCTCGATTTGCAGGGTAATCACAGCAG | |
| target 2-6 | GCCAAACTGTCACTAAGAAATCTGCTGCCCTATAGTGA | |
| (crRNA 10) | GTCGTATTAATTTC (SEQ ID NO: 50) | |
| Sense-mCas13-NS | GCTGGAGCAGCCCTCGATTTGCAGGGTAATCACAGCTT | Non-specific |
| (NS) | ATGCCTCTTCCGACCATCAAGCATTTTACCCTATAGTGA | targeting |
| | GTCGTATTAATTTC (SEQ ID NO: 51) | |
| Sense-mCas13-4- | GCTGGAGCAGCCCTCGATTTGCAGGGTAATCACAGCAA | |
| Mis-1 | ATTTGCCCCCAGCGCTTCAGCGTTCTTCCCTATAGTGAG | |
| | TCGTATTAATTTC (SEQ ID NO: 52) | |
| Sense-mCas13-4- | GCTGGAGCAGCCCTCGATTTGCAGGGTAATCACAGCCA | |
| Mis-2 | ACTTGCCCCCAGCGCTTCAGCGTTCTTCCCTATAGTGAG | |
| | TCGTATTAATTTC (SEQ ID NO: 53) | |
| Sense-mCas13-4- | GCTGGAGCAGCCCTCGATTTGCAGGGTAATCACAGCCA | |
| Mis-3 | ATTTACCCCCAGCGCTTCAGCGTTCTTCCCTATAGTGAG | |
| | TCGTATTAATTTC (SEQ ID NO: 54) | |
| Sense-mCas13-4- | GCTGGAGCAGCCCTCGATTTGCAGGGTAATCACAGCCA | |
| Mis-4 | ATTTGCCTCCAGCGCTTCAGCGTTCTTCCCTATAGTGAG | |
| | TCGTATTAATTTC (SEQ ID NO: 55) | |
| Sense-mCas13-4- | GCTGGAGCAGCCCTCGATTTGCAGGGTAATCACAGCCA | |
| Mis-5 | ATTTGCCCCCGGCGCTTCAGCGTTCTTCCCTATAGTGAG | |
| | TCGTATTAATTTC (SEQ ID NO: 56) | |
| Sense-mCas13-4- | GCTGGAGCAGCCCTCGATTTGCAGGGTAATCACAGCCA | |
| Mis-6 | ATTTGCCCCCAGCACTTCAGCGTTCTTCCCTATAGTGAG | |
| | TCGTATTAATTTC (SEQ ID NO: 57) | |
| Sense-mCas13-4- | GCTGGAGCAGCCCTCGATTTGCAGGGTAATCACAGCCA | |
| Mis-7 | ATTTGCCCCCAGCGCTCCAGCGTTCTTCCCTATAGTGAG | |
| | TCGTATTAATTTC (SEQ ID NO: 58) | |
| Sense-mCas13-4- | GCTGGAGCAGCCCTCGATTTGCAGGGTAATCACAGCCA | |
| Mis-8 | ATTTGCCCCCAGCGCTTCATCGTTCTTCCCTATAGTGAG | |
| | TCGTATTAATTTC (SEQ ID NO: 59) | |
| Sense-mCas13-4- | GCTGGAGCAGCCCTCGATTTGCAGGGTAATCACAGCCA | |
| Mis-9 | ATTTGCCCCCAGCGCTTCAGCGGTCTTCCCTATAGTGAG | |
| | TCGTATTAATTTC (SEQ ID NO: 60) | |
| Sense-mCas13-4- | GCTGGAGCAGCCCTCGATTTGCAGGGTAATCACAGCCA | |
| Mis-10 | ATTTGCCCCCAGCGCTTCAGCGTTCCTCCCTATAGTGAG | |
| | TCGTATTAATTTC (SEQ ID NO: 61) | |

TABLE 8
mCas13 protein sequence and tag sequences for protein
purification.

| Name | Protein sequence |
|---|---|
| mCas13 | MGSSHHHHHHSSGLVPRGSHMASWSHPQFEKGGGSGGGSGGSAWSHPQFEKMSD |
| | SEVNQEAKPEVKPEVKPETHINLKVSDGSSEIFFKIKKTTPLRRLMEAFAKRQGKEM |
| | DSLRFLYDGIRIQADQTPEDLDMEDNDIIEAHREQIGGSMGIDYSLTSDCYRGINKSC |
| | FAVALNIAYDNCDHKGCRTLLSEVLRSKGGISDEQIKSQVVDGIQKRLKDIRNYFSH |
| | YYHAEDCLRFGDQDAVKVFLEEIYKNAESKTVGATKESDYKGVVPPLFELHNGTY |
| | MITAAGVIFLASFFCHRSNVYRMLGAVKGFKHTGKEQLSDGQKRDYGFTRRLLAY |
| | YALRDSYSVGAEDKTRCFREILSYLSRVPQLAVDWLNEQQLLTPEEKEAFLNQPAE |
| | DEGGDISDSSSSDKNKKSKEKRRSLRRDEKFILFAIQFIEGWAAEQGLDVTFARYQK |
| | TVEKAENKNQDGKQARAVQLKYRNQGLNPDFNNEWMYYIQNEHAIIQIKLNNKK |
| | AVAARISENELKYLVLLIFEEKGNDAVQKLNCYIYSMSQKIEGEWKHRPEDERWMP |
| | SFTKRADRTVTPEAVQSRLSYIRKQLQETIEKIGQEEPRNNKWLIYKGKKISMILKFIS |
| | DSIRDIQRRPNVKQYHILRDALQRLDFDGFYKELQNYVNDGRIAVSLYDQIKGVNDI |
| | SGLCKKVCELTLERLAGLEAKNGSELRRYIGLEAQEKHPKYGEWNTLQEKAKRFLE |
| | SQFSIGKNFLRKMFYGDCCQKRCFDEEKGYNTQAKERKSLYSIVKEKLKDIKPIHDD |
| | RWYLIDRNPKNYDNKHSRIIRQMCNTYIQDVLCMKMAMWHYEKLISATEFRNKLE |
| | WNCIGQGNMGYERYSLWYKTGCGVVIQFTPADFLRLDIIEKPAMIENICQCFVLGN |
| | KKLNSGAEKKITWDKFNKDGIAKYRKRQAEA VRAIFAFEEGLKIQEDKWSHERYFP |
| | FCNILDEAVKQGKIKDTGKDKEALNRGRNDFFHEEFKSTEDQQAIFQKYFPIVERKD |
| | DTKKRRDKKQK* (SEQ ID NO: 62) |

6x His affinity tag: residues 5-10; Thrombin site: residues 14-19; Strep-tag II: residues 24-31 and 44-51; SUMO: residues 52-148; mCas13 protein: residues 151-1023.

### Results

### Identification, Design, Construction, and Expression of a Miniature Cas13 System

Xu et al. describes compact Cas13 effectors that are classified as CRISPR/Cas type VI-E and VI-F. These compact Cas13s are used for in vivo applications, including endogenous RNA interference, RNA editing, and as an anti-coronavirus approach that targets and combats SARS-CoV-2 and other influenza viruses in vivo [62].

Some of the compact Cas13 protein sequences from Xu et al. were used as queries in the Basic Local Alignment Search Tool (BLAST) to find potentially uncharacterized mCas13 proteins. The alignment identified various proteins, including a few uncharacterized putative Cas13 sequences with two predicted RxxxxH (SEQ ID NO:192) motifs of the conserved Cas13 Higher Eukaryotes and Prokaryotes Nucleotide-binding (HEPN) ribonuclease domains [62, 63]. One small (837 amino acids) candidate showed high similarity (>95% query coverage) to the most efficient Cas13 identified in Xu et al, namely Cas13e.1 [62]. Further analysis of the metagenomic contigs showed that the putative mCas13 protein has an associated CRISPR array in its immediate vicinity. In silico analysis of the associated CRISPR array predicted that the mCas13-associated crRNAs share high similarity with the length and architecture of the compact Cas13e.1 crRNA (30-nt long spacer sequence at the 5′ end of the crRNA followed by a 36-nt long direct repeat (DR) sequence at the 3′ end), and to previously reported crRNAs of the Cas13b family [55]. A schematic illustration of the computational strategy is shown in FIG. 10H. Furthermore, multiple sequence alignment analysis showed high similarity and the two predicted HEPN domains conserved among the putative mCas13 protein, Cas13e.1, and the other compact proteins, Cas13e and Cas13f (FIG. 10K). Based on these predictions, it was hypothesized that the putative mCas13 is functionally active. Next, the corresponding gene sequence was codon-optimized and synthesized and a bacterial expression plasmid for heterologous expression in BL21 Escherichia coli containing the mCas13 sequence was designed and constructed. Subsequently, the protein was produced, and its activity tested in vitro for diagnostic applications.

### Characterization of CRISPR-mCas13 Cis and Trans Catalytic Activities

When Cas13/crRNA ribonucleoproteins (RNP) recognize and cleave their target sequence, they also exhibit non-specific, collateral cleavage activity that degrades ssRNAs nearby [63, 64]. Such collateral activity can be used in nucleic acid detection applications, where a ssRNA probe (reporter) molecule provided in the Cas13 reaction is cleaved by target-dependent Cas13 collateral activity [61]. The ssRNA reporter can contain a fluorophore linked by a short ssRNA sequence to a quencher, which emits fluorescence after the ssRNA sequence is cleaved, indicating the presence, and therefore the detection, of the target sequence (FIG. 10A).

To test the mCas13's cis and in-trans activities in vitro and to determine the most effective crRNAs to use in the mCas13 SARS-COV-2-based detection assays, 10 different crRNAs targeting two different regions in the SARS-COV-2 nucleocapsid gene (N) were designed and screened. The in vitro cleavage activity of mCas13 was first evaluated with 4 different crRNAs targeting single-stranded RNA substrates harboring target sequences complementary to the crRNA spacers. mCas13 exhibited different cleavage efficiencies with different crRNAs, with crRNA 4 mediating the highest efficiency relative to other crRNAs and controls (FIG. 10L).

Because different Cas13 proteins can exhibit different cleavage preferences depending on ssRNA sequences [63], efforts were directed to identifying the best ssRNA reporter for the mCas13 SARS-COV-2 based detection module. Therefore, 5 different ssRNA probes, each conjugated to a 5′ fluorescent molecule (FAM) and a 3′ fluorescence quencher (FQ) were screened. mCas13 was incubated with each of the four targeting crRNAs and non-specific (NS) crRNA and reporters in the presence of the synthetic (N gene) ssRNA target. The screening consistently identified crRNA 4 with a significantly higher fluorescence signal relative to the NS crRNA control, indicating the cleavage preference of mCas13 for poly (U) reporter sequences (FIGS. 10B-10F). Using the poly (U) reporter molecule to screen six more crRNAs targeting different N gene regions indicated that different crRNAs exhibited overall different signal levels.

To determine the optimal concentration of mCas13 and crRNA for maximal detection signal, the reaction was performed with titrated mCas13 and crRNA concentrations. It was observed that the optimal concentration of Cas13/crRNA RNP for a true positive signal with no significant signal in NS crRNA control was 500 nM (FIG. 10I). In addition, to determine the optimal temperature for mCas13 catalytic activity, different cleavage temperature conditions were tested ranging from 37° C. to 55° C. The highest activity was observed at 37° C., which is similar to the optimal temperature of other known Cas13 enzymes used for nucleic acid detection (FIG. 10J).

Using optimized conditions and the most effective crRNA 4, the effect of single mismatches between crRNA and target RNA on mCas13 RNA detection activity was investigated. 10 crRNAs (based on crRNA 4) were designed to contain a single nucleotide mismatch at different sites, with one mismatch for every 3 nucleotides on the spacer sequence. A low mismatch tolerance should cause a much lower detection signal than the positive control (which is a perfect match). This analysis revealed different mismatch tolerances for different regions of the spacer sequence, where mismatches at the extreme 5′ or 3′ ends of the crRNA were not well tolerated. In contrast, mismatches at other regions were tolerated better (FIG. 10G, FIG. 10M, FIG. 10N). The observed different tolerances for mismatches may be advantageous to designing crRNAs useful for detecting variants that harbor different SNPs. Altogether, these data indicated that the identified mCas13 is catalytically active with robust trans-cleavage activity, and thus is suitable for developing nucleic acid detection platforms.

RT-LAMP Coupled with CRISPR-mCas13 for SARS-COV-2 Detection

To ensure sensitive detection, pre-amplifying the RNA target of interest is necessary [61]. RT-LAMP isothermal amplification was chosen because it possesses several advantages over other amplification methods, including high sensitivity, rapid turnaround time, simple operation, and low cost [65]. To initiate the RT-LAMP reaction, primer sets well-established in previous reports to target and amplify conserved regions in the SARS-COV-2 N gene were used (named here as STOPCovid and DETECTR primer sets). Because mCas13 targets RNA, these primers were modified by appending a T7 promoter sequence to the 5′ end of the first half of either the forward inner primer (FIP) or the backward inner primer (BIP). During LAMP amplification, the T7 promoter sequence integrates into the amplified DNA products, providing a suitable template for the T7 RNA polymerase to transcribe the amplified LAMP product in vitro and generate RNA targets for mCas13 detection (FIG. 11A). The performance of these modified primers was tested using a synthetic SARS-COV-2 viral genome at 500 copies/uL. Gel electrophoresis indicated that these modified primers successfully amplified the target RNA with no observed amplification in the no-template control (NTC) (not shown). In addition, no substantial differences were found between the amplifications using primer sets with T7-containing FIP primer (T7-FIP) or T7-containing BIP primer (T7-BIP). Therefore, T7-FIP primers were chosen to establish the RT-LAMP mCas13 detection platform.

Since mCas13 performs optimally at 37° C., a temperature also optimal for T7 RNA polymerase, the T7-mediated transcription of the RT-LAMP product was coupled with the Cas13-based detection of the transcribed RNA in a single tube. Therefore, after RT-LAMP pre-amplification of SARS-COV-2 synthetic RNA using STOPCovid or DETECTR T7-FIP modified primers, the RT-LAMP products were added to the T7 transcription and mCas13 detection reaction. Real-time measurement of the T7-coupled mCas13-based detection indicated robust detection of RT-LAMP product only when using targeting crRNA (crRNA 4) and T7 RNA polymerase, confirming that the amplification of the synthetic SARS-COV-2 genome was specific and that T7 promoters were successfully integrated into the amplified products (FIG. 11B). These results show the successful development of an mCas13-based two-pot SARS-COV-2 detection platform.

Next, the limit of detection (LoD) of the two-pot SARS-COV-2 detection assay was determined. When establishing the two-pot assay, it was verified that both STOPCovid and DETECTR primer sets could be used to effectively detect SARS-COV-2 RNA (FIGS. 11C-11D). Therefore, the performance and the LoD of both STOPCovid and DETECTR primer sets were assayed to determine the primer set that is most suitable for the mCas13 SARS-COV-2 detection platform. Serial dilutions of the synthetic SARS-CoV-2 viral genome were used as an input for the pre-amplification RT-LAMP reaction. It was observed that both primer sets allowed sensitive detection of the synthetic RNA, but the STOPCovid primers reproducibly detected as few as 4 copies/uL viral RNA, compared with 8 copies/uL for DETECTR primers (FIGS. 11C-11D). Although extending the RT-LAMP amplification time could enhance its sensitivity, the RT-LAMP pre-amplification step was limited to 35 mins and the mCas13 detection reaction was limited to 20-30 mins, resulting in a total detection time of ˜1 hour or less. Due to its outstanding LoD, the STOPCovid primer set was chosen for the mCas13 detection platform.

### The RT-LAMP Coupled CRISPR-mCas13 Detection Assay is Specific for SARS-COV-2

To test the assay's specificity and ensure no cross-reactivity with other common viruses, this system was challenged with other SARS-COV-2-related or non-related viruses, including SARS-COV-1, MERS, TMV, and TuMV, together with SARS-COV-2. All tested viruses (other than SARS-CoV-2) showed only near-background signals, indicating that the developed assay was highly specific (FIG. 11E). Collectively, these results indicated that the developed mCas13-based detection platform exhibits reliable, highly sensitive, and highly specific detection of SARS-COV-2 with a turnaround time of 1 hour from extracted RNA to results.

Validation of RT-LAMP Coupled with CRISPR-mCas13 SARS-COV-2 Detection Platform in a Clinical Setting

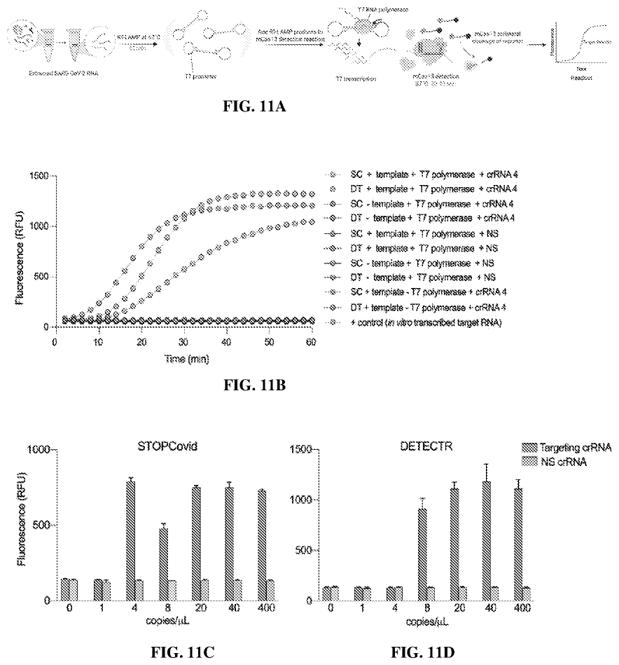

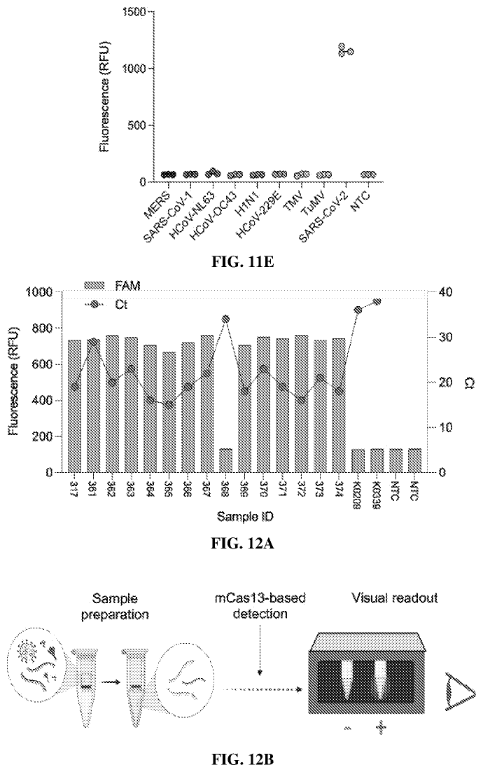

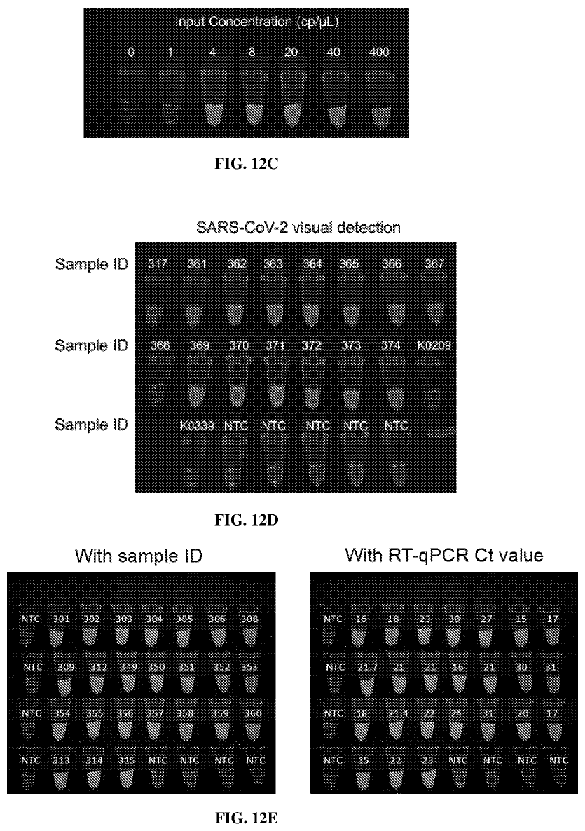

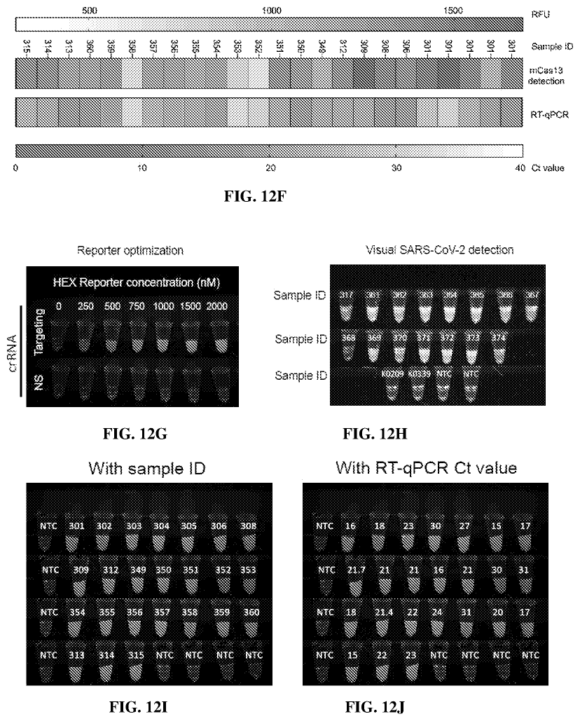

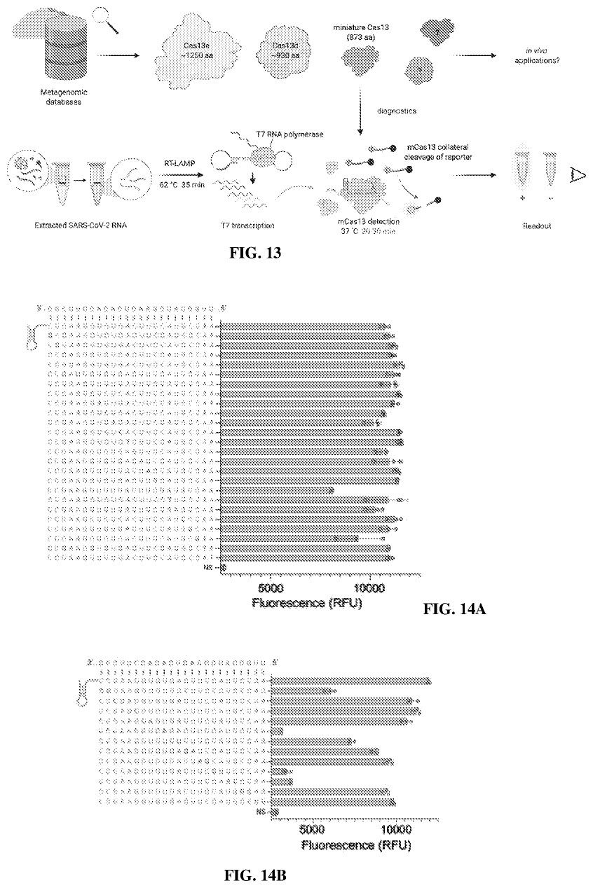

Next, the assay was validated with total RNA extracted from SARS-CoV-2 patient swab samples. Oropharyngeal or nasopharyngeal swab samples were collected from suspected COVID-19 patients. After RNA extraction following the CDC EUA-approved protocol, the samples were confirmed positive for SARS-COV-2 using RT-qPCR. The assay was first tested with 17 samples with Ct values of 15-39. Using the mCas13-based detection assay, all samples with Ct values of less than 34 were correctly identified within one hour (FIG. 12A). These results indicate that the system can reliably detected SARS-COV-2 in samples with Ct values up to 34.

To facilitate large-scale screening during the SARS-COV-2 pandemic, performing diagnostic assays at POC or outside of laboratory settings is critical. Therefore, it was determined whether the assay could be coupled with a portable device to permit a simple readout suitable for POC and routine diagnostics. A hand-held, inexpensive fluorescence visualizer (P51 Molecular Fluorescence Viewer) was adapted to easily visualize and interpret the results. This portable device illuminates reaction tubes with blue light, and fluorescent reactions are visible through a film used as an optical filter. Using this device, fluorescence is readily visible to the human eye without the need for sophisticated instruments like qPCR machines or plate readers (FIG. 12B). Using a modified RNA reporter molecule conjugated to a 5′ HEX fluorescent molecule instead of FAM, it was observed that mCas13 collateral cleavage of this reporter produced a bright signal visible with the P51 fluorescence visualizer. Investigation of the HEX reporter concentration needed for definitive visual detection of true positive samples indicated that 1 μM of HEX RNA reporters produced a clear signal with no substantial background in negative controls (FIG. 12G).

To ensure that the sensitivity of the assay was preserved with the change in fluorescent molecule, the LoD assay was repeated using the STOPCovid primer set. The same results as obtained previously with the machine-based readout in FIG. 11C were obtained, demonstrating that the 5′ HEX modification did not affect assay performance (FIG. 12C). Next, this assay's performance was evaluated with the same 17 SARS-COV-2 RNA samples used in FIG. 12A and the results from the visual-based detection assay were compared to the results in FIG. 12A. There was 100% concordance between the two assays, indicating that the developed mCas13 visual detection assay is reliable (FIG. 12D, FIG. 12H). Finally, to clinically validate and further test the reliability of the visual mCas13 detection assay, the assay's performance was tested with an additional 24 qPCR-confirmed positive clinical samples of total RNA extracted from patient swabs, along with no-template control reactions. The visual-readout mCas13-based detection of SARS-COV-2 from these 24 samples showed 100% concordance with RT-qPCR results. A clear fluorescence signal relative to the negative controls was detected in each positive sample (FIGS. 12E-12F, FIGS. 12I-12J). Altogether, this system's effectiveness was validated in 41 clinical samples, facilitating viral detection within 1 hour with a simple visual readout.

### DISCUSSION

To control the pandemic of COVID-19, rapid, accurate, reliable, and portable diagnostics for SARS-COV-2 are needed [21]. However, increasing sensitivity and specificity has remained a common challenge for recently developed diagnostics. Amplification can increase the sensitivity of assays and isothermal amplification techniques like RT-LAMP and RT-RPA are good alternatives for RT-PCR. Both are highly sensitive, provide rapid and more accessible platforms for viral nucleic acid detection, and are suitable for POC uses [66-68]. Despite many advantages of isothermal amplification in relation to other traditional amplification methods, their application is limited by the high rate of non-specific amplification and cross-contamination [69, 70]. Therefore, isothermal amplification methods are coupled with CRISPR systems to enhance specificity and LoD [36, 41, 71, 72].

Here, a previously unreported/uncharacterized Cas13 variant was identified and characterized and an RT-LAMP coupled with mCas13 assay for SARS-COV-2 detection was developed. Sequence similarity searching was used to identify the mCas13 variant. The corresponding mCas13 sequence was synthesized and subcloned into a protein expression vector in order to purify the protein and characterize its cleavage activity. The crRNA architecture was identified and synthesized for in vitro expression, and the crRNA and mCas13 were used to form an RNP complex for cleavage activity assays. multiple crRNAs targeting different regions in SARS-COV-2 were designed. One crRNA showed robust collateral activity against poly (U) reporters. Different regions of the crRNA exhibited different tolerances for mismatches, which can be advantageous to designing crRNAs useful for detecting variants that harbor different SNPs.

Because mCas13 targets RNA for cleavage and degradation, the LAMP primers were modified to add the T7 polymerase promoter sequence to permit the T7 RNA polymerase to generate transcripts for mCas13. This modified RT-LAMP enabled the coupling with mCas13 for virus detection, obviating the need for extra steps. This modification in the RT-LAMP primers did not interfere with the RT-LAMP reaction and resulted in robust amplification of the target sequences. The RT-LAMP coupled with mCas13 exhibited high specificity since sequences of other viruses, including SARS—Co-V1, MERS, TMV, and TuMV, did not trigger the collateral reaction; however, the SARS-COV2 sequence triggered a strong collateral cleavage reaction and fluorescence.

The usefulness of this modality (RT-LAMP coupled with mCas13) for SARS-COV-2 detection was tested and validated in 41 clinical samples. The data showed that mCas13 successfully detected SARS-COV-2 in clinical samples, exhibiting strong concordance with RT-qPCR. A simple LED-based visualizer was employed for straightforward and inexpensive detection of fluorescence in test results. With the affordable and portable P51 LED-based visualizer, a positive signal is observable as a bright green light. The simple fluorescence visualizer showed consistent detection results from fluorescence readers, verifying the effectiveness of the method. It is worth noting that several crRNAs were screened to identify functional ones that activated mCas13, thereby limiting the off-target activities of the mCas13 enzyme. Other CRISPR systems with robust activities can degrade the target and compromise the detection sensitivity. Because this mCas13 system is not overly robust, it may be quite useful for sensitive and specific detection. These features, however, could be quite beneficial in building a highly specific and powerful modality of virus and nucleic acid detection. Briefly, this mCas13-based detection platform enables rapid, accurate, simple, cost-effective, and efficient detection of SARS-COV-2, and shows potential for POC applications.

Due to its miniature size, the mCas13 variant can be used for a variety of in vivo RNA manipulations, including RNA knockdown, editing, splicing regulation, RNA imaging and localization. The recently identified compact Cas13, Cas13e.1, shows efficacy against SAR-COV-2 and influenza A virus, indicating that Cas13 can be useful as an antiviral therapeutic [56, 62]. These studies demonstrate that bacterial defense systems have untapped potential for diverse synthetic biology applications, diagnostics, and therapeutics as antiviral agents.

In summary, the data demonstrate successful identification and characterization of the catalytic activities of a previously unknown/uncharacterized miniature variant of Cas13, and harnessing of its collateral catalytic activities to develop a system for SARS-COV-2 detection. The modality coupling RT-LAMP and mCas13 demonstrates key features, including simplicity, specificity, sensitivity, and portability. The readout signal was measured using a low-cost P51 device. The P51 device can be paired with a cell phone camera that processes and shares data, facilitating the integration of this modality to large-scale testing. This work illustrates the usefulness of mCas13 systems for diagnostics as well as other potential in vivo RNA manipulations for diverse applications.

Mahas, et al., ACS Synthetic Biology, 10 (10), 2541-2551 (2021) DOI: 10.1021/acssynbio. 1c00181, and all of the 21 pages of Supplemental/Supplementary information associated therewith, is specifically incorporated therein.

### REFERENCES

1. Cui, J., F. Li, and Z. L. Shi, Origin and evolution of pathogenic coronaviruses. Nat Rev Microbiol, 2019. 17 (3): p. 181-192.
2. Kilic, T., R. Weissleder, and H. Lee, Molecular and Immunological Diagnostic Tests of COVID-19: Current Status and Challenges. iScience, 2020. 23 (8): p. 101406.
3. Mabey, D., et al., Diagnostics for the developing world. Nat Rev Microbiol, 2004. 2 (3): p. 231-40.
4. Broughton, J. P., et al., CRISPR-Cas12-based detection of SARS-COV-2. Nat Biotechnol, 2020. 38 (7): p. 870-874.
5. Kellner, M. J., et al., SHERLOCK: nucleic acid detection with CRISPR nucleases. Nature protocols, 2019. 14 (10): p. 2986-3012.
6. Gootenberg, J. S., et al., Multiplexed and portable nucleic acid detection platform with Cas13, Cas12a, and Csm6. Science, 2018. 360 (6387): p. 439-444.
7. Gootenberg, J. S., et al., Nucleic acid detection with CRISPR-Cas13a/C2c2. Science, 2017.
8. Abudayyeh, O. O., et al., C2c2 is a single-component programmable RNA-guided RNA-targeting CRISPR effector. Science, 2016. 353 (6299): p. aaf5573.
9. East-Seletsky, A., et al., Two distinct RNase activities of CRISPR-C2c2 enable guide-RNA processing and RNA detection. Nature, 2016. 538 (7624): p. 270-273.
10. East-Seletsky, A., et al., RNA Targeting by Functionally Orthogonal Type VI-A CRISPR-Cas Enzymes. Mol Cell, 2017. 66 (3): p. 373-383 e3.
11. Thompson, D. and Y. Lei, Mini review: Recent progress in RT-LAMP enabled COVID-19 detection. Sensors and Actuators Reports, 2020. 2 (1): p. 100017.
12. Joung, J., et al., Detection of SARS-COV-2 with SHERLOCK One-Pot Testing. N Engl J Med, 2020. 383 (15): p. 1492-1494.
13. Arizti-Sanz, J., et al., Streamlined inactivation, amplification, and Cas13-based detection of SARS-COV-2. Nat Commun, 2020. 11 (1): p. 5921.
14. Ali, Z., et al., iSCAN: An RT-LAMP-coupled CRISPR-Cas12 module for rapid, sensitive detection of SARS-COV-2. Virus Res, 2020. 288: p. 198129.
15. Sun, Y., et al., One-tube SARS-COV-2 detection platform based on RT-RPA and CRISPR/Cas12a. J Transl Med, 2021. 19 (1): p. 74.
16. Joung, J., et al., Detection of SARS-COV-2 with SHERLOCK One-Pot Testing. New England Journal of Medicine, 2020. 383 (15): p. 1492-1494.
17. Fozouni, P., et al., Amplification-free detection of SARS-COV-2 with CRISPR-Cas13a and mobile phone microscopy. Cell, 2021. 184 (2): p. 323-333 e9.
18. Liu, T. Y., et al., Accelerated RNA detection using tandem CRISPR nucleases. 2021.
19. Lopato, S., et al., atSRp30, one of two SF2/ASF-like proteins from Arabidopsis thaliana, regulates splicing of specific plant genes. Genes Dev, 1999 Regarding the significance of AS in plant development. 13 (8): p. 987-1001.
20. Kellner, M. J., et al., SHERLOCK: nucleic acid detection with CRISPR nucleases. Nat Protoc, 2019. 14 (10): p. 2986-3012.
21. Kelly-Cirino, C. D., et al., Importance of diagnostics in epidemic and pandemic preparedness. BMJ Glob Health, 2019. 4 (Suppl 2): p. e001179.
22. McDermott, J. H., et al., The rise of point-of-care genetics: how the SARS-COV-2 pandemic will accelerate adoption of genetic testing in the acute setting. Eur J Hum Genet, 2021.
23. Esbin, M. N., et al., Overcoming the bottleneck to widespread testing: a rapid review of nucleic acid testing approaches for COVID-19 detection. RNA, 2020. 26 (7): p. 771-783.
24. Niemz, A., T. M. Ferguson, and D. S. Boyle, Point-of-care nucleic acid testing for infectious diseases. Trends Biotechnol, 2011. 29 (5): p. 240-50.
25. Corman, V. M., et al., Detection of 2019 novel coronavirus (2019-nCoV) by real-time RT-PCR. Euro Surveill, 2020. 25 (3).
26. Morshed, M. G., et al., Molecular methods used in clinical laboratory: prospects and pitfalls. FEMS Immunol Med Microbiol, 2007. 49 (2): p. 184-91.
27. Vogels, C. B. F., et al., Analytical sensitivity and efficiency comparisons of SARS-COV-2 RT-qPCR primer-probe sets. Nat Microbiol, 2020. 5 (10): p. 1299-1305.
28. Larremore, D. B., et al., Test sensitivity is secondary to frequency and turnaround time for COVID-19 surveillance. medRxiv, 2020.
29. Piepenburg, O., et al., DNA detection using recombination proteins. PLOS Biol, 2006. 4 (7): p. e204.
30. Notomi, T., et al., Loop-mediated isothermal amplification of DNA. Nucleic Acids Res, 2000. 28 (12): p. E63.
31. Obande, G. A. and K. K. Banga Singh, Current and Future Perspectives on Isothermal Nucleic Acid Amplification Technologies for Diagnosing Infections. Infect Drug Resist, 2020. 13: p. 455-483.
32. Ning, B., et al., A smartphone-read ultrasensitive and quantitative saliva test for COVID-19. Sci Adv, 2021. 7 (2).
33. Patchsung, M., et al., Clinical validation of a Cas13-based assay for the detection of SARS-COV-2 RNA. Nat Biomed Eng, 2020. 4 (12): p. 1140-1149.
34. Arizti-Sanz, J., et al., Streamlined inactivation, amplification, and Cas13-based detection of SARS-COV-2. Nat Commun, 2020. 11 (1): p. 5921.
35. Ali, Z., et al., iSCAN: An RT-LAMP-coupled CRISPR-Cas12 module for rapid, sensitive detection of SARS-COV-2. Virus Res, 2020. 288: p. 198129.
36. Joung, J., et al., Detection of SARS-COV-2 with SHERLOCK One-Pot Testing. N Engl J Med, 2020. 383 (15): p. 1492-1494.
37. Broughton, J. P., et al., CRISPR-Cas12-based detection of SARS-CoV-2. Nat Biotechnol, 2020. 38 (7): p. 870-874.
38. Marsic, T., et al., Vigilant: An Engineered VirD2-Cas9 Complex for Lateral Flow Assay-Based Detection of SARS-COV2. Nano Lett, 2021.
39. Aman, R., A. Mahas, and M. Mahfouz, Nucleic Acid Detection Using CRISPR/Cas Biosensing Technologies. ACS Synth Biol, 2020.
40. Li, Y., et al., CRISPR/Cas Systems towards Next-Generation Biosensing. Trends Biotechnol, 2019. 37 (7): p. 730-743.
41. Gootenberg, J. S., et al., Nucleic acid detection with CRISPR-Cas13a/C2c2. Science, 2017.
42. Kellner, M. J., et al., SHERLOCK: nucleic acid detection with CRISPR nucleases. Nat Protoc, 2019. 14 (10): p. 2986-3012.
43. Gootenberg, J. S., et al., Multiplexed and portable nucleic acid detection platform with Cas13, Cas12a, and Csm6. Science, 2018. 360 (6387): p. 439-444.
44. Myhrvold, C., et al., Field-deployable viral diagnostics using CRISPR-Cas13. Science, 2018. 360 (6387): p. 444-448.
45. Mabey, D., et al., Diagnostics for the developing world. Nat Rev Microbiol, 2004. 2 (3): p. 231-40.
46. Liu, T. Y., et al., Accelerated RNA detection using tandem CRISPR nucleases. medRxiv, 2021.
47. Fozouni, P., et al., Amplification-free detection of SARS-COV-2 with CRISPR-Cas13a and mobile phone microscopy. Cell, 2021. 184 (2): p. 323-333 e9.
48. Burstein, D., et al., New CRISPR-Cas systems from uncultivated microbes. Nature, 2017. 542 (7640): p. 237-241.
49. Al-Shayeb, B., et al., Clades of huge phages from across Earth's ecosystems. Nature, 2020. 578 (7795): p. 425-431.
50. Smargon, A. A., et al., Cas13b Is a Type VI-B CRISPR-Associated RNA-Guided RNase Differentially Regulated by Accessory Proteins Csx27 and Csx28. Mol Cell, 2017. 65 (4): p. 618-630 e7.
51. Yan, W. X., et al., Cas13d Is a Compact RNA-Targeting Type VI CRISPR Effector Positively Modulated by a WYL-Domain-Containing Accessory Protein. Mol Cell, 2018. 70 (2): p. 327-339 e5.
52. Shmakov, S., et al., Discovery and Functional Characterization of Diverse Class 2 CRISPR-Cas Systems. Mol Cell, 2015. 60 (3): p. 385-97.
53. Konermann, S., et al., Transcriptome Engineering with RNA-Targeting Type VI-D CRISPR Effectors. Cell, 2018. 173 (3): p. 665-676 e14.
54. Abudayyeh, O. O., et al., RNA targeting with CRISPR-Cas13. Nature, 2017. 550 (7675): p. 280-284.
55. Cox, D. B. T., et al., RNA editing with CRISPR-Cas13. Science, 2017. Abbott, T. R., et al., Development of CRISPR as an Antiviral Strategy 56. to Combat SARS-COV-2 and Influenza. Cell, 2020. 181 (4): p. 865-876 e12.
57. Mahas, A., R. Aman, and M. Mahfouz, CRISPR-Cas13d mediates robust RNA virus interference in plants. Genome Biol, 2019. 20 (1): p. 263.
58. Aman, R., et al., RNA virus interference via CRISPR/Cas13a system in plants. Genome Biol, 2018. 19 (1): p. 1.
59. Ackerman, C. M., et al., Massively multiplexed nucleic acid detection with Cas13. Nature, 2020. 582 (7811): p. 277-282.
60. Freije, C. A., et al., Programmable Inhibition and Detection of RNA Viruses Using Cas13. Mol Cell, 2019. 76 (5): p. 826-837 e11.
61. Brogan, D. J., et al., A Sensitive, Rapid, and Portable CasRx-based Diagnostic Assay for SARS-COV-2. medRxiv, 2020.
62. Xu, C., et al., Novel miniature CRISPR-Cas13 systems from uncultivated microbes effective in degrading SARS-COV-2 sequences and influenza viruses. Research Square, 2021. DOI: 10.21203/rs.3.rs-30924/v1. https://www.researchsquare.com/article/rs-30924/v1.
63. Abudayyeh, O. O., et al., C2c2 is a single-component programmable RNA-guided RNA-targeting CRISPR effector. Science, 2016. 353 (6299): p. aaf5573.
64. East-Seletsky, A., et al., Two distinct RNase activities of CRISPR-C2c2 enable guide-RNA processing and RNA detection. Nature, 2016. 538 (7624): p. 270-273.
65. Thompson, D. and Y. Lei, Mini review: Recent progress in RT-LAMP enabled COVID-19 detection. Sensors and Actuators Reports, 2020. 2 (1): p. 100017.
66. Dao Thi, V. L., et al., A colorimetric RT-LAMP assay and LAMP-sequencing for detecting SARS-COV-2 RNA in clinical samples. Sci Transl Med, 2020. 12 (556).
67. Ganguli, A., et al., Rapid Isothermal Amplification and Portable Detection System for SARS-COV-2. bioRxiv, 2020.
68. Qian, J., et al., An enhanced isothermal amplification assay for viral detection. bioRxiv, 2020.
69. Nagai, K., et al., Diagnostic test accuracy of loop-mediated isothermal amplification assay for Mycobacterium tuberculosis: systematic review and meta-analysis. Sci Rep, 2016. 6: p. 39090.
70. Chou, P. H., et al., Real-time target-specific detection of loop-mediated isothermal amplification for white spot syndrome virus using fluorescence energy transfer-based probes. J Virol Methods, 2011. 173 (1): p. 67-74.
71. Aman, R., et al., Efficient, Rapid, and Sensitive Detection of Plant RNA Viruses With One-Pot RT-RPA-CRISPR/Cas12a Assay. Front Microbiol, 2020. 11: p. 610872.
72. Mahas, A., et al., LAMP-Coupled CRISPR-Cas12a Module for Rapid and Sensitive Detection of Plant DNA Viruses. Viruses, 2021. 13 (3). Larkin, M. A., et al., Clustal W and Clustal X version 2.0. 73. Bioinformatics, 2007. 23 (21): p. 2947-8.
74. Gouet, P., et al., ESPript: analysis of multiple sequence alignments in PostScript. Bioinformatics, 1999. 15 (4): p. 305-8.
75. Couvin, D., et al., CRISPRCasFinder, an update of CRISRFinder, includes a portable version, enhanced performance and integrates search for Cas proteins. Nucleic Acids Res, 2018. 46 (W1): p. W246-W251.
76. Biswas, A., et al., CRISPRDetect: A flexible algorithm to define CRISPR arrays. BMC Genomics, 2016. 17: p. 356.
77. E. A. Nalefski et al., Kinetic analysis of Cas12a and Cas13a RNA-Guided nucleases for development of improved CRISPR-Based diagnostics. iScience 24, 102996 (2021).
78. K. A. Curtis et al., A multiplexed RT-LAMP assay for detection of group M HIV-1 in plasma or whole blood. J Virol Methods 255, 91-97 (2018).
79. K. H. Ooi et al., An engineered CRISPR-Cas12a variant and DNA-RNA hybrid guides enable robust and rapid COVID-19 testing. Nat Commun 12, 1739 (2021).
80. M. M. Kaminski, O. O. Abudayyeh, J. S. Gootenberg, F. Zhang, J. J. Collins, CRISPR-784 based diagnostics. Nat Biomed Eng 5, 643-656 (2021).
81. D. J. Brogan et al., Development of a Rapid and Sensitive CasRx-Based Diagnostic Assay for SARS-COV-2. ACS Sens 6, 3957-3966 (2021).
82. J. S. Chen et al., CRISPR-Cas12a target binding unleashes indiscriminate single-stranded DNase activity. Science 360, 436-439 (2018).
83. T. R. Abbott et al., Development of CRISPR as an Antiviral Strategy to Combat SARS-COV-2 and Influenza. Cell 181, 865-876 e812 (2020).
84. M. Lotfi, N. Rezaei, CRISPR/Cas13: A potential therapeutic option of COVID-19. Biomed Pharmacother 131, 110738 (2020).
85. J. Gao, T. Luo, N. Lin, S. Zhang, J. Wang, A New Tool for CRISPR-Cas13a-Based Cancer Gene Therapy. Mol Ther Oncolytics 19, 79-92 (2020).
86. F. Palaz et al., CRISPR-Cas13 System as a Promising and Versatile Tool for Cancer Diagnosis, Therapy, and Research. ACS Synth Biol 10.1021/acssynbio.1c00107 (2021).
87. R. E. Kontermann, Strategies for extended serum half-life of protein therapeutics. Curr Opin Biotechnol 22, 868-876 (2011).
88. O. O. Abudayyeh et al., RNA targeting with CRISPR-Cas13. Nature 550, 280 (2017).
89. L. B. Harrington et al., A thermostable Cas9 with increased lifetime in human plasma. Nat Commun 8, 1424 (2017).
90. D. Narasimhan et al., Structural analysis of thermostabilizing mutations of cocaine esterase. Protein Eng Des Sel 23, 537-547 (2010).
91. I. Mougiakos et al., Characterizing a thermostable Cas9 for bacterial genome editing and silencing. Nat Commun 8, 1647 (2017).
92. M. P. Terns, CRISPR-Based Technologies: Impact of RNA-Targeting Systems. Mol Cell 72, 404-412 (2018).
93. D. J. Brogan et al., A Sensitive, Rapid, and Portable CasRx-based Diagnostic Assay for SARS-COV-2. medRxiv 10.1101/2020.10.14.20212795 (2020).
94. P. Fozouni et al., Amplification-free detection of SARS-COV-2 with CRISPR-Cas13a and mobile phone microscopy. Cell 184, 323-333 e329 (2021).
95. T. Y. Liu et al., Accelerated RNA detection using tandem CRISPR nucleases. Nat Chem Biol 17, 982-988 (2021).
96. Z. Ali et al., iSCAN: An RT-LAMP-coupled CRISPR-Cas12 module for rapid, sensitive detection of SARS-COV-2. Virus Res 288, 198129 (2020).
97. L. Li et al., HOLMESv2: A CRISPR-Cas12b-Assisted Platform for Nucleic Acid Detection and DNA Methylation Quantitation. ACS Synth Biol 8, 2228-2237 (2019).
98. D. Couvin et al., CRISPRCasFinder, an update of CRISRFinder, includes a portable version, enhanced performance and integrates search for Cas proteins. Nucleic Acids Res 46, W246-W251 (2018).
99. A. Biswas, R. H. Staals, S. E. Morales, P. C. Fineran, C. M. Brown, CRISPRDetect: A flexible algorithm to define CRISPR arrays. BMC Genomics 17, 356 (2016).
100. A. Mahas, C. Neal Stewart, Jr., M. M. Mahfouz, Harnessing CRISPR/Cas systems for 713 programmable transcriptional and post-transcriptional regulation. Biotechnol Adv 714 10.1016/j.biotechadv.2017.11.008 (2017).
101. G. J. Knott, J. A. Doudna, CRISPR-Cas guides the future of genetic engineering. Science 361, 866-869 (2018).
102. S. Agrawal et al., Rapid detection of SARS-COV-2 with Cas13. medRxiv 10.1101/2020.12.14.20247874 (2020).
103. M. Patchsung et al., Clinical validation of a Cas13-based assay for the detection of 762 SARS-COV-2 RNA. Nat Biomed Eng 4, 1140-1149 (2020).
104. J. Arizti-Sanz et al., Streamlined inactivation, amplification, and Cas13-based detection 764 of SARS-COV-2. Nat Commun 11, 5921 (2020).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Although the description of materials, compositions, components, steps, techniques, etc. may include numerous options and alternatives, this should not be construed as, and is not an admission that, such options and alternatives are equivalent to each other or, in particular, are obvious alternatives.

Every composition disclosed herein is intended to be and should be considered to be specifically disclosed herein. Further, every subgroup that can be identified within this disclosure is intended to be and should be considered to be specifically disclosed herein. As a result, it is specifically contemplated that any composition, or subgroup of compositions can be either specifically included for or excluded from use or included in or excluded from a list of compositions.

Those skilled in the art will recognize, or be able to ascertain using no more than routine experimentation, many equivalents to the specific embodiments of the method and compositions described herein. Such equivalents are intended to be encompassed by the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A polynucleotide comprising a nucleotide sequence
encoding a class II, type VI CRISPR/Cas effector protein (Cas13) and optionally a heterologous sequence, wherein the Cas effector protein comprises an amino acid sequence encoded by SEQ ID NO:63, SEQ ID NO:65 or SEQ ID NO:67, or a sequence with at least 70% sequence identity thereto;
optionally wherein the sequence encoding the Cas effector protein comprises SEQ ID NO: 63, SEQ ID NO:65 or SEQ ID NO:67, or a sequence with at least 70% sequence identity to SEQ ID NO:63, SEQ ID NO:65 or SEQ ID NO:67.

2. The polynucleotide of claim 1, wherein the sequence encoding the Cas effector protein is codon optimized for expression in a prokaryotic or eukaryotic cell.

3. The polynucleotide of claim 1, comprising a heterologous sequence, wherein the heterologous sequence comprises a promoter, transcription terminator, multiple cloning site, drug resistance marker, one or more protease recognition sites, one or more epitope tags, or a combination thereof.

4. The polynucleotide of claim 3, wherein the heterologous sequence is operably linked to the sequence encoding the Cas effector protein.

5. The polynucleotide of claim 1 further comprising a sequence encoding an RNA comprising a crRNA sequence, wherein the RNA is capable of complexing with the Cas effector protein and hybridizing to a target RNA sequence.

6. The polynucleotide of claim 1, wherein the Cas effector protein is derived from Thermoclostridium caenicola or a Proteobacteria bacterium.

7. A vector comprising the polynucleotide of claim 1, optionally wherein the vector comprises the nucleotide sequence of SEQ ID NO:69 or SEQ ID NO:70, or a sequence having at least 60% sequence identity to SEQ ID NO:69 or SEQ ID NO: 70.

8. (canceled)

9. A prokaryotic or eukaryotic cell comprising the vector of claim 7.

10. A method of producing a class II, type VI CRISPR/Cas effector protein comprising contacting the vector of claim 7 with a prokaryotic or eukaryotic cell under conditions suitable for expression of the sequence encoding the Cas effector protein.

11. (canceled)

12. An isolated class II, type VI CRISPR/Cas effector protein, wherein the Cas effector protein is produced by the method of claim 10.

13. An isolated class II, type VI CRISPR/Cas effector protein comprising the amino acid sequence of SEQ ID NO:64 or SEQ ID NO:66, or a sequence with at least 70% sequence identity to SEQ ID NO:64, SEQ ID NO:66 or SEQ ID NO:68.

14. A ribonucleoprotein complex comprising the Cas effector protein of claim 12 complexed with an RNA comprising crRNA sequence, optionally wherein the RNA is capable of hybridizing to a target RNA sequence.

15. A composition comprising a Cas13 protein, wherein the Cas13 protein comprises the amino acid sequence of SEQ ID NO:64, SEQ ID NO:66 or SEQ ID NO:68, or a sequence with at least 70% sequence identity to SEQ ID NO:64, SEQ ID NO:66, or SEQ ID NO: 68.

16. The composition of claim 15, wherein the Cas13 protein comprises one or more HEPN (Higher Eukaryotes and Prokaryotes Nucleotide-binding) domains, preferably two HEPN domains.

17. The composition of claim 16, wherein the HEPN domain comprises a RxxxxH (SEQ ID NO:192) motif sequence, wherein X represents any amino acid.

18. The composition of claim 15, wherein the Cas13 protein is complexed with and RNA comprising a crRNA sequence, optionally wherein the RNA is capable of hybridizing to a target RNA sequence.

19.-23. (canceled)

24. A method of performing targeted knockdown of an RNA transcript comprising introducing the composition of claim 18 to a cell, wherein the crRNA sequence hybridizes to the RNA transcript, thereby inducing cleavage of the RNA transcript by the Cas13 protein.

25. (canceled)

26. (canceled)

27. A method of detecting the presence of an RNA transcript in a nucleic acid sample comprising contacting the sample with the composition of claim 18 in the presence of an activatable single stranded RNA (ssRNA) oligonucleotide comprising a reporter moiety,
wherein the crRNA is designed to hybridize to the RNA transcript, wherein the Cas13 cleaves the ssRNA oligonucleotide upon binding of the Cas13 crRNA complex to the RNA transcript, wherein detection of the cleavage of the ssRNA oligonucleotide indicates the presence of the RNA transcript.

28. (canceled)

29. (canceled)

30. A method of determining the localization of an RNA transcript in a cell comprising introducing the composition of claim 18 to the cell,
wherein the Cas13 is catalytically inactive and further comprises a detectable marker,
wherein the crRNA is designed to hybridize to the RNA transcript, and wherein the Cas13 crRNA complex binds to the RNA transcript, thereby indicating the location of the RNA transcript.

31. (canceled)

32. A method for performing targeted editing of an RNA transcript comprising introducing the composition of claim 18 to a cell,
wherein the Cas13 is catalytically inactive and further comprises a deaminase domain of an RNA-dependent Adenosine Deaminase (ADAR),
wherein the crRNA is capable of hybridizing with a region in the RNA transcript comprising a target A nucleotide to form an RNA duplex, wherein the duplex comprises an A-C mismatch at the target A nucleotide,
wherein the target A nucleotide is deaminated by the deaminase domain.

33. (canceled)


SEQUENCE LISTING (215 sequences)

SEQ ID NO: 1 - DNA, 22 bp, Artificial Sequence
  synthetic DNA
gaaatttggatctttgtcatcc

SEQ ID NO: 2 - DNA, 22 bp, Artificial Sequence
  Synthetic DNA
gaaatttggatctttgtcatcc

SEQ ID NO: 3 - DNA, 41 bp, Artificial Sequence
  Synthetic DNA
tgcggccaatgtttgtaatcagccaaggaaattttggggac

SEQ ID NO: 4 - DNA, 39 bp, Artificial Sequence
  Synthetic DNA
cgcattggcatggaagtcactttgatggcacctgtgtag

SEQ ID NO: 5 - DNA, 19 bp, Artificial Sequence
  Synthetic DNA
ttccttgtctgattagttc

SEQ ID NO: 6 - DNA, 18 bp, Artificial Sequence
  Synthetic DNA
accttcgggaacgtggtt

SEQ ID NO: 7 - DNA, 60 bp, Artificial Sequence
  Synthetic DNA
tgcggccaatgtttgtaatcagtaatacgactcactataggccaaggaaattttggggac

SEQ ID NO: 8 - DNA, 58 bp, Artificial Sequence
  Synthetic DNA
cgcattggcatggaagttaatacgactcactataggcactttgatggcacctgtgtag

SEQ ID NO: 9 - DNA, 18 bp, Artificial Sequence
  Synthetic DNA
gctgctgaggcttctaag

SEQ ID NO: 10 - DNA, 21 bp, Artificial Sequence
  Synthetic DNA
gcgtcaatatgcttattcagc

SEQ ID NO: 11 - DNA, 40 bp, Artificial Sequence
  Synthetic DNA
gcggccaatgtttgtaatcagtagacgtggtccagaacaa

SEQ ID NO: 12 - DNA, 39 bp, Artificial Sequence
  Synthetic DNA
tcagcgttcttcggaatgtcgctgtgtaggtcaaccacg

SEQ ID NO: 13 - DNA, 22 bp, Artificial Sequence
  Synthetic DNA
ccttgtctgattagttcctggt

SEQ ID NO: 14 - DNA, 18 bp, Artificial Sequence
  Synthetic DNA
tggcatggaagtcacacc

SEQ ID NO: 15 - DNA, 60 bp, Artificial Sequence
  Synthetic DNA
gcggccaatgtttgtaatcagttaatacgactcactatagggagacgtggtccagaacaa

SEQ ID NO: 16 - DNA, 59 bp, Artificial Sequence
  Synthetic DNA
tcagcgttcttcggaatgtcgctaatacgactcactatagggtgtgtaggtcaaccacg

SEQ ID NO: 17 - DNA, 46 bp, Artificial Sequence
  Synthetic DNA
gaaattaatacgactcactatagggaggcttctaagaagcctcggc

SEQ ID NO: 18 - DNA, 23 bp, Artificial Sequence
  Synthetic DNA
ctgtctctgcggtaaggcttgag

SEQ ID NO: 19 - protein, 1375 aa, Unknown
  Thermoclostridium caenicola
MGSSHHHHHHSSGLVPRGSHMASWSHPQFEKGGGSGGGSGGSAWSHPQFEKMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSMKITKRKWGEHHPPLYFYRDEDSGRLLAQNDRKQDYTDTLFNDIAQDTFERSLRNRLLKTPEKGDKRFYSNEIVKLVEKLCQGADVAEIMKSMERNEKLRPKNEKEIKNLKKQLDGTLSEYGKRYTAPEGAMTLNDALFYLVEGNPLKQAMAKAELGKIREALIKEKENRINRVRYSIKNNKIPLRIQEDGGITPNNDRAAWLLGLMKPADPAKGITDCYPLLGELEEVFDFDKLSKTLHEKISRCQGRPRSIAMAVDEALKQYLRELWEKSPSRQQDLKYYFQAVQEYFKDNFPIRTKRMGARLRQELLKDKTSLSRLLEPKHMANAVRRRLINQSTQMHILYGKLYAYCCGEDGRLLVNSETLQRIQVHEAVKKQAMTAVLWSISRLRYFYQFEDGDILSNKNPIKDFRDKFLRDTNKYTHEDVEACKEKLQDFFPLKELQEKIKEDAKGLQETDNKQADTTDFKAIGHIVRDDRKLCNQLLAECVSCIGELRHHIFHYKNVTLIQALKRIADKVKPEDLSVLRAIYLLDRRNLKKAFAKRISSMNLPLYYREDLLSRIFKKEGTAFFLYSAKIQMTPSFQRVYERGKNLRREFECERMKAEASNGQNGQDGDRLKWFRQLAAGDSADTHFNWAVEAYAESAADVENNVEFDTDVDAQRALRNLLLLIYRHHFLPEVQKDETLVTGKIHKVLERNRQLSEGQGPNQGKAHGYSVIEELYHEGMPLSDLMKQLQRRISETERESRELAQEKTDYAQRFILDIFAEAFNDFLEAHYGEEYLEIMSPRKDAEAAKKWVKESKTVDLKTSIDEKEPEGHLLVLYPVLRLLDERELGELQQQMIRYRTSLASWQGESNFSEEIRIAGQIEELTELVKLTEPEPQFAEEVWGKRAKEAFEDFIEGNMKNYEAFYLQSDNNTPVYRRNMSRLLRSGLMGVYQKVLASHKQALKRDYLLWSEKHWNVKDENGADISSAEQAQCLLQRLHRKYAESPSRFTEEDCKLYEKVLRRLEDYNQAVKNLSFSSLYEICVLNLEILSRWVGFVQDWERDMYFLLLAWVRQGKLDGIKEEDVRDIFSEGNIIRNLVDTLKGENMNAFESVYFPENKGSKYLGVRNDVAHLDLMRKNGWRLEAGKTCSVMEDYINRLRFLLSYDQKRMNAVTKTLQQIFDRHKVKIRFTVEKGGMLKIEDVTADKIVHLKGSRLSGIEIPSHGERFIDTLKALMVYPRG

SEQ ID NO: 20 - DNA, 84 bp, Artificial Sequence
  Hhe-Sense-28-1 Synthetic DNA
atcagacaaggaactgattacaaacattgttgcagttcccctgtctacggggattgttaccctatagtgagtcgtattaatttc

SEQ ID NO: 21 - DNA, 84 bp, Artificial Sequence
  Hhe-Sense-28-2 Synthetic DNA
ccgcaaattgcacaatttgcccccagcggttgcagttcccctgtctacggggattgttaccctatagtgagtcgtattaatttc

SEQ ID NO: 22 - DNA, 84 bp, Artificial Sequence
  Hhe-Sense-28-3 Synthetic DNA
cgcttcagcgttcttcggaatgtcgcgcgttgcagttcccctgtctacggggattgttaccctatagtgagtcgtattaatttc

SEQ ID NO: 23 - DNA, 84 bp, Artificial Sequence
  Hhe-Sense-28-4 Synthetic DNA
cattggcatggaagtcacaccttcgggagttgcagttcccctgtctacggggattgttaccctatagtgagtcgtattaatttc

SEQ ID NO: 24 - DNA, 84 bp, Artificial Sequence
  Hhe-Sense-28-5 Synthetic DNA
gaacgtggttgacctacacaggtgccatgttgcagttcccctgtctacggggattgttaccctatagtgagtcgtattaatttc

SEQ ID NO: 25 - DNA, 84 bp, Artificial Sequence
  Hhe-Sense-28-6 Synthetic DNA
atcaaattggatgacaaagatccaaattgttgcagttcccctgtctacggggattgttaccctatagtgagtcgtattaatttc

SEQ ID NO: 26 - DNA, 84 bp, Artificial Sequence
  Hhe-NS Synthetic DNA
ctccgtgatatggctcttcgcatgctgagttgcagttcccctgtctacggggattgttaccctatagtgagtcgtattaatttc

SEQ ID NO: 27 - DNA, 84 bp, Artificial Sequence
  Hhe-AntiSense-28-4 Synthetic DNA
tcccgaaggtgtgacttccatgccaatggttgcagttcccctgtctacggggattgttaccctatagtgagtcgtattaatttc

SEQ ID NO: 28 - DNA, 84 bp, Artificial Sequence
  Hhe-AntiSense-28-6 Synthetic DNA
aatttggatctttgtcatccaatttgatgttgcagttcccctgtctacggggattgttaccctatagtgagtcgtattaatttc

SEQ ID NO: 29 - DNA, 80 bp, Artificial Sequence
  Tcc-Sense-24-1 Synthetic DNA
cagacaaggaactgattacaaacagttgcagtctccgcctacatgggagttgtgaccctatagtgagtcgtattaatttc

SEQ ID NO: 30 - DNA, 80 bp, Artificial Sequence
  Tcc-Sense-24-3 Synthetic DNA
cttcagcgttcttcggaatgtcgcgttgcagtctccgcctacatgggagttgtgaccctatagtgagtcgtattaatttc

SEQ ID NO: 31 - DNA, 80 bp, Artificial Sequence
  Tcc-Sense-24-6 Synthetic DNA
caaattggatgacaaagatccaaagttgcagtctccgcctacatgggagttgtgaccctatagtgagtcgtattaatttc

SEQ ID NO: 32 - DNA, 80 bp, Artificial Sequence
  Tcc-Sense-24-2 Synthetic DNA
gcaaattgcacaatttgcccccaggttgcagtctccgcctacatgggagttgtgaccctatagtgagtcgtattaatttc

SEQ ID NO: 33 - DNA, 80 bp, Artificial Sequence
  Tcc-Sense-24-4 Synthetic DNA
ttggcatggaagtcacaccttcgggttgcagtctccgcctacatgggagttgtgaccctatagtgagtcgtattaatttc

SEQ ID NO: 34 - DNA, 80 bp, Artificial Sequence
  Tcc-Sense-24-5 Synthetic DNA
acgtggttgacctacacaggtgccgttgcagtctccgcctacatgggagttgtgaccctatagtgagtcgtattaatttc

SEQ ID NO: 35 - DNA, 84 bp, Artificial Sequence
  Tcc-NS Synthetic DNA
actcagccatccacatccgagtcttcaggttgcagtctccgcctacatgggagttgtgaccctatagtgagtcgtattaatttc

SEQ ID NO: 36 - DNA, 80 bp, Artificial Sequence
  Tcc-AntiSense-24-1 Synthetic DNA
tgtttgtaatcagttccttgtctggttgcagtctccgcctacatgggagttgtgaccctatagtgagtcgtattaatttc

SEQ ID NO: 37 - DNA, 80 bp, Artificial Sequence
  Tcc-AntiSense-24-3 Synthetic DNA
gcgacattccgaagaacgctgaaggttgcagtctccgcctacatgggagttgtgaccctatagtgagtcgtattaatttc

SEQ ID NO: 38 - DNA, 25 bp, Artificial Sequence
  T7 oligo for IVT Synthetic DNA
gaaattaatacgactcactataggg

SEQ ID NO: 39 - DNA, 45 bp, Artificial Sequence
  N gene-target 2-T7 Forward Primer
gaaattaatacgactcactatagggttgcaactgagggagccttg

SEQ ID NO: 40 - DNA, 24 bp, Artificial Sequence
  N gene-target-2 Reverse Primer
gcttcttagaagcctcagcagcag

SEQ ID NO: 41 - DNA, 91 bp, Artificial Sequence
  Sense-mCas13-T1 (crRNA 1)
gctggagcagccctcgatttgcagggtaatcacagcacaaggaactgattacaaacattggccgcaccctatagtgagtcgtattaatttc

SEQ ID NO: 42 - DNA, 91 bp, Artificial Sequence
  Sense-mCas13-T2 (crRNA 2)
gctggagcagccctcgatttgcagggtaatcacagcactgattacaaacattggccgcaaattgcaccctatagtgagtcgtattaatttc

SEQ ID NO: 43 - DNA, 91 bp, Artificial Sequence
  Sense-mCas13-T3 (crRNA 3)
gctggagcagccctcgatttgcagggtaatcacagccgttcttcggaatgtcgcgcattggcatggccctatagtgagtcgtattaatttc

SEQ ID NO: 44 - DNA, 90 bp, Artificial Sequence
  Sense-mCas13-T4 (crRNA 4)
gctggagcagccctcgatttgcagggtaatcacagccaatttgcccccagcgcttcagcgttcttccctatagtgagtcgtattaatttc

SEQ ID NO: 45 - DNA, 91 bp, Artificial Sequence
  Sense-mCas13-N target 2-1 (crRNA 5)
gctggagcagccctcgatttgcagggtaatcacagcaagatcacattggcacccgcaatcctgctaccctatagtgagtcgtattaatttc

SEQ ID NO: 46 - DNA, 90 bp, Artificial Sequence
  Sense-mCas13-N target 2-2 (crRNA 6)
gctggagcagccctcgatttgcagggtaatcacagcacaatgctgcaatcgtgctacaacttcctccctatagtgagtcgtattaatttc

SEQ ID NO: 47 - DNA, 91 bp, Artificial Sequence
  Sense-mCas13-N target 2-3 (crRNA 7)
gctggagcagccctcgatttgcagggtaatcacagccgttcctcatcacgtagtcgcaacagttcaccctatagtgagtcgtattaatttc

SEQ ID NO: 48 - DNA, 91 bp, Artificial Sequence
  Sense-mCas13-N target 2-4 (crRNA 8)
gctggagcagccctcgatttgcagggtaatcacagcacagttcaagaaattcaactccaggcagcaccctatagtgagtcgtattaatttc

SEQ ID NO: 49 - DNA, 90 bp, Artificial Sequence
  Sense-mCas13-N target 2-5 (crRNA 9)
gctggagcagccctcgatttgcagggtaatcacagcgatgctgctcttgctttgctgctgcttgaccctatagtgagtcgtattaatttc

SEQ ID NO: 50 - DNA, 90 bp, Artificial Sequence
  Sense-mCas13-N target 2-6 (crRNA 10)
gctggagcagccctcgatttgcagggtaatcacagcaggccaaactgtcactaagaaatctgctgccctatagtgagtcgtattaatttc

SEQ ID NO: 51 - DNA, 91 bp, Artificial Sequence
  Sense-mCas13-NS (NS)
gctggagcagccctcgatttgcagggtaatcacagcttatgcctcttccgaccatcaagcattttaccctatagtgagtcgtattaatttc

SEQ ID NO: 52 - DNA, 90 bp, Artificial Sequence
  Sense-mCas13-4-Mis-1
gctggagcagccctcgatttgcagggtaatcacagcaaatttgcccccagcgcttcagcgttcttccctatagtgagtcgtattaatttc

SEQ ID NO: 53 - DNA, 90 bp, Artificial Sequence
  Sense-mCas13-4-Mis-2
gctggagcagccctcgatttgcagggtaatcacagccaacttgcccccagcgcttcagcgttcttccctatagtgagtcgtattaatttc

SEQ ID NO: 54 - DNA, 90 bp, Artificial Sequence
  Sense-mCas13-4-Mis-3
gctggagcagccctcgatttgcagggtaatcacagccaatttacccccagcgcttcagcgttcttccctatagtgagtcgtattaatttc

SEQ ID NO: 55 - DNA, 90 bp, Artificial Sequence
  Sense-mCas13-4-Mis-4
gctggagcagccctcgatttgcagggtaatcacagccaatttgcctccagcgcttcagcgttcttccctatagtgagtcgtattaatttc

SEQ ID NO: 56 - DNA, 90 bp, Artificial Sequence
  Sense-mCas13-4-Mis-5
gctggagcagccctcgatttgcagggtaatcacagccaatttgcccccggcgcttcagcgttcttccctatagtgagtcgtattaatttc

SEQ ID NO: 57 - DNA, 90 bp, Artificial Sequence
  Sense-mCas13-4-Mis-6
gctggagcagccctcgatttgcagggtaatcacagccaatttgcccccagcacttcagcgttcttccctatagtgagtcgtattaatttc

SEQ ID NO: 58 - DNA, 90 bp, Artificial Sequence
  Sense-mCas13-4-Mis-7
gctggagcagccctcgatttgcagggtaatcacagccaatttgcccccagcgctccagcgttcttccctatagtgagtcgtattaatttc

SEQ ID NO: 59 - DNA, 90 bp, Artificial Sequence
  Sense-mCas13-4-Mis-8
gctggagcagccctcgatttgcagggtaatcacagccaatttgcccccagcgcttcatcgttcttccctatagtgagtcgtattaatttc

SEQ ID NO: 60 - DNA, 90 bp, Artificial Sequence
  Sense-mCas13-4-Mis-9
gctggagcagccctcgatttgcagggtaatcacagccaatttgcccccagcgcttcagcggtcttccctatagtgagtcgtattaatttc

SEQ ID NO: 61 - DNA, 90 bp, Artificial Sequence
  Sense-mCas13-4-Mis-10
gctggagcagccctcgatttgcagggtaatcacagccaatttgcccccagcgcttcagcgttcctccctatagtgagtcgtattaatttc

SEQ ID NO: 62 - protein, 1023 aa, Artificial Sequence
  mCas13  Synthetic Protein
MGSSHHHHHHSSGLVPRGSHMASWSHPQFEKGGGSGGGSGGSAWSHPQFEKMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSMGIDYSLTSDCYRGINKSCFAVALNIAYDNCDHKGCRTLLSEVLRSKGGISDEQIKSQVVDGIQKRLKDIRNYFSHYYHAEDCLRFGDQDAVKVFLEEIYKNAESKTVGATKESDYKGVVPPLFELHNGTYMITAAGVIFLASFFCHRSNVYRMLGAVKGFKHTGKEQLSDGQKRDYGFTRRLLAYYALRDSYSVGAEDKTRCFREILSYLSRVPQLAVDWLNEQQLLTPEEKEAFLNQPAEDEGGDISDSSSSDKNKKSKEKRRSLRRDEKFILFAIQFIEGWAAEQGLDVTFARYQKTVEKAENKNQDGKQARAVQLKYRNQGLNPDFNNEWMYYIQNEHAIIQIKLNNKKAVAARISENELKYLVLLIFEEKGNDAVQKLNCYIYSMSQKIEGEWKHRPEDERWMPSFTKRADRTVTPEAVQSRLSYIRKQLQETIEKIGQEEPRNNKWLIYKGKKISMILKFISDSIRDIQRRPNVKQYHILRDALQRLDFDGFYKELQNYVNDGRIAVSLYDQIKGVNDISGLCKKVCELTLERLAGLEAKNGSELRRYIGLEAQEKHPKYGEWNTLQEKAKRFLESQFSIGKNFLRKMFYGDCCQKRCFDEEKGYNTQAKERKSLYSIVKEKLKDIKPIHDDRWYLIDRNPKNYDNKHSRIIRQMCNTYIQDVLCMKMAMWHYEKLISATEFRNKLEWNCIGQGNMGYERYSLWYKTGCGVVIQFTPADFLRLDIIEKPAMIENICQCFVLGNKKLNSGAEKKITWDKFNKDGIAKYRKRQAEAVRAIFAFEEGLKIQEDKWSHERYFPFCNILDEAVKQGKIKDTGKDKEALNRGRNDFFHEEFKSTEDQQAIFQKYFPIVERKDDTKKRRDKKQK

SEQ ID NO: 63 - DNA, 3678 bp, Artificial Sequence
  TccCas13a Synthetic DNA
atgaagatcacgaaaaggaaatggggagagcatcatccgccgctttacttctaccgggatgaggactccggcaggctcctggcacagaacgacagaaagcaggattataccgataccctgtttaatgatattgcgcaggatacatttgaaagatcgctgagaaaccggcttttgaaaacacccgaaaagggagacaaaagattctacagcaacgagatcgtcaagctggtggagaaactgtgccagggtgcggatgtggcggagatcatgaaaagcatggagaggaacgaaaagctgcgccccaagaatgaaaaagagattaaaaatctgaaaaagcaattggacggtaccctttccgaatacggtaaaaggtataccgccccagaaggcgccatgaccctcaacgatgccttgttttacctggtagaaggaaaccctttaaagcaggccatggccaaagctgaactgggcaaaatccgggaggcattaataaaggaaaaggaaaaccgcatcaaccgcgtccggtattccatcaagaacaataaaataccgctgagaatccaagaggatggcggcattacacccaataatgaccgtgcggcctggctgctggggctcatgaagccggcggacccggcaaaaggaataaccgactgctacccgctcttaggcgagttggaagaagtattcgactttgacaagctgtccaaaacgctgcacgaaaagataagccgttgccaaggccgaccccgttccatagccatggcggtcgatgaggccctgaagcaatatctccgggagctttgggagaagtctccctcgcgacagcaggatctgaagtattactttcaggccgtacaggagtactttaaggacaattttcccatccggacaaaacggatgggcgcccgcctgcggcaggaattgctcaaggataagacgtccctttcccgtctgctggaacccaagcatatggctaatgccgttcgccgcaggctgatcaaccagagtacccagatgcacatcctttatggaaagctttatgcatattgctgcggggaagacggcaggcttttggtaaacagcgagacgctgcaaaggatacaggtccatgaagcggtaaaaaagcaggccatgacggccgtgctgtggtccatatcccgtctgcgctatttttaccagtttgaagacggcgatatcttgagcaataaaaacccgattaaagatttcagagataaatttctcagagacacgaataaatatacccatgaagatgttgaggcctgcaaggaaaaactgcaggacttcttcccgctgaaagaattgcaggaaaagattaaggaggatgcaaaaggattacaggaaacagacaataagcaggctgatacaacggatttcaaagcgatcgggcacatcgtcagggatgatcggaagctctgcaaccagttgctggcggagtgcgtttcctgcatcggggagctgagacatcatatctttcattataaaaatgtgaccttgatacaggcgctcaaaaggatcgccgataaagtgaaaccggaggatttgtctgtgctccgggccatttacctgttggacaggagaaacctcaaaaaggcgtttgccaaaagaatcagcagcatgaaccttccgctgtattacagggaggatctattatcccgcattttcaaaaaagaagggacggcgtttttcctgtacagcgccaagatccagatgacaccgtcctttcagagggtctatgagcgtggtaaaaatctgcgccgggagttcgagtgcgaacgcatgaaagccgaggcatccaacggacagaacgggcaggatggcgaccggctgaaatggttccgacagcttgcggctggggatagcgccgatacccatttcaactgggctgtggaggcctatgcggaatcggcagccgatgtggaaaacaacgttgaattcgataccgatgtggatgcccagcgtgccctgcggaaccttctgctgctgatatataggcaccatttcctgccggaggtgcagaaggatgaaaccctggtgaccggcaagatccataaggttttggaaagaaacaggcagctgtctgaaggccaagggccaaatcaaggtaaggcccatggatacagcgttatagaagagctgtatcatgaaggcatgccgctttccgatctcatgaagcaattgcagcggaggatctccgaaaccgaaagggagagccgggaactggcacaggaaaaaacggattatgcccagcgctttatcctcgatatctttgccgaggcctttaacgatttcctggaggcgcactatggcgaagaataccttgagatcatgagccccaggaaagatgccgaagcggcgaagaaatgggtaaaagaaagcaaaacggtggatttgaaaacatctatagacgaaaaagaaccggaagggcatctcctggtgctctatcccgtcctgcgcctgctggacgaaagggaactgggagagcttcagcagcagatgatccgttatcggacatccctggccagttggcagggtgagagcaatttcagtgaagaaataaggatagccgggcagattgaggaattgaccgaactggtcaagctgacggaaccggagccccagtttgcggaggaagtatgggggaaacgggctaaagaagcgtttgaagactttattgaaggaaacatgaaaaattatgaggctttttatcttcagagcgacaacaacacgccggtatatcggaggaacatgagccggttgctgcgctcggggcttatgggagtgtaccaaaaggtgcttgccagccacaagcaggcgctcaaaagagattacttgctctggtcggaaaaacattggaacgtgaaggatgagaatggagcggatatctcttccgctgaacaggcccaatgccttttgcagcggctccataggaagtacgccgaatccccgtcccgatttacggaggaggactgcaaactgtatgagaaggttctccggcggcttgaggactataaccaggccgtgaaaaacctgtccttcagttcattgtatgaaatatgcgtcctcaatcttgaaatcctctcccgatgggtggggtttgtccaggactgggagcgggatatgtacttcctccttttggcgtgggtcagacagggcaaactggacgggataaaggaggaagatgtcagagatatcttctctgaaggcaacattattcgcaatctggtggatacgctgaaaggcgaaaacatgaatgcctttgaaagcgtttatttccctgagaataaggggtctaagtatttaggtgtgcggaatgatgttgcccatctggatctgatgcggaaaaacggatggcggttggaggctggcaaaacctgcagcgtgatggaagattacatcaaccgtttgaggtttctcctgtcctatgaccagaaacgcatgaacgccgtcaccaagaccttgcagcagatatttgataggcataaagtcaaaatccggttcaccgtggagaaaggaggaatgctgaagatagaggatgtgactgccgacaagatcgtgcatctgaaaggttccaggttgagcggcattgaaattccaagtcacggggagaggtttattgacacgttgaaagcgctgatggtatacccgagaggatga

SEQ ID NO: 64 - protein, 1225 aa, Artificial Sequence
  TccCas13a Protein
MKITKRKWGEHHPPLYFYRDEDSGRLLAQNDRKQDYTDTLFNDIAQDTFERSLRNRLLKTPEKGDKRFYSNEIVKLVEKLCQGADVAEIMKSMERNEKLRPKNEKEIKNLKKQLDGTLSEYGKRYTAPEGAMTLNDALFYLVEGNPLKQAMAKAELGKIREALIKEKENRINRVRYSIKNNKIPLRIQEDGGITPNNDRAAWLLGLMKPADPAKGITDCYPLLGELEEVFDFDKLSKTLHEKISRCQGRPRSIAMAVDEALKQYLRELWEKSPSRQQDLKYYFQAVQEYFKDNFPIRTKRMGARLRQELLKDKTSLSRLLEPKHMANAVRRRLINQSTQMHILYGKLYAYCCGEDGRLLVNSETLQRIQVHEAVKKQAMTAVLWSISRLRYFYQFEDGDILSNKNPIKDFRDKFLRDTNKYTHEDVEACKEKLQDFFPLKELQEKIKEDAKGLQETDNKQADTTDFKAIGHIVRDDRKLCNQLLAECVSCIGELRHHIFHYKNVTLIQALKRIADKVKPEDLSVLRAIYLLDRRNLKKAFAKRISSMNLPLYYREDLLSRIFKKEGTAFFLYSAKIQMTPSFQRVYERGKNLRREFECERMKAEASNGQNGQDGDRLKWFRQLAAGDSADTHFNWAVEAYAESAADVENNVEFDTDVDAQRALRNLLLLIYRHHFLPEVQKDETLVTGKIHKVLERNRQLSEGQGPNQGKAHGYSVIEELYHEGMPLSDLMKQLQRRISETERESRELAQEKTDYAQRFILDIFAEAFNDFLEAHYGEEYLEIMSPRKDAEAAKKWVKESKTVDLKTSIDEKEPEGHLLVLYPVLRLLDERELGELQQQMIRYRTSLASWQGESNFSEEIRIAGQIEELTELVKLTEPEPQFAEEVWGKRAKEAFEDFIEGNMKNYEAFYLQSDNNTPVYRRNMSRLLRSGLMGVYQKVLASHKQALKRDYLLWSEKHWNVKDENGADISSAEQAQCLLQRLHRKYAESPSRFTEEDCKLYEKVLRRLEDYNQAVKNLSFSSLYEICVLNLEILSRWVGFVQDWERDMYFLLLAWVRQGKLDGIKEEDVRDIFSEGNIIRNLVDTLKGENMNAFESVYFPENKGSKYLGVRNDVAHLDLMRKNGWRLEAGKTCSVMEDYINRLRFLLSYDQKRMNAVTKTLQQIFDRHKVKIRFTVEKGGMLKIEDVTADKIVHLKGSRLSGIEIPSHGERFIDTLKALMVYPRG

SEQ ID NO: 65 - DNA, 2622 bp, Artificial Sequence
  Proteobacteria-derived Cas13 Synthetic DNA
atgggtattgattattcgcttacaagtgactgttatcgaggcatcaacaagtcttgttttgcagttgctttgaatattgcatatgataactgtgatcataaaggatgtaggactcttctgagtgaggtgctgcgcagcaagggagggatttctgatgagcaaataaaatcacaagtagtggatggaattcagaagcgacttaaagacattcgcaattatttctcgcattattaccacgcagaagactgtctgcggtttggagaccaagatgccgtcaaggtttttctggaagaaatatacaagaacgcggaatcaaagactgtcggagcgacaaaagaaagcgactataaaggcgttgtgccgccattatttgaattgcataacggtacatatatgattacggcggcgggggttatttttctggcatcatttttctgccatcggagtaatgtctatcggatgctgggagcggtgaaaggatttaaacataccggaaaagagcaattgagcgatgggcagaaacgagattatggtttcactcgccggctgctggcttattatgcgctccgggacagctattctgtgggggcggaagacaagacacgatgtttccgcgagatattaagctatttgtcgagagtaccgcaattggcagtagattggctgaatgaacaacagctgcttacaccagaagaaaaagaagcttttttgaatcaacccgccgaagatgagggcggggatatttcggactcttcgtccagtgataaaaataaaaaaagcaaagaaaagaggcgtagtctccgcagagatgaaaaattcattttatttgctatccaatttattgaggggtgggcggctgaacagggattagatgtgacatttgcacgctaccaaaaaacagtggaaaaagcggagaataagaatcaggatggcaaacaggccagagcggtgcaattaaaatacagaaaccaaggactcaatccggatttcaataacgaatggatgtattacattcagaatgaacacgcaattatccagattaaactgaataataaaaaagccgttgctgcccgtatttctgaaaatgaattaaaatatctggtgctgctgatttttgaggagaagggcaatgacgccgtccaaaagctgaattgttatatttacagtatgagccaaaaaatcgagggcgaatggaaacacaggccggaggatgagcgatggatgccgtcgtttaccaagcgtgccgacaggacggttacgccggaggcagtgcagagccggttaagctatattcgcaaacaacttcaggagacgatagagaaaatcggtcaggaagagccgcggaataataagtggctgatatacaaaggcaaaaaaatatcaatgatactgaagtttatctccgacagcattcgcgatattcaaaggcggccgaatgtgaaacaatatcatattttacgcgatgcgcttcagaggctcgactttgatggattttataaggaacttcaaaattacgtcaatgacggccggattgcggtttcattatacgatcagatcaagggtgtcaatgacatcagcgggctttgtaaaaaagtctgcgaactgacacttgaaagactggctgggctggaggcaaagaatggctccgagctgaggcgttatattgggcttgaagcgcaggaaaaacatccgaagtacggagagtggaacacactgcaggaaaaggccaagcgatttctggagtcgcagttttctatcgggaaaaactttttgcggaaaatgttttatggcgattgttgtcaaaagcggtgctttgacgaagaaaaaggttacaatacacaagcgaaagagcgaaaaagtctgtatagcattgtgaaagaaaaactcaaggacatcaagcccatacacgatgaccggtggtatcttatcgacaggaatccgaagaactatgacaataaacacagcaggattatccgacagatgtgcaacacctatatacaagatgttctctgtatgaagatggcgatgtggcattatgagaaattaatctccgcgactgaattcaggaataaattggaatggaattgtattgggcagggaaatatgggttatgagcgatattcgctgtggtataagacaggctgcggggttgtcatacaatttacgccggcggattttttgcgactggatatcattgaaaaacctgcgatgatagaaaatatttgccagtgttttgtgctcggaaacaaaaaactcaattcgggtgccgaaaagaagataacctgggataaattcaataaagacggcattgcaaaatacagaaagcggcaggccgaggctgtgcgtgcgatatttgcgtttgaggaaggtttaaaaatacaagaggataaatggagtcatgagagatactttccattttgcaatattcttgacgaggcggtaaaacaaggtaaaataaaagatacaggcaaagacaaggaggctctgaatcgcggcagaaatgatttttttcatgaggaatttaagtcaacagaagatcagcaggcgatttttcagaaatactttccgattgtcgaacgaaaagacgacacaaaaaaacggcgagataaaaagcaaaagtga

SEQ ID NO: 66 - protein, 873 aa, Artificial Sequence
  mCas13 Synthetic Protein
MGIDYSLTSDCYRGINKSCFAVALNIAYDNCDHKGCRTLLSEVLRSKGGISDEQIKSQVVDGIQKRLKDIRNYFSHYYHAEDCLRFGDQDAVKVFLEEIYKNAESKTVGATKESDYKGVVPPLFELHNGTYMITAAGVIFLASFFCHRSNVYRMLGAVKGFKHTGKEQLSDGQKRDYGFTRRLLAYYALRDSYSVGAEDKTRCFREILSYLSRVPQLAVDWLNEQQLLTPEEKEAFLNQPAEDEGGDISDSSSSDKNKKSKEKRRSLRRDEKFILFAIQFIEGWAAEQGLDVTFARYQKTVEKAENKNQDGKQARAVQLKYRNQGLNPDFNNEWMYYIQNEHAIIQIKLNNKKAVAARISENELKYLVLLIFEEKGNDAVQKLNCYIYSMSQKIEGEWKHRPEDERWMPSFTKRADRTVTPEAVQSRLSYIRKQLQETIEKIGQEEPRNNKWLIYKGKKISMILKFISDSIRDIQRRPNVKQYHILRDALQRLDFDGFYKELQNYVNDGRIAVSLYDQIKGVNDISGLCKKVCELTLERLAGLEAKNGSELRRYIGLEAQEKHPKYGEWNTLQEKAKRFLESQFSIGKNFLRKMFYGDCCQKRCFDEEKGYNTQAKERKSLYSIVKEKLKDIKPIHDDRWYLIDRNPKNYDNKHSRIIRQMCNTYIQDVLCMKMAMWHYEKLISATEFRNKLEWNCIGQGNMGYERYSLWYKTGCGVVIQFTPADFLRLDIIEKPAMIENICQCFVLGNKKLNSGAEKKITWDKFNKDGIAKYRKRQAEAVRAIFAFEEGLKIQEDKWSHERYFPFCNILDEAVKQGKIKDTGKDKEALNRGRNDFFHEEFKSTEDQQAIFQKYFPIVERKDDTKKRRDKKQK

SEQ ID NO: 67 - DNA, 3855 bp, Artificial Sequence
  Cas13a  Synthetic DNA
atgaagttgacgcgtcgccgcatcagtggcaattcagtcgaccagaagattacagctgcgttttatcgtgatatgtcgcaagggttattatattatgattcggaggacaatgactgtacggacaaggtcatcgaaagtatggatttcgaacgctcttggcgtggacgcattctgaagaacggcgaggatgataagaatccgttttacatgttcgttaaaggtttagttggttccaacgataagatcgtgtgcgaacctatcgacgtcgactcagaccctgataacttagatatcttgattaacaaaaatctgacggggttcggacgcaacttgaaagcacctgacagtaacgataccttagaaaatcttatccgcaaaatccaggccggcattcccgaagaagaggttcttccggagctgaagaaaattaaagagatgatccagaaagacatcgttaaccgcaaagagcagttgcttaaatccatcaaaaacaaccgtattcctttttcattggaaggttccaagctggtcccttccaccaaaaagatgaagtggcttttcaagttgattgacgttcctaataaaaccttcaatgaaaaaatgttggagaagtactgggagatctacgactatgacaagcttaaggcgaatatcacgaaccgcctggacaaaacggataagaaggcacgttcaatttcgcgtgcggtaagcgaggagttacgtgagtaccataaaaacttgcgcacaaactataatcgcttcgtatctggagaccgtccagcagccgggcttgacaatggggggagtgctaaatataatcccgataaggaggagttcttattgtttcttaaagaagttgagcagtacttcaagaaatacttcccagtcaagagcaagcattcgaacaagtccaaagacaaatcattggttgataagtacaaaaattattgcagttacaaagtggtcaagaaagaggtcaaccgcagcatcatcaatcaattggtggctggacttattcagcagggtaagcttctgtattacttctactataacgatacctggcaggaggatttcctgaattcctacggtcttagttatattcaggttgaggaggcatttaaaaagagtgtaatgaccagtctttcgtggggaatcaaccgtctgacctcgttctttatcgacgactcgaacactgtgaagtttgatgatatcacaacgaaaaaggctaaggaggcgattgagtctaattatttcaacaaattacgcacatgctcccgtatgcaggaccacttcaaagagaaattggccttcttttacccggtgtatgtgaaggacaagaaggatcgcccggatgatgatatcgaaaacttaattgtactggttaagaacgccattgagagcgtgagctatttgcgcaatcgcacgtttcactttaaagagagctccttattggagctgcttaaagagttagacgataaaaattctggtcaaaataaaatcgactatagcgtagccgctgaatttattaaacgcgatatcgagaacttgtatgacgtcttccgtgaacagatccgctcacttggtattgctgagtactacaaggcggacatgatttcggactgttttaagacatgcgggttagaattcgcgctttattcgccaaaaaattcgttgatgcccgcttttaagaacgtttataaacgcggagcaaatcttaacaaggcgtatattcgcgataaagggccaaaggaaacaggagaccagggtcagaactcgtacaaagctttggaggaatatcgtgagcttacatggtatattgaggtgaagaataacgaccagtcgtacaacgcctacaagaatttgcttcaactgatttattatcatgcgtttcttccggaggttcgtgaaaatgaggctcttatcactgacttcattaaccgcactaaagagtggaatcgtaaggaaacagaggagcgtctgaacactaaaaacaataaaaaacacaagaatttcgacgagaatgacgatattacggtaaatacgtatcgttacgaatctattccggattaccagggggaatcccttgatgattatcttaaggtgttacagcgcaagcaaatggcgcgtgcgaaggaggtaaatgagaaggaggagggcaacaataactatatccaattcattcgtgacgtcgtagtatgggcgttcggggcctacttggaaaacaagctgaagaattataagaacgaactgcaaccaccgttgagcaaagagaacattggcttaaatgataccttaaaggaattgtttcccgaagaaaaagtaaagtcaccatttaatatcaagtgccgtttctccattagtacatttatcgacaacaaaggtaagtccaccgataatacctcggccgaggccgtcaagactgatggcaaggaggacgaaaaggacaaaaagaatattaaacgcaaagacttgctttgcttttacctgttcttacgcttgcttgatgaaaacgaaatttgcaaattacaacaccaatttattaaataccgttgcagtttaaaagagcgtcgttttccaggtaaccgcaccaaactggagaaggaaactgaacttttggccgagctggaagaactgatggaactggtacgttttacgatgccatcaattcctgaaatctctgcaaaggcggaatcaggttatgacacaatgattaagaagtattttaaagacttcatcgagaaaaaagtctttaagaatccgaagacgtccaacctgtactaccactccgactctaaaactccggtcacccgcaaatatatggctctgctgatgcgttctgcgcctttgcatctctacaaggatatcttcaagggctactatttgatcactaagaaggagtgtttggagtatattaaattaagcaacatcattaaagactatcaaaactcattgaatgaattgcacgagcagctggagcgcattaaattaaagagtgagaaacaaaacgggaaagactctctgtatcttgacaaaaaggacttctataaagtaaaggagtacgtcgaaaatttagaacaagtggcgtgttacaagcacttgcagcacaaaatcaacttcgaaagcttataccgtatttttcgtattcacgtggacatcgctgcgcgtatggtcggttatacccaagactgggagcgtgatatgcactttcttttcaaggcgttggtctataatggcgtacttgaggagcgccgttttgaagcaatctttaacaataacgatgacaacaacgacgggcgtatcgtaaaaaagatccagaataacttaaataacaagaaccgcgaattagtatctatgttatgttggaataaaaagctgaacaagaacgaatttggtgctattatttggaagcgtaaccctatcgcccatctgaaccactttacacaaacggaacaaaactcaaagtcgagccttgaatctctgatcaactcgctgcgcatcctgttagcgtacgaccgtaaacgtcaaaacgctgtgacaaagactatcaatgaccttctgttaaacgactatcacatccgcattaagtgggaagggcgcgtggatgaagggcagatctactttaatatcaaggaaaaggaagacatcgagaacgagcctattatccacttgaaacacctgcataaaaaggactgctacatttacaagaacagctatatgttcgacaagcaaaaggagtggatttgtaatggcattaaagaagaagtttatgacaagtccattcttaagtgtatcggaaacttgttcaagtttgactatgaggacaaaaacaaatcctcagcaaatcctaagcacacc

SEQ ID NO: 68 - protein, 1285 aa, Artificial Sequence
  Herbinix hemicellulosilytica Cas13a  Synthetic Protein
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVACYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 69 - DNA, 9761 bp, Artificial Sequence
  TccCas13a Synthetic Protein
tcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagctggagccatccgcagtttgaaaaaggtggtggtagcggtggtggttcaggtggtagtgcatggtcacaccctcagtttgagaaaatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaatccaagctgatcagacccctgaagatttggacatggaggataacgatattattgaggctcacagagaacagattggtggatccatgaagattaccaaacgcaagtggggtgaacaccatccgccgctgtatttctaccgtgatgaagatagcggtcgtctgctggcgcaaaacgaccgtaagcaagattacaccgacaccctgttcaacgacatcgcgcaggacacctttgaacgtagcctgcgtaaccgtctgctgaaaaccccggagaagggcgacaaacgtttctatagcaacgaaatcgtgaaactggttgagaagctgtgccagggtgcggatgttgcggagattatgaagagcatggaacgtaacgagaaactgcgtccgaagaacgagaaagaaattaagaacctgaagaaacagctggacggcaccctgagcgagtacggcaagcgttataccgcgccggaaggtgcgatgaccctgaacgatgcgctgttttacctggttgagggcaacccgctgaaacaggcgatggcgaaggcggaactgggtaaaatccgtgaggcgctgattaaggagaaagaaaaccgtatcaaccgtgtgcgttatagcattaaaaacaacaagatcccgctgcgtattcaggaagatggtggcatcaccccgaacaacgaccgtgcggcgtggctgctgggcctgatgaagccggcggacccggcgaaaggcattaccgactgctacccgctgctgggtgaactggaggaagttttcgactttgataaactgagcaagaccctgcacgagaaaatcagccgttgccaaggtcgtccgcgtagcattgcgatggcggtggacgaggcgctgaaacagtacctgcgtgaactgtgggagaagagcccgagccgtcagcaagacctgaaatactatttccaagcggttcaggaatatttcaaggataactttccgatccgtaccaaacgtatgggcgcgcgtctgcgtcaggaactgctgaaggacaaaaccagcctgagccgtctgctggagccgaagcacatggcgaacgcggtgcgtcgtcgtctgatcaaccaaagcacccagatgcacattctgtacggtaaactgtacgcgtattgctgcggcgaggatggccgtctgctggttaacagcgaaaccctgcaacgtatccaggtgcacgaggcggttaagaaacaagcgatgaccgcggtgctgtggagcattagccgtctgcgttacttctatcagtttgaggacggcgacatcctgagcaacaaaaacccgattaaggatttccgtgacaagtttctgcgtgataccaacaaatacacccacgaagacgttgaggcgtgcaaggaaaaactgcaagatttctttccgctgaaggaactgcaggagaagatcaaagaggatgcgaagggcctgcaagaaaccgacaacaaacaggcggataccaccgacttcaaggcgatcggtcacattgttcgtgacgatcgtaaactgtgcaaccaactgctggcggagtgcgtgagctgcatcggtgaactgcgtcaccacatttttcactacaagaacgtgaccctgatccaggcgctgaaacgtattgcggataaggtgaaaccggaggacctgagcgttctgcgtgcgatctatctgctggaccgtcgtaacctgaagaaagcgttcgcgaagcgtattagcagcatgaacctgccgctgtactatcgtgaggacctgctgagccgtatctttaagaaagagggcaccgcgttctttctgtacagcgcgaaaattcaaatgaccccgagcttccagcgtgtttatgaacgtggcaagaacctgcgtcgtgagtttgaatgcgagcgtatgaaagcggaggcgagcaacggtcaaaacggccaggacggtgatcgtctgaagtggtttcgtcagctggcggcgggtgatagcgcggacacccactttaactgggcggttgaagcgtacgcggagagcgcggcggatgtggaaaacaacgttgagttcgataccgatgtggacgcgcagcgtgcgctgcgtaacctgctgctgctgatctatcgtcaccactttctgccggaagtgcaaaaggatgaaaccctggttaccggcaaaattcacaaggtgctggagcgtaaccgtcagctgagcgaaggtcaaggcccgaaccagggcaaagcgcacggttacagcgtgatcgaggaactgtatcacgagggtatgccgctgagcgacctgatgaagcaactgcagcgtcgtattagcgaaaccgagcgtgaaagccgtgaactggcgcaagagaaaaccgattacgcgcagcgtttcatcctggacatttttgcggaggcgttcaacgattttctggaagcgcactacggcgaggaatatctggaaatcatgagcccgcgtaaagacgcggaggcggcgaagaaatgggtgaaagaaagcaagaccgttgatctgaagaccagcattgacgagaaagaaccggagggtcacctgctggtgctgtacccggttctgcgtctgctggatgaacgtgagctgggtgaactgcagcaacagatgatccgttatcgtaccagcctggcgagctggcaaggcgagagcaacttcagcgaggaaatccgtattgcgggtcagattgaggaactgaccgaactggttaagctgaccgaaccggagccgcaatttgcggaggaagtgtggggcaaacgtgcgaaggaagcgttcgaggactttatcgagggtaacatgaaaaactacgaagcgttctatctgcagagcgataacaacaccccggtttaccgtcgtaacatgagccgtctgctgcgtagcggcctgatgggtgtgtaccaaaaggttctggcgagccacaaacaggcgctgaagcgtgactatctgctgtggagcgagaagcactggaacgtgaaagatgaaaacggcgcggacatcagcagcgcggaacaagcgcagtgcctgctgcaacgtctgcaccgtaagtacgcggagagcccgagccgtttcaccgaggaagactgcaaactgtacgagaaggttctgcgtcgtctggaagattataaccaggcggtgaaaaacctgagctttagcagcctgtacgaaatctgcgtgctgaacctggaaattctgagccgttgggtgggtttcgttcaagattgggagcgtgacatgtattttctgctgctggcgtgggttcgtcagggcaagctggacggtatcaaagaggaagatgtgcgtgacattttcagcgaaggcaacatcattcgtaacctggttgataccctgaaaggtgaaaacatgaacgcgttcgagagcgtgtactttccggaaaacaaaggcagcaagtatctgggtgtgcgtaacgatgttgcgcacctggacctgatgcgtaagaacggctggcgtctggaagcgggtaaaacctgcagcgttatggaggactacatcaaccgtctgcgtttcctgctgagctatgatcagaagcgtatgaacgcggtgaccaaaaccctgcaacagattttcgaccgtcacaaggtgaaaattcgttttaccgttgagaagggtggcatgctgaaaatcgaagatgtgaccgcggacaagattgttcacctgaaaggcagccgtctgagcggtatcgagattccgagccacggcgagcgttttattgacaccctgaaggcgctgatggtttacccgcgtggttaagcggccgcactcgaggcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatc

SEQ ID NO: 70 - DNA, 8705 bp, Artificial Sequence
  mCas13a  Synthetic DNA
tttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagctggagccatccgcagtttgaaaaaggtggtggtagcggtggtggttcaggtggtagtgcatggtcacaccctcagtttgagaaaatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaatccaagctgatcagacccctgaagatttggacatggaggataacgatattattgaggctcacagagaacagattggtggatccatgggaatagactatagtctaacatcagattgctaccgtggtatcaataaatcttgtttcgctgtggcgctgaacattgcttacgacaactgcgatcataaaggctgccgtaccctgctgtcggaggttttgcgctctaaaggcggtatttcggacgaacagattaagtcccaagttgtcgatggtatccagaagcgcctgaaggatatccgtaactatttcagccattattatcatgcagaggattgcctgcgctttggtgaccaggatgcggtgaaagtgtttctggaggagatttataagaatgcagagagcaaaacggtcggtgcgaccaaagagtctgactacaagggcgttgtacctccgctctttgaattacataatggtacatacatgattaccgcagcgggtgtcatctttctcgccagcttcttctgccatcgtagcaacgtttatcgcatgctgggtgccgtcaaaggcttcaaacacaccggcaaagagcagctgtccgatggccagaaacgtgactatggctttacgcgtcgcctgctcgcttactatgctctgcgtgactcctacagcgttggcgcagaagataaaacgcgttgctttcgtgaaattctgtcgtatctgtcacgtgttccacagttggcggtggactggctgaacgaacaacaattgttgacgccggaagaaaaggaggcgtttctgaaccagccggcagaggacgagggcggtgacatctctgacagcagtagctccgacaaaaacaaaaagtccaaagagaaaagacgcagcctgcgccgtgatgagaagttcattttgtttgccatccaatttatcgagggctgggcagcagagcagggtctggatgttaccttcgctcgttaccagaagaccgttgaaaaggctgagaacaaaaaccaggacggtaaacaagcgcgtgcagttcagctcaagtaccgcaaccaaggcctgaacccggatttcaacaacgagtggatgtattacatccagaacgagcacgcgattattcagattaaactgaataataagaaagcggttgcagcgcgtattagcgaaaatgaactgaagtatttggtcctgttgatcttcgaggagaagggtaatgacgcggtgcagaagttgaattgctacatctacagcatgagccagaagattgagggtgaatggaaacaccgtccggaagatgagcgttggatgccgagcttcaccaaacgagcggaccgcaccgtgaccccggaagccgtccaaagccgtctgagctatatccgcaaacagctgcaagaaaccattgagaagattggtcaagaagaaccgcgtaacaacaagtggctgatctacaagggcaaaaagatctctatgattttgaagttcatcagtgattcgatccgtgatattcaacgccgtccgaatgttaaacaatatcacattttgcgtgacgcactgcaacgtctcgacttcgatggtttttataaggaacttcagaattatgttaatgacggccgcattgccgtaagcctgtatgaccaaatcaagggcgtgaatgacatcagcggtctgtgtaaaaaagtttgtgaactgaccttggaaaggctagcgggtctggaggctaaaaacggcagcgaactgagacgttacatcggcctggaagcgcaggagaagcacccgaagtacggtgaatggaataccctgcaagaaaaagcgaaacgctttctggagtctcagttctccattggtaagaattttttgcgtaaaatgttttacggcgattgttgtcaaaagcgctgcttcgatgaggagaaaggctacaacacccaggcgaaggagcgcaaaagcctgtattccatcgttaaggagaagctgaaggatatcaagccgatccatgatgaccgttggtatctgattgatcgtaacccgaaaaactacgataataagcacagccgcatcatccgtcagatgtgcaacacgtacatccaagatgtgttatgcatgaagatggcgatgtggcactacgagaaattaatctccgcgactgagttccgtaacaagttggagtggaactgcattggtcagggtaacatgggttatgaacgttactcactgtggtacaagaccggttgcggtgtggtgatccaattcacgccagctgatttcctgcggctggacattattgaaaaaccggcaatgatcgagaacatctgccagtgtttcgtgctgggcaacaaaaagctaaacagcggcgcggaaaagaaaatcacttgggataaattcaacaaggacggcatagcgaaataccgtaagcgccaggccgaggcggtgcgtgcgatcttcgcgtttgaagagggtctcaagatccaagaagacaaatggtcacacgaacgttacttcccgttttgtaatatcttggatgaagccgtgaagcaggggaaaatcaaagataccggtaaggacaaagaagccttaaatcgtggtcgtaacgacttttttcatgaagaatttaaaagcaccgaagaccagcaagcgatttttcaaaaatacttcccgattgttgaacggaaggacgacaccaaaaaacgtcgcgataagaaacagaagtaagcggccgcactcgaggcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttt

SEQ ID NO: 71 - DNA, 17 bp, Artificial Sequence
  RNase P Forward Primer
ttgatgagctggagcca

SEQ ID NO: 72 - DNA, 18 bp, Artificial Sequence
  RNase P Synthetic DNA
caccctcaatgcagagtc

SEQ ID NO: 73 - DNA, 41 bp, Artificial Sequence
  RNase P- FIP Synthetic DNA
gtgtgaccctgaagactcggttttagccactgactcggatc

SEQ ID NO: 74 - DNA, 45 bp, Artificial Sequence
  RNase P- BIP Synthetic DNA
cctccgtgatatggctcttcgtttttttcttacatggctctggtc

SEQ ID NO: 75 - DNA, 20 bp, Artificial Sequence
  RNase P- LF Synthetic DNA
atgtggatggctgagttgtt

SEQ ID NO: 76 - DNA, 20 bp, Artificial Sequence
  RNase P- LB Synthetic DNA
catgctgagtactggacctc

SEQ ID NO: 77 - DNA, 61 bp, Artificial Sequence
  RNase P- T7-FIP Synthetic DNA
gtgtgaccctgaagactcggtttttaatacgactcactatagggagccactgactcggatc

SEQ ID NO: 78 - DNA, 19 bp, Artificial Sequence
  HCV-F3 Synthetic DNA
tgtcttcacgcagaaagcg

SEQ ID NO: 79 - DNA, 18 bp, Artificial Sequence
  HCV-B3 Synthetic DNA
taccacaaggcctttcgc

SEQ ID NO: 80 - DNA, 61 bp, Artificial Sequence
  HCV- T7-FIP Synthetic DNA
tccgcagaccactatggctctctaatacgactcactatagggccatggcgttagtatgagt

SEQ ID NO: 81 - DNA, 40 bp, Artificial Sequence
  HCV- BIP Synthetic DNA
aggacgaccgggtcctttctactactcggctagcagtctt

SEQ ID NO: 82 - DNA, 20 bp, Artificial Sequence
  HCV-LF Synthetic DNA
ggtcctggaggctgcacgac

SEQ ID NO: 83 - DNA, 21 bp, Artificial Sequence
  HCV-LB Synthetic DNA
gagatttgggcgtgcccccgc

SEQ ID NO: 84 - DNA, 20 bp, Artificial Sequence
  TYLCV- F3 Synthetic DNA
ggtaaagtctggatggatga

SEQ ID NO: 85 - DNA, 19 bp, Artificial Sequence
  TYLCV- B3 Synthetic DNA
tgttccttcattccagagg

SEQ ID NO: 86 - DNA, 60 bp, Artificial Sequence
  TYLCV- T7-FIP Synthetic DNA
acctgtccaaaatccattgtaatacgactcactatagggcagaatcacactaatcaggtc

SEQ ID NO: 87 - DNA, 45 bp, Artificial Sequence
  TYLCV- BIP Synthetic DNA
cagtaccgcaaccgtgaagacaataactgtagcatgaaatttcct

SEQ ID NO: 88 - DNA, 21 bp, Artificial Sequence
  TYLCV- LF Synthetic DNA
ctatcacggaccaagaagaac

SEQ ID NO: 89 - DNA, 22 bp, Artificial Sequence
  TYLCV- LB Synthetic DNA
gatttgcgggataggtttcaag

SEQ ID NO: 90 - DNA, 84 bp, Artificial Sequence
  1104-Mis 1-18 Synthetic DNA
cattggcatggaagtcagaccttcgggagttgcagttcccctgtctacggggattgttaccctatagtgagtcgtattaatttc

SEQ ID NO: 91 - DNA, 84 bp, Artificial Sequence
  1104-Mis 1 19 Synthetic DNA
cattggcatggaagtcactccttcgggagttgcagttcccctgtctacggggattgttaccctatagtgagtcgtattaatttc

SEQ ID NO: 92 - DNA, 84 bp, Artificial Sequence
  1104-Mis 1-20
cattggcatggaagtcacagcttcgggagttgcagttcccctgtctacggggattgttaccctatagtgagtcgtattaatttc

SEQ ID NO: 93 - DNA, 84 bp, Artificial Sequence
  1104-Mis 1-21
cattggcatggaagtcacacgttcgggagttgcagttcccctgtctacggggattgttaccctatagtgagtcgtattaatttc

SEQ ID NO: 94 - DNA, 84 bp, Artificial Sequence
  1104-Mis 1-22
cattggcatggaagtcacaccatcgggagttgcagttcccctgtctacggggattgttaccctatagtgagtcgtattaatttc

SEQ ID NO: 95 - DNA, 84 bp, Artificial Sequence
  1104-Mis 1-23
cattggcatggaagtcacacctacgggagttgcagttcccctgtctacggggattgttaccctatagtgagtcgtattaatttc

SEQ ID NO: 96 - DNA, 84 bp, Artificial Sequence
  1104-Mis 1-24
cattggcatggaagtcacaccttggggagttgcagttcccctgtctacggggattgttaccctatagtgagtcgtattaatttc

SEQ ID NO: 97 - DNA, 84 bp, Artificial Sequence
  1104-Mis 1-25
cattggcatggaagtcacaccttccggagttgcagttcccctgtctacggggattgttaccctatagtgagtcgtattaatttc

SEQ ID NO: 98 - DNA, 84 bp, Artificial Sequence
  1104-Mis 1-26
cattggcatggaagtcacaccttcgcgagttgcagttcccctgtctacggggattgttaccctatagtgagtcgtattaatttc

SEQ ID NO: 99 - DNA, 84 bp, Artificial Sequence
  1104-Mis 1- 27
cattggcatggaagtcacaccttcggcagttgcagttcccctgtctacggggattgttaccctatagtgagtcgtattaatttc

SEQ ID NO: 100 - DNA, 84 bp, Artificial Sequence
  1104-Mis 1-28
cattggcatggaagtcacaccttcgggtgttgcagttcccctgtctacggggattgttaccctatagtgagtcgtattaatttc

SEQ ID NO: 101 - DNA, 84 bp, Artificial Sequence
  1104-Mis-2-1
gtttggcatggaagtcacaccttcgggagttgcagttcccctgtctacggggattgttaccctatagtgagtcgtattaatttc

SEQ ID NO: 102 - DNA, 84 bp, Artificial Sequence
  1104-Mis-2-2
caaaggcatggaagtcacaccttcgggagttgcagttcccctgtctacggggattgttaccctatagtgagtcgtattaatttc

SEQ ID NO: 103 - DNA, 84 bp, Artificial Sequence
  1104-Mis-2-3
cattcccatggaagtcacaccttcgggagttgcagttcccctgtctacggggattgttaccctatagtgagtcgtattaatttc

SEQ ID NO: 104 - DNA, 84 bp, Artificial Sequence
  1104-Mis-2-4
cattgggttggaagtcacaccttcgggagttgcagttcccctgtctacggggattgttaccctatagtgagtcgtattaatttc

SEQ ID NO: 105 - DNA, 84 bp, Artificial Sequence
  1104-Mis-2-5 Synthetic DNA
cattggcaacgaagtcacaccttcgggagttgcagttcccctgtctacggggattgttaccctatagtgagtcgtattaatttc

SEQ ID NO: 106 - DNA, 84 bp, Artificial Sequence
  1104-Mis-2-6  Synthetic DNA
cattggcatgctagtcacaccttcgggagttgcagttcccctgtctacggggattgttaccctatagtgagtcgtattaatttc

SEQ ID NO: 107 - DNA, 84 bp, Artificial Sequence
  1104-Mis-2-7  Synthetic DNA
cattggcatggatctcacaccttcgggagttgcagttcccctgtctacggggattgttaccctatagtgagtcgtattaatttc

SEQ ID NO: 108 - DNA, 84 bp, Artificial Sequence
  1104-Mis-2-8  Synthetic DNA
cattggcatggaagagacaccttcgggagttgcagttcccctgtctacggggattgttaccctatagtgagtcgtattaatttc

SEQ ID NO: 109 - DNA, 84 bp, Artificial Sequence
  1104-Mis-2-9  Synthetic DNA
cattggcatggaagtctgaccttcgggagttgcagttcccctgtctacggggattgttaccctatagtgagtcgtattaatttc

SEQ ID NO: 110 - DNA, 84 bp, Artificial Sequence
  1104-Mis-2-10  Synthetic DNA
cattggcatggaagtcactgcttcgggagttgcagttcccctgtctacggggattgttaccctatagtgagtcgtattaatttc

SEQ ID NO: 111 - DNA, 84 bp, Artificial Sequence
  1104-Mis-2-11  Synthetic DNA
cattggcatggaagtcacacgatcgggagttgcagttcccctgtctacggggattgttaccctatagtgagtcgtattaatttc

SEQ ID NO: 112 - DNA, 84 bp, Artificial Sequence
  1104-Mis- 2-12  Synthetic DNA
cattggcatggaagtcacacctaggggagttgcagttcccctgtctacggggattgttaccctatagtgagtcgtattaatttc

SEQ ID NO: 113 - DNA, 84 bp, Artificial Sequence
  1104-Mis-2-13  Synthetic DNA
cattggcatggaagtcacaccttcccgagttgcagttcccctgtctacggggattgttaccctatagtgagtcgtattaatttc

SEQ ID NO: 114 - DNA, 84 bp, Artificial Sequence
  1104-Mis-2-14  Synthetic DNA
cattggcatggaagtcacaccttcggctgttgcagttcccctgtctacggggattgttaccctatagtgagtcgtattaatttc

SEQ ID NO: 115 - DNA, 84 bp, Artificial Sequence
  1104-Seed- 1  Synthetic DNA
gtaaggcatggaagtcacaccttcgggagttgcagttcccctgtctacggggattgttaccctatagtgagtcgtattaatttc

SEQ ID NO: 116 - DNA, 84 bp, Artificial Sequence
  1104-Seed- 2  Synthetic DNA
cattccgttggaagtcacaccttcgggagttgcagttcccctgtctacggggattgttaccctatagtgagtcgtattaatttc

SEQ ID NO: 117 - DNA, 84 bp, Artificial Sequence
  1104-Seed- 3  Synthetic DNA
cattggcaacctagtcacaccttcgggagttgcagttcccctgtctacggggattgttaccctatagtgagtcgtattaatttc

SEQ ID NO: 118 - DNA, 84 bp, Artificial Sequence
  1104-Seed- 4  Synthetic DNA
cattggcatggatcagacaccttcgggagttgcagttcccctgtctacggggattgttaccctatagtgagtcgtattaatttc

SEQ ID NO: 119 - DNA, 84 bp, Artificial Sequence
  1104-Seed- 5  Synthetic DNA
cattggcatggaagtctgtgcttcgggagttgcagttcccctgtctacggggattgttaccctatagtgagtcgtattaatttc

SEQ ID NO: 120 - DNA, 84 bp, Artificial Sequence
  1104-Seed- 6  Synthetic DNA
cattggcatggaagtcacacgaaggggagttgcagttcccctgtctacggggattgttaccctatagtgagtcgtattaatttc

SEQ ID NO: 121 - DNA, 84 bp, Artificial Sequence
  1104-Seed- 7  Synthetic DNA
cattggcatggaagtcacaccttcccctgttgcagttcccctgtctacggggattgttaccctatagtgagtcgtattaatttc

SEQ ID NO: 122 - DNA, 72 bp, Artificial Sequence
  1104-Spacer-16  Synthetic DNA
catggaagtcacacctgttgcagttcccctgtctacggggattgttaccctatagtgagtcgtattaatttc

SEQ ID NO: 123 - DNA, 74 bp, Artificial Sequence
  1104-Spacer-18  Synthetic DNA
gcatggaagtcacaccttgttgcagttcccctgtctacggggattgttaccctatagtgagtcgtattaatttc

SEQ ID NO: 124 - DNA, 76 bp, Artificial Sequence
  1104-Spacer-20  Synthetic DNA
ggcatggaagtcacaccttcgttgcagttcccctgtctacggggattgttaccctatagtgagtcgtattaatttc

SEQ ID NO: 125 - DNA, 78 bp, Artificial Sequence
  1104-Spacer-22  Synthetic DNA
tggcatggaagtcacaccttcggttgcagttcccctgtctacggggattgttaccctatagtgagtcgtattaatttc

SEQ ID NO: 126 - DNA, 80 bp, Artificial Sequence
  1104-Spacer-24  Synthetic DNA
ttggcatggaagtcacaccttcgggttgcagttcccctgtctacggggattgttaccctatagtgagtcgtattaatttc

SEQ ID NO: 127 - DNA, 82 bp, Artificial Sequence
  1104-Spacer-26  Synthetic DNA
attggcatggaagtcacaccttcggggttgcagttcccctgtctacggggattgttaccctatagtgagtcgtattaatttc

SEQ ID NO: 128 - DNA, 86 bp, Artificial Sequence
  1104-Spacer-30  Synthetic DNA
gcattggcatggaagtcacaccttcgggaagttgcagttcccctgtctacggggattgttaccctatagtgagtcgtattaatttc

SEQ ID NO: 129 - DNA, 80 bp, Artificial Sequence
  1172-Mis 1-1  Synthetic DNA
atggcatggaagtcacaccttcgggttgcagtctccgcctacatgggagttgtgaccctatagtgagtcgtattaatttc

SEQ ID NO: 130 - DNA, 80 bp, Artificial Sequence
  1172-Mis 1-2  Synthetic DNA
taggcatggaagtcacaccttcgggttgcagtctccgcctacatgggagttgtgaccctatagtgagtcgtattaatttc

SEQ ID NO: 131 - DNA, 80 bp, Artificial Sequence
  1172-Mis 1-3  Synthetic DNA
ttcgcatggaagtcacaccttcgggttgcagtctccgcctacatgggagttgtgaccctatagtgagtcgtattaatttc

SEQ ID NO: 132 - DNA, 80 bp, Artificial Sequence
  1172-Mis 1-4  Synthetic DNA
ttgccatggaagtcacaccttcgggttgcagtctccgcctacatgggagttgtgaccctatagtgagtcgtattaatttc

SEQ ID NO: 133 - skipped

SEQ ID NO: 134 - DNA, 80 bp, Artificial Sequence
  1172-Mis 1-5  Synthetic DNA
ttgggatggaagtcacaccttcgggttgcagtctccgcctacatgggagttgtgaccctatagtgagtcgtattaatttc

SEQ ID NO: 135 - DNA, 80 bp, Artificial Sequence
  1172-Mis 1-6  Synthetic DNA
ttggcttggaagtcacaccttcgggttgcagtctccgcctacatgggagttgtgaccctatagtgagtcgtattaatttc

SEQ ID NO: 136 - DNA, 80 bp, Artificial Sequence
  1172-Mis 1-7  Synthetic DNA
ttggcaaggaagtcacaccttcgggttgcagtctccgcctacatgggagttgtgaccctatagtgagtcgtattaatttc

SEQ ID NO: 137 - DNA, 80 bp, Artificial Sequence
  1172-Mis 1-8  Synthetic DNA
ttggcatcgaagtcacaccttcgggttgcagtctccgcctacatgggagttgtgaccctatagtgagtcgtattaatttc

SEQ ID NO: 138 - DNA, 80 bp, Artificial Sequence
  1172-Mis 1-9  Synthetic DNA
ttggcatgcaagtcacaccttcgggttgcagtctccgcctacatgggagttgtgaccctatagtgagtcgtattaatttc

SEQ ID NO: 139 - DNA, 80 bp, Artificial Sequence
  1172-Mis 1-10  Synthetic DNA
ttggcatggtagtcacaccttcgggttgcagtctccgcctacatgggagttgtgaccctatagtgagtcgtattaatttc

SEQ ID NO: 140 - DNA, 80 bp, Artificial Sequence
  1172-Mis 1-11  Synthetic DNA
ttggcatggatgtcacaccttcgggttgcagtctccgcctacatgggagttgtgaccctatagtgagtcgtattaatttc

SEQ ID NO: 141 - DNA, 80 bp, Artificial Sequence
  1172-Mis 1-12  Synthetic DNA
ttggcatggaactcacaccttcgggttgcagtctccgcctacatgggagttgtgaccctatagtgagtcgtattaatttc

SEQ ID NO: 142 - DNA, 80 bp, Artificial Sequence
  1172-Mis 1-13  Synthetic DNA
ttggcatggaagacacaccttcgggttgcagtctccgcctacatgggagttgtgaccctatagtgagtcgtattaatttc

SEQ ID NO: 143 - DNA, 80 bp, Artificial Sequence
  1172-Mis 1-14  Synthetic DNA
ttggcatggaagtgacaccttcgggttgcagtctccgcctacatgggagttgtgaccctatagtgagtcgtattaatttc

SEQ ID NO: 144 - DNA, 80 bp, Artificial Sequence
  1172-Mis 1-15  Synthetic DNA
ttggcatggaagtctcaccttcgggttgcagtctccgcctacatgggagttgtgaccctatagtgagtcgtattaatttc

SEQ ID NO: 145 - DNA, 80 bp, Artificial Sequence
  1172-Mis 1-16  Synthetic DNA
ttggcatggaagtcagaccttcgggttgcagtctccgcctacatgggagttgtgaccctatagtgagtcgtattaatttc

SEQ ID NO: 146 - DNA, 80 bp, Artificial Sequence
  1172-Mis 1-17  Synthetic DNA
ttggcatggaagtcactccttcgggttgcagtctccgcctacatgggagttgtgaccctatagtgagtcgtattaatttc

SEQ ID NO: 147 - DNA, 80 bp, Artificial Sequence
  1172-Mis 1-18  Synthetic DNA
ttggcatggaagtcacagcttcgggttgcagtctccgcctacatgggagttgtgaccctatagtgagtcgtattaatttc

SEQ ID NO: 148 - DNA, 80 bp, Artificial Sequence
  1172-Mis 1-19 Synthetic DNA
ttggcatggaagtcacacgttcgggttgcagtctccgcctacatgggagttgtgaccctatagtgagtcgtattaatttc

SEQ ID NO: 149 - DNA, 80 bp, Artificial Sequence
  1172-Mis 1-20  Synthetic DNA
ttggcatggaagtcacaccatcgggttgcagtctccgcctacatgggagttgtgaccctatagtgagtcgtattaatttc

SEQ ID NO: 150 - DNA, 80 bp, Artificial Sequence
  1172-Mis 1-21  Synthetic DNA
ttggcatggaagtcacacctacgggttgcagtctccgcctacatgggagttgtgaccctatagtgagtcgtattaatttc

SEQ ID NO: 151 - DNA, 80 bp, Artificial Sequence
  1172-Mis 1-22  Synthetic DNA
ttggcatggaagtcacaccttggggttgcagtctccgcctacatgggagttgtgaccctatagtgagtcgtattaatttc

SEQ ID NO: 152 - DNA, 80 bp, Artificial Sequence
  1172-Mis 1-23  Synthetic DNA
ttggcatggaagtcacaccttccggttgcagtctccgcctacatgggagttgtgaccctatagtgagtcgtattaatttc

SEQ ID NO: 153 - DNA, 80 bp, Artificial Sequence
  1172-Mis 1-24  Synthetic DNA
ttggcatggaagtcacaccttcgcgttgcagtctccgcctacatgggagttgtgaccctatagtgagtcgtattaatttc

SEQ ID NO: 154 - DNA, 80 bp, Artificial Sequence
  1172-Mis-2-1  Synthetic DNA
aaggcatggaagtcacaccttcgggttgcagtctccgcctacatgggagttgtgaccctatagtgagtcgtattaatttc

SEQ ID NO: 155 - DNA, 80 bp, Artificial Sequence
  1172-Mis-2-2  Synthetic DNA
ttcccatggaagtcacaccttcgggttgcagtctccgcctacatgggagttgtgaccctatagtgagtcgtattaatttc

SEQ ID NO: 156 - DNA, 80 bp, Artificial Sequence
  1172-Mis-2-3  Synthetic DNA
ttgggttggaagtcacaccttcgggttgcagtctccgcctacatgggagttgtgaccctatagtgagtcgtattaatttc

SEQ ID NO: 157 - DNA, 80 bp, Artificial Sequence
  1172-Mis-2-4  Synthetic DNA
ttggcaacgaagtcacaccttcgggttgcagtctccgcctacatgggagttgtgaccctatagtgagtcgtattaatttc

SEQ ID NO: 158 - DNA, 80 bp, Artificial Sequence
  1172-Mis-2-5  Synthetic DNA
ttggcatgctagtcacaccttcgggttgcagtctccgcctacatgggagttgtgaccctatagtgagtcgtattaatttc

SEQ ID NO: 159 - DNA, 80 bp, Artificial Sequence
  1172-Mis-2-6  Synthetic DNA
ttggcatggatctcacaccttcgggttgcagtctccgcctacatgggagttgtgaccctatagtgagtcgtattaatttc

SEQ ID NO: 160 - DNA, 80 bp, Artificial Sequence
  1172-Mis-2-7  Synthetic DNA
ttggcatggaagagacaccttcgggttgcagtctccgcctacatgggagttgtgaccctatagtgagtcgtattaatttc

SEQ ID NO: 161 - DNA, 80 bp, Artificial Sequence
  1172-Mis-2-8  Synthetic DNA
ttggcatggaagtctgaccttcgggttgcagtctccgcctacatgggagttgtgaccctatagtgagtcgtattaatttc

SEQ ID NO: 162 - DNA, 80 bp, Artificial Sequence
  1172-Mis-2-9  Synthetic DNA
ttggcatggaagtcactgcttcgggttgcagtctccgcctacatgggagttgtgaccctatagtgagtcgtattaatttc

SEQ ID NO: 163 - DNA, 80 bp, Artificial Sequence
  1172-Mis-2-10  Synthetic DNA
ttggcatggaagtcacacgatcgggttgcagtctccgcctacatgggagttgtgaccctatagtgagtcgtattaatttc

SEQ ID NO: 164 - DNA, 80 bp, Artificial Sequence
  1172-Mis-2-11  Synthetic DNA
ttggcatggaagtcacacctaggggttgcagtctccgcctacatgggagttgtgaccctatagtgagtcgtattaatttc

SEQ ID NO: 165 - DNA, 80 bp, Artificial Sequence
  1172-Mis-2-12  Synthetic DNA
ttggcatggaagtcacaccttcccgttgcagtctccgcctacatgggagttgtgaccctatagtgagtcgtattaatttc

SEQ ID NO: 166 - DNA, 80 bp, Artificial Sequence
  1172-Seed- 1  Synthetic DNA
aacccatggaagtcacaccttcgggttgcagtctccgcctacatgggagttgtgaccctatagtgagtcgtattaatttc

SEQ ID NO: 167 - DNA, 80 bp, Artificial Sequence
  1172-Seed- 2  Synthetic DNA
ttgggtacgaagtcacaccttcgggttgcagtctccgcctacatgggagttgtgaccctatagtgagtcgtattaatttc

SEQ ID NO: 168 - DNA, 80 bp, Artificial Sequence
  1172-Seed- 3  Synthetic DNA
ttggcatgcttctcacaccttcgggttgcagtctccgcctacatgggagttgtgaccctatagtgagtcgtattaatttc

SEQ ID NO: 169 - DNA, 80 bp, Artificial Sequence
  1172-Seed- 4  Synthetic DNA
ttggcatggaagagtgaccttcgggttgcagtctccgcctacatgggagttgtgaccctatagtgagtcgtattaatttc

SEQ ID NO: 170 - DNA, 80 bp, Artificial Sequence
  1172-Seed- 5  Synthetic DNA
ttggcatggaagtcactggatcgggttgcagtctccgcctacatgggagttgtgaccctatagtgagtcgtattaatttc

SEQ ID NO: 171 - DNA, 80 bp, Artificial Sequence
  1172-Seed- 6  Synthetic DNA
ttggcatggaagtcacacctagccgttgcagtctccgcctacatgggagttgtgaccctatagtgagtcgtattaatttc

SEQ ID NO: 172 - DNA, 72 bp, Artificial Sequence
  1172-Spacer-16  Synthetic DNA
catggaagtcacacctgttgcagtctccgcctacatgggagttgtgaccctatagtgagtcgtattaatttc

SEQ ID NO: 173 - DNA, 74 bp, Artificial Sequence
  1172-Spacer-18  Synthetic DNA
gcatggaagtcacaccttgttgcagtctccgcctacatgggagttgtgaccctatagtgagtcgtattaatttc

SEQ ID NO: 174 - DNA, 76 bp, Artificial Sequence
  1172-Spacer-20  Synthetic DNA
ggcatggaagtcacaccttcgttgcagtctccgcctacatgggagttgtgaccctatagtgagtcgtattaatttc

SEQ ID NO: 175 - DNA, 78 bp, Artificial Sequence
  1172-Spacer-22  Synthetic DNA
tggcatggaagtcacaccttcggttgcagtctccgcctacatgggagttgtgaccctatagtgagtcgtattaatttc

SEQ ID NO: 176 - DNA, 82 bp, Artificial Sequence
  1172-Spacer-26  Synthetic DNA
attggcatggaagtcacaccttcggggttgcagtctccgcctacatgggagttgtgaccctatagtgagtcgtattaatttc

SEQ ID NO: 177 - DNA, 84 bp, Artificial Sequence
  1172-Spacer-28  Synthetic DNA
cattggcatggaagtcacaccttcgggagttgcagtctccgcctacatgggagttgtgaccctatagtgagtcgtattaatttc

SEQ ID NO: 178 - DNA, 86 bp, Artificial Sequence
  1172-Spacer-30  Synthetic DNA
gcattggcatggaagtcacaccttcgggaagttgcagtctccgcctacatgggagttgtgaccctatagtgagtcgtattaatttc

SEQ ID NO: 179 - DNA, 91 bp, Artificial Sequence
  ORF1 LwaCas13a crRNA  Synthetic DNA
gttaaaccaggtggaacctcatcaggagatgttttagtccccttcgtttttggggtagtctaaatcccctatagtgagtcgtattaatttc

SEQ ID NO: 180 - DNA, 80 bp, Artificial Sequence
  Tcc- crRNA- HCV-1  Synthetic DNA
tagtggtctgcggaaccggtgagtgttgcagtctccgcctacatgggagttgtgaccctatagtgagtcgtattaatttc

SEQ ID NO: 181 - DNA, 80 bp, Artificial Sequence
  Tcc- crRNA- TYLCV-2  Synthetic DNA
aaggccttatggaagcagcccaatgttgcagtctccgcctacatgggagttgtgaccctatagtgagtcgtattaatttc

SEQ ID NO: 182 - DNA, 41 bp, Artificial Sequence
  AapCas12b- RnaseP- sgRNA-1  Synthetic DNA
agggtcacacccaagtaattgtgccacttctcagatttgag

SEQ ID NO: 183 - DNA, 41 bp, Artificial Sequence
  AapCas12b- RnaseP- sgRNA-2  Synthetic DNA
ctcggaccagagccatgtaagtgccacttctcagatttgag

SEQ ID NO: 184 - DNA, 41 bp, Artificial Sequence
  AapCas12b- RnaseP- sgRNA-3  Synthetic DNA
aattacttgggtgtgaccctgtgccacttctcagatttgag

SEQ ID NO: 185 - DNA, 115 bp, Artificial Sequence
  AapCas12b sgRNA scaffold-  Synthetic DNA
gaaattaatacgactcactatagggtctagaggacagaatttttcaacgggtgtgccaatggccactttccaggtggcaaagcccgttgagcttctcaaatctgagaagtggcac

SEQ ID NO: 186 - RNA, 72 nt, Artificial Sequence
  LwaCas13a-pre- crRNA-5` FAM labeled  Synthetic DNA
  misc_feature (1)..(1): fluorescein (fam) probe
aucaugcgaggauuuagacuaccccaaaaacgaaggggacuaaaacuucagauauagccuggugguucaggc

SEQ ID NO: 187 - RNA, 66 nt, Artificial Sequence
  HheCas13a- pre-crRNA- 5` FAM labeled Synthetic DNA
  misc_feature (1)..(1): fluoroscein (FAM) probe
acugagguauguaacaauccccguagacaggggaacugcaaccagauauagccuggugguucaggc

SEQ ID NO: 188 - RNA, 66 nt, Artificial Sequence
  TccCas13a- pre-crRNA- 5`  FAM labeled Synthetic DNA
gucgacacgggucacaacucccauguaggcggagacugcaacuauuaccacucaggguaauguccg

SEQ ID NO: 189 - DNA, 285 bp, Artificial Sequence
  SARS-CoV-2  ORF1 gBlock  Synthetic DNA
gaaattaatacgactcactatagggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtc

SEQ ID NO: 190 - DNA, 492 bp, Artificial Sequence
  HCV genotype 1a  Synthetic DNA
gaaattaatacgactcactatagggttgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaaccccaaagaaaaaccaaacgtaacaccaaccgtcgcccacaggacgtcaagttcccgggtggcggtcagatcgttggtggagtttacttgttgccgcgcaggggccgtcatagctgtttc

SEQ ID NO: 191 - DNA, 495 bp, Artificial Sequence
  HCV genotype 2b  Synthetic DNA
gaaattaatacgactcactatagggttgggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattaccggaaagactgggtcctttcttggataaacccactctatgtccggtcatttgggcacgcccccgcaagactgctagccgagtagcgttgggttgcgaaaggccttgtggtactgcctgatagggtgcttgcgagtgccccgggaggtctcgtagaccgtgcatcatgagcacaaatcctaaacctcaaagaaaaaccaaaagaaacacaaaccgccgcccacaggacgttaagttcccgggtggcggtcagatcgttggcggagtttacttgctgccgcgcaggggccgtcatagctgtttcctg

SEQ ID NO: 192 - protein, 6 aa, Artificial Sequence
  Higher Eukaryotes and Prokaryotes Nucleotide- binding (HEPN) domain Synthetic Protein
  misc_feature (2)..(5): Xaa can be any naturally occurring amino acid
RXXXXH

SEQ ID NO: 193 - protein, 6 aa, Artificial Sequence
  Synthetic Protein
  SITE (2)..(2): N or H
  misc_feature (3)..(5): Xaa can be any naturally occurring amino acid
RXXXXH

SEQ ID NO: 194 - protein, 6 aa, Artificial Sequence
  Synthetic Protein
  site (2)..(2): N or K
  misc_feature (3)..(5): Xaa can be any naturally occurring amino acid
RXXXXH

SEQ ID NO: 195 - DNA, 27 bp, Artificial Sequence
  ORF1-R Synthetic DNA
gacatacttatcggcaattttgttacc

SEQ ID NO: 196 - DNA, 17 bp, Artificial Sequence
  M13-R Synthetic DNA
caggaaacagctatgac

SEQ ID NO: 197 - DNA, 80 bp, Artificial Sequence
  Tcc-RNase P-24-1  Synthetic DNA
tcagccatccacatccgagtcttcgttgcagtctccgcctacatgggagttgtgaccctatagtgagtcgtattaatttc

SEQ ID NO: 198 - DNA, 80 bp, Artificial Sequence
  Tcc-RNase P-24-2  Synthetic DNA
ccgtgatatggctcttcgcatgctgttgcagtctccgcctacatgggagttgtgaccctatagtgagtcgtattaatttc

SEQ ID NO: 199 - DNA, 84 bp, Artificial Sequence
  1104-Mis 1-1  Synthetic DNA
gattggcatggaagtcacaccttcgggagttgcagttcccctgtctacggggattgttaccctatagtgagtcgtattaatttc

SEQ ID NO: 200 - DNA, 84 bp, Artificial Sequence
  1104-Mis 1-2 Synthetic DNA
ctttggcatggaagtcacaccttcgggagttgcagttcccctgtctacggggattgttaccctatagtgagtcgtattaatttc

SEQ ID NO: 201 - DNA, 84 bp, Artificial Sequence
  1104-Mis 1-3  Synthetic DNA
caatggcatggaagtcacaccttcgggagttgcagttcccctgtctacggggattgttaccctatagtgagtcgtattaatttc

SEQ ID NO: 202 - DNA, 84 bp, Artificial Sequence
  1104-Mis 1-4  Synthetic DNA
cataggcatggaagtcacaccttcgggagttgcagttcccctgtctacggggattgttaccctatagtgagtcgtattaatttc

SEQ ID NO: 203 - DNA, 84 bp, Artificial Sequence
  1104-Mis 1-5  Synthetic DNA
cattcgcatggaagtcacaccttcgggagttgcagttcccctgtctacggggattgttaccctatagtgagtcgtattaatttc

SEQ ID NO: 204 - DNA, 84 bp, Artificial Sequence
  1104-Mis 1-6  Synthetic DNA
cattgccatggaagtcacaccttcgggagttgcagttcccctgtctacggggattgttaccctatagtgagtcgtattaatttc

SEQ ID NO: 205 - DNA, 84 bp, Artificial Sequence
  1104-Mis 1-7  Synthetic DNA
cattgggatggaagtcacaccttcgggagttgcagttcccctgtctacggggattgttaccctatagtgagtcgtattaatttc

SEQ ID NO: 206 - DNA, 84 bp, Artificial Sequence
  1104-Mis 1-8  Synthetic DNA
cattggcttggaagtcacaccttcgggagttgcagttcccctgtctacggggattgttaccctatagtgagtcgtattaatttc

SEQ ID NO: 207 - DNA, 84 bp, Artificial Sequence
  1104-Mis 1-9  Synthetic DNA
cattggcaaggaagtcacaccttcgggagttgcagttcccctgtctacggggattgttaccctatagtgagtcgtattaatttc

SEQ ID NO: 208 - DNA, 84 bp, Artificial Sequence
  1104-Mis 1-10  Synthetic DNA
cattggcatcgaagtcacaccttcgggagttgcagttcccctgtctacggggattgttaccctatagtgagtcgtattaatttc

SEQ ID NO: 209 - DNA, 84 bp, Artificial Sequence
  1104-Mis 1-11  Synthetic DNA
cattggcatgcaagtcacaccttcgggagttgcagttcccctgtctacggggattgttaccctatagtgagtcgtattaatttc

SEQ ID NO: 210 - DNA, 84 bp, Artificial Sequence
  1104-Mis 1-12  Synthetic DNA
cattggcatggtagtcacaccttcgggagttgcagttcccctgtctacggggattgttaccctatagtgagtcgtattaatttc

SEQ ID NO: 211 - DNA, 84 bp, Artificial Sequence
  1104-Mis 1-13  Synthetic DNA
cattggcatggatgtcacaccttcgggagttgcagttcccctgtctacggggattgttaccctatagtgagtcgtattaatttc

SEQ ID NO: 212 - DNA, 84 bp, Artificial Sequence
  1104-Mis 1-14  Synthetic DNA
cattggcatggaactcacaccttcgggagttgcagttcccctgtctacggggattgttaccctatagtgagtcgtattaatttc

SEQ ID NO: 213 - DNA, 84 bp, Artificial Sequence
  1104-Mis 1-15  Synthetic DNA
cattggcatggaagacacaccttcgggagttgcagttcccctgtctacggggattgttaccctatagtgagtcgtattaatttc

SEQ ID NO: 214 - DNA, 84 bp, Artificial Sequence
  1104-Mis 1-16  Synthetic DNA
cattggcatggaagtgacaccttcgggagttgcagttcccctgtctacggggattgttaccctatagtgagtcgtattaatttc

SEQ ID NO: 215 - DNA, 84 bp, Artificial Sequence
  1104-Mis 1-17 Synthetic DNA
cattggcatggaagtctcaccttcgggagttgcagttcccctgtctacggggattgttaccctatagtgagtcgtattaatttc